(19) United States
(12) Patent Application Publication
(10) Pub. No.: US 2024/0294952 A1
(43) Pub. Date: Sep. 5, 2024

(54) LABYRINTHULID MICROORGANISM CAPABLE OF PRODUCING MICROBIAL OIL, MICROBIAL OIL, METHODS FOR PRODUCING SAID MICROORGANISM AND FOR PRODUCING SAID MICROBIAL OIL, AND USES OF SAID MICROORGANISM AND SAID MICROBIAL OIL

(71) Applicants: KYUSHU UNIVERSITY, NATIONAL UNIVERSITY CORPORATION, Fukuoka-shi (JP); KONAN GAKUEN, Kobe-shi (JP); NIPPON SUISAN KAISHA, LTD., Tokyo (JP)

(72) Inventors: Yuji Okita, Tokyo (JP); Makoto Ito, Fukuoka-shi (JP); Rie Hamaguchi, Fukuoka-shi (JP); Hatsumi Goda, Fukuoka-shi (JP); Seiya Mochinaga, Fukuoka-shi (JP); Daisuke Honda, Kobe-shi (JP)

(73) Assignees: KYUSHU UNIVERSITY, NAT'L UNIVERSITY CORPORATION, Fukuoka-shi (JP); KONAN GAKUEN, Kobe-shi (JP); NIPPON SUISAN KAISHA, LTD., Tokyo (JP)

(21) Appl. No.: 18/411,156

(22) Filed: Jan. 12, 2024

Related U.S. Application Data
(62) Division of application No. 17/401,894, filed on Aug. 13, 2021, now Pat. No. 11,898,190.
Application No. 17/401,894 is a continuation of application No. 15/740,969, filed on Apr. 10, 2018, now abandoned.

(30) Foreign Application Priority Data
Jul. 3, 2015 (JP) 2015-134715

(51) Int. Cl. C12P 7/6432; C10G 3/00; C12N 1/00; C12N 1/12; C12N 15/09; C12N 15/52; C12N 15/79; C12P 1/00; C12P 7/64; C12P 7/6427; C12P 7/6472; C12P 19/34; C12R 1/00; C12R 1/89

(52) U.S. Cl. CPC C12P 7/6432; C12N 15/09; C12N 15/79; C12P 1/00; C12P 7/64; C12P 7/6427; C12P 7/6472; C12P 19/34; C10G 3/00; C12N 1/00; C12N 1/125; C12N 15/52; C12R 2001/00; C12R 2001/89

(57) ABSTRACT

A method for producing a microbial oil includes genetically modifying a labyrinthulid which belongs to the genus Parietichytrium or the genus Schizochytrium by disrupting and/or silencing a Δ4 desaturase gene, a C20 elongase gene, and/or a C18 elongase gene, or by being transformed with an ω3 desaturase gene; culturing the labyrinthulid; and collecting the microbial oil from the labyrinthulid. The labyrinthulid has an activity of synthesizing DHA via a PUFA-PKS pathway in an amount of not more than 1/100 of a total amount of DHA synthesized in the labyrinthulid or no activity of producing PUFAs via the PUFA-PKS pathway, and the labyrinthulid has an activity of producing PUFAs via an endogenous elongase-desaturase pathway.

### CROSS-REFERENCE OF RELATED APPLICATIONS

This application is a Divisional of application Ser. No. 17/401,894, filed Aug. 13, 2021 which is a Divisional of application Ser. No. 15/740,969, filed Apr. 10, 2018, which is a 371 of International Application No. PCT/JP2016/069825, filed Jul. 4, 2016, which claims priority of Japanese Patent Application No. 2015-134715, filed Jul. 3, 2015, the entire contents of which are incorporated herein by reference.

The sequence listing that is contained in the file named “P171406US02_seq_listing.xml”, which is 362 KB and was created on Apr. 17, 2024, is incorporated herein by reference in its entirety.

### TECHNICAL FIELD

The present invention relates to microbial oil obtained from a labyrinthulid, a labyrinthulid capable of producing microbial oil, a method for producing the microbial oil and the labyrinthulid, and a use of the microbial oil and the labyrinthulid.

More specifically, the present invention relates to polyunsaturated fatty acids (PUFAs) and the production thereof, and relates to a labyrinthulid genetically modified such that the fatty acid composition is modified, preferably a labyrinthulid that produces PUFAs only via the elongase-desaturase pathway, a method for producing PUFAs using the same, lipids (microbial oils) containing PUFAs produced using the same, and uses thereof.

### BACKGROUND ART

Labyrinthulids are eukaryotic microorganisms belonging to the class Labyrinthulomycetes, which includes the order Labyrinthulales and the order Thraustochytriales Labyrinthulids are known to be universally present in oceans. Microorganisms belonging to the order Thraustochytriales are also generically called thraustochytrids.

Labyrinthulids have drawn attention as industrial oil-producing microorganisms. DHA produced by labyrinthulids has been productized as DHA-containing lipid raw materials, high DHA-containing animal feeds, and the like (Non-patent Document 1). Specific examples include breeding techniques for the genus Thraustochytrium and genus Schizochytrium (Patent Document 1), and techniques for using thraustochytrids and ω-3 HUFAs (highly unsaturated fatty acids) extracted from thraustochytrids (Patent Document 2).

It is generally known that PUFAs are biosynthesized via the elongase-desaturase pathway (also called the standard pathway), but it has also been shown that certain labyrinthulids produce PUFAs via a different pathway, namely a metabolic pathway using polyketide synthase (PKS) (Non-patent Document 2). In the present invention, this pathway will hereinafter be called the PUFA-PKS pathway or the PKS pathway. The compositions of PUFAs produced via this pathway are mostly DHA and n-6 DPA.

There are some scientists who think that labyrinthulids (particularly thraustochytrids) have only the PUFA-PKS pathway as a PUFA biosynthesis pathway, and do not have the general elongase-desaturase pathway found in other organisms (Non-patent Document 3). Actually, it has been reported that by gene-isruption of the PUFA-PKS pathway, some labyrinthulids are rendered lethal and such labyrinthulids cannot be grown unless PUFAs are added to the culture medium (Non-patent Document 3). This result means that since PUFAs are required for growth of these labyrinthulids and the gene of the PUFA-PKS pathway, which is the sole PUFA biosynthesis pathway, has been disrupted, the nature of the labyrinthulids has changed into exogenous PUFAs requirement for growth.

However, contrary to common knowledge of persons skilled in the art, some labyrinthulids having both the PUFA-PKS pathway and the elongase-desaturase pathway as PUFA biosynthesis pathways have been found through our examinations. These are specifically described in the specification of Patent Document 3 and in Non-patent Document 5.

Citing Thraustochytrium aureum ATCC 34304 as an example, it has been found that this strain has a Δ12 desaturase gene, which is an entry enzyme of the elongase-desaturase pathway. Furthermore, it has been found that a strain in which this gene was disrupted by homologous recombination accumulates predominantly oleic acid, which is the substrate of Δ12 desaturase, compared to the wild-type strain, and that decreases in both linoleic acid of the product and PUFAs located downstream of the biosynthesis system thereof were observed. Additionally, it has been found that because this strain is capable of producing PUFAs via the elongase-desaturase pathway, by disruption of the PUFA-PKS pathway gene it is not rendered lethal. This report was the first to show that the elongase-desaturase pathway also functions as a PUFA biosynthesis pathway in labyrinthulids, and was discussed in Non-patent Document 6 as well.

At the start, Non-patent Document 6 states that labyrinthulids that produce DHA as the main fatty acid are widely used industrially. In contrast, the creation of labyrinthulids containing desired PUFAs other than DHA as the main fatty acid was first enabled by the discovery of labyrinthulids having both the PUFA-PKS pathway and the elongase-desaturase pathway and by application of transformation techniques to these labyrinthulids (Patent Document 3, Non-patent Document 7). That is, it is possible to create strains containing certain PUFAs other than DHA as the main fatty acid by first disrupting the genes of the PUFA-PKS pathway with homologous recombination technique, and then disrupting or overexpressing the genes of the enzymes constituting the elongase-desaturase pathway appropriately.

Example 12 of Patent Document 3 is cited as a specific example. In this example, the PUFA-PKS pathway genes of Thraustochytrium aureum ATCC 34304 were disrupted, and then the C20 elongase gene was disrupted. An ω3 desaturase gene derived from Saprolegnia diclina was then transformed, thereby successfully creating a strain in which arachidonic acid increased approximately 6-fold, EPA increased approximately 10-fold, and DHA decreased approximately 1/16 in comparison with the wild-type strain.

### CITATION LIST

### Patent Documents

Patent Document 1: JP 3127161 B
Patent Document 2: JP 3669372 B
Patent Document 3: WO 2012/043826
Patent Document 4: US 2005/0,014,231 A

### Non-Patent Documents

Non-patent Document 1: Zvi Cohen et al. editors, “Single Cell Oils Microbial and Algal Oils 2nd edition”, (U.S.), AOCS Press, 2010, p. 88
Non-patent Document 2: Metz J G, Roessler P, Faccioti D, et al. Production of polyunsaturated fatty acids by polyketide synthesis in both prokaryotes and eukaryotes. Science 2001; 293: 290-293
Non-patent Document 3: Ratledge C. Omega-3 biotechnology: Errors and omissions, Biotechnology Advances 30 (2012) 1746-1747
Non-patent Document 4: Lippmeier J. C. et al., Lipids, 44 (7), 621-630 (2009)
Non-patent Document 5: Matsuda T, Sakaguchi K, Hamaguchi R, Kobayashi T, Abe E, Hama Y, Hayashi M, Honda D, Okita Y, Sugimoto S, Okino N, Ito M. The analysis of delta12 fatty acid desaturase function revealed that two distinct pathways are active for the synthesis of polyunsaturated fatty acids in Thraustochytrium aureum ATCC 34304. J. Lipid Res. 53 (6): 1210-1222 (2012)
Non-patent Document 6: ASBMB Today, June 2012, p. 30
Non-patent Document 7: Sakaguchi K, et al., Versatile Transformation System That Is Applicable to both Multiple Transgene Expression and Gene Targeting for Thraustochytrids. Appl. Environ. Microbiol. 78 (9): 3193-3202 (2012)
Non-patent Document 8: Yazawa K., Lipids, 31, Supple. 297-300 (1996)
Non-patent Document 9: Journal of the Japan Society for Bioscience, Biotechnology and Agrochem, 77, 2, 150-153 (2003)
Non-patent Document 10: “Illustrated Bio Experiments Vol. 2 Fundamentals of Gene Analysis”, p. 63-68, Shujunsha, published 1995
Non-patent Document 11: Sanger, F. et al., Proc. Natl. Acad. Sci., 74, 5463 (1997)
Non-patent Document 12: Cigan and Donahue, 1987; Romanos et al., 1992
Non-patent Document 13: Ausubel F. M. et al., Current Protocols in Molecular Biology, Unit 13 (1994)
Non-patent Document 14: Guthrie C., Fink G. et al., Methods in Enzymology: Guide to Yeast Genetics and Molecular Biology, Volume 194 (1991)
Non-patent Document 15: Qiu, X. et al. J. Biol. Chem., 276, 31561-6 (2001)
Non-patent Document 16: Abe E., et al., J. Biochem., 140, 247-253 (2006)
Non-patent Document 17: “Illustrated Bio Experiments Vol. 2 Fundamentals of Gene Analysis”, p. 117-128, Shujunsha, published 1995 Non-patent Document 18: DIG Manual (Japanese Edition) 8th, Roche Applied Science

### SUMMARY OF THE INVENTION

### Problem that the Invention is to Solve

The present invention is directed to provide a labyrinthulid genetically modified such that the fatty acid composition is modified, preferably a labyrinthulid that produces PUFAs only via the elongase-desaturase pathway. the invention provides a method for producing PUFAs using the labyrinthulid thereof, lipids (microbial oils) containing PUFAs produced using the method thereof, and uses thereof.

To create a strain containing certain PUFAs other than DHA as the main fatty acids using labyrinthulids having both the PUFA-PKS pathway and the elongase-desaturase pathway, first of all, DHA production via the PUFA-PKS pathway needs to be stopped or inhibited by any means. Specifically, methods such as disrupting PUFA-PKS pathway gene with homologous recombination technique, acquisition of a spontaneous mutant by UV irradiation, drug treatment, or the like, and silencing of PUFA-PKS genes by RNAi (RNA interference), and the like may be considered.

Among these methods, gene-disruption with homologous recombination technique previously described has been technically established. However, the number of drug resistance genes that can be used as markers is generally limited, and there is the problem that when a marker is used in PUFA-PKS pathway gene disruption, the number of markers that can be used in further gene disruption or gene transfer will decrease. This means a decrease in the number of times that gene-disruption and gene-transfer can be implemented after PUFA-PKS pathway gene is disrupted, and thus it becomes an obstacle to create higher-performance strains.

Furthermore, spontaneous mutation and silencing by RNAi are methods with a track record in other organisms but it is unknown whether a desired strain in labyrinthulids can be acquired by these methods.

The present invention is directed to provide a labyrinthulid having very weak or no PUFA-producing activity via the endogenous PUFA-PKS pathway and having PUFA producing activity via the endogenous elongase-desaturase pathway, a method for producing lipids containing PUFAs produced using the labyrinthulid thereof, and lipids containing PUFAs produced using the labyrinthulid thereof.

### Means for Solving the Problems

The present inventors conducted a diligent research, and discovered the existence of labyrinthulids having very weak or no PUFA producing activity via the endogenous PUFA-PKS pathway and having PUFA-producing activity via the endogenous elongase-desaturase pathway. And the present invention was accomplished pertaining to microbial oil obtained from labyrinthulids thereof, microbial oil-producing labyrinthulids, methods for producing microbial oil thereof, and uses of microbial oils.

In one aspect of the present invention, a method for producing a microbial oil includes the steps of genetically modifying a labyrinthulid by disrupting and/or silencing a gene, or by transforming another gene in addition to the disruption and/or gene silencing of the gene, and culturing the labyrinthulid, such that a fatty acid composition accumulated in the labyrinthulid comprises an increased EPA content; and collecting the microbial oil having the increased EPA content from the labyrinthulid. The labyrinthulid before the modification is selected from the group consisting of (A) and (B): (A) a labyrinthulid belonging to the genus Parietichytrium or genus Schizochytrium as disclosed below in detail, and having very weak or no activity of producing PUFAs via a PUFA-PKS pathway; and (B) a labyrinthulid belonging to the genus Thraustochytrium as disclosed below in detail, in which a host PUFA-PKS gene is disrupted or silenced to a very weak level. The disrupted and/or silenced gene is a fatty acid elongase gene and/or a fatty acid desaturase gene. The transformed another gene is a fatty acid elongase gene and/or a fatty acid desaturase gene. Typically, the increased EPA content is not less than 11.5% of a total fatty acid composition in labyrinthulid. The fatty acid elongase gene is a C20 elongase gene, and/or wherein the fatty acid desaturase gene is a Δ4 desaturase gene and/or an ω3 desaturase gene. The step of disrupting or transforming the gene of a labyrinthulid may utilize electroporation, a gene gun method, or gene editing, and/or wherein the step of silencing the gene utilizes an antisense method or RNA. The present invention can provide microbial oil produced by the method according to claim 1, wherein the microbial oil satisfies not less than one condition selected from the group consisting of (a) to (d):
(a) ARA is not less than 13.2% of the total fatty acid composition;
(b) The total of ARA and EPA is not less than 36.8% of the total fatty acid composition;
(c) DHA is not greater than 1.3% of the total fatty acid composition; and
(d) the total of DHA and n-6 DPA is not greater than 2.9% of the total fatty acid composition.

Further, the microbial oil may satisfy not less than one condition selected from the group consisting of (e) to (h):
(e) ARA is not less than 21.3% of the total fatty acid composition;
(f) EPA is not less than 23.8% of the total fatty acid composition;
(g) DHA is not greater than 0.5% of the total fatty acid composition; and
(h) The total of DHA and n-6 DPA is not greater than 0.7% of the total fatty acid composition.

Preferably, the microbial oil satisfies a condition that a GC area ratio of ARA after modification is not less than 4 times greater than before modification, the GC area is a peak area in a GC chart obtained by gas chromatography analysis. The microbial oil satisfies a condition that a GC area ratio of EPA after modification is not less than 8 times greater than before modification, the GC area is a peak area in a GC chart obtained by gas chromatography analysis.

In another aspect of the present invention, a labyrinthulid has been genetically modified by disrupting and/or silencing a gene, or by transforming another gene in addition to the disruption and/or gene silencing of the gene such that a fatty acid composition accumulated in the labyrinthulid comprises an increased EPA content. The labyrinthulid before the modification is selected from the group consisting of (A) and (B): (A) a labyrinthulid belonging to the genus Parietichytrium or genus Schizochytrium and having very weak or no activity of producing PUFAs via a PUFA-PKS pathway; and (B) a labyrinthulid belonging to the genus Thraustochytrium in which a host PUFA-PKS gene is disrupted or silenced to a very weak level. The disrupted and/or silenced gene is a fatty acid elongase gene and/or a fatty acid desaturase gene. The transformed another gene is a fatty acid elongase gene and/or a fatty acid desaturase gene. The increased EPA content is not less than 11.5% of a total fatty acid composition. The fatty acid composition may satisfy not less than one condition selected from the group consisting of (a) to (d):
(a) ARA is not less than 13.2% of the total fatty acid composition;
(b) The total of ARA and EPA is not less than 36.8% of the total fatty acid composition;
(c) DHA is not greater than 1.3% of the total fatty acid composition; and
(d) the total of DHA and n-6 DPA is not greater than 2.9% of the total fatty acid composition.

Further, the fatty acid composition satisfies not less than one condition selected from the group consisting of (e) to (h):
(e) ARA is not less than 21.3% of the total fatty acid composition;
(f) EPA is not less than 23.8% of the total fatty acid composition;
(g) DHA is not greater than 0.5% of the total fatty acid composition; and
(h) The total of DHA and n-6 DPA is not greater than 0.7% of the total fatty acid composition.

In yet another aspect, the present disclosure includes the following microbial oils (1) to (28).

(1) Microbial oil satisfying at least one condition selected from the group consisting of (a) to (g) below.
(a) ARA is not less than 5% of the total fatty acid composition.
(b) DGLA is not less than 2.5% of the total fatty acid composition.
(c) ETA is not less than 0.35% of the total fatty acid composition.
(d) EPA is not less than 4% of the total fatty acid composition.
(e) n-6 DPA is not greater than 0.20% of the total fatty acid composition.
(f) DHA is not greater than 0.50% of the total fatty acid composition.
(g) The total of DHA and n-6 DPA is not greater than 0.7% of the total fatty acid composition.

(2) The microbial oil according to (1), wherein the microbial oil satisfies at least one condition selected from the group consisting of (h) to (1) below.
(h) The value of LA/DHA by GC area is not less than 0.6 and not greater than 10.
(i) The value of GLA/DHA by GC area is not less than 0.35 and not greater than 10.
(j) The value of DGLA/DHA by GC area is not less than 0.35 and not greater than 10.
(k) The value of ARA/DHA by GC area is not less than 0.7 and not greater than 50.
(l) The value of EPA/DHA by GC area is not less than 0.35 and not greater than 50.

(3) The microbial oil according to (1) or (2), wherein the microbial oil satisfies at least one condition selected from the group consisting of (m) to (o) below.
(m) The value of LA/EPA by GC area is not less than 0.06 and not greater than 0.17.
(n) The value of GLA/EPA by GC area is not less than 0.04 and not greater than 0.12.
(o) The value of DTA/EPA by GC area is not less than 0.01 and not greater than 0.4.

(4) The microbial oil according to any one of (1) to (3), wherein the microbial oil satisfies the condition that the value of DTA/ARA by GC area is not less than 0.01 and not greater than 0.45.

(5) The microbial oil according to any one of (1) to (4), wherein the microbial oil satisfies the condition that the value of DTA/DGLA by GC area is not less than 0.01 and not greater than 1.45.

(6) The microbial oil according to any one of (1) to (5), wherein the microbial oil satisfies at least one condition selected from the group consisting of (p) to (t) below.
(p) The value of LA/n-6 DPA by GC area is not less than 0.4 and not greater than 20.
(q) The value of GLA/n-6 DPA by GC area is not less than 0.2 and not greater than 10.
(r) The value of DGLA/n-6 DPA by GC area is not less than 0.35 and not greater than 30.
(s) The value of ARA/n-6 DPA by GC area is not less than 0.7 and not greater than 60.
(t) The value of EPA/n-6 DPA by GC area is not less than 1.0 and not greater than 70.

(7) The microbial oil according to any one of (1) to (6), wherein the microbial oil satisfies at least one condition selected from the group consisting of (u) to (x) below.
(u) The value of DGLA/LA by GC area is not less than 1.4 and not greater than 10.
(v) The value of ARA/LA by GC area is not less than 5.1 and not greater than 20.
(w) The value of EPA/LA by GC area is not less than 5.5 and not greater than 30.
(x) The value of DTA/LA by GC area is not less than 0.01 and not greater than 0.4.

(8) The microbial oil according to any one of (1) to (7), wherein the microbial oil satisfies at least one condition selected from the group consisting of (y) and (z) below.
(y) The value of DGLA/GLA by GC area is not less than 4.5 and not greater than 20.
(z) The value of ARA/GLA by GC area is not less than 9 and not greater than 30.

(9) The microbial oil according to any one of (1) to (8), wherein the microbial oil satisfies the condition that the value of n-6 DPA/DTA by GC area is not greater than 1.5.

(10) The microbial oil according to any one of (1) to (9), wherein the microbial oil satisfies the condition that the value of DHA/n-3 DPA by GC area is not greater than 4.

(11) The microbial oil according to any one of (1) to (10), wherein the microbial oil satisfies the condition that the value of C20 PUFA/C22 PUFA by GC area is not less than 0.5 and not greater than 50.

(12) The microbial oil according to any one of (1) to (11), wherein the microbial oil satisfies the condition that the value of n-6 PUFA/n-3 PUFA by GC area is not less than 1.8.

(13) A microbial oil obtained from a labyrinthulid genetically modified such that the fatty acid composition is modified, the labyrinthulid being selected from the group consisting of (A) and (B) below.
(A) A labyrinthulid in which the fatty acid composition is modified by disruption and/or gene silencing.
(B) A labyrinthulid in which the fatty acid composition is modified by transforming a gene in addition to disruption and/or gene silencing.

(14) The microbial oil according to (13), wherein the disrupted and/or silenced gene is a PKS gene, a fatty acid elongase gene, and/or a fatty acid desaturase gene.

(15) The microbial oil according to (13) or (14), wherein the transformed gene is a fatty acid elongase gene and/or a fatty acid desaturase gene.

(16) The microbial oil according to (14) or (15), wherein the fatty acid elongase gene is the C20 elongase gene.

(17) The microbial oil according to any one of (13) to (16), wherein the fatty acid desaturase gene is the Δ4 desaturase gene and/or the ω3 desaturase gene.

(18) The microbial oil according to any one of (13) to (17), wherein the method for disrupting or transforming a gene of a labyrinthulid is electroporation, a gene gun method, or gene editing.

(19) The microbial oil according to any one of (13) to (18), wherein the method for gene silencing of a labyrinthulid is an antisense method or RNA interference.

(20) A microbial oil obtained from a labyrinthulid selected from the group consisting of (C) and (D) below.
(C) A labyrinthulid having very weak or no activity of producing PUFAs via the PUFA-PKS pathway.
(D) A labyrinthulid in which the host PUFA-PKS gene is disrupted or silenced to a very weak level.

(21) The microbial oil according to (20), wherein the labyrinthulid having very weak or no activity of producing PUFAs via the PUFA-PKS pathway is a labyrinthulid belonging to the genus Parietichytrium or genus Schizochytrium.

(22) The microbial oil according to (21), wherein the labyrinthulid belonging to the genus Parietichytrium is a labyrinthulid belonging to Parietichytrium sarkarianum.

(23) The microbial oil according to (21), wherein the labyrinthulid belonging to the genus Schizochytrium is a labyrinthulid belonging to Schizochytrium aggregatum.

(24) The microbial oil according to (22), wherein the microorganism belonging to Parietichytrium sarkarianum is Parietichytrium sp. SEK358 (FERM BP-11405), Parietichytrium sarkarianum SEK364 (FERM BP-11298), or Parietichytrium sp. SEK517 (FERM BP-11406).

(25) The microbial oil according to (23), wherein the microorganism belonging to Schizochytrium aggregatum is Schizochytrium aggregatum ATCC 28209.

(26) The microbial oil according to (20), wherein the labyrinthulid in which the host PUFA-PKS is disrupted or silenced to a very weak level belongs to the genus Thraustochytrium.

(27) The microbial oil according to (26), wherein the labyrinthulid belonging to the genus Thraustochytrium is Thraustochytrium aureum.

(28) A microbial oil that satisfies at least one condition selected from the group consisting of (E) to (H) below.
(E) A GC area ratio of ARA after modification is not less than 3 times greater than before modification.
(F) A GC area ratio of DGLA after modification is not less than 4 times greater than before modification.
(G) A GC area ratio of ETA after modification is not less than 7 times greater than before modification.
(H) A GC area ratio of EPA after modification is not less than 7 times greater than before modification.

The gist of the present invention includes the following methods for producing microbial oil (29) to (56).

(29) A method for producing microbial oil satisfying at least one condition selected from the group consisting of (a) to (g) below.
(a) ARA is not less than 5% of the total fatty acid composition.
(b) DGLA is not less than 2.5% of the total fatty acid composition.
(c) ETA is not less than 0.35% of the total fatty acid composition.
(d) EPA is not less than 4% of the total fatty acid composition.
(e) n-6 DPA is not greater than 0.20% of the total fatty acid composition.
(f) DHA is not greater than 0.50% of the total fatty acid composition.
(g) The total of DHA and n-6 DPA is not greater than 0.7% of the total fatty acid composition.

(30) The method for producing microbial oil according to (29), wherein the microbial oil satisfies at least one condition selected from the group consisting of (h) to (1) below.
(h) The value of LA/DHA by GC area is not less than 0.6 and not greater than 10.
(i) The value of GLA/DHA by GC area is not less than 0.35 and not greater than 10.
(j) The value of DGLA/DHA by GC area is not less than 0.35 and not greater than 10.
(k) The value of ARA/DHA by GC area is not less than 0.7 and not greater than 50.
(l) The value of EPA/DHA by GC area is not less than 0.35 and not greater than 50.

(31) The method for producing microbial oil according to (29) or (30), wherein the microbial oil satisfies at least one condition selected from the group consisting of (m) to (o) below.
(m) The value of LA/EPA by GC area is not less than 0.06 and not greater than 0.17.
(n) The value of GLA/EPA by GC area is not less than 0.04 and not greater than 0.12.
(o) The value of DTA/EPA by GC area is not less than 0.01 and not greater than 0.4.

(32) The method for producing microbial oil according to any one of (29) to (31), wherein the microbial oil satisfies the condition that the value of DTA/ARA by GC area is not less than 0.01 and not greater than 0.45.

(33) The method for producing microbial oil according to any one of (29) to (32), wherein the microbial oil satisfies the condition that the value of DTA/DGLA by GC area is not less than 0.01 and not greater than 1.8.

(34) The method for producing microbial oil according to any one of (29) to (33), wherein the microbial oil satisfies at least one condition selected from the group consisting of (p) to (t) below.
(p) The value of LA/n-6 DPA by GC area is not less than 0.4 and not greater than 20.
(q) The value of GLA/n-6 DPA by GC area is not less than 0.2 and not greater than 10.
(r) The value of DGLA/n-6 DPA by GC area is not less than 0.35 and not greater than 30.
(s) The value of ARA/n-6 DPA by GC area is not less than 0.7 and not greater than 60.
(t) The value of EPA/n-6 DPA by GC area is not less than 0.4 and not greater than 70.

(35) The method for producing microbial oil according to any one of (29) to (34), wherein the microbial oil satisfies at least one condition selected from the group consisting of (u) to (x) below.
(u) The value of DGLA/LA by GC area is not less than 1.4 and not greater than 10.
(v) The value of ARA/LA by GC area is not less than 5.1 and not greater than 20.
(w) The value of EPA/LA by GC area is not less than 5.5 and not greater than 30.
(x) The value of DTA/LA by GC area is not less than 0.01 and not greater than 0.4.

(36) The method for producing microbial oil according to any one of (29) to (35), wherein the microbial oil satisfies at least one condition selected from the group consisting of (y) and (z) below.
(y) The value of DGLA/GLA by GC area is not less than 4.5 and not greater than 20.
(z) The value of ARA/GLA by GC area is not less than 9 and not greater than 30.

(37) The method for producing microbial oil according to any one of (29) to (36), wherein the microbial oil has a value of n-6 DPA/DTA by GC area of not greater than 1.5.

(38) The method for producing microbial oil according to any one of (29) to (37), wherein the microbial oil has a value of DHA/n-3 DPA by GC area of not greater than 4.

(39) The method for producing microbial oil according to any one of (29) to (38), wherein the microbial oil satisfies the condition that the value of C20 PUFA/C22 PUFA by GC area is not less than 0.5 and not greater than 50.

(40) The method for producing microbial oil according to any one of (29) to (39), wherein the microbial oil satisfies the condition that the value of n-6 PUFA/n-3 PUFA by GC area is not less than 1.8.

(41) A method for producing microbial oil whereby microbial oil is caused to be produced in a labyrinthulid genetically modified such that the fatty acid composition is modified, the labyrinthulid being selected from the group consisting of (A) and (B) below.
(A) A labyrinthulid in which the fatty acid composition is modified by gene-disruption and/or gene silencing.
(B) A labyrinthulid in which the fatty acid composition is modified by transforming a gene in addition to disruption and/or gene silencing.

(42) The method for producing microbial oil according to (41), wherein the disrupted and/or silenced gene is a PKS gene, a fatty acid elongase gene, and/or a fatty acid desaturase gene.

(43) The method for producing microbial oil according to (41) or (42), wherein the transformed gene is a fatty acid elongase gene and/or a fatty acid desaturase gene.

(44) The method for producing microbial oil according to (42) or (43), wherein the fatty acid elongase gene is the C20 elongase gene.

(45) The method for producing microbial oil according to any one of claims 41 to 44, wherein the fatty acid desaturase gene is the Δ4 desaturase gene and/or the ω3 desaturase gene.

(46) The method for producing microbial oil according to any one of (41) to (45), wherein the method for disrupting or transforming a gene of a labyrinthulid is electroporation, a gene gun method, or gene editing.

(47) The method for producing microbial oil according to any one of (41) to (46), wherein the method for gene silencing of a labyrinthulid is an antisense method or RNA interference.

(48) A method for producing microbial oil, wherein microbial oil is caused to be produced in a labyrinthulid selected from the group consisting of (C) and (D) below.
(C) A labyrinthulid having very weak or no activity of producing PUFAs via the PUFA-PKS pathway.
(D) A labyrinthulid in which the host PUFA-PKS gene is disrupted or silenced to a very weak level.

(49) The method for producing microbial oil according to (48), wherein the labyrinthulid having very weak or no activity of producing PUFAs via the PUFA-PKS pathway is a labyrinthulid belonging to the genus Parietichytrium or genus Schizochytrium.

(50) The method for producing microbial oil according to (49), wherein the labyrinthulid belonging to the genus Parietichytrium is a labyrinthulid belonging to Parietichytrium sarkarianum.

(51) The method for producing microbial oil according to (50), wherein the labyrinthulid belonging to the genus Schizochytrium is a labyrinthulid belonging to Schizochytrium aggregatum.

(52) The method for producing microbial oil according to (50), wherein the microorganism belonging to Parietichytrium sarkarianum is Parietichytrium sp. SEK358 (FERM BP-11405), Parietichytrium sarkarianum SEK364 (FERM BP-11298), or Parietichytrium sp. SEK517 (FERM BP-11406).

(53) The method for producing microbial oil according to (51), wherein the microorganism belonging to Schizochytrium aggregatum is Schizochytrium aggregatum ATCC 28209.

(54) The method for producing microbial oil according to (48), wherein the labyrinthulid in which the host PUFA-PKS gene is disrupted or silenced to a very weak level belongs to the genus Thraustochytrium.

(55) The method for producing microbial oil according to (54), wherein the labyrinthulid belonging to the genus Thraustochytrium is Thraustochytrium aureum.

(56) A method for producing microbial oil satisfying at least one condition selected from the group consisting of (E) to (H) below.
(E) The GC area ratio of ARA after modification is not less than 3 times greater than before modification.
(F) The GC area ratio of DGLA after modification is not less than 4 times greater than before modification.
(G) The GC area ratio of ETA after modification is not less than 7 times greater than before modification.
(H) The GC area ratio of EPA after modification is not less than 7 times greater than before modification.

Furthermore, the gist of the present invention includes the following food, animal feed, medication, or industrial product (57), the following genetically modified labyrinthulid (58), and the following method for creating the genetically modified labyrinthulid (59).

(57) A food, animal feed, medication, or industrial product including the microbial oil described in any one of (1) to (28) as a lipid composition.

(58) A labyrinthulid genetically modified such that a produced fatty acid composition is modified, the labyrinthulid producing the microbial oil described in any one of (1) to (28).

(59) A method for creating the labyrinthulid genetically modified such that the produced fatty acid composition described in (58) is modified.

### BRIEF DESCRIPTION OF DRAWINGS

FIG. 1 illustrates the elongase-desaturase pathway and enzymes related to this biosynthesis pathway. (Brief description of symbols) C16:0: Palmitic acid; C18:0: Stearic acid; C18:1n-9: Oleic acid; C18:2n-6: Linoleic acid (LA); C18:3n-3: α-Linolenic acid (ALA); C18:3n-6: γ-Linolenic acid (GLA); C18:4n-3: Stearidonic acid (STA); C20:2n-6: Eicosadienoic acid (EDA); C20:3n-3: Eicosatrienoic acid (ETrA); C20:3n-6: Dihomo-γ-linolenic acid (DGLA); C20:4n-3: Eicosatetraenoic acid (ETA); C20:4n-6: Arachidonic acid (ARA); 20:5n-3: Eicosapentaenoic acid (EPA); C22:4n-6: Docosatetraenoic acid (DTA); C22:5n-3: n-3 Docosapentaenoic acid (n-3 DPA); C22:5n-6: n-6 Docosapentaenoic acid (n-6 DPA); C22:6n-3: Docosahexaenoic acid (DHA); C16e: C16 elongase; Δ9d: Δ9 desaturase; Δ12d: Δ12 desaturase; Δ15d: Δ15 desaturase; Δ9e: Δ9 elongase; Δ6d: Δ6 desaturase; Δ8d: Δ8 desaturase; C18e: C18 elongase; Δ5d: Δ5 desaturase; C20e: C20 elongase; Δ17d: Δ17 desaturase; Δ4d: Δ4 desaturase. Δ15 desaturase and Δ17 desaturase are each sometimes called ω3 desaturase.

FIG. 2 illustrates a plasmid containing an SV40 terminator sequence derived from a subcloned pcDNA 3.1 Myc-His vector.

FIG. 3 is a schematic diagram of the primers used in fusion PCR and the products. The final product is a fused sequence of a ubiquitin promoter derived from Thraustochytrium aureum ATCC 34304 and an artificially synthesized neomycin resistance gene.

FIG. 4 illustrates the BglII cassette of the produced artificially synthesized neomycin resistance gene.

FIG. 5 is a schematic diagram of the primers used in fusion PCR and the products. The final product is a fused sequence of a ubiquitin promoter derived from Thraustochytrium aureum ATCC 34304 and a hygromycin resistance gene derived from pcDNA 3.1/Hygro.

FIG. 6 illustrates the BglII cassette of the produced hygromycin resistance gene derived from pcDNA 3.1/Hygro.

FIG. 7 illustrates a plasmid containing a C20 elongase sequence of cloned genus of Parietichytrium.

FIG. 8 illustrates a plasmid in which a BglII site has been inserted in a C20 elongase sequence of genus of Parietichytrium of the plasmid illustrated in FIG. 7.

FIG. 9 illustrates the produced genus of Parietichytrium C20 elongase gene targeting vectors (two types). As a drug resistance marker, the vectors have a neomycin resistance gene (pRH85) or a hygromycin resistance gene (pRH86).

FIG. 10 is a schematic diagram illustrating the positions of the PCR primers used in identification of a C20 elongase gene disruption strain of Parietichytrium sarkarianum SEK364, and the expected product.

FIG. 11 illustrates an evaluation of C20 elongase gene disruption by PCR using Parietichytrium sarkarianum SEK364 genome DNA as a template. (Description of symbols) +/+: Parietichytrium sarkarianum SEK364 wild-type strain; +/−: C20 elongase gene first allele homologous recombinant derived from Parietichytrium sarkarianum SEK364; −/−: C20 elongase disruption strain derived from Parietichytrium sarkarianum SEK364

FIG. 12 illustrates a comparison of fatty acid compositions of the Parietichytrium sarkarianum SEK364 wild-type strain and the C20 elongase gene disruption strain. The black bars and white bars represent the fatty acid composition of the wild-type strain and the gene disruption strain, respectively. The values are mean±standard deviation.

FIG. 13 lists the fatty acid proportions of the C20 elongase gene disruption strain when the Parietichytrium sarkarianum SEK364 wild-type strain is taken as 100%.

FIG. 14 illustrates a plasmid containing a cloned Parietichytrium sp. SEK571 Δ4 desaturase gene sequence and the peripheral sequence.

FIG. 15 illustrates a plasmid in which the Parietichytrium sp. SEK571 Δ4 desaturase gene sequence and a 600 bp downstream of the Δ4 desaturase gene sequence have been deleted from the plasmid illustrated in FIG. 14 and a BglII site has been inserted.

FIG. 16 illustrates a plasmid in which a DNA fragment containing an artificially synthesized neomycin resistance gene cassette has been bound at the BglII site of the plasmid illustrated in FIG. 15.

FIG. 17 illustrates the produced genus of Parietichytrium Δ4 desaturase gene targeting vector. As a drug resistance marker, the vector has a neomycin resistance gene.

FIG. 18 is a schematic diagram illustrating the positions of the PCR primers used in identification of a Δ4 desaturase gene disruption strain of a genus of Parietichytrium labyrinthulid, and the expected product (primers are set within the homologous recombination region).

FIG. 19 illustrates evaluation results of Δ4 desaturase gene disruption by PCR using Parietichytrium sarkarianum SEK364 genome DNA as a template.

FIG. 20 is a gas chromatograph analysis chart of the fatty acid compositions of the Parietichytrium sarkarianum SEK364 wild-type strain and the Δ4 desaturase gene disruption strain thereof.

FIG. 21 is a partial enlarged diagram of FIG. 20.

FIG. 22 shows a comparison of the fatty acid compositions of the Parietichytrium sarkarianum SEK364 wild-type strain and the Δ4 desaturase gene disruption strain thereof. This table is a quantification of the chart of FIG. 20. In the table, the less-than sign (<) indicates less than or equal to the number following it.

FIG. 23 illustrates an evaluation of C20 elongase gene disruption by PCR using Parietichytrium sp. SEK358 strain genome DNA as a template. (Description of symbols) +/+: Parietichytrium sp. SEK358 wild-type strain; −/−: C20 elongase gene disruption strain derived from Parietichytrium sp. SEK358 strain

FIG. 24 illustrates a comparison of fatty acid compositions of the Parietichytrium sp. SEK358 wild-type strain and the C20 elongase gene disruption strain derived from Parietichytrium sp. SEK358 strain. The white bars and black bars represent the fatty acid composition of the wild-type strain and the gene disruption strain, respectively.

FIG. 25 lists the fatty acid proportions of the C20 elongase gene disruption strain derived from the Parietichytrium sp. SEK358 strain when the Parietichytrium sp. SEK358 wild-type strain is taken as 100%. In the Parietichytrium sp. SEK358 wild-type strain, cases where the relevant fatty acid is below the detection limit are indicated by a diagonal line.

FIG. 26 is a schematic diagram illustrating the positions of the PCR primers used in identification of a Δ4 desaturase gene disruption strain of a genus of Parietichytrium labyrinthulid, and the expected product (primers are set outside the homologous recombination region).

FIG. 27 illustrates evaluation results of Δ4 desaturase gene disruption by PCR using Parietichytrium sp. SEK358 genome DNA as a template in the case where the primers are set within the homologous recombination region.

FIG. 28 illustrates evaluation results of Δ4 desaturase gene disruption by PCR using Parietichytrium sp. SEK358 genome DNA as a template in the case where the primers are set outside the homologous recombination region.

FIG. 29 is a gas chromatograph analysis chart of the fatty acid compositions of the Parietichytrium sp. SEK358 wild-type strain and the Δ4 desaturase gene disruption strain thereof.

FIG. 30 is a partial enlarged diagram of FIG. 29.

FIG. 31 shows a comparison of the fatty acid compositions of the Parietichytrium sp. SEK358 wild-type strain and the Δ4 desaturase gene disruption strain thereof. This table is a quantification of the chart of FIG. 29. In the table, the less-than sign (<) indicates less than or equal to the number following it.

FIG. 32 illustrates an evaluation of C20 elongase gene disruption by PCR using Parietichytrium sp. SEK571 strain genome DNA as a template. (Description of symbols) +/+: Parietichytrium sp. SEK571 wild-type strain; −/−: C20 elongase gene disruption strain derived from Parietichytrium sp. SEK571 strain

FIG. 33 illustrates a comparison of fatty acid compositions of the Parietichytrium sp. SEK571 wild-type strain and the C20 elongase gene disruption strain derived from Parietichytrium sp. SEK571 strain. The white bars and black bars represent the fatty acid composition of the wild-type strain and the gene disruption strain, respectively.

FIG. 34 lists the fatty acid proportions of the C20 elongase gene disruption strain derived from the Parietichytrium sp. SEK571 strain when the Parietichytrium sp. SEK571 wild-type strain is taken as 100%.

FIG. 35 illustrates results of RACE in which an elongase gene derived from T. aureum ATCC 34304 is amplified in Comparative Example 1-2. (Brief description of symbols) 1: 5′-RACE using synthetic adapter-specific oligonucleotide and degenerate oligonucleotide elo-R; 2: 3′-RACE using synthetic adapter-specific oligonucleotide and degenerate oligonucleotide elo-F; 3: 5′-RACE using only elo-R (negative control); 4: 3′-RACE using only elo-F (negative control); 5: 5′-RACE using only synthetic adapter-specific oligonucleotide (negative control); 6: 3′-RACE using only synthetic adapter-specific oligonucleotide (negative control)

FIGS. 36A and 36B illustrate an evaluation of a transformant into which KONeor was transformed in Comparative Example 1-7. FIG. 36A illustrates the oligonucleotide primer pair used in evaluation of the transformant by PCR using genome DNA as a template. (Brief description of symbols) (1) Neor detection primers (SNeoF and SNeoR); (2) KO confirmation 1 (KO Pro F SmaI and KO Term R SmaI); (3) KO confirmation 2 (E2 KO ProF EcoRV and SNeoR); (4) KO confirmation 3 (SNeoF and E2 KO Term R EcoRV); (5) TaELG2 detection (E2 HindIII and E2 XbaI). FIG. 36B illustrates the agarose electrophoresis diagram in evaluation of the transformant by PCR using genome DNA as a template. (Brief description of symbols) 1, 5, 9, 13, 17: Transformant; 2, 6, 10, 14, 18: Wild-type strain; 3, 7, 11, 15, 19: Using KONeor as a template; 4, 8, 12, 16: No template. Furthermore, the used oligonucleotide primer pairs (1) to (5) are the lane numbers.

FIG. 37 illustrates the results of confirmation of TaELG2 copy number by southern blotting in Comparative Example 1-8. (Brief description of symbols) 1: Genome DNA (2.5 μg), BamHI treatment; 2: BglII treatment; 3: EcoRI treatment; 4: EcoRV treatment; 5: HindIII treatment; 6: KpnI treatment; 7: SmaI treatment; 8: XbaI treatment; 9: Positive control (PCR product amplified with 1 ng of E2 KO Pro F EcoRV and E2 KO Term R EcoRV. Including TaELG2.)

FIGS. 38A and 38B illustrate an evaluation by southern blotting of a transformant into which TKONeor was transformed in Comparative Example 1-9. FIG. 38A is a schematic diagram of southern blotting to detect a wild-type allele or mutant allele by TKONeor transfer. FIG. 38B illustrates the results of southern blotting. (Brief description of symbols) 1: T. aureum wild-type strain (2.5 μg of genome DNA); 2, 3: TKONeor transfer transformant (2.5 μg of genome DNA); 4: Positive control (PCR product amplified with 50 ng of E2 KO ProF EcoRV and E2 KO Term R EcoRV. Includes TaELG2.)

FIGS. 39A to 39C illustrate an evaluation by PCR using as a template genome DNA of a transformant obtained by retransfer of KOub600Hygr in Comparative Example 1-11. FIG. 39A illustrates the oligonucleotide primer pair used. (Brief description of symbols) (1) TaELO2 ORF detection (SNeoF and SNeoR); (2) KO confirmation (E2 KO Pro F EcoRV and ubi-hygro R)

FIG. 39B illustrates the agarose electrophoresis diagram of PCR using oligonucleotide primer pair (1) of KO confirmation. The arrows indicate the transformant of which specific product amplification was confirmed and which was estimated to be TaELO2 deletion homozygote.

FIG. 39C illustrates the agarose electrophoresis diagram of PCR using oligonucleotide primer pair (2) of TaELO2 ORF detection in a transformant identified as a TaELO2 deletion homozygote. (Brief description of symbols) 1: Using KOub600Hygr as a template; 2: Wild-type strain

FIGS. 40A and 40B illustrate an evaluation by southern blotting of a transformant obtained by retransfer of KOub600Hygr in Comparative Example 1-11. FIG. 40A is a schematic diagram of southern blotting to detect a wild-type allele, a mutant allele by KONeor transfer, and a mutant allele by KOub600Hygr transfer. FIG. 40B illustrates the results of southern blotting. (Brief description of symbols) 1, 9: Wild-type strain; 2 to 8 and 10 to 16: TaELO2 deletion homozygote

FIG. 41 illustrates the results of southern blotting to detect TaELO2 in Comparative Example 1-11. (Brief description of symbols) 1: Wild-type strain; 2 to 5: T TaELO2 deletion homozygote

FIG. 42 illustrates the results of agarose electrophoresis of RT-PCR to detect TaELO2 mRNA in Comparative Example 1-11. (Brief description of symbols) 1 to 4: TaELO2 deletion homozygote; 5: Wild-type strain; 6 to 9: TaELO2 deletion homozygote, using total RNA as a template (negative control); 10: Wild-type strain, using total RNA as a template (negative control); 11: Using wild-type genome DNA as a template (positive control)

FIG. 43 illustrates the results of a fatty acid composition comparison of the wild-type strain and TaELO2 deletion homozygote in Comparative Example 1-12.

FIG. 44 is a schematic diagram of the primers used in fusion PCR and the products. The final product is a fused sequence of 18S rDNA derived from Thraustochytrium aureum ATCC 34304, an EF1α promoter derived from Thraustochytrium aureum ATCC 34304, an artificially synthesized neomycin resistance gene, and an EF1α terminator derived from Thraustochytrium aureum ATCC 34304.

FIG. 45 illustrates a plasmid in which a portion of the ligated DNA fragment in FIG. 42 was cloned. The plasmid contains a partial sequence of the 3′ side from the EcoRI site of 18S rDNA derived from Thraustochytrium aureum ATCC 34304, an EF1α promoter derived from Thraustochytrium aureum ATCC 34304, an artificially synthesized neomycin resistance gene, and a partial sequence of the 5′ side from the NcoI site of the EF1α terminator derived from Thraustochytrium aureum ATCC 34304.

FIG. 46 illustrates the produced targeting vector of Thraustochytrium aureum ATCC 34304 PKS pathway associated gene orfA. As a drug resistance marker, the vector has a neomycin resistance gene.

FIG. 47 illustrates a plasmid containing an upstream sequence of the Thraustochytrium aureum ATCC 34304 PKS pathway associated gene orfA, a ubiquitin promoter derived from Thraustochytrium aureum ATCC 34304, and a hygromycin resistance gene.

FIG. 48 illustrates the produced targeting vector of Thraustochytrium aureum ATCC 34304 PKS pathway associated gene orfA. As a drug resistance marker, the vector has a hygromycin resistance gene.

FIG. 49 is a schematic diagram illustrating the positions of the southern hybridization analysis probes used in identification of the PKS pathway associated gene orfA disruption strain of Thraustochytrium aureum ATCC 34304, and the expected size of the gene fragment.

FIG. 50 illustrates an evaluation of PKS pathway associated gene orfA disruption by southern hybridization using Thraustochytrium aureum ATCC 34304 genome DNA. (Brief description of symbols) T. au: Thraustochytrium aureum ATCC 34304 wild-type strain; +/−: PKS pathway associated gene orfA first allele homologous recombinant derived from Thraustochytrium aureum ATCC 34304; −/−: PKS pathway associated gene orfA disruption strain derived from Thraustochytrium aureum ATCC 34304

FIG. 51 illustrates a comparison of fatty acid compositions of the Thraustochytrium aureum ATCC 34304 wild-type strain and the PKS pathway associated gene orfA disruption strain. The white bars and black bars represent the fatty acid composition of the wild-type strain and the gene disruption strain, respectively. The values are mean±standard deviation.

FIG. 52 lists the fatty acid proportions of the PKS pathway associated gene orfA disruption strain when the Thraustochytrium aureum ATCC 34304 wild-type strain is taken as 100%.

FIG. 53 is a schematic diagram of the primers used in fusion PCR and the products. The final product is a fused sequence of a ubiquitin promoter derived from Thraustochytrium aureum ATCC 34304 and a blasticidin resistance gene derived from pTracer-CMV/Bsd/lacZ.

FIG. 54 illustrates the produced BglII cassette of the blasticidin resistance gene derived from pTracer-CMV/Bsd/lac.

FIG. 55 is a schematic diagram of the primers used in fusion PCR and the products. The final product is a fused sequence of a ubiquitin promoter derived from Thraustochytrium aureum ATCC 34304 and an enhanced GFP gene (available from Clontech Laboratories, Inc.).

FIG. 56 is a schematic diagram of the primers used in fusion PCR and the products. The final product is a fused sequence of a ubiquitin promoter derived from Thraustochytrium aureum ATCC 34304, an enhanced GFP gene (available from Clontech Laboratories, Inc.), and a zeocin resistance gene derived from pcDNA 3.1 Zeo(+).

FIG. 57 illustrates the produced BglII cassette of the enhanced GFP-zeocin resistance fusion gene.

FIG. 58 illustrates a plasmid containing a cloned Thraustochytrium aureum ATCC 34304 C20 elongase sequence and the peripheral sequence.

FIG. 59 illustrates a plasmid in which the Thraustochytrium aureum ATCC 34304 C20 elongase sequence has been completely deleted from the plasmid illustrated in FIG. 56 and a BglII site has been inserted.

FIG. 60 illustrates the produced Thraustochytrium aureum ATCC 34304 C20 elongase gene targeting vectors (two types). As a drug resistance marker, the vectors have a blasticidin resistance gene (pRH43) or an enhanced GFP-zeocin resistance fusion gene (pRH54).

FIG. 61 is a schematic diagram illustrating the position of the southern hybridization analysis probe used in identification of the C20 elongase gene disruption strain of the Thraustochytrium aureum ATCC 34304 PKS pathway (orfA gene) disruption strain, and the expected size of the gene fragment.

FIG. 62 illustrates an evaluation of C20 elongase gene disruption by southern hybridization using Thraustochytrium aureum ATCC 34304 genome DNA. (Brief description of symbols) T. au: Thraustochytrium aureum ATCC 34304 wild-type strain; −/−: PKS pathway (orfA gene) and C20 elongase gene double disruption strain derived from Thraustochytrium aureum ATCC 34304

FIG. 63 illustrates a comparison of fatty acid compositions of the Thraustochytrium aureum ATCC 34304 wild-type strain and the PKS pathway (orfA gene) and C20 elongase gene double disruption strain. The white bars and black bars represent the fatty acid composition of the wild-type strain and the gene disruption strain, respectively. The values are mean±standard deviation.

FIG. 64 lists the fatty acid proportions of the PKS pathway (orfA gene) and C20 elongase gene double disruption strain when Thraustochytrium aureum ATCC 34304 wild-type strain is taken as 100%.

FIG. 65 illustrates a plasmid containing from 1071 bp upstream of the Δ4 desaturase gene to 1500 bp within the Δ4 desaturase gene of a cloned Thraustochytrium aureum ATCC 34304 strain.

FIG. 66 illustrates a plasmid in which a sequence of 60 bp upstream of the Δ4 desaturase gene of the plasmid illustrated in FIG. 63 and a sequence of 556 bp containing the start codon within the Δ4 desaturase gene (616 bp, SEQ ID NO: 205) have been deleted and a BglII site has been inserted in the deleted portion.

FIG. 67 illustrates the produced Thraustochytrium aureum ATCC 34304 strain Δ4 desaturase gene targeting vectors (two types). As a drug resistance marker, the vectors have a blasticidin resistance gene (pTM6) or an enhanced GFP-zeocin resistance fusion gene (pTM8).

FIG. 68 is a schematic diagram illustrating the positions of the PCR primers used in identification of the Δ4 desaturase gene disruption strain of the Thraustochytrium aureum ATCC 34304 PKS pathway (orfA gene) disruption strain, and the expected products.

FIG. 69 illustrates an evaluation of Δ4 desaturase gene disruption by PCR using Thraustochytrium aureum ATCC 34304 strain genome DNA as a template. (Brief description of symbols) +/+: PKS pathway (orfA gene) disruption strain derived from Thraustochytrium aureum ATCC 34304; +/−: Δ4 desaturase first allele homologous recombinant derived from PKS pathway (orfA gene) disruption strain derived from Thraustochytrium aureum ATCC 34304; −/−: PKS pathway (orfA gene) and Δ4 desaturase gene double disruption strain derived from Thraustochytrium aureum ATCC 34304

FIG. 70 illustrates a comparison of fatty acid compositions of the Thraustochytrium aureum ATCC 34304 wild-type strain and the PKS pathway (orfA gene) and Δ4 desaturase gene double disruption strain. The white bars and black bars represent the fatty acid composition of the wild-type strain and the gene disruption strain, respectively.

FIG. 71 lists the fatty acid proportions of the PKS pathway (orfA gene) and Δ4 desaturase gene double disruption strain when the Thraustochytrium aureum ATCC 34304 wild-type strain is taken as 100%.

### MODE FOR CARRYING OUT THE INVENTION

The opportunity for the present invention came about due to the discovery of a new pattern of biosynthesis pathway of polyunsaturated fatty acids (PUFAs) in microorganisms called labyrinthulids. Specifically, it is known that PUFAs are generally biosynthesized via the elongase-desaturase pathway, but in some organisms. PUFAs are also biosynthesized via another pathway called the PUFA-PKS pathway. In the past, two types of labyrinthulids were found to exist, namely (I) the type that produces only via the PUFA-PKS pathway and (II) the type that produces via the elongase-desaturase pathway and PUFA-PKS pathway. This time, a third type, (III) a type that produces only via the elongase-desaturase pathway, was newly discovered. In short, a new “pattern” of PUFA biosynthesis pathway was discovered.

Examples are as follows. (1) Labyrinthulids having very weak or no PUFA-producing activity via the endogenous PUFA-PKS pathway and having PUFA-producing activity via the endogenous elongase-desaturase pathway. The present invention relates to labyrinthulids of the above (III) type that produces only via the elongase-desaturase pathway. These labyrinthulids encompass labyrinthulids of type (III) produced by isolating, culturing, and amplifying a wild-type strain having a PUFA biosynthesis pathway, and labyrinthulids having “very weak or no” PUFA-producing activity via the PUFA-PKS pathway.

(2) The labyrinthulids according to (1) above that do not have the endogenous PUFA-PKS pathway. The present invention is limited to (1) above, which are labyrinthulids having no genes or enzymes themselves that constitute the PUFA-PKS pathway.

(3) The labyrinthulids according to (1) above that have very weak or no endogenous PUFA-PKS pathway activity. The present invention is limited to (1) above that have information related to genes of enzymes constituting the PUFA-PKS pathway on the genome but do not express them (and therefore have no activity), or that express them only very weakly or not at all.

(4) The labyrinthulids according to any of (1) to (3) above, wherein DHA and/or n-6 DPA production activity has been lost or the produced quantity of DHA and/or n-6 DPA has been markedly decreased by endogenous Δ4 desaturase gene disruption.

Labyrinthulids of type (III) which produce PUFAs only via the elongase-desaturase pathway differ from those of type (I) which produce only via the PUFA-PKS pathway and those of type (II) which produce via the elongase-desaturase pathway and PUFA-PKS pathway in that they have no PUFA-PKS pathway. Based on this viewpoint, the present invention defines labyrinthulids of the type that produces only via the elongase-desaturase pathway. In other words, the above endogenous Δ4 desaturase gene disruption can be considered a method for determining whether a labyrinthulid is of the type that produces only via the elongase-desaturase pathway.

(5) The labyrinthulids according to (1) to (3) above, wherein DHA and/or n-6 DPA production activity has been lost or the produced quantity of DHA and/or n-6 DPA has been markedly decreased through endogenous C20 elongase gene disruption.

Labyrinthulids of type (III) which produce only via the elongase-desaturase pathway differ from those of type (I) which produce only via the PUFA-PKS pathway and those of type (II) which produce via the elongase-desaturase pathway and PUFA-PKS pathway in that they have no PUFA-PKS pathway. Based on this viewpoint, the present invention defines labyrinthulids of the type that produces only via the elongase-desaturase pathway. In other words, the above endogenous C20 elongase gene disruption can be considered a method for determining whether a labyrinthulid is of the type that produces only via the elongase-desaturase pathway.

(6) The labyrinthulids according to (1) to (5) above that is a microorganism belonging to either the genus Parietichytrium or the genus Schizochytrium.

Labyrinthulids belonging to the genus Parietichytrium or the genus Schizochytrium were known before the filing date of the present application, but it was not known that they are labyrinthulids of type (III) which produce PUFAs only via the elongase-desaturase pathway.

(7) The labyrinthulids according to (6) above, wherein the microorganism is Parietichytrium sp. SEK358 (FERM BP-11405), Parietichytrium sarkarianum SEK364 (FERM BP-11298), Parietichytrium sp. SEK517 (FERM BP-11406), or Schizochytrium aggregatum ATCC 28209.

(8) A method for producing lipids containing PUFAs, the method including culturing the labyrinthulids described in any of (1) to (7) above in a culture medium and collecting the lipids from the culture.

(9) A method for producing lipids containing PUFAs, the method including culturing labyrinthulids, that were transformed using the labyrinthulids described in any of (1) to (7) above as hosts with the objective of modifying the fatty acid composition and/or highly accumulating fatty acids, in a culture medium and collecting the lipids from the culture.

(10) Lipids containing PUFAs, the lipids being produced by the method of (8) or (9) above.

The present invention can provide labyrinthulea that produce PUFAs via only the elongase-desaturase pathway.

The creation of labyrinthulids equivalent to type (III) which produce only via the elongase-desaturase pathway is also possible, by spontaneous mutation or genetic recombination from type (II) which produce via the elongase-desaturase pathway and PUFA-PKS pathway. Examples of these include microorganisms belonging to the genus Thraustochytrium. By using these microorganisms, it is possible to obtain the same polyunsaturated fatty acids as by labyrinthulids of type (III).

### [Microorganisms]

Labyrinthulids having very weak or no activity of producing PUFAs via the PUFA-PKS pathway includes labyrinthulids that produce PUFAs only via the elongase-desaturase pathway. A labyrinthulid having very weak PUFA-producing activity means a labyrinthulid in which the elongase-desaturase pathway gene has been disrupted, and which cannot produce PUFAs, and cannot be cultured without supplementing the culture medium with PUFAs. It means that not greater than 1/100 of the DHA synthesized in the organism is DHA synthesized via the PUFA-PKS pathway. The labyrinthulid that has very weak or no activity of producing PUFAs via the endogenous PUFA-PKS pathway and is capable of producing PUFAs via the endogenous elongase-desaturase pathway is not particularly limited, but particularly preferred examples are labyrinthulids belonging to the genus Parietichytrium or the genus Schizochytrium. Particularly preferred among these are Parietichytrium sp. SEK358 (FERM BP-11405), Parietichytrium sarkarianum SEK364 (FERM BP-11298), Parietichytrium sp. SEK517 (FERM BP-11406), or Schizochytrium aggregatum ATCC 28209.

Parietichytrium sp. SEK358 was obtained by the method described below. First, 10 mL of surface water collected in the Ishigakijima-Miyaragawa estuary region was placed in a test tube. Pine pollen was added, and the test tube was left to stand at room temperature. After 7 days, a sterilized agar culture medium (2 g of glucose, 1 g of peptone, 0.5 g of yeast extract, 0.2 g of chloramphenicol, 15 g of agar, 100 mL of distilled water, 900 mL of sea water) was swabbed with this pine pollen, and colonies that emerged after 5 days were separated and cultured. This was repeated several times, and cells were separated. This strain was internationally deposited on Aug. 11, 2011 as accession number FERM BP-11405 at the International Patent Organism Depositary, National Institute of Advanced Industrial Science and Technology (Tsukuba Central 6, 1-1-1 Higashi, Tsukuba City, Ibaraki Prefecture), and can be procured therefrom.

Parietichytrium sarkarianum SEK364 was obtained by culturing a sea water sample collected in the Ishigakijima-Fukidogawa estuary region and separating cells in the same manner as above. This strain was internationally deposited on Sep. 24, 2010 as accession number FERM BP-11298 at the International Patent Organism Depositary, National Institute of Advanced Industrial Science and Technology (Tsukuba Central 6, 1-1-1 Higashi, Tsukuba City, Ibaraki Prefecture), and can be procured therefrom.

Parietichytrium sp. SEK571 was obtained by culturing a sea water sample collected in the Iriomotejima-Shiiragawa estuary region and separating cells in the same manner as above. This strain was internationally deposited on Aug. 11, 2011 as accession number FERM BP-11406 at the International Patent Organism Depositary, National Institute of Advanced Industrial Science and Technology (Tsukuba Central 6, 1-1-1 Higashi, Tsukuba City, Ibaraki Prefecture), and can be procured therefrom.

Schizochytrium aggregatum ATCC 28209 has been deposited at ATCC, and can be procured therefrom. Labyrinthulids in which PUFA-PKS has been disrupted or expression has been inhibited to a very weak level include labyrinthulids that produce PUFAs via the elongase-desaturase pathway and PUFA-PKS pathway. The PUFA-PKS pathway is required to exist in labyrinthulids that produce PUFAs only via the PUFA-PKS pathway, and when disrupted, there is a requirement for PUFAs. Labyrinthulids that produce PUFAs via the elongase-desaturase pathway and PUFA-PKS pathway, even in a case where the PUFA-PKS pathway has been disrupted, differ in that there is no requirement for PUFAs.

Disruption of the Δ4-desaturase gene using the present invention can dramatically reduce the produced quantities of DHA and n-6 DPA while maintaining the produced quantity of PUFAs overall.

The properties of the microorganisms obtained by the present invention may be combined as desired.

### [Microbial Oil]

The present invention relates to microbial oil containing the fatty acid profile of the present invention. The microorganism of the present invention contains not less than 15 wt. %, preferably not less than 30 wt. %, more preferably not less than 50 wt. %, and even more preferably not less than 70 wt. % of lipid components per gram of cells. The lipid components contain not less than 30 wt. %, preferably not less than 50 wt. %, and more preferably not less than 70 wt. % of fatty acid components. The fatty acid components accumulate as not less than 70 wt. %, preferably not less than 80 wt. %, and more preferably not less than 90 wt. % of triglycerides. The microbial oil of the present invention is “crude oil” or “refined oil” containing at least approximately 35 wt. % of triacylglycerol fraction. “Crude oil” is oil extracted from microbial biomass which has not undergone further treatment. “Refined oil” is oil obtained by treating crude oil by standard purification, decoloration, and/or deodorizing processes. The microbial oil further contains “final oil”, which is refined oil diluted with vegetable oil. The “microorganisms” are not limited, but include classifications taxonomically associated with any “microalgae”, “labyrinthulids”, and the deposited microorganisms described in the present specification. The terms “labyrinthulid”, “genus Parietichytrium”, “genus Schizochytrium”, and “genus Thraustochytrium” when used in association with any microbial oils of a deposited microorganism described herein are based on current taxonomic classification including phylogenetically usable information, and are not intended to be limiting if the taxonomic classification is revised after the filing date of the present application. Lipids

In the present invention, lipids are lipids produced by labyrinthulids, and are mainly triglycerides, diglycerides, monoglycerides, phospholipids, free fatty acids, sterols, carotenoids, hydrocarbons, and the like.

### [Lipids]

In the present invention, lipids are lipids produced by labyrinthulids, and are mainly triglycerides, diglycerides, monoglycerides, phospholipids, free fatty acids, sterols, carotenoids, hydrocarbons, and the like.

### [Polyunsaturated Fatty Acids]

In the present invention, a polyunsaturated fatty acid (PUFA) is a fatty acid having not less than 18 carbon atoms and not less than three double bonds, and more preferably a fatty acid having not less than 20 carbon atoms and not less than three double bonds. Specific examples include linoleic acid (LA, 18:2n-6), α-linolenic acid (ALA, 18:3n-3), γ-linolenic acid (GLA, 18:3n-6), stearidonic acid (STA, 18:4n-3), dihomo-γ-linolenic acid (DGLA, 20:3n-6), eicosatetraenoic acid (ETA, 20:4n-3), arachidonic acid (ARA, 20:4n-6), eicosapentaenoic acid (EPA, 20:5n-3), docosatetraenoic acid (DTA, 22:4n-6), n-3 docosapentaenoic acid (n-3 DPA, 22:5n-3), n-6 docosapentaenoic acid (n-6 DPA, 22:5n-6), and docosahexaenoic acid (DHA, 22:6n-3). In the present specification, arachidonic acid is also expressed as ARA. The total fatty acid composition means the composition of fatty acids detected after a microorganism is cultured and freeze dried and the fatty acids are then methyl-esterified and analyzed by GC. Specifically, the total fatty acid composition is the composition of fatty acids having from 14 to 22 carbon chains. In the present invention, GC area means the peak area of the GC chart. A proportion relative to the total fatty acid composition means the proportion of the peak area of the targeting fatty acid relative to the total of peak areas of the entire fatty acid composition, and is expressed as a percentage. In the present invention, C20 PUFA/C22 PUFA means the value obtained by dividing the total of GC peak areas of polyunsaturated fatty acids having 20 carbon chains by the total of GC peak areas of polyunsaturated fatty acids having 22 carbon chains. In the present invention, n-6 PUFA/n-3 PUFA means the value obtained by dividing the total of peak areas of the GC chart of ω6 fatty acids having not less than 20 carbon chains by the total of peak areas of the GC chart of ω3 fatty acids having not less than 20 carbon chains.

### [PUFA Biosynthesis Pathways]

Two different pathways associated with PUFA biosynthesis are known. One is a pathway that produces polyunsaturated fatty acids (PUFAs) using polyketide synthase (PKS). In the present invention, this metabolic pathway will be called the PUFA-PKS pathway or the PKS pathway. In the present invention, a PKS gene means a gene that encodes a protein that constitutes polyketide synthase. Polyketide synthase is an enzyme that catalyzes a reaction in which a long-chain substrate such as malonyl-CoA is condensed multiple times in a starter substrate such as acetyl-CoA. Polyketide synthase has generally been known as an enzyme involved in biosynthesis of secondary metabolites of plants, fungi, and the like, but it has also been reported to be involved in PUFA biosynthesis in certain organisms. For example, the marine Bacillus Shewanella produces eicosapentaenoic acid (EPA) using this enzyme (Non-patent Document 8). Polyketide synthase is known to be similarly involved in PUFA biosynthesis in certain labyrinthulids as well.

The other is a pathway in which, starting from a fatty acid such as palmitic acid, desaturation by desaturase and chain elongation by elongase are repeated to produce PUFAs such as EPA and DHA. In the present invention, this is called the elongase-desaturase pathway. Examples of the enzymes that constitute this system include fatty acid synthesis associated enzymes such as C20 elongase and Δ4 desaturase.

### [PUFA Biosynthesis Pathway Differentiation Method]

A method for differentiating between PUFA biosynthesis pathways of labyrinthulids will be described below. However, this is only one example, and needless to say, differentiation does not have to be performed by this method.

As stated above, two PUFA biosynthesis pathways in labyrinthulids are known. One is the PUFA-PKS pathway, by which DHA and n-6 DPA are produced. In this pathway, no PUFAs other than DHA and n-6 DPA are substantially produced such configuration differs greatly from the elongase-desaturase pathway, which is the other PUFA biosynthesis pathway.

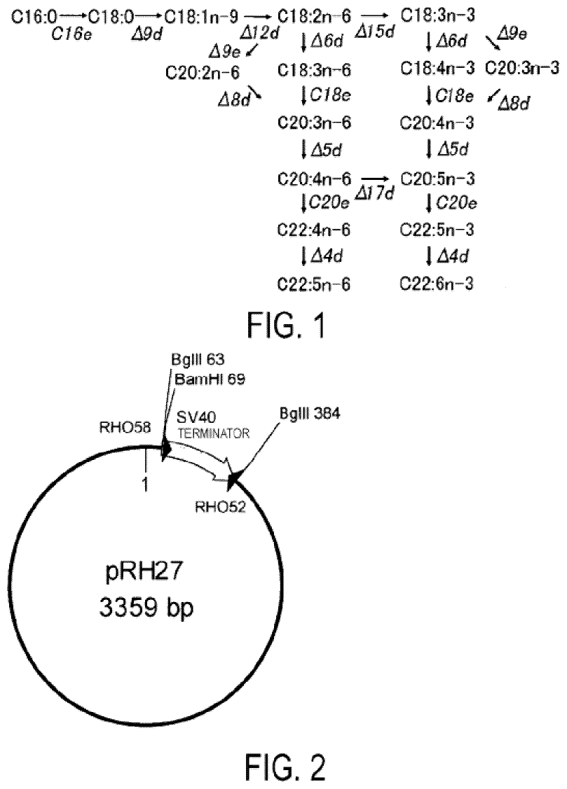

The other pathway, namely the elongase-desaturase pathway, is illustrated in FIG. 1. Among the enzymes constituting this pathway, Δ4 desaturase is an enzyme involved in converting n-3 DPA to DHA. This enzyme is also involved in converting DTA to n-6 DPA. Thus, since these conversions do not take place when the gene of this enzyme is disrupted, n-3 DPA and DTA, which are substrates of this enzyme, accumulate, and conversely, DHA and n-6 DPA, which are products of this enzyme, decrease. In a labyrinthulid in which this Δ4 desaturase gene has been disrupted, in a case where the products DHA and/or n-6 DPA are completely undetected or markedly decreased, it is judged that the labyrinthulid has very weak or no PUFA (particularly DHA and/or n-6 DPA) production activity via the endogenous PUFA-PKS pathway.

It is also possible to select C20 elongase rather than Δ4 desaturase and disruption the gene of that enzyme instead. Specifically, C20 elongase is an enzyme involved in converting EPA to n-3 DPA. This enzyme is also involved in converting ARA to DTA. Thus, since these conversions do not take place when the gene of this enzyme is disrupted, EPA and ARA, which are substrates of this enzyme, accumulate, and conversely, n-3 DPA and DTA, which are products of this enzyme, decrease. As a result, DHA and n-6 DPA, which have n-3 DPA and DTA as substrates, also decrease. In a labyrinthulid in which this C20 elongase gene has been disrupted, when the products DHA and/or n-6 DPA are completely undetected or markedly decreased, it is judged that the labyrinthulid has very weak or no PUFA (particularly DHA and/or n-6 DPA) production activity via the endogenous PUFA-PKS pathway.

Gene disruption is performed by transforming something designed such that gene products do not produce activity using an antibiotic resistance gene or the like in all or a portion of the target gene, into a target cell by a method such as a gene gun, electroporation, or gene editing. Gene silencing is performed by transforming an antisense gene designed such that expression of the target gene is inhibited or a gene in which RNAi is expressed, into a cell by a method such as a gene gun, electroporation, or gene editing. Gene disruption and silencing are not limited to these methods provided that expression of the target gene is hindered. When disruption or silencing is performed with a PKS gene as the target, the target is not limited provided that enzyme activity can be eliminated or inhibited, but OrfA is preferably used as the target.

The present invention provides labyrinthulids having very weak or no PUFA producing activity via the endogenous PUFA-PKS pathway and having PUFA-producing activity via the endogenous elongase-desaturase pathway.

Furthermore, the present invention also includes changing the fatty acid composition produced by a labyrinthulid by manipulating the genes of enzymes constituting the elongase-desaturase pathway of the labyrinthulid. In particular, the fatty acid composition produced by a labyrinthulid can be modified by: (1) disruption and/or silencing of a fatty acid elongase gene, (2) disruption and/or silencing of a fatty acid desaturase gene, (3) transfer of a fatty acid elongase gene, (4) transfer of a fatty acid desaturase gene, and (5) a combination of the above. For example, when stearidonic acid (STA) is desired, a fatty acid elongase gene involved in conversion of stearidonic acid to eicosatetraenoic acid (ETA), specifically the C18 elongase gene, may be disrupted and/or silenced. Furthermore, for example, when eicosapentaenoic acid (EPA) is desired, a fatty acid elongase gene involved in conversion of eicosatetraenoic acid to docosapentaenoic acid (DPA), specifically the C20 elongase gene, may be disrupted and/or silenced. As another example, when eicosapentaenoic acid is desired, a fatty acid desaturase gene that converts arachidonic acid (ARA) to eicosapentaenoic acid, specifically the ω3 desaturase gene, may be transformed. For a labyrinthulid that produces via the elongase-desaturase pathway and PUFA-PKS pathway, the fatty acid composition produced by the labyrinthulid can be modified by: (1) disruption and/or silencing of a fatty acid elongase gene, (2) disruption and/or silencing of a fatty acid desaturase gene, (3) transfer of a fatty acid elongase gene, (4) transfer of a fatty acid desaturase gene, and (5) a combination of the above, using a microorganism in which the PUFA-PKS pathway has been disrupted or silenced. For example, when stearidonic acid (STA) is desired, a fatty acid elongase gene involved in conversion of stearidonic acid to eicosatetraenoic acid (ETA), specifically the C18 elongase gene, may be disrupted and/or silenced. Furthermore, for example, when eicosapentaenoic acid (EPA) is desired, a fatty acid elongase gene involved in conversion of eicosatetraenoic acid to docosapentaenoic acid (DPA), specifically the C20 elongase gene, may be disrupted and/or silenced. As another example, when eicosapentaenoic acid is desired, a fatty acid desaturase gene that converts arachidonic acid (ARA) to eicosapentaenoic acid, specifically the ω3 desaturase gene, may be transformed.

A labyrinthulid (microorganism) in which the produced fatty acid composition has been modified is obtained by transformation of the labyrinthulid. A labyrinthulid in which a fatty acid biosynthesis associated gene has been transformed and/or disrupted can be used in, for example, production of unsaturated fatty acids. In production of unsaturated fatty acids, the conditions of other steps, production equipment and instruments and the like are not particularly limited provided that the labyrinthulid that is used has very weak or no PUFA production activity via the endogenous PUFA-PKS pathway and has PUFA production activity via the endogenous elongase-desaturase pathway, or is a labyrinthulid in which the produced fatty acid composition has been modified as described above. Production of unsaturated fatty acids includes a step of culturing a labyrinthulid that has very weak or no PUFA production activity via the endogenous PUFA-PKS pathway and has PUFA production activity via the endogenous elongase-desaturase pathway, or a labyrinthulid in which the produced fatty acid composition has been modified as described above. Unsaturated fatty acids are produced using these microorganisms and their culture medium.

The above cell culturing conditions (culture medium, culture temperature, ventilation status, and the like) may be set as appropriate according to the type of cell, the targeted type and quantity of unsaturated fatty acids, and the like. Furthermore, an unsaturated fatty acid in the present invention means a substance containing an unsaturated fatty acid, without limitation on its content, purity, shape, composition, and the like. In other words, in the present invention, cells in which the fatty acid composition has been modified or their culture media are themselves considered to be unsaturated fatty acids. Additionally, a step of purifying the unsaturated fatty acids from these cells or culture media may be further included. As the method of purifying the unsaturated fatty acids, a method known as a purification method for lipids (including complex lipids) such as unsaturated fatty acids may be applied.

### [Method for Highly Accumulating Unsaturated Fatty Acids in Labyrinthulids]

Accumulation of unsaturated fatty acids in a labyrinthulid is achieved by culturing a labyrinthulid having very weak or no PUFA production activity via the endogenous PUFA-PKS pathway and having PUFA production activity via the endogenous elongase-desaturase pathway, or a transformant thereof. The labyrinthulid may be cultured in, for example, a solid culture medium, liquid culture medium, or the like. The culture medium used at this time is not particularly limited provided that it is a medium commonly used for culturing labyrinthulids and appropriately combines, for example, glucose, fructose, saccharose, starch, glycerin, or the like as a carbon source, yeast extract, corn steep liquor, polypeptone, sodium glutamate, urea, ammonium acetate, ammonium sulfate, ammonium nitrate, ammonium chloride, sodium nitrate, or the like as a nitrogen source, potassium phosphate or the like as an inorganic salt, and other necessary components. However, yeast extract-glucose medium (GY medium) is particularly preferably used. After preparation of the culture medium, the pH is adjusted to within the range of 3.0 to 8.0, and then the culture medium is sterilized by autoclave or the like. Culturing may be performed byaerated and agitated culturing, shake culturing, or static culturing at 10 to 40° C., preferably 15 to 35° C., for 1 to 14 days.

To collect the produced unsaturated fatty acids, the labyrinthulids are grown in a culture medium and the microorganism cells obtained from that culture medium are treated, the lipids (polyunsaturated fatty acids or oil- and fat-containing matter containing polyunsaturated fatty acids) inside the cells are released and collected. Specifically, lipids containing PUFAs can be obtained by collecting the labyrinthulids cultured in this manner by centrifugation or the like, performing treatment such as drying and cell crushing as necessary, and performing extraction using an appropriate organic solvent or supercritical carbon dioxide, liquefied dimethylether, or the like according to conventional methods.

The microbial oil obtained in the present invention is that which satisfies any of the following conditions. The microbial oil obtained in the present invention contains ARA in a proportion of not less than 5%, not less than 7%, not less than 10%, or not less than 15% of the total fatty acid composition. Oils and fats with high ARA obtained in this manner may be used in applications such as nutritional supplements for infants and health foods and medications for adults. ARA may be not greater than 80%, not greater than 70%, not greater than 60%, or not greater than 50% of the total fatty acid composition. The microbial oil obtained in the present invention contains DGLA in a proportion of not less than 2.5%, not less than 5%, or not less than 10% of the total fatty acid composition. Microbial oil with high DGLA obtained in this manner may be used in medicinal applications such as anti-inflammatory agents. DGLA may be not greater than 80%, not greater than 70%, not greater than 60%, or not greater than 50% of the total fatty acid composition. The microbial oil obtained in the present invention contains ETA in a proportion of not less than 0.35%, not less than 0.5%, not less than 0.75%, or not less than 1% of the total fatty acid composition. Microbial oil with high ETA obtained in this manner may be used in medicinal applications such as arthritis treatment. ETA may be not greater than 50%, not greater than 40%, not greater than 30%, or not greater than 20% of the total fatty acid composition. The microbial oil obtained in the present invention contains EPA in a proportion of not less than 4%, not less than 6%, not less than 8%, not less than 10%, or not less than 12% of the total fatty acid composition. Microbial oil with high EPA obtained in this manner may be used in nutritional supplement applications and medicinal applications. EPA may be not greater than 80%, not greater than 70%, not greater than 60%, or not greater than 50% of the total fatty acid composition. The microbial oil obtained in the present invention contains n-6 DPA in a proportion of not greater than 0.20%, not greater than 0.15%, not greater than 0.1%, or not greater than 0.05% of the total fatty acid composition. The microbial oil with low n-6 DPA obtained in this manner does not tend to hinder the functions of other fatty acids. Furthermore, it is advantageous in cases where removal of n-6 DPA by refinement is desired. n-6 DPA may be not less than 0.001%, not less than 0.005%, or not less than 0.01% of the total fatty acid composition. The microbial oil obtained in the present invention contains DHA in a proportion of not greater than 0.50%, not greater than 0.3%, not greater than 0.2%, or not greater than 0.1% of the total fatty acid composition. The microbial oil with low DHA obtained in this manner does not tend to hinder the functions of other fatty acids. Furthermore, it is advantageous in cases where removal of DHA by refinement is desired. DHA may be not less than 0.005%, not less than 0.01%, or not less than 0.05% of the total fatty acid composition. The microbial oil obtained in the present invention contains a total of DHA and n-6 DPA in a proportion of not greater than 0.7%, not greater than 0.8%, not greater than 0.9%, or not greater than 1.0% of the total fatty acid composition. The microbial oil with low DHA and n-6 DPA obtained in this manner does not tend to hinder the functions of other fatty acids and is stable against oxidation. Furthermore, it is advantageous in cases where removal of DHA and n-6 DPA by refinement is desired. The total of DHA and n-6 DPA may be not less than 0.05%, not less than 0.1%, or not less than 0.5% of the total fatty acid composition. The desired concentrations of each of these fatty acids may be combined as desired, limited to a total of 100%.

In another aspect, the microbial oil obtained in the present invention is that which satisfies any of the following conditions. In the microbial oil obtained in the present invention, the value of LA/DHA by GC area is not less than 0.6, not less than 0.7, not less than 0.8, or not less than 0.9, and not greater than 10, not greater than 9, not greater than 8, not greater than 7, or not greater than 6. The microbial oil with a high LA/DHA value obtained in this manner is stable against oxidation, and the functions of LA do not tend to be hindered by DHA. In the microbial oil obtained in the present invention, the value of GLA/DHA by GC area is not less than 0.35, not less than 0.4, not less than 0.5, not less than 0.6, or not less than 0.7, and not greater than 10, not greater than 9, not greater than 8, or not greater than 7. The microbial oil with a high GLA/DHA value obtained in this manner is stable against oxidation, and the functions of GLA do not tend to be hindered by DHA.

In the microbial oil obtained in the present invention, the value of DGLA/DHA by GC area is not less than 0.35, not less than 0.4, not less than 0.5, not less than 0.6, or not less than 0.7, and not greater than 10, not greater than 9, not greater than 8, not greater than 7, or not greater than 6. Microbial oil with a high DGLA/DHA value obtained in this manner may be used in medications having an anti-inflammatory action. In the microbial oil obtained in the present invention, the value of ARA/DHA by GC area is not less than 0.7, not less than 0.8, not less than 0.9, or not less than 1.0, and not greater than 50, not greater than 45, not greater than 40, not greater than 35, or not greater than 30. Microbial oil with a high ARA/DHA value obtained in this manner may be used in modified milk for infants. The value of EPA/DHA by GC area is not less than 0.35, not less than 0.4, not less than 0.5, not less than 0.6, or not less than 0.7, and not greater than 50, not greater than 45, not greater than 40, not greater than 35, or not greater than 30. EPA used in typical medications and the like are mainly esters containing DHA, but the microbial oil with a high EPA/DHA value obtained in this manner contains EPA with a high degree of purity, and can be used in health foods, medications, and the like.

The desired ratios of each of these fatty acids may be combined as desired. In the microbial oil obtained in the present invention, the value of LA/EPA by GC area is not less than 0.06, not less than 0.07, not less than 0.08, or not less than 0.09, and not greater than 0.12, not greater than 0.1, not greater than 0.08, or not greater than 0.06. Microbial oil with a low LA/EPA value obtained in this manner may be used in infusion fluids with a low anti-inflammatory action and the like. In the microbial oil obtained in the present invention, the value of GLA/EPA by GC area is not less than 0.04 or not less than 0.45, and not greater than 0.12, not greater than 0.1, or not greater than 0.8. Microbial oil with a low GLA/EPA value obtained in this manner may be used in infusion fluids with a low anti-inflammatory action and the like. In the microbial oil obtained in the present invention, the value of DTA/EPA by GC area is not less than 0.01, not less than 0.02, not less than 0.03, not less than 0.04, or not less than 0.05, and not greater than 0.4, not greater than 0.35, or not greater than 0.3. The microbial oil with a low DTA/EPA value obtained in this manner is useful for separating high-purity EPA since DTA and EPA are close to each other in GC. The desired ratios of each of these fatty acids may be combined as desired.

In the microbial oil obtained in the present invention, the value of DTA/ARA by GC area is not less than 0.01, not less than 0.03, not less than 0.05, not less than 0.07, or not less than 0.1, and not greater than 0.45, not greater than 0.4, not greater than 0.35, or not greater than 0.3. Microbial oil with a low DTA/ARA value obtained in this manner may be used in formulated milk for infants. In the microbial oil obtained in the present invention, the value of DTA/DGLA by GC area is not less than 0.01, not less than 0.05, not less than 0.1, not less than 0.15, or not less than 0.2, and not greater than 1.45, not greater than 1.4, not greater than 1.3, not greater than 1.2, or not greater than 1.1. Microbial oil with a low DTA/DGLA value obtained in this manner may be used in medications having an anti-inflammatory action and the like. In the microbial oil obtained in the present invention, the value of LA/n-6 DPA by GC area is not less than 0.4, not less than 0.5, not less than 0.6, not less than 0.7, or not less than 0.8, and not greater than 20, not greater than 18, not greater than 16, not greater than 14, or not greater than 12. Microbial oil with a high LA/n-6 DPA value obtained in this manner may be used in edible oils. In the microbial oil obtained in the present invention, the value of GLA/n-6 DPA by GC area is not less than 0.2, not less than 0.4, not less than 0.6, or not less than 0.8, and not greater than 10, not greater than 8, not greater than 6, or not greater than 4. Microbial oil with a high GLA/n-6 DPA value obtained in this manner may be used in health foods and supplements. In the microbial oil obtained in the present invention, the value of DGLA/n-6 DPA by GC area is not less than 0.35, not less than 0.5, not less than 0.75, or not less than 1.0, and not greater than 30, not greater than 27, not greater than 25, not greater than 22, or not greater than 20. Microbial oil with a high DGLA/n-6 DPA value obtained in this manner may be used in medications having an anti-inflammatory action. In the microbial oil obtained in the present invention, the value of ARA/n-6 DPA by GC area is not less than 0.7, not less than 1.0, not less than 2.0, or not less than 3.0, and not greater than 60, not greater than 50, not greater than 40, or not greater than 30. Microbial oil with a high LA/n-6 DPA value obtained in this manner may be used in formulated milk for infants. In the microbial oil obtained in the present invention, the value of EPA/n-6 DPA by GC area is not less than 0.4, not less than 0.6, not less than 0.8, not less than 1, not less than 2, or not less than 5, and not greater than 70, not greater than 60, not greater than 50, not greater than 40, or not greater than 30. Microbial oil with a high EPA/n-6 DPA value obtained in this manner may be used in health foods, supplements, and the like. The desired ratios of each of these fatty acids may be combined as desired.

In the microbial oil obtained in the present invention, the value of DGLA/LA by GC area is not less than 1.4, not less than 2, or not less than 3, and not greater than 10, not greater than 9, not greater than 8, or not greater than 7. The microbial oil obtained in this manner may be used in foods and supplements having an anti-inflammatory action. In the microbial oil obtained in the present invention, the value of ARA/LA by GC area is not less than 5.1, not less than 7, not less than 9, or not less than 11, and not greater than 20, not greater than 17, not greater than 15, or not greater than 12. Microbial oil with a high ARA/LA value obtained in this manner may be used in animal feed and formulated milk for infants. In the microbial oil obtained in the present invention, the value of EPA/LA by GC area is not less than 5.5, not less than 7, not less than 9, not less than 11, or not less than 13, and not greater than 30, not greater than 25, not greater than 22, or not greater than 20. Microbial oil with a high EPA/LA value obtained in this manner may be used in medications and health foods. In the microbial oil obtained in the present invention, the value of DTA/LA by GC area is not less than 0.01, not less than 0.05, not less than 0.07, or not less than 0.1, and not greater than 0.4, not greater than 0.35, not greater than 0.33, not greater than 0.3, or not greater than 0.28. Microbial oil with a low DTA/LA value obtained in this manner may be used in animal feed and foods. The desired ratios of each of these fatty acids may be combined as desired.

In the microbial oil obtained in the present invention, the value of DGLA/GLA by GC area is not less than 4.5, not less than 5, not less than 6, or not less than 7, and not greater than 20, not greater than 17, not greater than 15, or not greater than 12. Microbial oil with a high DGLA/GLA value obtained in this manner may be used in medications having an anti-inflammatory action and the like. In the microbial oil obtained in the present invention, the value of ARA/GLA by GC area is not less than 9, not less than 10, not less than 12, not less than 13, or not less than 14, and not greater than 30, not greater than 28, not greater than 26, not greater than 24, or not greater than 22. Microbial oil with a high ARA/GLA value obtained in this manner may be used in formulated milk for infants. The desired ratios of each of these fatty acids may be combined as desired.

In the microbial oil obtained in the present invention, the value of n-6 DPA/DTA by GC area is not less than 0.001, not less than 0.01, or not less than 0.02, and not greater than 1.5, not greater than 1.4, not greater than 1.3, not greater than 1.2, or not greater than 1.1. The microbial oil obtained in this manner may be used in foods that prevent arteriosclerosis and the like. In the microbial oil obtained in the present invention, the value of DHA/n-3 DPA by GC area is not less than 0.001, not less than 0.01, or not less than 0.02, and not greater than 4, not greater than 4.5, not greater than 4, or not greater than 3.5.

The microbial oil obtained in this manner may be used in animal feed or health foods. In the microbial oil obtained in the present invention, the value of C20 PUFA/C22 PUFA by GC area is not less than 0.5, not less than 0.7, not less than 1, or not less than 1.2, and not greater than 50, not greater than 45, not greater than 40, or not greater than 35. Microbial oil with a high C20 PUFA/C22 PUFA value obtained in this manner may be used in health foods and supplements. In the microbial oil obtained in the present invention, the value of n-6 PUFA/n-3 PUFA by GC area is not less than 1.8, not less than 2, not less than 2.5, or not less than 3, and not greater than 100, not greater than 80, not greater than 70, not greater than 60, or not greater than 50. Microbial oil with a high n-6 PUFA/n-3 PUFA value obtained in this manner may be used in edible oils and animal feed. The desired ratios of each of these fatty acids may be combined as desired.

The desired ratios of each of these fatty acids may be combined as desired. The desired concentrations of each of these fatty acids may be combined as desired, limited to a total of 100%.

The unsaturated fatty acids of the present invention also include various medications, foods, animal feeds, and industrial products, and the fields of use thereof are not particularly limited. The foods containing the unsaturated fatty acid-containing oils and fats of the present invention also include health foods such as supplements and food additives and the like. Examples of the industrial products include animal feed for organisms other than humans, films, biodegradable plastics, functional fibers, lubricating oils, and detergents.

Next, the present invention will be specifically described based on examples. Furthermore, in the present specification, the features of each invention described in embodiments related to each aspect of the invention may be combined as desired to form new embodiments, and it is to be understood that such new embodiments may be included in each of the aspects of the present invention.

### Example 1

### [Labyrinthulids and Culturing Method/Storage Method Thereof]

### (1) Strains Used in the Present Invention

Parietichytrium sp. SEK358 (FERM BP-11405), Parietichytrium sarkarianum SEK364 (FERM BP-11298), and Parietichytrium sp. SEK571 (FERM BP-11406) were shared from the Department of Engineering at Konan University. Thraustochytrium aureum ATCC 34304 was shared from ATCC.

### (2) Culture Medium Composition

i. Agar Plate Culture Medium Composition

### PDA Agar Plate Culture Medium

0.78% (w/v) of potato dextrose agar medium (available from Nissui Pharmaceutical Co., Ltd.), 1.75% (w/v) of Sealife (available from Marintec Co., Ltd.), and 1.21% (w/v) of agar powder (available from Nacalai Tesque, Inc.) were mixed and then sterilized by autoclave for 20 min at 121° C. After sufficient cooling, ampicillin sodium salt (available from Nacalai Tesque, Inc.) was added so as to result in a final concentration of 100 μg/mL. This was dispensed into a Petri dish and left to stand at a level location to solidify.

ii. Liquid Culture Medium Composition

### GY Liquid Culture Medium

3.18% (w/v) of glucose (available from Nacalai Tesque, Inc.), 1.06% (w/v) of dry yeast extract (available from Nacalai Tesque, Inc.), and 1.75% (w/v) of Sealife (available from Marintec Co., Ltd.) were mixed and then sterilized by autoclave for 20 min at 121° C. Ampicillin sodium salt (available from Nacalai Tesque, Inc.) was then added so as to result in a final concentration of 100 μg/mL.

### PD Liquid Culture Medium

0.48% (w/v) of potato dextrose (available from Difco Laboratories Inc.) and 1.75% (w/v) of Sealife (available from Marintec Co., Ltd.) were mixed and then sterilized by autoclave for 20 min at 121° C. Ampicillin sodium salt (available from Nacalai Tesque, Inc.) was then added so as to result in a final concentration of 100 μg/mL.

### (3) Culturing Method

i. Agar Plate Culturing

Labyrinthulea cells were inoculated using a platinum loop or a spreader and then static cultured at 25° C., thereby causing emergence of colonies. Subculturing was performed by extracting colonies using a platinum loop and suspending them in sterilized physiological saline solution, and then spreading this suspension using a platinum loop or a spreader. Furthermore, as necessary, it was transformed to a liquid culture by inoculating cells in a liquid culture medium on a flat plate.

ii. Liquid Culturing

Labyrinthulea cells were inoculated, and suspension culturing was performed with stirring at 150 rpm at 25° C. using an Erlenmeyer flask or a test tube. Subculturing was performed by adding a culture solution in which growth was confirmed from the logarithmic growth phase to the stationary phase, in a volume ratio of 1/200 to 1/10 to a fresh GY or PD liquid culture medium.

Furthermore, as necessary, it was transformed to an agar plate culture by spreading the cell culture solution on a PDA agar plate culture medium.

### (4) Preservation/Storage Method of Labyrinthulids

In addition to subculturing, cryopreservation was performed by producing glycerol stock. Specifically, glycerol (available from Nacalai Tesque, Inc.) was added to a cell suspension that used a GY liquid culture medium from the logarithmic growth phase to the stationary phase, so as to result in a final concentration of 15% (v/v), and this was stored in a deep freezer at −80° C.

### Example 2

[Measurement of Fatty Acid Composition of Lipids Produced by C20 Elongase Gene Disruption and Transformation Strain of Parietichytrium sarkarianum SEK364]

### [Example 2-1]: Subcloning of SV40 Terminator Sequence

An SV40 terminator sequence was amplified with PrimeSTAR HS DNA Polymerase (available from Takara Bio Inc.) using a pcDNA 3.1 myc-His vector (available from Invitrogen Corp.) as a template. The PCR primers used were as shown below. RH058 was set on the SV40 terminator sequence, and includes BglII and BamHI linker sequences. RH052 was set on the SV40 terminator sequence, and includes a BglII sequence. [RH058: 34mer: 5′-CAG ATC TGG ATC CGC GAA ATG ACC GAC CAA GCG A-3′ (SEQ ID NO: 1), RH052: 24mer: 5′-ACG CAA TTAATG TGA GAT CTA GCT-3′ (SEQ ID NO: 2)]. After amplification under the following conditions, it was cloned in pGEM-T Easy Vector (available from Promega Corporation). [PCR cycles: 98° C. 2 min/98° C. 30 sec, 60° C. 30 sec, 72° C. 1 min, 30 cycles/72° C. 1 min]. After amplification with E. coli, the sequence was confirmed using a Dye Terminator Cycle Sequencing Kit (available from Beckman Coulter Inc.). This was named pRH27.

The plasmid (pRH27) containing the subcloned SV40 terminator sequence (342 bp, SEQ ID NO: 3) is illustrated in FIG. 2.

### [Example 2-2]: Production of Artificially Synthesized Neomycin Resistance Gene Cassette

Thraustochytrium aureum ATCC 34304 strain was cultured in a GY culture medium, and cells of the latter logarithmic growth phase were centrifuged for 5 min at 4° C. at 3500×g to form pellets, and the pellets were frozen with liquid nitrogen and then crushed. After phenol extraction of the crushed cell liquid, ethanol precipitation was performed and the precipitate was dissolved in a TE solution. The nucleic acids dissolved in the TE solution were treated with RNase for 30 min at 37° C., and after further phenol extraction, ethanol precipitation was performed and the precipitate was dissolved in a TE solution. A260/280 was measured and the DNA concentration was calculated.

Using this as a template, an ubiquitin promoter sequence (619 bp, SEQ ID NO: 4) was amplified with PrimeSTAR HS DNA Polymerase with GC Buffer (available from Takara Bio Inc.). The PCR primers used were as shown below. RHO53 was set on the ubiquitin promoter sequence, and includes a BglII linker sequence. TKO1 includes the ubiquitin promoter sequence and an artificially synthesized neomycin resistance gene sequence. [RHO53: 36mer: 5′-CCC AGA TCT GCC GCA GCG CCT GGT GCA CCC GCC GGG-3′ (SEQ ID NO: 5), TKO1: 58mer: 5′-CGT GAA GGC CGT CCT GTT CAA TCA TGT TGG CTA GTG TTG CTT AGG TCG CTT GCT GCT G-3′ (SEQ ID NO: 6)]. [PCR cycles: 98° C. 2 min/98° C. 10 sec, 68°1 min, 30 cycles/68° C. 1 min].

Using the artificially synthesized neomycin resistance gene sequence as a template, an artificially synthesized neomycin resistance gene sequence (826 bp, SEQ ID NO: 7) was amplified with PrimeSTAR HS DNA Polymerase with GC Buffer (available from Takara Bio Inc.). The PCR primers used were as shown below. TKO2 includes the ubiquitin promoter sequence and the artificially synthesized neomycin resistance gene sequence. RHO57 includes the artificially synthesized neomycin resistance gene sequence and has a BglII linker sequence. [TKO2: 54mer: 5′-AGC GAC CTA AGC AAC ACT AGC CAA CAT GAT TGA ACA GGA CGG CCT TCA CGC TGG-3′ (SEQ ID NO: 8), RHO57: 26mer: 5′-CAG ATC TCA AAA GAA CTC GTC CAG GA-3′ (SEQ ID NO: 9)] [PCR cycles: 98° C. 2 min/98° C. 10 sec, 68° 1 min, 30 cycles/68° C. 1 min].

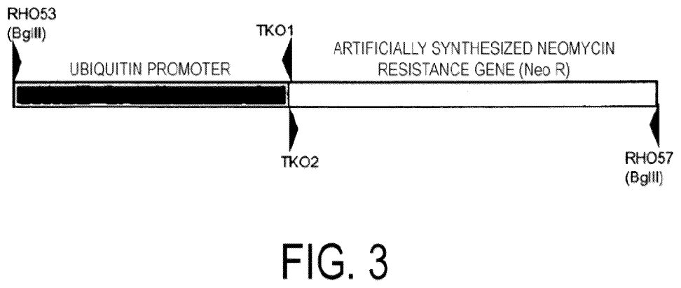

Using SEQ ID NO: 4 and SEQ ID NO: 7 as templates, fusion PCR was performed using RHO53 (SEQ ID NO: 5) and RH057 (SEQ ID NO: 9) according to the method described in Non-patent Document 9. Amplification was performed using LA Taq Hot Start Version (available from Takara Bio Inc.) as the enzyme with PCR cycles under conditions below: 94° C. 2 min/94° C. 20 sec, 55° C. 30 sec, 68° C. 1 min, 30 cycles/68° C. 1 min (1° C./10 sec from 55° C. to 68° C.), and then the amplified product was digested with BglII. (FIG. 3).

The Thraustochytrium aureum ATCC 34304-derived ubiquitin promoter—artificially synthesized neomycin resistance gene sequence (1395 bp, SEQ ID NO: 10) fused as described above was digested with BglII, and the resultant was bound to the BamHI site of pRH27 described in Example 2-1. After amplification of the produced plasmid with E. coli, the sequence was confirmed using a Dye Terminator Cycle Sequencing Kit (available from Beckman Coulter Inc.). This was named pRH31.

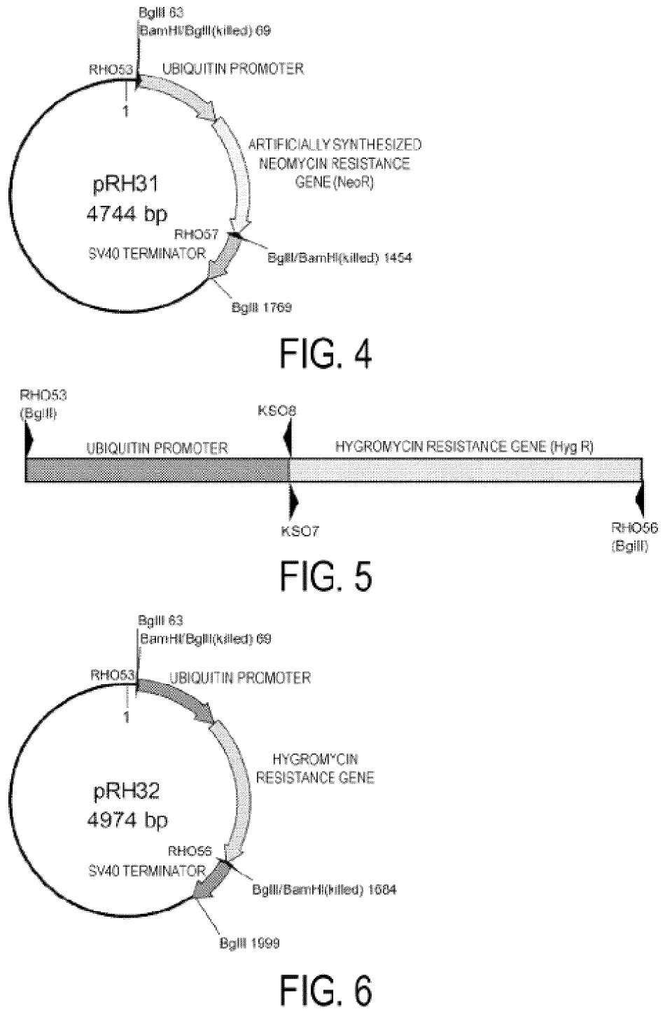

The produced artificially synthesized neomycin resistance gene cassette (pRH31) is illustrated in FIG. 4.

### [Example 2-3]: Production of Hygromycin Resistance Gene Cassette

Using Thraustochytrium aureum ATCC 34304 genome DNA as a template, a ubiquitin promoter sequence (617 bp, SEQ ID NO: 11) was amplified with PrimeSTAR HS DNA Polymerase with GC Buffer (available from Takara Bio Inc.). The PCR primers used were as shown below. RH053 was set on the ubiquitin promoter sequence, and includes a BglII linker sequence. KSO8 includes the ubiquitin promoter sequence and a hygromycin resistance gene sequence. [RH053: 36mer: 5′-CCC AGA TCT GCC GCA GCG CCT GGT GCA CCC GCC GGG-3′ (described in Example 2-2, SEQ ID NO: 5), KSO8: 58mer: 5′-TCG CGG TGA GTT CAG GCT TTT TCA TGT TGG CTA GTG TTG CTT AGG TCG CTT GCT GCT G-3′ (SEQ ID NO: 12)] [PCR cycles: 98° C. 2 min/98° C. 30 sec, 68° 2 min, 30 cycles/68° C. 2 min]

Using pcDNA 3.1/Hygro (available from Invitrogen Corp.) as a template, a hygromycin resistance gene (1058 bp, SEQ ID NO: 13) was amplified with PrimeSTAR HS DNA Polymerase with GC Buffer (available from Takara Bio Inc.). The PCR primers used were as shown below. KSO7 includes the ubiquitin promoter sequence and the hygromycin resistance gene sequence. RH056 includes the hygromycin resistance gene sequence and has a BglII linker sequence. [KSO7: 56mer: 5′-AGC GAC CTAAGC AAC ACT AGC CAA CAT GAA AAA GCC TGA ACT CAC CGC GAC GTC TG-3′ (SEQ ID NO: 14), RH056: 36mer: 5′-CAG ATC TCT ATT CCT TTG CCC TCG GAC GAG TGC TGG-3′ (SEQ ID NO: 15)]. [PCR cycles: 98° C. 2 min/98° C. 30 sec, 68° 2 min, 30 cycles/68° C. 2 min]

Using SEQ ID NO: 11 and SEQ ID NO: 13 as templates, fusion PCR was performed using RHO53 (described in Example 2-2, SEQ ID NO: 5) and RHO56 (SEQ ID NO: 15) according to the method described in Non-patent Document 9. Amplification was performed using LA Taq Hot Start Version (available from Takara Bio Inc.) as the enzyme under the following conditions, and then the amplified product was digested with BglII. [PCR cycles: 94° C. 2 min/94° C. 20 sec, 55° C. 30 sec, 68° C. 1 min, 30 cycles/68° C. 1 min (1° C./10 sec from 55° C. to 68° C.)] (FIG. 5).

The Thraustochytrium aureum ATCC 34304-derived ubiquitin promoter—pcDNA 3.1/Hygro (available from Invitrogen Corp.)-derived hygromycin resistance gene (1625 bp, SEQ ID NO: 16) fused as described above was digested with BglII, and the resultant was bound to the BamHI site of pRH27 described in Example 2-1, FIG. 2. After amplification of the produced plasmid with E. coli, the sequence was confirmed using a Dye Terminator Cycle Sequencing Kit (available from Beckman Coulter Inc.). This was named pRH32.

The produced hygromycin resistance gene cassette (pRH32) is illustrated in FIG. 6.

### [Example 2-4]: Cloning of Genus Parietichytrium C20 Elongase Gene

Genome DNA of Parietichytrium sarkarianum SEK364 was extracted by the method described in Example 2-2, and the genome was read.

With the region conserved in the C20 elongase gene as a target, a forward oligonucleotide (PsTaELO2 F1; 5′-CCT TCG GCG CTC CTC TTA TGT ATG T-3′) (SEQ ID NO: 17) and a reverse oligonucleotide (PsTaELO2 R2; 5′-CAA TGC AAG AGG CGA ACT GGG AGA G-3′) (SEQ ID NO: 18) were synthesized. Next, using the Parietichytrium sarkarianum SEK364 genome DNA prepared by the method described in Example 2-2 as a template, PCR was performed using the oligonucleotides PsTaELO2 F1 and PsTaELO2 R2 using LA Taq Hot Start Version (available from Takara Bio Inc.). [PCR cycles: 98° C. 1 min/98° C. 10 sec, 60° C. 30 sec, 72° C. 1 min, 30 cycles/72° C. 7 min/4° C. ∞]. The obtained specific amplification product underwent gel purification, and when the base sequence thereof was analyzed by direct sequencing, it exhibited significant homology to a known C20 elongase gene sequence. This shows that it was a partial sequence of the C20 elongase gene derived from Parietichytrium sarkarianum SEK364.

Then, the C20 elongase gene derived from Parietichytrium sarkarianum SEK364 was cloned by 3′- and 5′-RACE in the same manner as Comparative Example 1-2 to be described later. First, forward oligonucleotide primers (PsRACE F1; 5′-TGG GGC TCT GGA ACC GCT GCT TAC G-3′) (SEQ ID NO: 19) and (PsRACE F2; 5′-CTT CCA GCT CTC CCA GTT CGC CTC T-3′) (SEQ ID NO: 20), and reverse oligonucleotide primers (PsRACE R1; 5′-CGG GTT GTT GAT GTT GAG CGA GGT G-3′) (SEQ ID NO: 21) and (PsRACE R2; 5′-CCC ACG CCA TCC ACG AGC ACA CCA C-3′) (SEQ ID NO: 22) were designed. Next, using a cDNA library produced by SMART RACE cDNA Amplification Kit (trade name; available from Clontech Laboratories, Inc.) as a template, 3′- and 5′-RACE were performed using a synthetic adapter-specific oligonucleotide and the above oligonucleotide PsRACE F1 or PsRACE R1. [PCR cycles: 94° C. 30 sec 5 cycles/94° C. 30 sec, 70° C. 30 sec, 72° C. 3 min, 5 cycles/94° C. 30 sec, 68° C. 30 sec, 72° C. 3 min, 25 cycles/4° C. ∞]. Then, using the two obtained RACE products as templates, nested PCR was performed using a synthetic adapter-specific oligonucleotide and the above oligonucleotide PsRACE F2 or PsRACE R2. [PCR cycles: 94° C. 1 min/94° C. 30 sec, 68° C. 30 sec, 72° C. 3 min, 25 cycles/72° C. 10 min/4° C. ∞]. The obtained specific amplification product underwent gel purification, and when the base sequence thereof was analyzed after TA cloning using pGEM-Easy Vector (available from Promega Corporation), it was confirmed to be the C20 elongase gene derived from Parietichytrium sarkarianum SEK364.

Additionally, using the genus Parietichytrium genome DNA extracted by the method described in Example 2-2 as a template, a sequence containing a C20 elongase gene sequence (957 bp, SEQ ID NO: 23) was amplified with LA Taq Hot Start Version (available from Takara Bio Inc.). The PCR primers used were as shown below. RHO153 includes a start codon, and has a BamHI site as a linker sequence. RHO154 includes a stop codon, and has a BamHI site as a linker sequence. [RHO153: 32mer: 5′-CCC GGA TCC ATG GCA GCT CGC GTG GAG AAA CA-3′ (SEQ ID NO: 24), RHO154: 33mer: 5′-CCC GGA TCC TTA CTG AGC CTT CTT GGA GGT CTC-3′ (SEQ ID NO: 25)]. [PCR cycles: 98° C. 2 min/98° C. 10 sec, 68° 1 min, 30 cycles/68° C. 2 min].

The obtained DNA fragment was cloned in pGEM-T Easy Vector, and after amplification with E. coli, the sequence was confirmed using a Dye Terminator Cycle Sequencing Kit (available from Beckman Coulter Inc.).

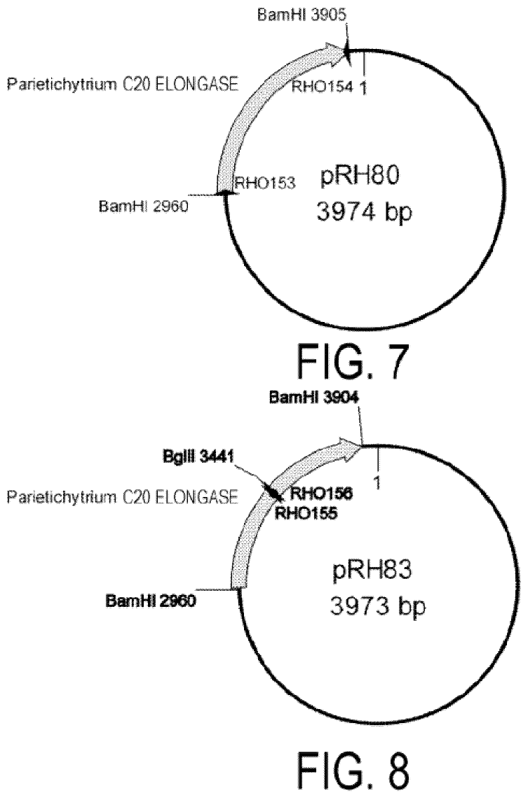

The genus Parietichytrium C20 elongase gene (936 bp, SEQ ID NO: 26) was cloned. This was named pRH80 (FIG. 7). The amino acid sequence is shown SEQ ID NO: 27.

### [Example 2-5]: Production of Base Plasmid for Production of Genus Parietichytrium C20 Elongase Gene Targeting Vector

A primer set that was set up in the reverse direction so as to insert a BglII site into the core portion of the C20 elongase gene sequence was prepared using pRH80 (FIG. 7) produced in Example 2-4 as a template, and the resultant was amplified with PrimeSTAR Max DNA Polymerase (available from Takara Bio Inc.). The PCR primers used were as shown below and have a BglII linker sequence. [RHO155: 26mer: 5′-ACA AAG ATC TCG ACT GGA CCG ACA CC-3′ (SEQ ID NO: 28), RHO156: 27mer: 5′-AGT CGA GAT CTT TGT CAG GAG GTG GAC-3′ (SEQ ID NO: 29)]. [PCR cycles: 98° C. 2 min/98° C. 10 sec, 56° C. 15 sec, 72° C. 1 min, 30 cycles/72° C. 1 min]. After amplification under the above conditions, it was digested with BglII and then self-ligated. After the ligated sample was amplified with E. coli, the sequence was confirmed using a Dye Terminator Cycle Sequencing Kit (available from Beckman Coulter Inc.). This was named pRH83. The C20 elongase gene sequence (935 bp) in which a BglII site was inserted is shown in SEQ ID NO: 30.

FIG. 8 illustrates the produced base plasmid (pRH83) for producing a genus Parietichytrium C20 elongase gene targeting vector.

### [Example 2-6]: Production of Targeting Vectors (Artificially Synthesized Neomycin Gene and Hygromycin Resistance Gene)

pRH31 (FIG. 4) described in Example 2-2 was digested with BglII, and a DNA fragment containing an artificially synthesized neomycin resistance gene cassette was bound to the BglII site of pRH83 (FIG. 8) described in Example 2-5. This was named pRH85.

pRH32 (FIG. 6) described in Example 2-3 was digested with BglII, and a DNA fragment containing a hygromycin resistance gene cassette was bound to the BglII site of pRH83 (FIG. 8) described in Example 2-5. This was named pRH86.

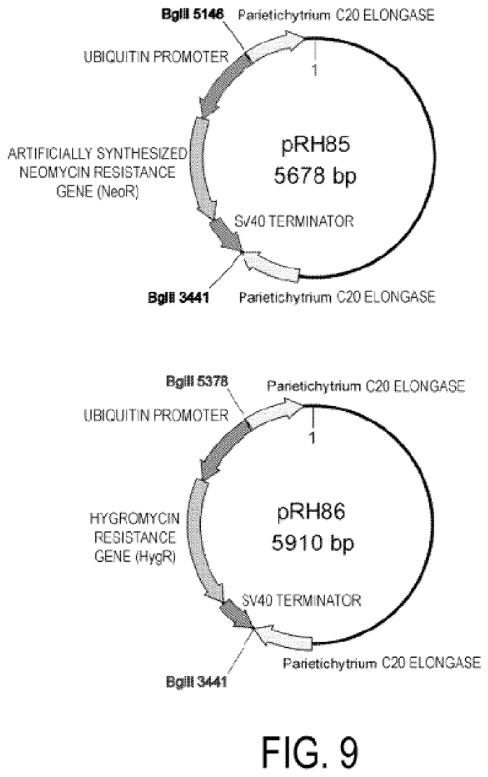

The two produced targeting vectors (pRH85 and 86) are illustrated in FIG. 9.

### [Example 2-7]: C20 Elongase Gene Targeting Vector Transfer

Using the two targeting vectors produced in Example 2-6 as templates, the genes were amplified with PrimeSTAR Max DNA Polymerase (available from Takara Bio Inc.) using RHO153 (described in Example 2-4, SEQ ID NO: 24) and RHO154 (described in Example 2-4, SEQ ID NO: 25) as primers. [PCR cycles: 98° C. 2 min/98° C. 30 sec, 68° 2 min, 30 cycles/68° C. 2 min]. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH85 (FIG. 9) described in Example 2-6 was used as a template was 2661 bp, and resulted in a sequence composed of genus Parietichytrium C20 elongase gene front half—SV40 terminator sequence—artificially synthesized neomycin resistance gene sequence—ubiquitin promoter sequence—genus Parietichytrium C20 elongase gene back half (SEQ ID NO: 31). The transfer fragment obtained when pRH86 (FIG. 9) described in Example 2-6 was used as a template was 2892 bp, and resulted in a sequence composed of genus Parietichytrium C20 elongase gene front half—SV40 terminator sequence—hygromycin resistance gene sequence—ubiquitin promoter sequence—genus Parietichytrium C20 elongase gene back half (SEQ ID NO: 32).

The Parietichytrium sarkarianum SEK364 strain was cultured for 4 days in a GY culture medium, and cells in the logarithmic growth phase were used for gene transfer. To cells corresponding to OD600=1 to 1.5, 0.625 μg of DNA fragment was transformed by the gene gun method (microcarrier: 0.6 micron gold particles, target distance: 6 cm, chamber vacuum: 26 mmHg, rupture disk: 1550 psi). After a recovery time of 24 hr, the transgenic cells were spread on a PDA agar plate culture medium (containing 2 mg/mL of G418 or containing 2 mg/mL of hygromycin). As a result, from 10 to 20 cells of drug resistant strain per shot were obtained.

### [Example 2-8]: Identification of C20 Elongase Gene Targeting Homologous Recombinant

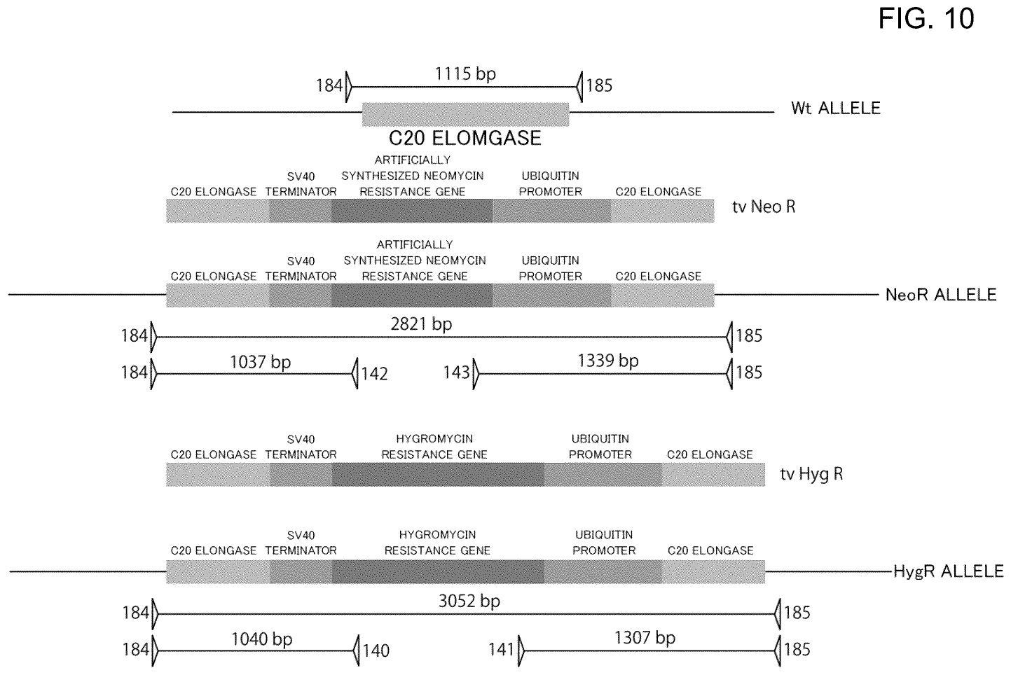

Genome DNA of the Parietichytrium sarkarianum SEK364 strain, a C20 elongase gene hetero homologous recombinant, and a C20 elongase gene homo homologous recombinant (gene disruption strain) were extracted by the method described in Example 2-2, and then A260/280 was measured and the DNA concentration was calculated. Using the genome DNA as templates, PCR for genome structure confirmation was performed using LA Taq Hot Start Version (available from Takara Bio Inc.). The positions of the primers used, the combinations used in amplification, and the expected sizes of the amplification products are illustrated in FIG. 10. RHO184 was set upstream of C20 elongase; RHO185 was set downstream; RHO142 and RHO143 were set on the artificially synthesized neomycin resistance gene; and RHO140 and RHO141 were set on the hygromycin resistance gene. [RHO140: 20mer: 5′-GGT TGA CGG CAA TTT CGA TG-3′ (SEQ ID NO: 33), RHO141: 22mer: 5′-CCT CCT ACA TCG AAG CTG AAA G-3′ (SEQ ID NO: 34), RHO142: 21mer: 5′-CTT CTC GGG CTT TAT CGA CTG-3′ (SEQ ID NO: 35), RHO143: 22mer: 5′-TAA GGT CGG TCT TGA CAA ACA G-3′ (SEQ ID NO: 36), RHO184: 24mer: 5′-AGT AGT CCC CGA TTT GGT AGT TGA-3′ (SEQ ID NO: 37), RHO185: 22mer: 5′-GGC AGA GAG CAA AAA CAC GAG C-3′ (SEQ ID NO: 38)]. [PCR cycles: 98° C. 2 min/98° C. 10 sec, 68° 4 min, 30 cycles/68° C. 7 min].

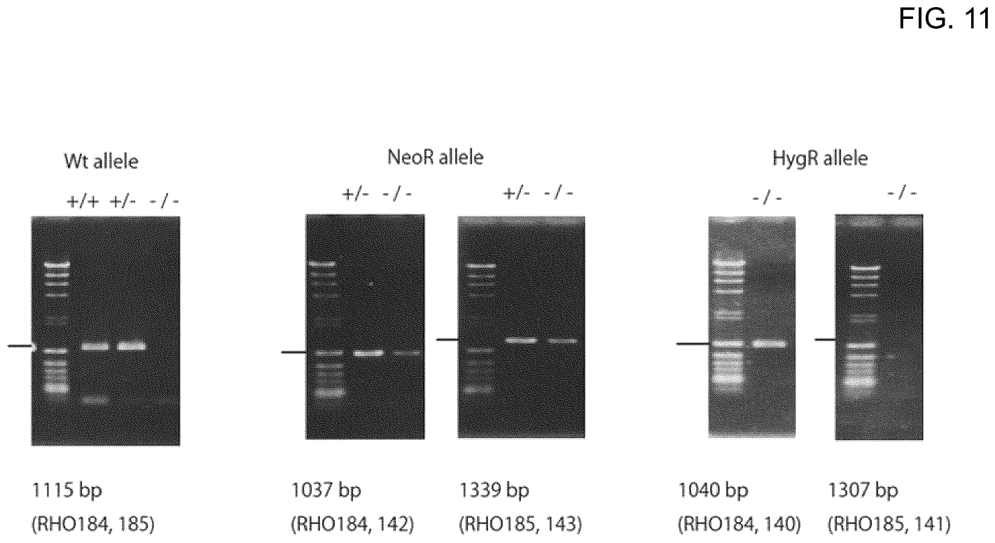

A C20 elongase gene disruption strain in which there is no amplification in the wild-type allele (Wt allele) and there is amplification in the artificially synthesized neomycin resistance gene allele (NeoR allele) and the hygromycin resistance gene allele (HygR allele) was obtained (FIG. 11).

### Example 2-9: Change in Fatty Acid Composition by C20 Elongase Gene Disruption

The Parietichytrium sarkarianum SEK364 wild-type strain and the gene disruption strain thereof (C20 elongase gene disruption strain, C20 −/−) were cultured in a GY culture medium. The cells of the latter phase of the logarithmic growth phase were centrifuged for 10 min at 4° C. at 3000 rpm to form pellets, and the obtained pellets were suspended in 0.9% NaCl and washed. Then, the resultant was centrifuged for 10 min at 4° C. at 3000 rpm, and the pellets were suspended in sterilized water and washed. The resultant was further centrifuged for 10 min at 3000 rpm, the supernatant was removed and the precipitate was freeze-dried.

To the freeze-dried cells, 2 mL of methanolic KOH (7.5% KOH in 95% methanol) was added, and after vortexing, the cells were crushed by ultrasound (80° C., 30 min). Then, 500 μL of sterilized water was added and vortexing was performed, and then 2 mL of n-hexane was added and vortexing was performed. The resultant was then centrifuged for 10 min at 3000 rpm, and the top layer was discarded. Another 2 mL of n-hexane was added and vortexing was performed. The resultant was centrifuged for 10 min at 3000 rpm, and the top layer was discarded. One mL of 6 N HCl was added to the remaining bottom layer and vortexing was performed, and then 2 mL of n-hexane was added and vortexing was performed. The resultant was then centrifuged for 10 min at 3000 rpm, and the top layer was collected. Another 2 mL of n-hexane was added and vortexing was performed. The resultant was centrifuged for 10 min at 3000 rpm, and the top layer was collected. The collected top layer was concentrated and dried with nitrogen gas. Two mL of 3 N methanolic HCl was added to the concentrated and dried sample, and the resultant was incubated overnight at 80° C.

The sample was cooled to room temperature, and 1 mL of 0.9% NaCl was added. Then, 2 mL of n-hexane was added and vortexing was performed. The resultant was centrifuged for 10 min at 3000 rpm, and the top layer was collected. Another 2 mL of n-hexane was added and vortexing was performed. The resultant was centrifuged for 10 min at 3000 rpm, and the top layer was collected. A small amount of anhydrous sodium sulfate was added to the collected top layer and then vortexing was performed. The resultant was centrifuged for 10 min at 3000 rpm, and the top layer was collected. The collected top layer was concentrated and dried with nitrogen gas. The concentrated and dried sample was dissolved in 0.5 mL of n-hexane, and 1 μL of the resultant was subjected to GC analysis. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min).

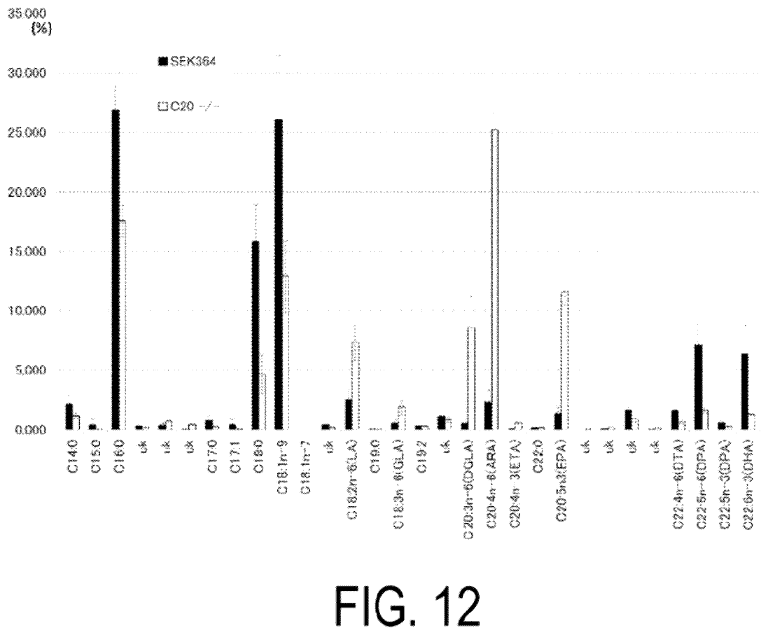

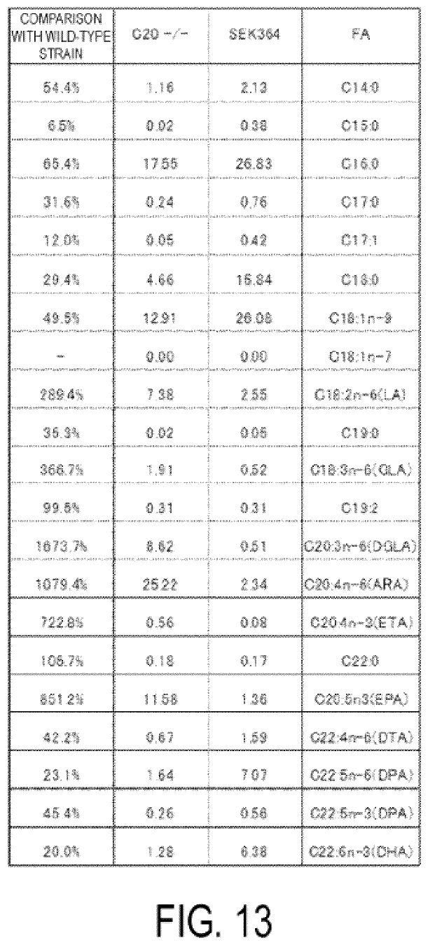

As a result, when the C20 elongase gene was disrupted in Parietichytrium sarkarianum SEK364, fatty acids having not less than 22 carbon chains decreased while fatty acids having 20 carbon chains increased (FIG. 12). FIG. 13 shows the proportion when the wild-type strain is taken as 100%. FIG. 13 shows that, of the total fatty acid composition, ARA is 25.22%, DGLA is 8.62%, ETA is 0.56%, EPA is 11.58%, n-6 DPA is 1.64%, and DHA is 1.28%. FIG. 13 shows that, by GC area, LA/DHA is 5.8, GLA/DHA is 1.5, DGLA/DHA is 6.7, ARA/DHA is 19.7, EPA/DHA is 9.0, LA/EPA is 0.64, GLA/EPA is 0.16, DTA/EPA is 0.06, DTA/ARA is 0.03, DTA/DGLA is 0.08, LA/n-6 DPA is 4.5, GLA/n-6 DPA is 1.2, DGLA/n-6 DPA is 5.3, ARA/n-6 DPA is 15.4, EPA/n-6 DPA is 7.1, DGLA/LA is 1.2, ARA/LA is 3.4, EPA/LA is 1.6, DTA/LA is 0.09, DGLA/GLA is 4.5, ARA/GLA is 13.2, n-6 DPA/DTA is 2.4, DHA/n-3 DPA is 4.9, C20 PUFA/C22 PUFA is 11.94, and n-6 PUFA/n-3 PUFA is 2.67.

In these results, arachidonic acid increased approximately 10-fold, EPA approximately 8-fold, and DGLA approximately 16-fold, while DPA decreased to approximately ¼ and DHA to approximately ⅕.

By selecting the labyrinthulid Parietichytrium sarkarianum SEK364 having no PUFA-PKS pathway in this manner, a strain that accumulates PUFAs other than DHA and n-6 DPA can be produced without PUFA-PKS pathway gene disruption. This strain may also be used as a strain that produces EPA and/or ARA, and further disruption or transforming elongase or desaturase genes can create strains that produce desired PUFAs.

### Example 3

[Measurement of Fatty Acid Composition of Lipids Produced by Δ4 Desaturase Gene Disruption and Transformation Strain of Parietichytrium sarkarianum SEK364]

### [Example 3-1]: Cloning of Genus Parietichytrium Δ4 Desaturase Gene

Genome DNA of Parietichytrium sp. SEK571 was extracted by the method described in Example 2-2. Using the extracted genome DNA as a template, a sequence containing the Δ4 desaturase gene sequence (5003 bp, SEQ ID NO: 39) was amplified with LA Taq Hot Start Version (available from Takara Bio Inc.). The PCR primers used were as shown below. [RHO241: 23mer: 5′-GTT TGA GGA GCG AGG CAT TTC TT-3′ (SEQ ID NO: 40), RHO242: 23mer: 5′-AGT GCT CGT ACA ATG ACT GGC GT-3′ (SEQ ID NO: 41)].

The obtained DNA fragment was cloned in pGEM-T Easy Vector, and after amplification with E. coli, the sequence was confirmed using a Dye Terminator Cycle Sequencing Kit (available from Beckman Coulter Inc.). This was named pRH112 (SEQ ID NO: 42).

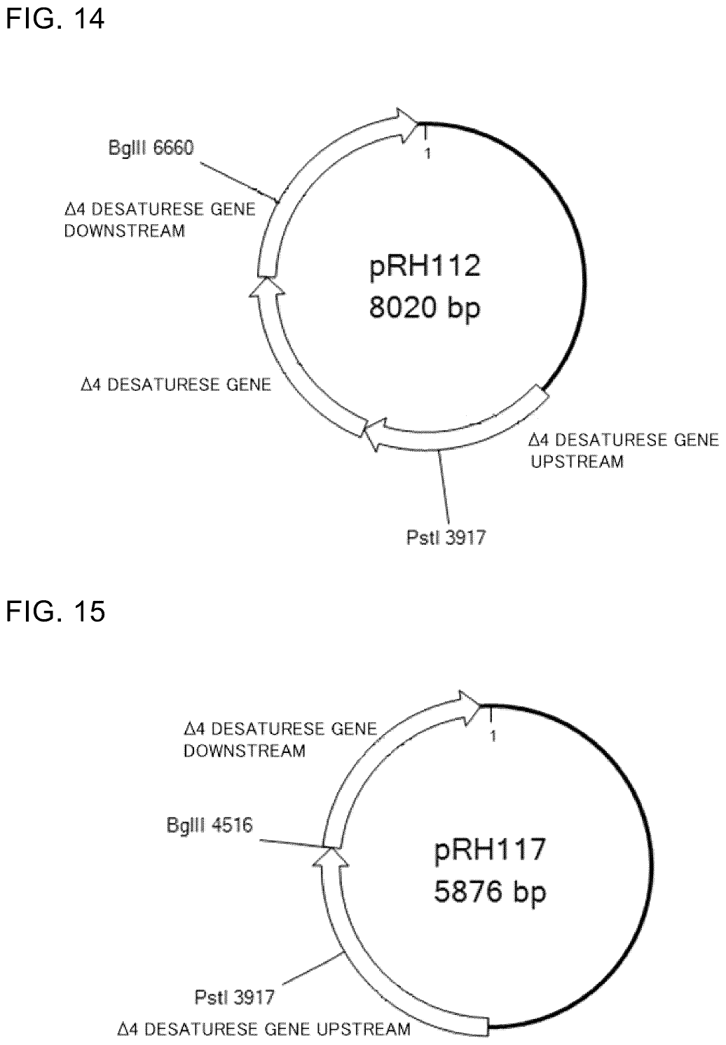

The plasmid (pRH112) containing the genus of Parietichytrium Δ4 desaturase gene sequence (1542 bp, SEQ ID NO: 43) is illustrated in FIG. 14.

### [Example 3-2]: Production of Plasmid Serving as Base for Production of Δ4 Desaturase Gene Targeting Vector

Using pRH112 (FIG. 14) produced in Example 3-1 as a template, a primer set designed so as to delete the Δ4 desaturase gene and 600 bp downstream of the Δ4 desaturase gene and to produce a BglII site in the deleted portion was prepared. [RHO243: 26mer: 5′-GGC AAG ATC TAA CTT TCT GAG GCT CT-3′ (SEQ ID NO: 44), RH0244: 26mer: 5′-AAG TTA GAT CTT GCC TAT TCC ACG AT-3′ (SEQ ID NO: 45)]. PrimeSTAR Max DNA Polymerase (available from Takara Bio Inc.) was used in amplification. After the amplified sample was digested with BglII, the resultant was self-ligated. After the ligated sample was amplified with E. coli, the sequence was confirmed using a Dye Terminator Cycle Sequencing Kit (available from Beckman Coulter Inc.). This was named pRH117.

The produced plasmid (pRH117) serving as a base for production of a Δ4 desaturase gene targeting vector is illustrated in FIG. 15.

### [Example 3-3]: Production of Δ4 Desaturase Gene Targeting Vector

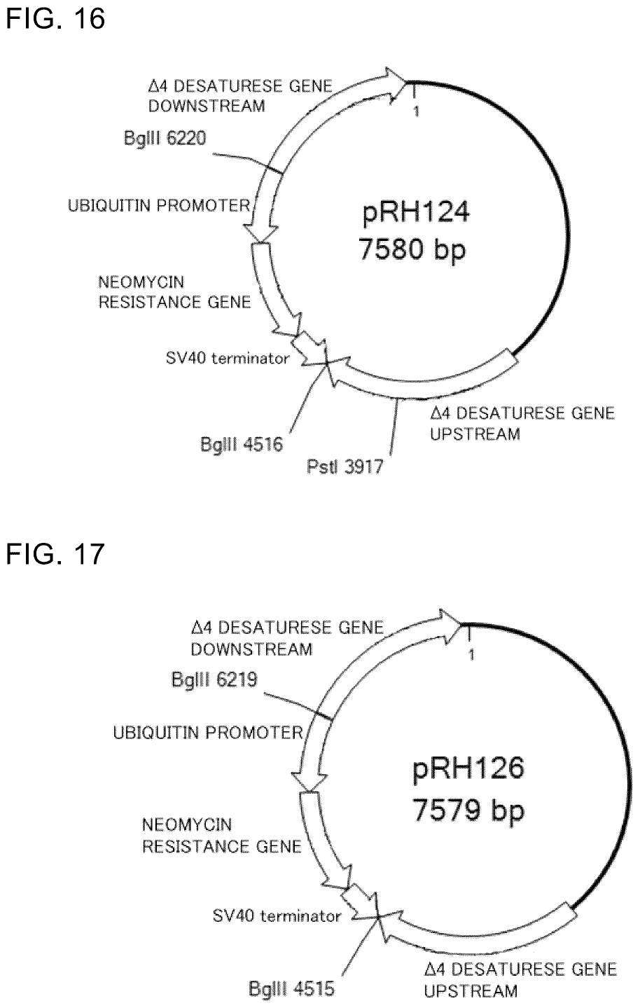

pRH31 (FIG. 4) described in Example 2-2 was digested with BglII, and a DNA fragment containing an artificially synthesized neomycin resistance gene cassette was bound to the BglII site of pRH117 (FIG. 15) described in Example 3-2. This was named pRH124 (FIG. 16). Using this pRH124 as a template, a primer set that was set up so as to delete PstI was prepared. [RHO261: 26mer: 5′-GTG CAG ACG CAG AAG AAG ACT GAC AA-3′ (SEQ ID NO: 46), RHO262: 25mer: 5′-CTT CTG CGT CTG CAC GAG GAA TCG A-3′ (SEQ ID NO: 47)]. PrimeSTAR Max DNA Polymerase (available from Takara Bio Inc.) was used in amplification. After transforming the PCR product into E. coli and amplifying, the sequence was confirmed using a Dye Terminator Cycle Sequencing Kit (available from Beckman Coulter Inc.). This was named pRH126 (SEQ ID NO: 48).

The produced Δ4 desaturase gene targeting vector (pRH126) is illustrated in FIG. 17.

### [Example 3-4]: Transfer of Δ4 Desaturase Gene Targeting Vector to Parietichytrium sarkarianum SEK364

Using the targeting vector pRH126 (FIG. 17) produced in Example 3-3 as a template, the gene was amplified with PrimeSTAR Max DNA Polymerase (available from Takara Bio Inc.) using RHO241 (described in Example 3-1, SEQ ID NO: 40) and RHO242 (described in Example 3-1, SEQ ID NO: 41) as primers. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH126 (FIG. 17) described in Example 3-3 was used as a template was 4562 bp.

The Parietichytrium sarkarianum SEK364 strain was cultured for 1 to 2 days in a GY culture medium, and cells in the logarithmic growth phase were used for gene transfer. To cells corresponding to OD600=1 to 2, 0.625 μg of DNA fragment was transformed by the gene gun method (microcarrier: 0.6 micron gold particles, target distance: 6 cm, chamber vacuum: 26 mmHg, rupture disk: 1550 psi). After a recovery time of 24 hr, the transgenic cells were spread on a PDA agar plate culture medium containing 1 mg/mL of G418. As a result, from 0 to 2 cells of drug resistant strain per shot were obtained.

### [Example 3-5]: Identification of Δ4 Desaturase Gene Targeting Homologous Recombinant

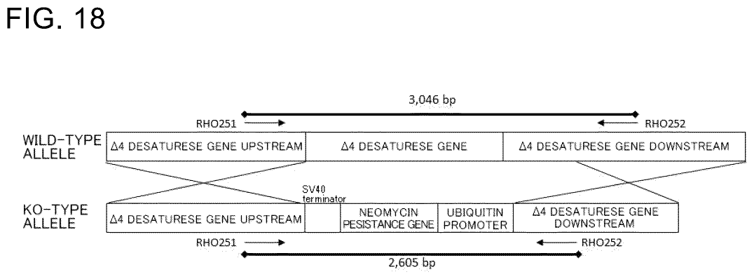

Genome DNA of the Parietichytrium sarkarianum SEK364 strain and the Δ4 desaturase gene disruption strain were extracted by the method described in Example 2-2, and then A260/280 was measured and the DNA concentration was calculated. Using the genome DNA as templates, PCR for genome structure confirmation was performed using PrimeSTAR GXL DNA Polymerase (available from Takara Bio Inc.). The positions of the primers used, the combinations used in amplification, and the expected sizes of the amplification products are illustrated in FIG. 18 (within homologous region). In the primer set designed within the homologous recombination region, 3046 bp was amplified in the Parietichytrium sarkarianum SEK364 strain, and 2605 bp was amplified in the Δ4 desaturase gene disruption strain. [RH0251: 20mer: 5′-GTG GTC GAA GTG GAG TAT CT-3′ (SEQ ID NO: 49), RH0252: 20mer: 5′-ACT CGC CAT ACA ACT TTA CA-3′ (SEQ ID NO: 50)].

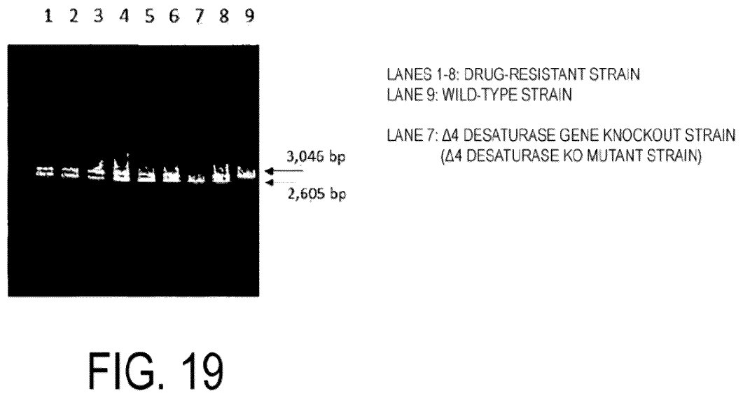

As a result, a Δ4 desaturase gene disruption strain in which there is no amplification derived from the wild-type allele (Wt allele) and there is amplification derived from the Δ4 desaturase gene KO allele (NeoR allele) was obtained (FIG. 19 lane 7: Δ4 desaturase KO mutant strain).

### Example 3-6: Change in Fatty Acid Composition by Δ4 Desaturase Gene Disruption

The Parietichytrium sarkarianum SEK364 wild-type strain and the Δ4 desaturase gene disruption strain thereof were cultured according to the method described in Example 2-9, and after freeze drying, the fatty acids were methyl-esterified and analyzed using GC. In culturing, the GY liquid culture medium described in Example 1 supplemented with 0.1% of a vitamin solution (vitamin B₁ 200 mg, vitamin B₂ 1 mg, and vitamin B₁₂ 1 mg are dissolved in 100 mL of distilled water) and 0.2% of a trace element solution (EDTA disodium salt 30.0 g, FeCl₃·6H₂O 1.45 g, H₂BO₃ 34.2 g, MnCl₂·4H₂O 4.3 g, ZnCl₂ 1.335 g, CoCl₂·6H₂O 0.13 g, NiSO₄·6H₂O 0.26 g, CuSO₄·5H₂O 0.01 g, and NaMoO₄·2H₂O 0.025 g are dissolved in 1 L of distilled water) was used. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(2° C./min)→220° C. (10 min); carrier gas: He (1.3 mL/min).

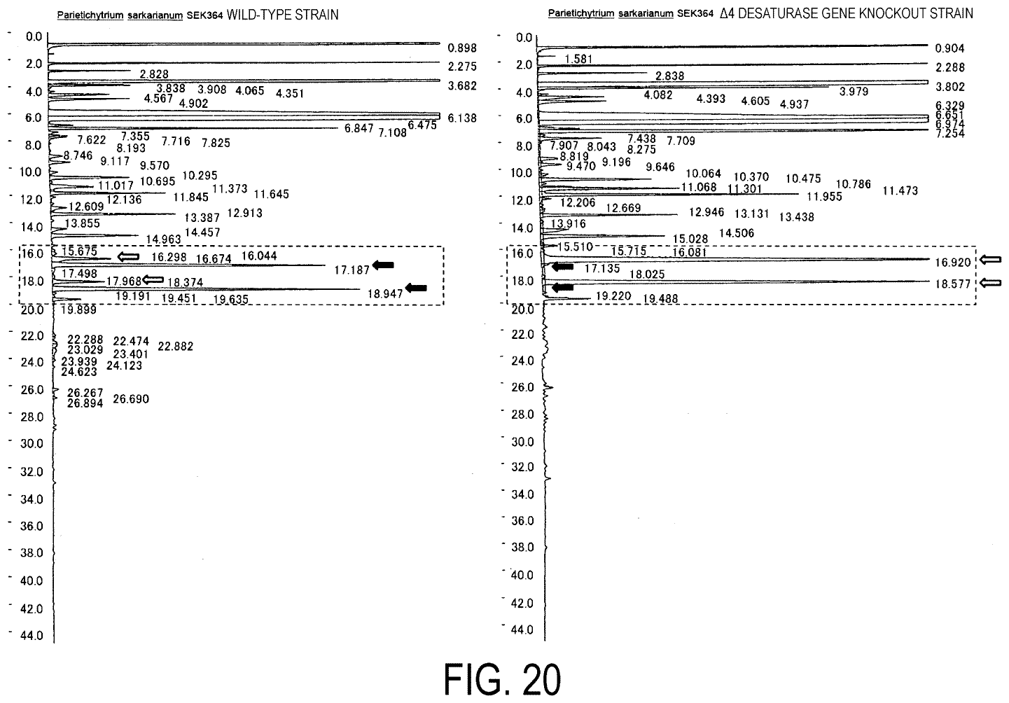

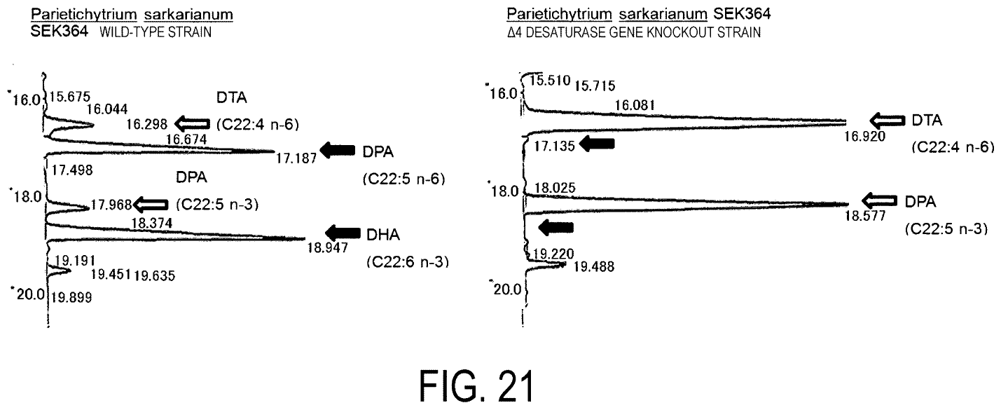

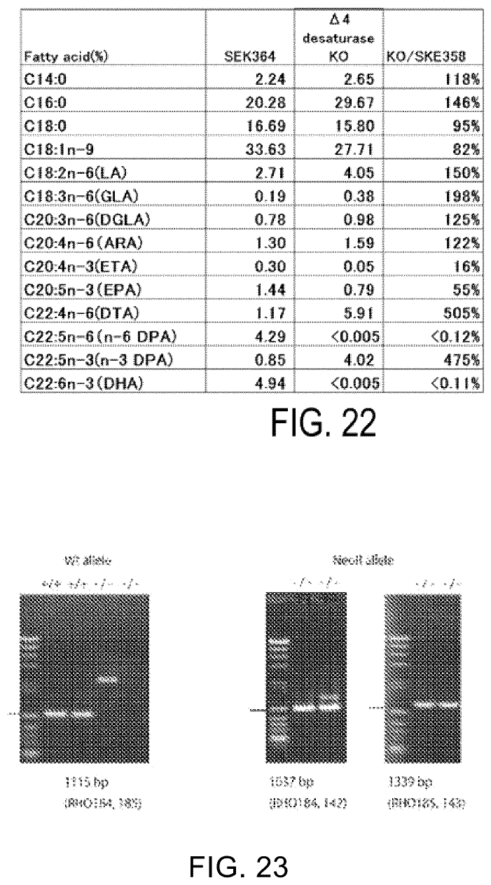

The analysis results chart is shown in FIG. 20, and a partial enlarged diagram thereof is shown in FIG. 21. FIG. 22 shows a quantification of the chart of FIG. 20. This table shows a comparison of the fatty acid compositions of the Parietichytrium sarkarianum SEK364 wild-type strain and the Δ4 desaturase gene disruption strain thereof. This table is the quantification of the chart of FIG. 20. The table shows that, of the total fatty acid composition, ARA is 1.59%, DGLA is 0.98%, ETA is 0.05%, EPA is 0.79%, n-6 DPA is 0.00%, and DHA is 0.00%. The table shows that, by GC area, LA/EPA is 5.09, GLA/EPA is 0.48, DTA/EPA is 7.44, DTA/ARA is 3.73, DTA/DGLA is 6.06, DGLA/LA is 0.24, ARA/LA is 0.39, EPA/LA is 0.20, DTA/LA is 1.46, DGLA/GLA is 2.57, ARA/GLA is 4.19, and n-6 DPA/DTA is 0.00.

These results show that when the Δ4 desaturase gene is disrupted in the Parietichytrium sarkarianum SEK364 strain, DHA and DPA n-6 cannot be substantially biosynthesized, and conversely, DPA n-3 and DTA, which are substrates thereof, increase.

By selecting the labyrinthulid Parietichytrium sarkarianum SEK364 having no PUFA-PKS pathway in this manner, a strain that accumulates PUFAs other than DHA and DPA n-6 can be produced without PUFA-PKS pathway gene disruption. This strain may also be used as a strain that produces n-3 DPA and/or DTA, and further disruption or transforming elongase or desaturase genes can create strains that produce desired PUFAs.

### Example 4

[Measurement of Fatty Acid Composition of Lipids Produced by C20 Elongase Gene Disruption and Transformation Strain of Parietichytrium sp. SEK358]

### [Example 4-1]: Transfer of C20 Elongase Gene Targeting Vector to Parietichytrium sp. SEK358 Strain

Using the targeting vector produced with pRH85 (FIG. 9) described in Example 2-6 as a template, the gene was amplified with PrimeSTAR Max DNA Polymerase (available from Takara Bio Inc.) using RHO153 (described in Example 2-4, SEQ ID NO: 24) and RHO154 (described in Example 2-4, SEQ ID NO: 25) as primers. [PCR cycles: 98° C. 2 min/98° C. 30 sec, 68° 2 min, 30 cycles/68° C. 2 min]. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH85 (FIG. 9) described in Example 2-6 was used as a template was 2661 bp, and resulted in a sequence composed of genus Parietichytrium C20 elongase gene front half—SV40 terminator sequence—artificially synthesized neomycin resistance gene sequence—ubiquitin promoter sequence—genus Parietichytrium C20 elongase gene back half (described in Example 2-7, SEQ ID NO: 31).

The Parietichytrium sp. SEK358 strain was cultured for 3 days in a GY culture medium, and cells in the logarithmic growth phase were used for gene transfer. To cells corresponding to OD600=1 to 1.5, 0.625 μg of DNA fragment was transformed by the gene gun method (microcarrier: 0.6 micron gold particles, target distance: 6 cm, chamber vacuum: 26 mmHg, rupture disk: 900 psi). After a recovery time of 24 hr, the transgenic cells were spread on a PDA agar plate culture medium containing 0.5 mg/mL of G418. As a result, from 10 to 30 cells of drug resistant strain per shot were obtained.

### [Example 4-2]: Identification of C20 Elongase Gene Targeting Homologous Recombinant

Genome DNA of the Parietichytrium sp. SEK358 strain and the C20 elongase gene disruption strain were extracted by the method described in Example 2-2, and then A260/280 was measured and the DNA concentration was calculated. Using the genome DNA as templates, PCR for genome structure confirmation was performed using Mighty Amp DNA Polymerase (available from Takara Bio Inc.). The positions of the primers used, the combinations used in amplification, and the expected sizes of the amplification products are illustrated in FIG. 10 described in Example 2-8.

RHO184 (described in Example 2-8, SEQ ID NO: 37) was set upstream of C20 elongase; RHO185 (described in Example 2-8, SEQ ID NO: 38) was set downstream; RHO142 (described in Example 2-8, SEQ ID NO: 35) and RHO143 (described in Example 2-8, SEQ ID NO: 36) were set on the artificially synthesized neomycin resistance gene. [PCR cycles: 98° C. 2 min/98° C. 10 sec, 68° 2 min, 30 cycles/68° C. 7 min].

A C20 elongase gene disruption strain in which there is no amplification in the wild-type allele (Wt allele) and there is amplification in the artificially synthesized neomycin resistance gene allele (NeoR allele) was obtained (FIG. 23).

### [Example 4-3]: Change in Fatty Acid Composition by C20 Elongase Gene Disruption

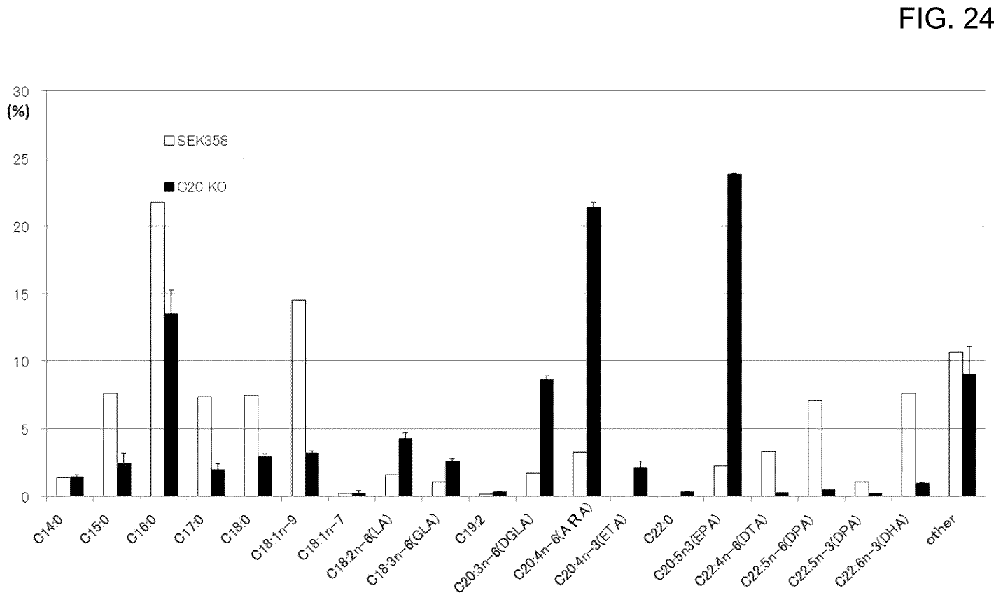

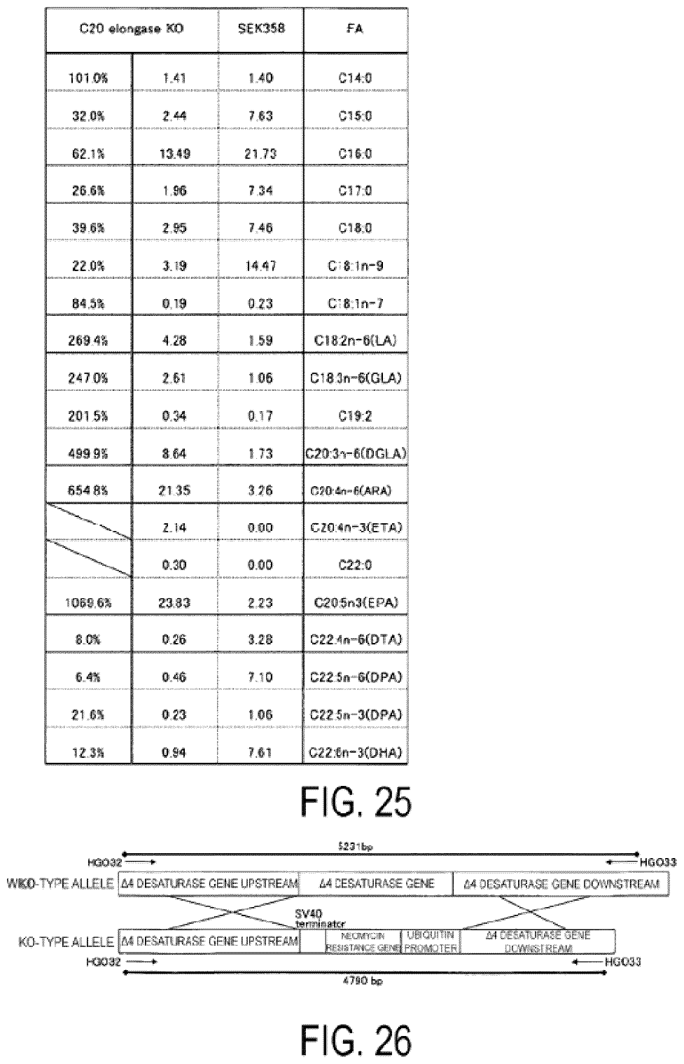

The Parietichytrium sp. SEK358 wild-type strain and the gene disruption strain thereof (C20 elongase gene disruption strain, C20 KO) were cultured according to the method described in Example 2-9, and after freeze drying, the fatty acids were methyl-esterified and analyzed using GC. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min). The changes in the fatty acid composition are shown in FIG. 24. Furthermore, FIG. 25 shows the proportion when the wild-type strain is taken as 100%.

FIG. 25 shows that, of the total fatty acid composition, ARA is 21.35%, DGLA is 8.64%, ETA is 2.14%, EPA is 23.83%, n-6 DPA is 0.46%, and DHA is 0.94%. FIG. 25 shows that, by GC area, LA/DHA is 4.6, GLA/DHA is 2.8, DGLA/DHA is 9.19, ARA/DHA is 22.7, EPA/DHA is 25.4, LA/EPA is 0.18, GLA/EPA is 0.11, DTA/EPA is 0.01, DTA/ARA is 0.01, DTA/DGLA is 0.03, LA/n-6 DPA is 9.3, GLA/n-6 DPA is 5.7, DGLA/n-6 DPA is 18.8, R/n-6 DPA is 46.4, EPA/n-6 DPA is 51.8, DGLA/LA is 2.0, ARA/LA is 5.0, EPA/LA is 5.6, DTA/LA is 0.06, DGLA/GLA is 3.3, ARA/GLA is 8.2, n-6 DPA/DTA is 1.8, DHA/n-3 DPA is 4.1, C20 PUFA/C22 PUFA is 29.61, and n-6 PUFA/n-3 PUFA is 1.1.

As a result, when the C20 elongase gene was disrupted in the Parietichytrium sp. SEK358 strain, fatty acids having not less than 22 carbon chains decreased while fatty acids having 20 carbon chains increased. Specifically, arachidonic acid increased approximately 7-fold and EPA increased approximately 11-fold, while DPA decreased to approximately 1/15 and DHA decreased to approximately ⅛.

By selecting the labyrinthulid Parietichytrium sp. SEK358 having no PUFA-PKS pathway in this manner, a strain that accumulates PUFAs other than DHA and DPA n-6 can be produced without PUFA-PKS pathway gene disruption. This strain may also be used as a strain that produces EPA and/or ARA, and further disruption or transforming elongase or desaturase genes can create strains that produce desired PUFAs.

### Example 5

[Measurement of Fatty Acid Composition of Lipids Produced by Δ4 Desaturase Gene Disruption and Transformation Strain of Parietichytrium sp. SEK358]

### [Example 5-1]: Transfer of Δ4 Desaturase Gene Targeting Vector to Parietichytrium sp. SEK358 Strain

Using the targeting vector produced with pRH126 (FIG. 17) described in Example 3-3 as a template, the gene was amplified with PrimeSTAR Max DNA Polymerase (available from Takara Bio Inc.) using RH0241 (described in Example 3-1, SEQ ID NO: 40) and RH0242 (described in Example 3-1, SEQ ID NO: 41) as primers. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH126 (FIG. 17) described in Example 3-3 was used as a template was 4562 bp.

The Parietichytrium sp. SEK358 strain was cultured for 1 to 2 days in a GY culture medium, and cells in the logarithmic growth phase were used in gene transfer. To cells corresponding to OD600=1 to 2, 0.625 μg of DNA fragment was transformed by the gene gun method (microcarrier: 0.6 micron gold particles, target distance: 6 cm, chamber vacuum: 26 mmHg, rupture disk: 1550 psi). After a recovery time of 24 hr, the transgenic cells were spread on a PDA agar plate culture medium containing 1 mg/mL of G418. As a result, from 0 to 2 cells of drug resistant strain per shot were obtained.

### [Example 5-2]: Identification of Δ4 Desaturase Gene Targeting Homologous Recombinant

Genome DNA from the Parietichytrium sp. SEK358 strain and the Δ4 desaturase gene disruption strain were extracted by the method described in Example 2-2, and then A260/280 was measured and the DNA concentration was calculated. Using the genome DNA as templates, PCR for genome structure confirmation was performed using PrimeSTAR GXL DNA Polymerase (available from Takara Bio Inc.). The positions of the primers used, the combinations used in amplification, and the expected sizes of the amplification products are illustrated in FIG. 18 (within homologous region) and FIG. 26 (outside homologous region). In the primer set designed within the homologous recombination region, 3046 bp was amplified in the Parietichytrium sp. SEK358 strain, and 2605 bp was amplified in the Δ4 desaturase gene disruption strain. [RHO251: 20mer: 5′-GTG GTC GAA GTG GAG TAT CT-3′ (SEQ ID NO: 49), RHO252: 20mer: 5′-ACT CGC CAT ACA ACT TTA CA-3′ (SEQ ID NO: 50)]. In the primer set designed outside the homologous recombination region, 5231 bp was amplified in the Parietichytrium sp. SEK358 strain, and 4790 bp was amplified in the Δ4 desaturase gene disruption strain. [HGO32: 25mer: 5′-CGG AGC TCG GAG AAC AAC ATA GAA G-3′ (SEQ ID NO: 51), HGO33: 23mer: 5′-GTG CAA CCA GGT GGC AAG ATT GT-3′ (SEQ ID NO: 52)].

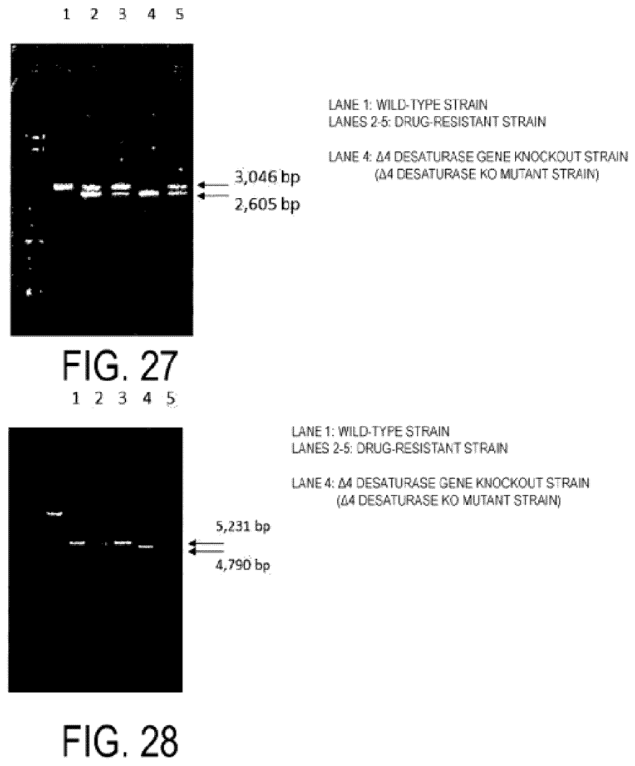

As a result, a Δ4 desaturase gene disruption strain in which there is no amplification derived from the wild-type allele (Wt allele) and there is amplification derived from the Δ4 desaturase gene KO allele (NeoR allele) was obtained (FIG. 27 lane 4, FIG. 28 lane 4: Δ4 desaturase KO mutant strain).

### [Example 5-3]: Change in Fatty Acid Composition by Δ4 Desaturase Gene Disruption

The Parietichytrium sp. SEK358 wild-type strain and the Δ4 desaturase gene disruption strain thereof (SEK358 delta4 des. KO mutant strain) were cultured according to the method described in Example 2-9, and after freeze drying, the fatty acids were methyl-esterified and analyzed using GC. In culturing, the GY liquid culture medium described in Example 1 supplemented with 0.1% of a vitamin solution (vitamin B₁ 200 mg, vitamin B₂ 1 mg, and vitamin B₁₂ 1 mg are dissolved in 100 mL of distilled water) and 0.2% of a trace element solution (EDTA disodium salt 30.0 g, FeCl₃·6H₂O 1.45 g, H₂BO₃ 34.2 g, MnCl₂·4H₂O 4.3 g, ZnCl₂ 1.335 g, CoCl₂·6H₂O 0.13 g, NiSO₄·6·H₂O 0.26 g, CuSO₄·5H₂O 0.01 g, and NaMoO₄·2H₂O 0.025 g are dissolved in 1 L of distilled water) was used. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(2° C./min)→220° C. (10 min); carrier gas: He (1.3 mL/min).

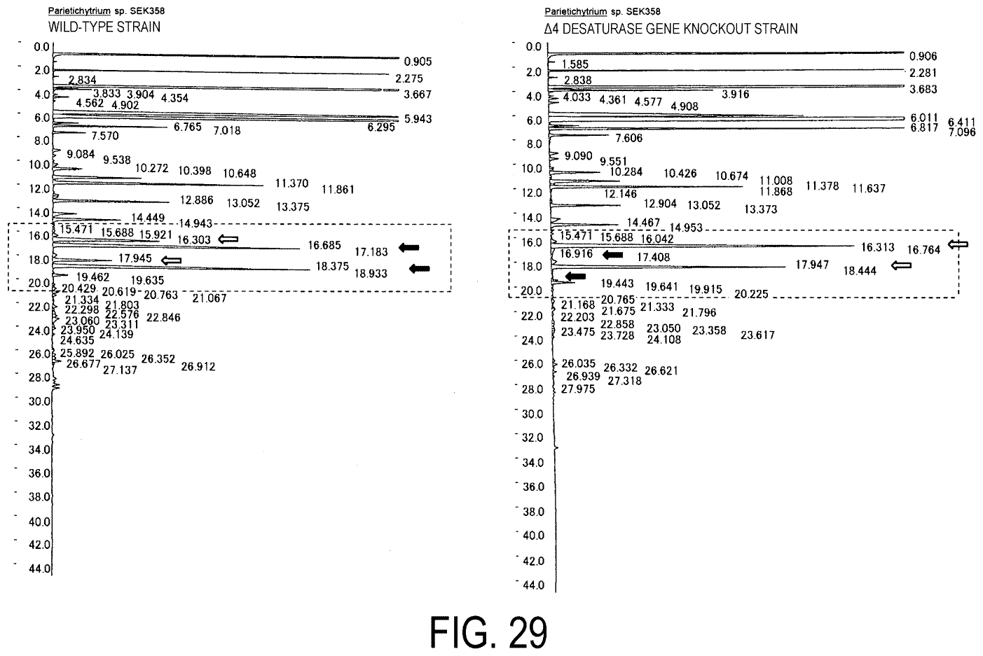

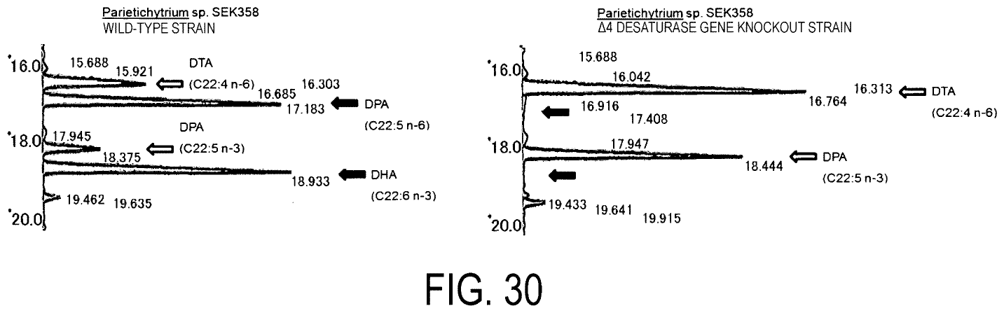

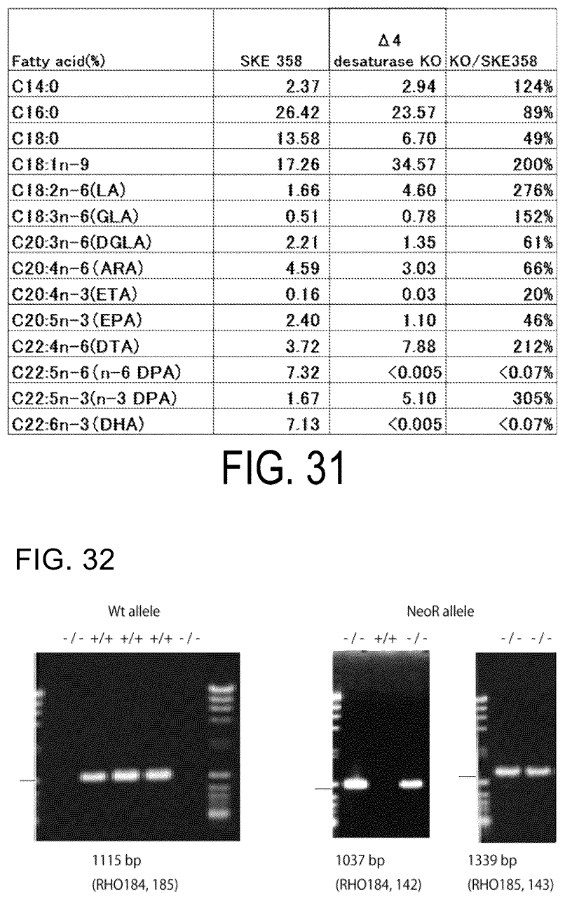

The analysis results chart is shown in FIG. 29, and a partial enlarged diagram of the chart is shown in FIG. 30. The table in FIG. 31 shows a quantification of the chart of FIG. 29. This table shows a comparison of the fatty acid compositions of the Parietichytrium sp. SEK358 wild-type strain and the Δ4 desaturase gene disruption strain thereof. The table shows that, of the total fatty acid composition, ARA is 3.03%, DGLA is 1.35%, ETA is 0.03%, EPA is 1.10%, n-6 DPA is 0.00%, and DHA is 0.00%. FIG. 31 shows that, by GC area, LA/EPA is 4.2, GLA/EPA is 0.71, DTA/EPA is 7.19, DTA/ARA is 2.60, DTA/DGLA is 5.85, DGLA/LA is 0.29, ARA/LA is 0.66, EPA/LA is 0.24, DTA/LA is 1.71, DGLA/GLA is 1.72, ARA/GLA is 3.87, C20 PUFA/C22 PUFA is 0.42, and n-6 PUFA/n-3 PUFA is 2.0.

The results showed that when the Δ4 desaturase gene was disrupted in the Parietichytrium sp. SEK358 strain, DHA and DPA n-6 cannot be substantially biosynthesized, and conversely, n-3 DPA and DTA, which are substrates thereof, increase.

By selecting the labyrinthulid Parietichytrium sp. SEK358 having no PUFA-PKS pathway in this manner, a strain that accumulates PUFAs other than DHA and n-6 DPA can be produced without PUFA-PKS pathway gene disruption. This strain may also be used as a strain that produces n-3 DPA and/or DTA, and further disruption or transforming elongase or desaturase genes can create strains that produce desired PUFAs.

### Example 6

[Measurement of Fatty Acid Composition of Lipids Produced by C20 Elongase Gene Disruption and Transformation Strain of Parietichytrium sp. SEK571]

### [Example 6-1]: Transfer of C20 Elongase Gene Targeting Vector to Parietichytrium sp. SEK571 Strain

Using the targeting vector produced with pRH85 (FIG. 9) described in Example 2-6 as a template, the gene was amplified with PrimeSTAR Max DNA Polymerase (available from Takara Bio Inc.) using RHO153 (described in Example 2-4, SEQ ID NO: 24) and RHO154 (described in Example 2-4, SEQ ID NO: 25) as primers. [PCR cycles: 98° C. 2 min/98° C. 30 sec, 68° C. 2 min, 30 cycles/68° C. 2 min]. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH85 (FIG. 9) described in Example 2-6 was used as a template was 2661 bp, and resulted in a sequence including genus Parietichytrium C20 elongase gene front half—SV40 terminator sequence—artificially synthesized neomycin resistance gene sequence—ubiquitin promoter sequence—genus Parietichytrium C20 elongase gene back half (described in Example 2-7, SEQ ID NO: 31).

The Parietichytrium sp. SEK571 strain was cultured for 3 days in a GY culture medium, and cells in the logarithmic growth phase were used in gene transfer. To cells corresponding to OD600=1 to 1.5, 0.625 μg of DNA fragment was transformed by the gene gun method (microcarrier: 0.6 micron gold particles, target distance: 6 cm, chamber vacuum: 26 mmHg, rupture disk: 1550 psi). After a recovery time of 24 hr, the transgenic cells were spread on a PDA agar plate culture medium containing 0.5 mg/mL of G418. As a result, from 5 to 15 cells of drug resistant strain per shot were obtained.

### [Example 6-2]: Identification of C20 Elongase Gene Targeting Homologous Recombinant

Genome DNA of the Parietichytrium sp. SEK571 strain and the C20 elongase gene disruption strain were extracted by the method described in Example 2-2, and then A260/280 was measured and the DNA concentration was calculated. Using the genome DNA as templates, PCR for genome structure confirmation was performed using Mighty Amp DNA Polymerase (available from Takara Bio Inc.). The positions of the primers used, the combinations used in amplification, and the expected sizes of the amplification products are illustrated in FIG. 10 described in Example 2-8.

RHO184 (described in Example 2-8, SEQ ID NO: 37) was set upstream of C20 elongase; RHO185 (described in Example 2-8, SEQ ID NO: 38) was set downstream; RHO142 (described in Example 2-8, SEQ ID NO: 35) and RHO143 (described in Example 2-8, SEQ ID NO: 36) were set on the artificially synthesized neomycin resistance gene. [PCR cycles: 98° C. 2 min/98° C. 10 sec, 68° 2 min, 30 cycles/68° C. 7 min].

A C20 elongase gene disruption strain in which there is no amplification in the wild-type allele (Wt allele) and there is amplification in the artificially synthesized neomycin resistance gene allele (NeoR allele) was obtained (FIG. 32).

### [Example 6-3]: Change in Fatty Acid Composition by C20 Elongase Gene Disruption

The Parietichytrium sp. SEK571 strain and the gene disruption strain thereof (C20 elongase gene disruption strain, C20 KO) were cultured according to the method described in Example 2-9, and after freeze drying, the fatty acids were methyl-esterified and analyzed using GC. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min).

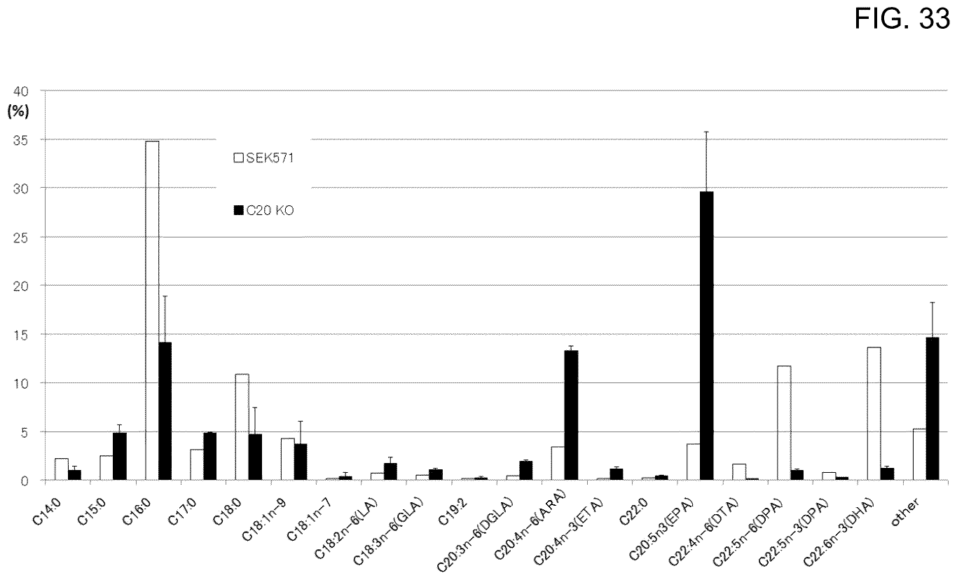

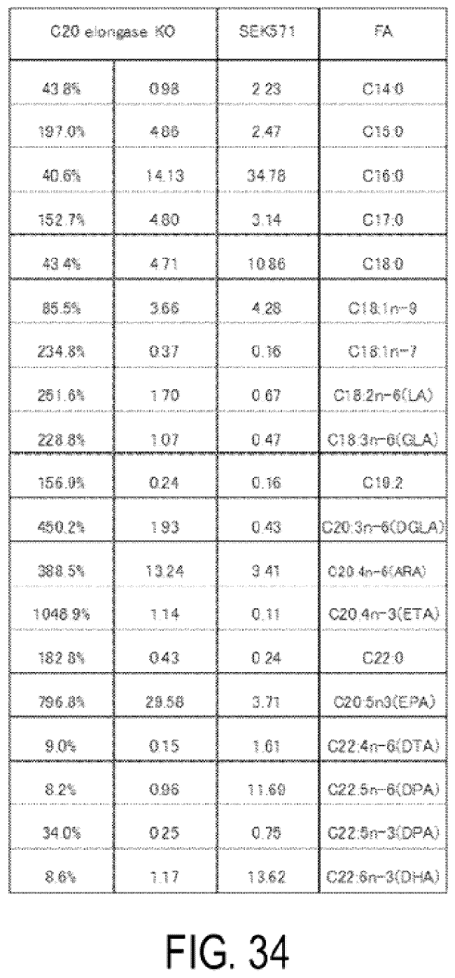

The changes in the fatty acid composition are shown in FIG. 33. Furthermore, FIG. 34 shows the proportion when the wild-type strain is taken as 100%.

FIG. 34 shows that, of the total fatty acid composition, ARA is 13.24%, DGLA is 1.93%, ETA is 1.14%, EPA is 29.58%, n-6 DPA is 0.96%, and DHA is 1.17%. FIG. 34 shows that, by GC area, LA/DHA is 1.5, GLA/DHA is 0.9, DGLA/DHA is 1.65, ARA/DHA is 11.3, EPA/DHA is 25.3, LA/EPA is 0.06, GLA/EPA is 0.04, DTA/EPA is 0.01, DTA/ARA is 0.01, DTA/DGLA is 0.08, LA/n-6 DPA is 1.8, GLA/n-6 DPA is 1.1, DGLA/n-6 DPA is 2.0, ARA/n-6 DPA is 13.8, EPA/n-6 DPA is 30.8, DGLA/LA is 1.1, ARA/LA is 7.8, EPA/LA is 17.4, DTA/LA is 0.09, DGLA/GLA is 1.8, ARA/GLA is 12.4, n-6 DPA/DTA is 6.4, DHA/n-3 DPA is 4.7, C20 PUFA/C22 PUFA is 18.1, and n-6 PUFA/n-3 PUFA is 0.51.

As a result, when the C20 elongase gene was disrupted in the Parietichytrium sp. SEK571 strain, fatty acids having not less than 22 carbon chains decreased while fatty acids having 20 carbon chains increased. Specifically, arachidonic acid increased approximately 4-fold and EPA increased approximately 8-fold, while DPA decreased to approximately 1/12 and DHA decreased to approximately 1/12.

The results show that Parietichytrium sp. SEK571 has very weak or no PUFA production activity via the PUFA-PKS pathway, and by selecting the labyrinthulid Parietichytrium sp. SEK571, a strain that accumulates PUFAs other than DHA and n-6 DPA can be produced without PUFA-PKS pathway gene disruption. This strain may also be used as a strain that produces EPA and/or ARA, and further disruption or transforming elongase or desaturase genes can create strains that produce desired PUFAs.

### Comparative Example 1

[Measurement of Fatty Acid Composition of Lipids Produced by C20 Elongase Gene Disruption and Transformation Strain of Thraustochytrium aureum ATCC 34304]

### [Comparative Example 1-1]: Extraction of Total RNA Derived from T. aureum ATCC 34304, and mRNA Purification

A T. aureum ATCC 34304 culture solution on the third day of culturing using a GY liquid culture medium was centrifuged for 15 min at 3500×g, and the cells were collected. The obtained cells were washed by suspending in a sterilized physiological saline solution and then centrifuging again, and were then rapidly frozen with liquid nitrogen, and then ground into powder form in a mortar. Total RNA was extracted from the obtained crushed cell solution using Sepasol-RNA I Super (available from Nacalai Tesque, Inc.). Then, mRNA was purified from the total RNA according to manufacturer's instructions using Oligotex-dT30 <Super> mRNA Purification Kit (trade name; available from Takara Bio Inc.). The obtained total RNA and mRNA were dissolved in an appropriate amount of TE, and then subjected to electrophoresis using formalin-modified gel (1% agarose/MOPS buffer). The result showed that total RNA extraction was successful, that mRNA was purified from the total RNA, and that the RNA was not decomposed by RNase. Furthermore, to proactively avoid RNA decomposition, rubber gloves, a mask, and the like were donned through the experimental operation, and the instruments used were completely RNase-free or the RNase used was inactivated by treatment with diethylpyrocarbonate (available from Nacalai Tesque, Inc.). Furthermore, when decomposing RNA, a solution obtained by adding the recombinant RNase inhibitor RNaseOUT (trade name; available from Invitrogen Corp.) to sterilized MilliQ water treated with diethylpyrocarbonate was used.

### [Comparative Example 1-2]: Isolation of T. aureum ATCC 34304-Derived Elongase Gene by RACE

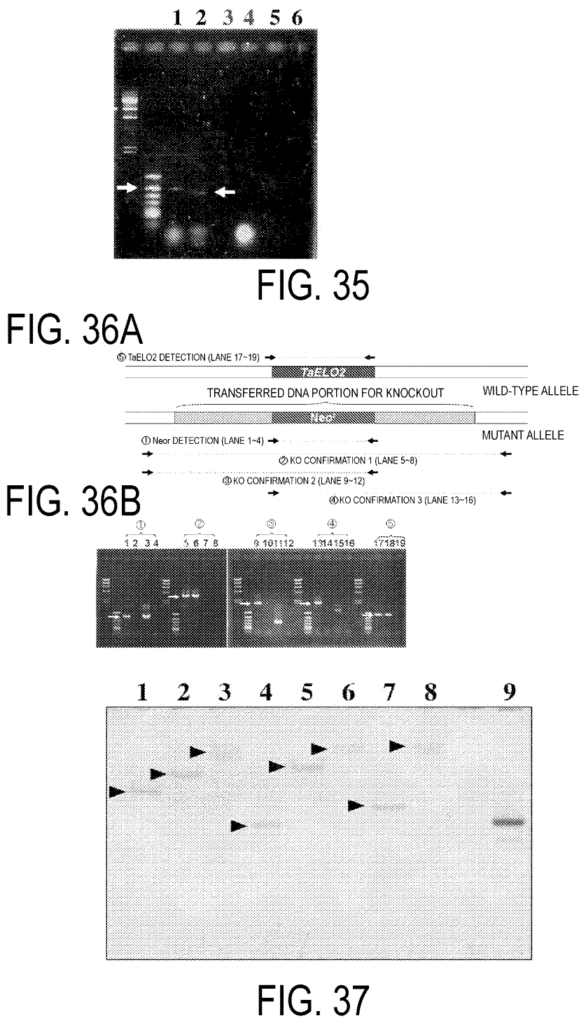

Using a histidine box (His box) in which the elongase gene was conserved to a high degree as a target, forward (elo-F; 5′-TTY YTN CAY GTN TAY CAY CAY-3′) (SEQ ID NO: 53) and reverse (elo-R; 5′-GCR TGR TGR TAN ACR TGN ARR AA-3′) (SEQ ID NO: 54) degenerate oligonucleotides were synthesized. The oligonucleotides were synthesized using a DNA synthesizer (available from Applied Biosystems Corp.). Next, 3′- and 5′-RACE cDNA libraries in which synthetic adapters were appended to the 3′ and 5′ terminals were produced according to manufacturer's instructions using SMART RACE cDNA Amplification Kit (trade name; available from Clontech Laboratories, Inc.). Using these as templates, 3′- and 5′-RACE were performed using the synthetic adapter-specific oligonucleotides and the above degenerate oligonucleotides elo-F and elo-R. [PCR cycles: 94° C. 1 min/94° C. 30 sec, 60° C. 30 sec, 72° C. 3 min, 30 cycles/72° C. 10 min/4° C. ∞]. As a result, bands of specifically amplified 3′- and 5′-RACE products were confirmed (FIG. 35). Next, the entire amounts of the RACE products were subjected to electrophoresis using 1% agarose gel, the separated DNA fragments were cut with a clean cutter or the like, and DNA fragments were extracted according to the method described in Non-patent Document 10. Then, TA cloning of the DNA fragments was performed using pGEM-T Easy Vector (available from Promega Corporation), and the base sequences thereof were determined according to the method of Sanger et al. (Non-patent Document 11). Specifically, using BigDye (trade name) Terminator v3.1 Cycle Sequencing Kit and 3130 Genetic Analyzer (available from Applied Biosystems Corp.), the base sequence was determined by the dye terminator method according to manufacturer's instructions.

As a result, two respective sequences of 190 bp and 210 bp named elo1 (SEQ ID NO: 55) and elo2 (SEQ ID NO: 56) were successfully identified in the 3′-RACE product, and one 200 bp sequence named elo3 (SEQ ID NO: 57) was successfully identified in the 5′-RACE product. The fact that these elo1, elo2, and elo3 sequences exhibited significant homology to various elongase gene sequences shows that these sequences are partial sequences of elongase genes derived from T. aureum ATCC 34304. Additionally, respective oligonucleotide primers were again designed for elo1, elo2, and elo3, and acquisition of cDNA sequences was attempted by RACE. The produced oligonucleotide primers are shown below. elo1 forward oligonucleotide primer (elo1-F1; 5′-TAT GAT CGC CAA GTA CGC CCC-3′) (SEQ ID NO: 58) and reverse oligonucleotide primer (elo1-R1; 5′-GAA CTG CGT CAT CTG CAG CGA-3′) (SEQ ID NO: 59), elo2 forward oligonucleotide primer (elo2-F1; 5′-TCT CGC CCT CGA CCA CCA AC-3′) (SEQ ID NO: 60) and reverse oligonucleotide primer (elo2-R1: 5′-CGG TGA CCG AGT TGA GGT AGC C-3′) (SEQ ID NO: 61), elo3 forward oligonucleotide primer (elo3-F1; 5′-CAA CCC TTT CGG CCT CAA CAA G-3′) (SEQ ID NO: 62) and reverse oligonucleotide primer (elo3-R1; 5′-TTC TTG AGG ATC ATC ATG AAC GTG TC-3′) (SEQ ID NO: 63).

Using the produced forward and reverse oligonucleotide primers, RACE and base sequence analysis of the amplified products were performed by the same methods as described above. As a result, for elo1, specifically amplified 3′- and 5′-RACE products were obtained, and since the duplicate portions thereof matched completely, they were proved to be an 1139 bp elo1 cDNA sequence (SEQ ID NO: 64). Similarly, for elo3, specifically amplified 3′- and 5′-RACE products were obtained, and since the duplicate portions thereof matched completely, they were proved to be a 1261 bp elo3 cDNA sequence (SEQ ID NO: 65).

As a result of sequence analysis, it was found that elo1 is composed of an 825 bp translation sequence (SEQ ID NO: 67) that encodes 275 amino acid residues (SEQ ID NO: 66), and as a result of a BLAST search, it was found not only that elo1 exhibited significant homology to various elongase genes, but also that elo1 completely matches a known presumed Δ5 elongase gene sequence derived from T. aureum (NCBI accession no. CS486301). On the other hand, elo3 was estimated to constituted of a 951 bp translation region (SEQ ID NO: 69) that encodes 317 amino acid residues (SEQ ID NO: 68), and as a result of a BLAST search, elo3 was found to exhibit significant homology to various elongase genes, and thus was confirmed to be a presumed elongase gene derived from T. aureum ATCC 34304. Furthermore, a His box in which the elongase gene was conserved to a high degree was found within the presumed amino acid sequences of the two genes. From the above results, the elo1 and elo3 gene were considered to be presumed elongase genes derived from T. aureum ATCC 34304 and were designated as TaELO1 and TaELO2, respectively.

### [Comparative Example 1-3]: Expression of TaELO1 and TaELO2 with Brewer's Yeast Saccharomyces cerevisiae as Host, and Analysis of Fatty Acid Composition of Transgenic Strains

Respective expression vectors were constructed in order to cause expression of TaELO1 and TaELO2 using brewer's yeast S. cerevisiae as a host. An outline thereof is given below. A pair of oligonucleotide primers (E1 HindIII; 5′-ATA AGC TTA AAA TGT CTA GCA ACA TGA GCG CGT GGG GC-3′) (SEQ ID NO: 70) and (E1 XbaI; 5′-TGT CTA GAA CGC GCG GAC GGT CGC GAA A-3′) (SEQ ID NO: 71) were produced based on the sequence of the TaELO1 translation region. E1 HindIII is a forward oligonucleotide primer and has a restriction enzyme HindIII site (AAGCTT) at the 5′ terminal. Furthermore, the sequence near the start codon of TaELO1 has been modified in reference to the yeast consensus sequence ((A/Y) A (A/U) A AUG UCU; underlined portion is start codon) (Non-patent Document 12). E1 XbaI is a reverse oligonucleotide primer, and has an XbaI site (TCTAGA) at the 5′ terminal.

Similarly, a pair of oligonucleotide primers (E2 HindIII; 5′-TAAAGC TTA AAA TGT CTA CGC GCA CCT CGA AGA GCG CTC C-3′) (SEQ ID NO: 72) and (E2 XbaI; 5′-CAT CTA GAC TCG GAC TTG GTG GGG GCG CTT G-3′) (SEQ ID NO: 73) were produced based on the sequence of the TaELO2 translation region. E2 HindIII is a forward oligonucleotide primer and has a restriction enzyme HindIII site at the 5′ terminal. Furthermore, the sequence near the start codon of TaELO2 has been modified in reference to the yeast consensus sequence. E2 XbaI is a reverse oligonucleotide primer, and has an XbaI site at the 5′ terminal.

With the 5′-RACE cDNA library described in Comparative Example 1-2 as a template, PCR was performed using the above two oligonucleotide primer pairs. A 949 bp TaELO1 translation region (SEQ ID NO: 74) and a 967 bp TaELO2 translation region (SEQ ID NO: 75), in which the vicinity of the start codon was modified to the consensus sequence and having the restriction enzyme HindII at the 5′ terminal and the restriction enzyme XbaI site at the 3′ terminal, were amplified. Furthermore, to avoid elongation mistakes, PrimeSTAR DNA Polymerase (trade name; available from Takara Bio Inc.) having high correction activity was used. [PCR cycles: 98° C. 2 min/98° C. 5 sec, 60° C. 5 sec, 72° C. 1.5 min, 30 cycles/72° C. 7 min/4° C. ∞].

Next, the amplified PCR products were separated with 1% agarose gel, and then DNA fragments were cut and extracted from the agarose gel. Additionally, after treatment with restriction enzymes HindIII and XbaI, the DNG fragments were again purified using agarose gel, and a cyclized vector was constructed by ligation using DNA Ligation Kit “Mighty Mix” (available from Takara Bio Inc.) to a brewer's yeast expression vector pYES2/CT (available from Invitrogen Corp.) which was made into a straight chain by treatment with restriction enzymes HindIII and XbaI. Furthermore, by analyzing the base sequence, it was confirmed that mutations due to PCR elongation mistakes had not been introduced into the sequences of the TaELO1 and TaELO2 translation regions transformed into pYES2/CT. From the above result, a TaELO1 expression vector pYEELO1 and a TaELO2 expression vector pYEELO2 were successfully constructed.

Transformants in which the two constructed expression vectors and pYES2/CT had been transformed into brewer's yeast S. cerevisiae by the lithium acetate method were selected according to the methods described in Non-patent Document 13 and Non-patent Document 14. Next, the obtained transformants (pYEELO1 transgenic strain, pYEELO2 transgenic strain, and mock transgenic strain) were cultured according to the method of Qiu et al. (Non-patent Document 15), and cell-derived fatty acid extraction and methyl-esterification were performed. However, culturing was carried out after adding 0.02 mM each of the following acids in respective final concentrations: α-linolenic acid (ALA, C18:3Δ9, 12, 15) and linoleic acid (LA, C18:2Δ9, 12) as Δ9 elongase substrates; stearidonic acid (STA, C18:4Δ6, 9, 12, 15) and γ-linolenic acid (GLA, C18:3Δ6, 9, 12) as Δ6 elongase substrates; eicosapentaenoic acid (EPA, C20:5Δ5, 8, 11, 14, 17) and arachidonic acid (AA, C20:4Δ5, 8, 11, 14) as Δ5 elongase substrates. Then, gas chromatography (GC) analysis of the methyl-esterified fatty acids was performed according to the method of Abe et al. (Non-patent Document 16). In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min).

As a result, the pYEELO1 transgenic strain exhibited Δ6 elongase activity, by which stearidonic acid (STA) is converted to eicosatetraenoic acid (ETA, 20:4Δ8, 11, 14, 17) and 7-linolenic acid (GLA) is converted to dihomo-7-linolenic acid (DGLA, C20:3Δ8, 11, 14), which was not seen in the host (mock transgenic strain). On the other hand, the pYEELO1 transgenic strain exhibited Δ9 elongase activity, by which α-linolenic acid (ALA) is converted to eicosatrienoic acid (ETrA, C20:3Δ11, 14, 17) and linoleic acid (LA) is converted to eicosadienoic acid (EDA, C20:3Δ11, 14), as well as Δ5 elongase (=C20 elongase) activity, by which eicosapentaenoic acid (EPA) is converted to ω3 docosapentaenoic acid (ω3 DPA, C22:5Δ7, 10, 13, 16, 19) and arachidonic acid (ARA) is converted to docosatetraenoic acid (DTA, C22: 4Δ7, 10, 13, 16) (Table 1).

Furthermore, the pYEELO2 transgenic strain exhibited Δ5 elongase (C20 elongase) activity by which EPA is converted to ω3 DPA (C22: 5Δ7, 10, 13, 16, 19) and ARA is converted to DTA, which was not seen in the host. On the other hand, the pYEELO2 transgenic strain exhibited slight Δ6 elongase activity, by which STA is converted to ETA and GLA is converted to DGLA (Table 1). The above result shows that TaELO1 is Δ6/Δ9/Δ5 elongase, and TaELO2 is Δ5/Δ6 elongase.

TABLE 1

| | mock | TaELO1 | TaELO2 | | mock | TaELO1 | TaELO2 |
|---|---|---|---|---|---|---|---|
| LA supplemented (0.2 mM) | | | | ALA supplemented (0.2 mM) | | | |
| LA | 30.5 | 23.5 | 36.3 | ALA | 49.1 | 25.8 | 47.1 |
| EDA | 0.2 | 8.9 | 0.2 | ETrA | 0.2 | 17.9 | 0.3 |
| Conversion | | 27.4 | | Conversion | | 41 | |
| efficiency | | | | efficiency | | | |
| (%) | | | | (%) | | | |
| GLA supplemented (0.2 mM) | | | | STA supplemented (0.2 mM) | | | |
| GLA | 44.0 | 7.6 | 43.6 | STA | 46.2 | 8.3 | 40.5 |
| DGLA | 0.2 | 29.0 | 0.8 | ETA | 0.3 | 28.1 | 1.7 |
| Conversion | | 79.3 | 1.9 | Conversion | | 77.2 | 4.0 |
| efficiency | | | | efficiency | | | |
| (%) | | | | (%) | | | |
| ARA supplemented (0.2 mM) | | | | EPA supplemented (0.2 mM) | | | |
| ARA | 30.9 | 23.2 | 8.9 | EPA | 42.0 | 31.2 | 13.1 |
| ADA | — | 5.8 | 13.6 | DPA | 0.1 | 10.6 | 24.5 |
| Conversion | | 20.1 | 60.3 | Conversion | | 25.3 | 65.1 |
| efficiency | | | | efficiency | | | |
| (%) | | | | (%) | | | |

Conversion efficiency (%) = 100 × product (area)/substrate (area) + product (area)
(n = 1)

### [Comparative Example 1-4]: Acquisition of TaELO2 ORF Upstream and Downstream Regions by PCR Genome Walking

In the targeting vector for TaELO2 disruption, the regions upstream and downstream of TaELO2 ORF serving as homologous recombination sites were acquired by PCR genome walking. An overview is given below.

Cells of the T. aureum ATCC 34304 strain on the third day of culturing in a GY liquid culture medium were rapidly frozen with liquid nitrogen, and then ground into powder form in a mortar. After genome DNA was extracted according to the method described in Non-patent Document 17, and was dissolved in an appropriate quantity of TE. The quantity and purity of the genome DNA were tested by O.D. 260 and O.D. 280 measurement. Next, a genome DNA library was constructed, wherein a cassette sequence having restriction enzyme sites was appended to genome DNA cut with various restriction enzymes according to the manufacturer's protocol using TaKaRa LA PCR (trade name) in vitro Cloning Kit (available from Takara Bio Inc.). Then, using the produced genome DNA library as a template, nested PCR was performed according to the manufacturer's protocol using forward oligonucleotide primers E2 XbaI (described in Comparative Example 1-3, SEQ ID NO: 73) produced based on the sequence of TaELO2 and elo3-F1 (described in Comparative Example 1-2, SEQ ID NO: 62), or the reverse oligonucleotide primers E2 HindIII (described in Comparative Example 1-3, SEQ ID NO: 72) and elo3-R1 (described in Comparative Example 1-2, SEQ ID NO: 63), together with oligonucleotide primers complementary to the sequences of cassettes included in the kit. As a result, an 1122 bp TaELO2 ORF upstream sequence (SEQ ID NO: 76) and a 1204 bp TaELO2 ORF downstream sequence (SEQ ID NO: 77) were successfully acquired.

### [Comparative Example 1-5]: Construction of TaELO2 Targeting Vector with Neor as Selection Marker

A DNA fragment in which TaELO2 ORF upstream sequence/artificially synthesized Neor/TaELO2 ORF downstream sequence were ligated and produced by fusion PCR. The oligonucleotide primers used were as shown below.

KO Pro F SmaI
(SEQ ID NO: 78)
(31mer: 5′-CTC CCG GGT GGA CCT AGC GCG TGT GTC
ACC T-3′)
(SEQ ID NO: 79)
Pro R (25mer: 5′-GGT CGC GTT TAC AAA GCA GCG CAG
C-3′)
(SEQ ID NO: 80)
SNeo F (52mer: 5′-GCT GCG CTG CTT TGT AAA CGC GAC
CAT GAT TGA ACA GGA CGG CCT TCA CGC T-3′)
(SEQ ID NO: 81)
SNeo R (52mer: 5′-TCG GGA GCC AGC CGG AAA CAG GTT
CAA AAG AAC TCG TCC AGG AGG CGG TAG A-3′)
(SEQ ID NO: 82)
Term F (23mer: 5′-ACC TGT TTC CGG CTG GCT CCC
GA-3′)
(SEQ ID NO: 83)
KO Term R SmaI (27mer: 5′-ATC CCG GGG CCG AGA ACG
GGG TCG CCC-3′)

Of these oligonucleotide primers, KO Pro F SmaI/Pro R were used in amplification of the TaELO2 ORF upstream sequence using the T. aureum ATCC 34304 genome DNA described in Comparative Example 1-4 as a template, SNeo F/SNeo R were used in amplification of artificially synthesized Neor using artificially synthesized Neor as a template, and Term F/KO Term R SmaI were used in amplification of the TaELO2 ORF downstream sequence using the T. aureum ATCC 34304 genome DNA described in Comparative Example 1-4 as a template. As PCR conditions, denaturation was performed at 98° C. for 10 sec, and annealing and the elongation reaction were performed while adjusting as appropriate according to Tm of the primers and the lengths of the amplification products.

As a result, a 2696 bp (SEQ ID NO: 84) TaELO2 ORF upstream sequence/artificially synthesized Neor/TaELO2 ORF downstream sequence was successfully ligated. The result of TA cloning of this sequence using pGEM-T Easy Vector (available from Promega Corporation) was used as a disruption vector, and was named pTKONeor.

### [Comparative Example 1-6]: Transfer of TKONeor into T. aureum ATCC 34304

Using, as a template, pTKONeor, which is the TaELO2 targeting vector with the artificially synthesized Neor as a selection marker produced in Comparative Example 1-5, TaELO2 ORF upstream sequence/artificially synthesized Neor/TaELO2 ORF downstream sequence was amplified using a pair of oligonucleotide primers KO Pro F SmaI (Comparative Example 1-5, SEQ ID NO: 78)/KO Term R SmaI (Comparative Example 1-5, SEQ ID NO: 83) and using PrimeSTAR (trade name) HS DNA Polymerase (available from Takara Bio Inc.). [PCR cycles: 98° C. 2 min/98° C. 10 sec, 68° C. 3 min, 30 cycles/68° C. 10 min/4° C. ∞]. After electrophoresis using 1% agarose gel, the DNA fragments were extracted, and after ethanol precipitation, the extracted DNA were dissolved in an appropriate quantity of TE. The quantity and purity of the DNA fragments were tested by O.D. 260 and O.D. 280 measurement. The obtained DNA fragment is called TKONeor hereinafter.

Next, the introduction of DNA was performed by the gene gun method. Specifically, the T. aureum ATCC 34304 strain was cultured at 25° C. at 150 rpm using a GY liquid culture medium, and cells of the middle to latter logarithmic growth phase were centrifuged for 10 min at 4° C. at 3500×g, and the supernatant was removed. Then, the obtained cells were resuspended in a GY liquid culture medium so as to result in 100 times the concentration of the original culture solution. 20 μL of this cell suspension was thinly spread uniformly and dried in a diameter of approximately 3 cm on PDA agar plate culture medium 5 cm in diameter containing 1 mg/mL of G418 (available from Nacalai Tesque, Inc.). Using a PDS-1000/He System (available from Bio-Rad Laboratories, Inc.), implantation was performed on under the conditions of target distance: 6 cm, vacuum: 26 inches Hg, microcarrier size: 0.6 μm, rupture disk (implantation pressure): 1100 psi. After that, 100 μL of PD liquid culture medium was added drop-wise to the PDA agar plate culture medium, and the cells were spread out again and static culturing was performed. As a result, transformants conferred with G418 resistance were obtained with efficiency of 4.7×10¹ cfu/μg DNA.

### [Comparative Example 1-7]: PCR Using Genome DNA of Transformant in which TKONeor was Transformed as a Template

Seven colonies of transformants were extracted with a toothpick, and were then inoculated in a GY liquid culture medium containing 0.5 mg/mL of G418 (available from Nacalai Tesque, Inc.). After subculturing multiple times, genome DNA was extracted from the cells by the method described in Comparative Example 1-4, and after ethanol precipitation, the extracted genome DNA was dissolved in an appropriate amount of TE. The quantity and purity of the extracted genome DNA were tested by O.D. 260 and O.D. 280 measurement. Then, with the obtained genome DNA of the obtained transformant and the wild-type as templates, PCR was performed using various oligonucleotide primer pairs. The used oligonucleotide primer pairs were as follows:
(1) Neor detection—Sneo F (described in Comparative Example 1-5, SEQ ID NO: 80) and SNeo R (described in Comparative Example 1-5, SEQ ID NO: 81);
(2) KO confirmation 1—KO Pro F SmaI (described in Comparative Example 1-5, SEQ ID NO: 78) and KO Term R SmaI (described in Comparative Example 1-5, SEQ ID NO: 83);
(3) KO confirmation 2—E2 KO ProF EcoRV (30mer: 5′-GGA TAT CCC CCG CGA GGC GAT GGC TGC TCC-3′) (SEQ ID NO: 85) and SNeo R (described in Comparative Example 1-5, SEQ ID NO: 81);
(4) KO confirmation 3—Sneo F (described in Comparative Example 1-5, SEQ ID NO: 80) and E2 KO Term R EcoRV (30mer: 5′-TGA TAT CGG GCC GCG CCC TGG GCC GTA GAT-3′) (SEQ ID NO: 86);
(5) TaELO2 amplification—E2 HindIII (described in Comparative Example 1-3, SEQ ID NO: 72) and E2 XbaI (described in Comparative Example 1-3, SEQ ID NO: 73) (FIG. 36A).

As a result, it was confirmed that six of the seven analyzed clones were transformants by random integration, but in one clone, TaELO2 ORF was substituted for Neor by homologous recombination (FIG. 36B, lanes 9, 13). At the same time, however, it was found that TaELO2 ORF was amplified (FIG. 36B, lane 17). This suggests the possibility that T. aureum ATCC 34304 is at least diploid or TaELO2 is a multicopy gene.

### [Comparative Example 1-8]: Confirmation of TaELO2 Copy Number by Southern Blotting

The following experiment was conducted in accordance with the method described in “DIG Manual [Japanese Edition] 8th, Roche Applied Science” (Non-patent Document 18). Specifically, wild-type genome DNA was cut with various restriction enzymes and then subjected to electrophoresis using 2.5 μg of 0.7% SeaKem (trade name) GTG (trade name) agarose (available from Takara Bio Inc.) per lane. This was transformed to a nylon membrane (Hybond (trade name)-N+, available from GE Healthcare Inc.), and hybridized at 48° C. for 16 hr with a DIG labeled probe produced using PCR DIG Probe Synthesis Kit (available from Roche Applied Science, Inc.). The pair of oligonucleotide primers used in producing the DIG labeled probe were TaELO2 det F (25mer: 5′-GTA CGT GCT CGG TGT GAT GCT GCT C-3′) (SEQ ID NO: 87) and TaELO2 det R (24mer: 5′-GCG GCG TCC GAA CAG GTA GAG CAT-3′) (SEQ ID NO: 88). [PCR cycles: 98° C. 2 min/98° C. 30 sec, 65° C. 30 sec, 72° C. 1 min, 30 cycles/72° C. 7 min/4° C. ∞]. The hybridized probes were detected using the color development method (NBT/BCIP solution).

The results demonstrated that TaELO2 is a single copy gene from the fact that single bands were detected in all lanes treated with the various restriction enzymes (FIG. 37). This suggests that T. aureum ATCC 34304 is at least diploid.

### [Comparative Example 1-9]: Evaluation by Southern Blotting of Transformant in which TKONeor was Transformed

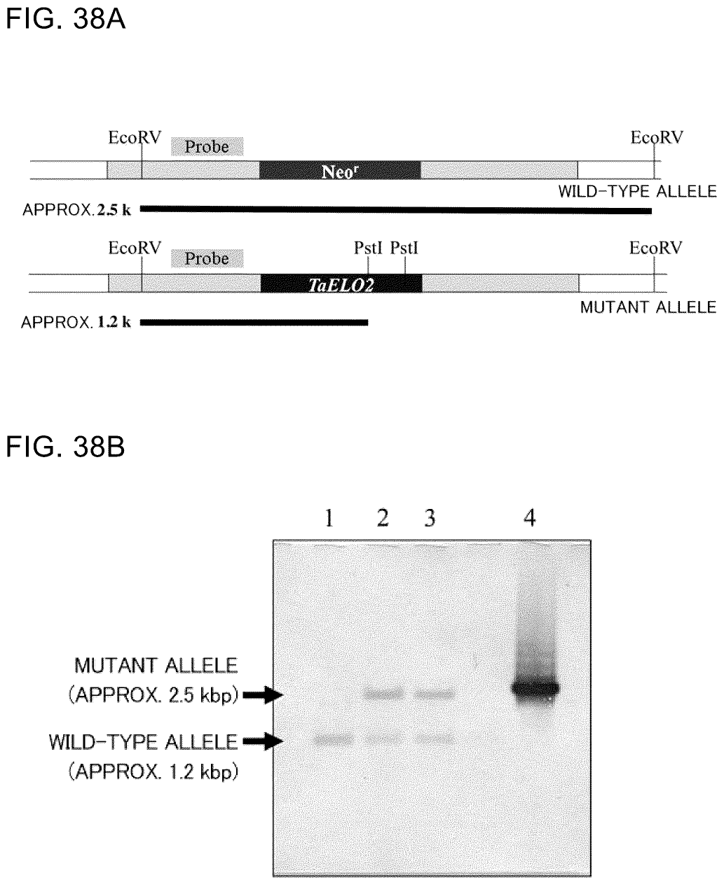

Southern blotting was performed by the method described in Comparative Example 1-8. Specifically, southern blotting was performed using the color development method (NBT/BCIP solution) relative to genome DNA of the transformant and a wild-type strain digested with EcoRV and PstI, using a DIG labeled probe amplified using the pair of oligonucleotide primers uprobe F (35mer: 5′-ATC CGC GTA TAT ATC CGT AAA CAA CGG AAC ATT CT-3′) (SEQ ID NO: 89) and uprobe R (26mer: 5′-CTT CGG GTG GAT CAG CGA GCG ACA GC-3′) (SEQ ID NO: 90). [PCR cycles: 98° C. 2 min/98° C. 30 sec, 65° C. 30 sec, 72° C. 1 min, 30 cycles/72° C. 7 min/4° C. ∞]. In this case, in the wild-type allele, a DNA fragment of approximately 1.2 kbp was detected, but in the mutant allele in which TaELO2 ORF was substituted for Neor by homologous recombination, a DNA fragment of approximately 2.5 kbp was detected (FIG. 38A).

The result of analysis shows that T. aureum ATCC 34304 is at least diploid, since the wild-type allele band was also detected at the same time as the mutant allele.

### [Comparative Example 1-10]: Construction of TaELO2 Targeting Vector with Hygr as Selection Marker

To disruption the remaining wild-type allele, a TaELO2 targeting vector with Hygr as a selection marker was constructed.

First, a ubiquitin promoter sequence derived from T. aureum ATCC 34304 and Hygr were ligated by fusion PCR. The oligonucleotide primers used were as shown below

ubi-600p F
(SEQ ID NO: 91)
(27mer: 5′-GCC GCA GCG CCT GGT GCA CCC GCC GGG-3′)
ubi-hygro R
(SEQ ID NO: 92)
(59mer: 5′-TCG CGGG TGA GTT CAG GCT TTT TCA TGT
TGG CTA GTG TTG CTT AGG TCG CTT GCT GCT G-3′)
ubi-hygro F
(SEQ ID NO: 93)
(57mer: 5′-AGC GAC CTA AGC AAC ACT AGGC CAA CAT
GAA AAA GCC TGA ACT CAC CGC GAC GTC TG-3′)
hygro R
(SEQ ID NO: 94)
(29mer: 5′-CTA TTC CTT TGC CCT CGG ACG AGT GCT
GG-3′)

Of these oligonucleotide primers, ubi-600p F/ubi-hygro R were used in amplification of the T. aureum ATCC 34304-derived ubiquitin promoter sequence using the T. aureum ATCC 34304 genome DNA described in Comparative Example 1-4 as a template. Ubi-hygro F/hygro R were used in amplification of artificially synthesized Hygr using pcDNA 3.1 Zeo (available from Invitrogen Corp.) as a template. As PCR conditions, denaturation was performed at 98° C. for 10 sec, and annealing and the elongation reaction were performed while adjusting as appropriate according to Tm of the primers and the lengths of the amplification products.

As a result, 1636 bp (SEQ ID NO: 95) of T. aureum ATCC 34304-derived ubiquitin promoter sequence/Hygr was successfully ligated. The result of TA cloning of this sequence using pGEM-T Easy Vector (available from Promega Corporation) was named pTub600Hygr.

Then, using pTub600Hygr as a template, PCR was performed using a pair of oligonucleotide primers ubi-600p F NheI (33mer: 5′-GTG CTA GCC GCA GCG CCT GGT GCA CCC GCC GGG-3′) (SEQ ID NO: 96) and hygro R XbaI (37mer: 5′-GTT CTA GAC TAT TCC TTT GCC CTC GGA CGA GTG CTG G-3′) (SEQ ID NO: 97) and using PrimeSTAR HS DNA Polymerase (available from Takara Bio Inc.). [PCR cycles: 98° C. 2 min/98° C. 10 sec, 68° C. 3 min, 30 cycles/68° C. 10 min/4° C. ∞]. As a result, a T. aureum ATCC 34304-derived ubiquitin promoter sequence/Hygr DNA fragment with NheI appended at the 5′ terminal and an XbaI site appended at the 3′ terminal was prepared. Furthermore, using pTKONeor described in Comparative Example 1-5 as a template, PCR was performed using a pair of oligonucleotide primers KO vec F XbaI (37mer: 5′-GTT CTA GAC CTG TTT CCG GCT GGC TCC CGA GCC ATG C-3′) (SEQ ID NO: 98) and KO vec R NheI (40mer: 5′-GTG CTA GCG GTC GCG TTT ACA AAG CAG CGC AGC AAC AGA A-3′) (SEQ ID NO: 99) and using PrimeSTAR HS DNA Polymerase (available from Takara Bio Inc.). [PCR cycles: 98° C. 2 min/98° C. 10 sec, 68° C. 3 min, 30 cycles/68° C. 10 min/4° C. ∞]. As a result, a linear vector in which Neor of pTKONeor described in Comparative Example 1-5 was removed and having NheI appended at the 3′ terminal and an XbaI site appended at the 5′ terminal was prepared. After the two DNA fragments were digested with NheI and XbaI, the DNA fragments were purified using agarose gel, and cyclic vectors were constructed using Ligation Convenience Kit (available from Nippon Gene Co., Ltd.).

The constructed TaELO2 targeting vector having Hygr as a selection marker includes pGEM-T Easy Vector (available from Promega Corporation) as a basic framework, and as an insertion sequence, had a 3537 bp (SEQ ID NO: 100) TaELO2 ORF upstream sequence/T. aureum ATCC 34304-derived ubiquitin promoter sequence/Hygr/TaELO2 ORF downstream sequence. This was named pTKOub600Hygr.

### [Comparative Example 1-11]: Evaluation of Transformant by PCR Using KOub600Hygr Retransfer and Genome DNA as Templates, Southern Blotting, and RT-PCR

Using, as a template, pTKOub600Hygr (described in Comparative Example 1-10), which is the constructed TaELO2 targeting vector with Hygr as a selection marker, TaELO2 ORF upstream sequence/T. aureum ATCC 34304-derived ubiquitin promoter sequence/Hygr/TaELO2 ORF downstream sequence was amplified using a pair of oligonucleotide primers KO Pro F SmaI (Comparative Example 1-5, SEQ ID NO: 78)/KO Term R SmaI (Comparative Example 1-5, SEQ ID NO: 83) and using PrimeSTAR HS DNA Polymerase (available from Takara Bio Inc.). [PCR cycles: 98° C. 2 min/98° C. 10 sec, 68° C. 3.5 min, 30 cycles/68° C. 10 min/4° C. ∞]. The obtained DNA fragment was named KOub600Hygr. This was transformed into the transformant obtained in Comparative Example 1-7 by the same technique, and after static culturing for 24 hr on PDA agar plate culture medium containing 1 mg/mL of G418 (available from Nacalai Tesque, Inc.), the cells were collected. The static culturing was continued on PDA agar plate culture medium containing 1 mg/mL of G418 (available from Nacalai Tesque, Inc.) and 2 mg/mL of hygromycin B (available from Wako Pure Chemical Industries, Ltd.) to obtain numerous transformants (transfer efficiency: 1.02×10³ cfu/μg DNA).

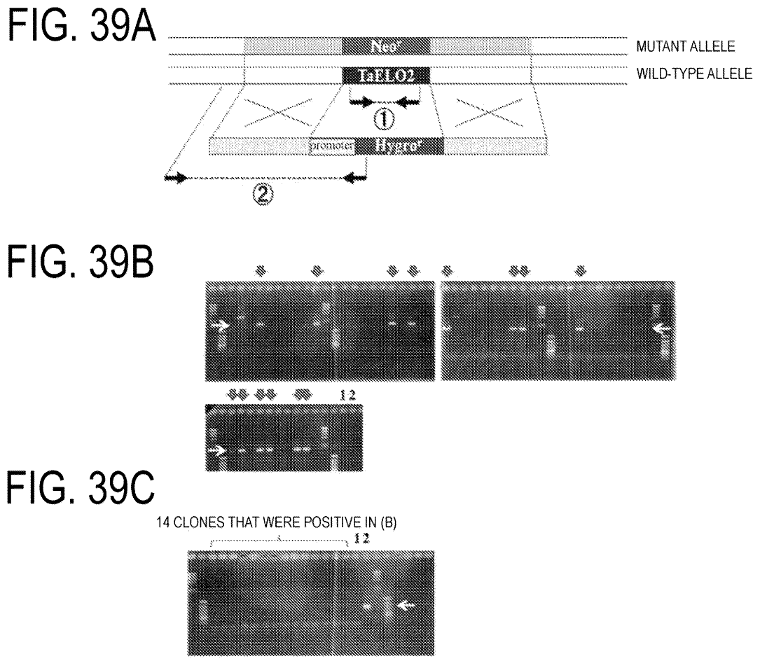

Among them, 50 clones were extracted, and after subculturing multiple times in a GY liquid culture medium containing 1 mg/mL of G418 (available from Nacalai Tesque, Inc.) and 2 mg/mL of hygromycin B (available from Wako Pure Chemical Industries, Ltd.), genome DNA was extracted by the same technique as described in Comparative Example 1-4, and after ethanol precipitation, the extracted genome DNA was dissolved in an appropriate quantity of TE. The quantity and purity of the extracted genome DNA were tested by OD260 and OD280 measurement. Then, with the obtained transformant and wild-type genome DNA as templates, PCR was performed using various oligonucleotide primer pairs. [PCR cycles: 98° C. 2 min/98° C. 10 sec, 68° C. 1 min, 30 cycles/68° C. 10 min/4° C. ∞]. The used oligonucleotide primer pairs were as follows:
(1) TaELG2 ORF detection—Sneo F (described in Comparative Example 1-5, SEQ ID NO: 80) and SNeo R (described in Comparative Example 1-5, SEQ ID NO: 81);
(2) KO confirmation—E2 KO Pro F EcoRV (described in Comparative Example 1-7, SEQ ID NO: 85) and ubi-hygro R (described in Comparative Example 1-10, SEQ ID NO: 92) (FIG. 39A).

The results showed that of the 50 analyzed clones, 14 clones were transformants which caused homologous recombination in the form of substituting TaELO2 ORF (FIG. 39B, arrows). For these clones, it was confirmed that TaELO2 ORF was not amplified (FIG. 39C).

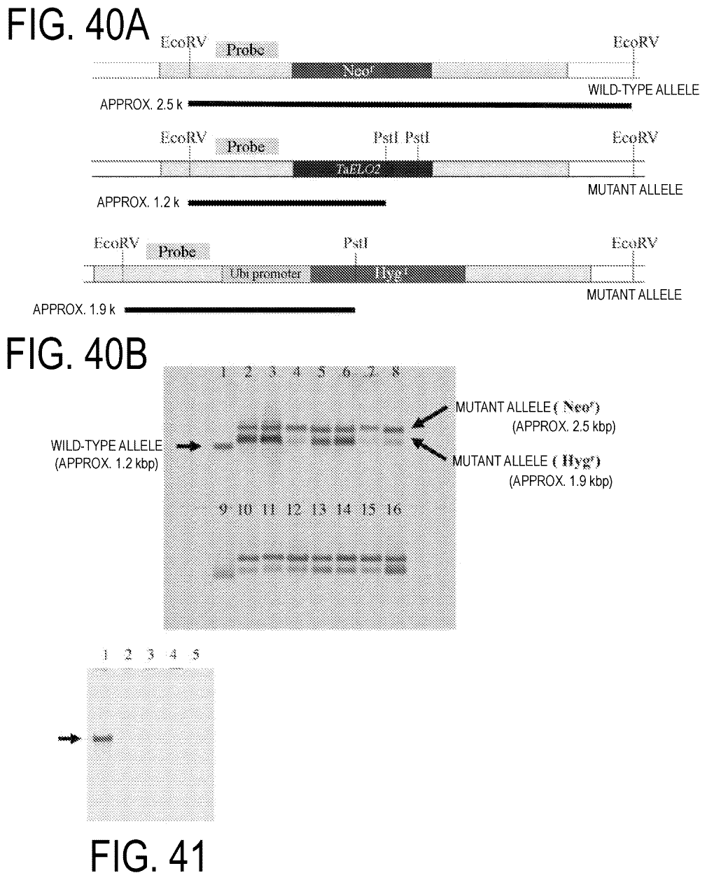

Then, southern blotting was performed using the technique described in Comparative Example 1-9. Specifically, southern blotting was performed by the color development method (NBT/BCIP solution) relative to genome DNA of the transformant and a wild-type strain digested with EcoRV and PstI, using a DIG labeled probe prepared using the pair of oligonucleotide primers uprobe F (SEQ ID NO: 89) and uprobe R (SEQ ID NO: 90). In this case, in the wild-type allele, a DNA fragment of approximately 1.2 kbp was detected; in the mutant allele in which TaELO2 ORF was substituted for Neor by homologous recombination, a DNA fragment of approximately 2.5 kbp was detected; in the mutant allele in which TaELO2 ORF was substituted for Hygr, a DNA fragment of approximately 1.9 kbp was detected (FIG. 40A).

As a result of analysis, a band of the wild-type allele of approximately 2.5 kbp was disappeared, and instead, a band of the mutant allele of approximately 1.9 kbp in which TaELO2 ORF was substituted for Hygr was detected (FIG. 40B).

Similarly, a DIG labeled probe that detects TaELO2 was prepared by PCR using the pair of oligonucleotide primers TaELO2 probe F (30mer: 5′-ATG GCG ACG CGC ACC TCG AAG AGC GCT CCG-3′) (SEQ ID NO: 101) and TaELO2 probe R (30mer: 5′-AGG ATC ATC ATG AAC GTG TCG CTC CAG TCG-3′) (SEQ ID NO: 102). [PCR cycles: 98° C. 2 min/98° C. 30 sec, 65° C. 30 sec, 72° C. 1 min, 30 cycles/72° C. 7 min/4° C. ∞]. Southern blotting was performed by the color development method (NBT/BCIP solution) relative to genome DNA of transformants (clones 1, 8, 9, 10) and a wild-type strain digested with EcoRV. In this case, TaELO2 was detected as a DNA fragment of approximately 2.5 kbp (FIG. 38A).

The result of analysis showed that TaELO2 was detected in the wild-type strain (FIG. 41, lane 1), whereas in the transformant, TaELO2 was not detected at all (FIG. 41, lanes 2 to 5).

Furthermore, to verify TaELO2 disruption at an mRNA level, TaELO2 mRNA was detected by RT-PCR. From the cells of the transformant (clones 1, 8, 9, 10) and the wild-type strain on the third day of culturing using a GY liquid culture medium, total RNA was extracted using Sepasol-RNA I Super (available from Nacalai Tesque, Inc.) in the same manner as Comparative Example 1-1. Then, 50 μg of total RNA cleaned up using RNeasy Mini Kit (available from QIAGEN N.V.) according to the manufacturer's protocol was treated for 1 hr at 37° C. using 50 U of Recombinant DNase I (available from Takara Bio Inc.), and contaminated genome DNA was decomposed and removed. Then, using the obtained total RNA as a template, a single-strand cDNA library was prepared according to manufacturer's instructions using oligo (dT) primer (available from Novagen Corp.) and PrimeScript Reverse Transcriptase (available from Takara Bio Inc.). Additionally, using the obtained single-strand cDNA library as a template, TaELO2 ORF was amplified using a pair of oligonucleotide primers E2 HindIII (described in Comparative Example 1-3, SEQ ID NO: 72) and E2 XbaI (described in Comparative Example 1-3, SEQ ID NO: 73) and using LA Taq Hot Start Version (available from Takara Bio Inc.). [PCR cycles: 98° C. 2 min/98° C. 10 sec, 68° C. 1 min, 30 cycles/68° C. 10 min/4° C. ∞].

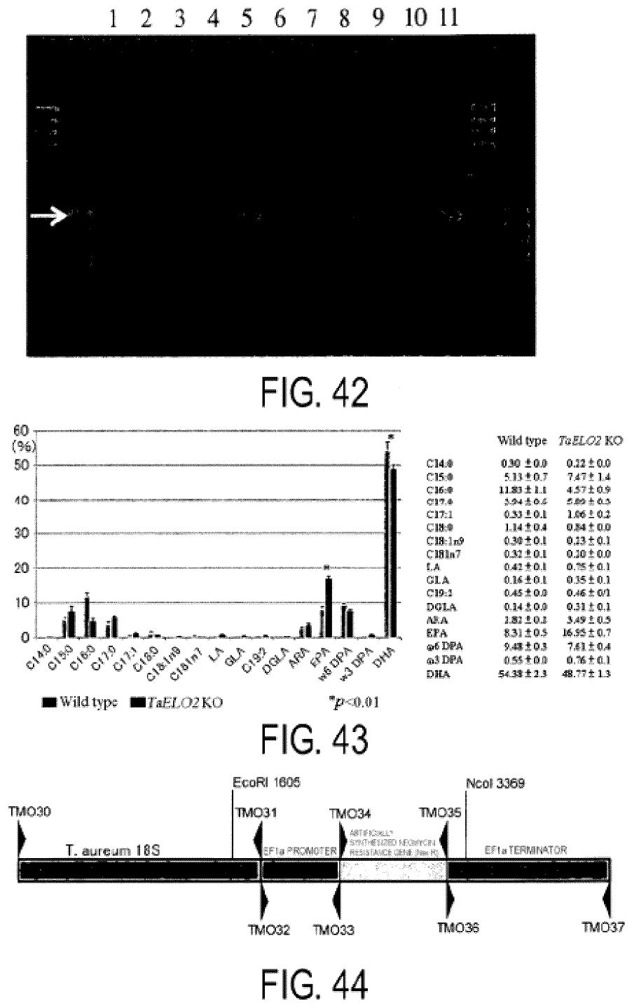

The result showed that TaELO2 mRNA was detected in the wild-type strain (FIG. 42, lane 5), whereas TaELO2 mRNA was not detected at all in the transformant (clones 1, 8, 9, 10) (FIG. 42, lanes 1 to 4).

The above results showed that a TaELO2 deletion homozygote in which TaELO2 had been completely disrupted was successfully obtained. The above results demonstrate that T. aureum ATCC 34304 is diploid.

### [Comparative Example 1-12]: Comparison of Fatty Acid Compositions of Wild-Type Strain and TaELO2 Deletion Homozygote

The fatty acid compositions of the TaELO2 deletion homozygote obtained in Comparative Example 1-11 and the wild-type strain were compared by GC analysis of the methyl-esterified fatty acids. Specifically, the cells of the wild-type strain and the TaELO2 deletion homozygote after 5 days of culturing in a GY liquid culture medium were collected. The fungus body-derived fatty acids were extracted and methyl-esterified by the method described in Comparative Example 1-3, and GC analysis was performed. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min).

As a result, the quantity of EPA serving as a substrate of TaELO2 increased up to approximately 2-fold in the TaELO2 deletion homozygote (TaELO2 KO) compared to the wild-type strain (Wild type), and a decrease in the amount of the downstream metabolite DHA was observed (FIG. 43).

As described above, it was confirmed that due to C20 elongase gene disruption in T. aureum ATCC 34304, similar to genus Parietichytrium labyrinthulids, the quantity of the C20 elongase substrate EPA increased compared to the wild-type strain, and conversely, the downstream metabolite DHA and the like decreased. However, unlike the genus Parietichytrium labyrinthulids, the proportion of DHA did not really decrease in T. aureum ATCC 34304 even when the C20 elongase gene was disrupted. Specifically, the proportion of DHA in the wild-type strain was 54.38%, whereas the proportion of DHA was 48.77%, which is only slightly less in the C20 elongase gene KO strain. A similar trend was seen for n-6 DPA as well.

As described in Comparative Example 1-2, in T. aureum ATCC 34304, TaELO1 is present in addition to TaELO2, which was disrupted this time. In Comparative Example 1-3, it was shown that both have Δ5 elongase activity (=C20 elongase activity). However, it was also clear that the Δ5 elongase activity of TaELO1 is considerably lower than the Δ5 elongase activity of TaELO2, and the reason that DHA and n-6 DPA did not really decrease in the TaELO2 deletion homozygote (TaELO2 KO) is difficult to explain by the Δ5 elongase activity (=C20 elongase activity) of TaELO1. This suggests the possibility that in Thraustochytrium aureum ATCC 34304, DHA and n-6 DPA are produced via another biosynthesis pathway in addition to the elongase-desaturase pathway.

When such a labyrinthulid is selected, unlike Examples 2, 4, and 6, it is not possible to create a strain which accumulates PUFAs other than DHA and n-6 DPA even by disruption the C20 elongase gene. Therefore, creating such a strain requires disruption of a gene associated with a DHA and n-6 DPA biosynthesis pathway other than the elongase-desaturase pathway.

### Comparative Example 2

[Measurement of Fatty Acid Composition of Lipids Produced by PUFA-PKS Gene Disruption and Transformation Strain of Thraustochytrium aureum ATCC 34304]

### [Comparative Example 2-1]: PUFA-PKS Pathway Associated Gene: OrfA Upstream Sequence Cloning

After genome DNA was extracted from Thraustochytrium aureum ATCC 34304 by the method described in Example 2-2, A260/280 was measured and the DNA concentration was calculated. Using this extracted genome DNA, a genome cassette library was produced using LA PCR (trade name) in vitro Cloning Kit (available from Takara Bio Inc.). A PCR lower primer [RHO20: 23mer: 5′-CGA TGAAAG GTC ACA GAA GAG TC-3′ (SEQ ID NO: 103)] was set on the PUFA-PKS pathway associated gene: OrfA described in Patent Document 4, and DNA was amplified by combining with the cassette primers contained in the above kit. [1st PCR cycles: 98° C. 2 min/98° C. 30 sec, 56° C. 30 sec, 72° C. 4 min, 30 cycles/72° C. 5 min]. Then, the 1st PCR amplification product was diluted 100-fold, and the DNA was amplified by combining the PCR lower primer [RH020] and the nested primers contained in the above kit. [2nd PCR cycles: 98° C. 2 min/98° C. 30 sec, 56° C. 30 sec, 72° C. 4 min, 30 cycles/72° C. 5 min]. The obtained DNA fragment was cloned in pGEM-T Easy Vector, and after amplification with E. coli, the sequence was confirmed using a Dye Terminator Cycle Sequencing Kit (available from Beckman Coulter Inc.).

A DNA fragment of 3377 bp (SEQ ID NO: 105) containing 3181 bp (SEQ ID NO: 104) upstream of OrfA was cloned. It became clear that the OrfA upstream DNA sequence information was a total of 3181 bp.

### [Comparative Example 2-2]: PUFA-PKS Pathway Associated Gene: OrfA Downstream Sequence Cloning

The genome cassette library produced in Comparative Example 2-1 was used as a template. A PCR upper primer [RHO21: 21mer: 5′-CAG GGC GAG CGA GTG TGG TTC-3′ (SEQ ID NO: 106)] was set on the PUFA-PKS pathway associated gene: OrfA described in Patent Document 4, and DNA was amplified by the method described in Comparative Example 2-1. The obtained DNA fragment was cloned in pGEM-T Easy Vector, and after amplification with E. coli, the sequence was confirmed using a Dye Terminator Cycle Sequencing Kit (available from Beckman Coulter Inc.). A DNA fragment of 1204 bp (SEQ ID NO: 108) containing 1160 bp (SEQ ID NO: 107) downstream of OrfA was cloned.

A PCR upper primer [RH028: 20mer: 5′-TGA TGC CGA TGC TAC AAA AG-3′ (SEQ ID NO: 109)] was again produced on SEQ ID NO: 94, and DNA was amplified by the method described in Comparative Example 1-2. The obtained DNA fragment was cloned in pGEM-T Easy Vector, and after amplification with E. coli, the sequence was confirmed using a Dye Terminator Cycle Sequencing Kit (available from Beckman Coulter Inc.).

Furthermore, a 1488 bp DNA fragment (SEQ ID NO: 110) containing the downstream sequence was cloned. It became clear that the OrfA downstream DNA sequence information is a total of 2551 bp (SEQ ID NO: 111).

### [Comparative Example 2-3]: Production of PUFA-PKS Pathway Associated Gene: OrfA Targeting Vector

Using Thraustochytrium aureum ATCC 34304 genome DNA as a template, an 18S rDNA sequence (1835 bp, SEQ ID NO: 112) was amplified with PrimeSTAR HS DNA Polymerase (available from Takara Bio Inc.). The PCR primers used were as shown below. TMO30 was set on the 18S rDNA sequence. TMO31 includes the 18S rDNA sequence and the EF1α promoter sequence. [TMO30: 30mer: 5′-CGA ATA TTC CTG GTT GAT CCT GCC AGT AGT-3′ (SEQ ID NO: 113), TMO31: 46mer: 5′-GTAACG GCT TTT TTT GAA TTG CAG GTT CAC TAC GCT TGT TAG AAA C-3′ (SEQ ID NO: 114)]. [PCR cycles: 98° C. 10 sec/98° C. 10 sec, 58° C. 30 sec, 72° C. 2 min, 30 cycles/72° C. 2 min].

Furthermore, using Thraustochytrium aureum ATCC 34304 genome DNA as a template, an EF1α promoter sequence (661 bp, SEQ ID NO: 115) was amplified with PrimeSTAR HS DNA Polymerase (available from Takara Bio Inc.). The PCR primers used were as shown below. TMO32 includes the 18S rDNA sequence and the EF1α promoter sequence. TMO33 includes the EF1α promoter sequence and an artificially synthesized neomycin resistance gene sequence. [TMO32: 46mer: 5′-GGT TTC CGT AGT GAA CCT GCAATT CAA AAA AAG CCG TTA CTC ACA T-3′ (SEQ ID NO: 116), TMO33: 46mer: 5′-GCG TGA AGG CCG TCC TGT TCA ATC ATC TAG CCT TCC TTT GCC GCT G-3′ (SEQ ID NO: 117)]. [PCR cycles: 98° C. 10 sec/98° C. 10 sec, 58° C. 30 sec, 72° C. 1 min, 30 cycles/72° C. 1 min].

Using the artificially synthesized neomycin resistance gene sequence as a template, an artificially synthesized neomycin resistance gene sequence (835 bp, SEQ ID NO: 118) was amplified with PrimeSTAR HS DNA Polymerase (available from Takara Bio Inc.). The PCR primers used were as shown below. TMO34 includes the EF1α promoter sequence and the artificially synthesized neomycin resistance gene sequence. TMO35 includes the artificially synthesized neomycin resistance gene sequence and the EF1α terminator sequence. [TMO34: 45mer: 5′-CAT CGG CAA AGG AAG GCT AGA TGA TTG AAC AGG ACG GCC TTC ACG-3′ (SEQ ID NO: 119), TMO35: 46mer: 5′-GCG CAT AGC CGG CGC GGA TCT CAA AAG AAC TCG TCC AGG AGG CGG T-3′ (SEQ ID NO: 120)]. [PCR cycles: 98° C. 10 sec/98° C. 10 sec, 58° C. 30 sec, 72° C. 1 min, 30 cycles/72° C. 1 min].

Using Thraustochytrium aureum ATCC 34304 genome DNA as a template, an EF1α terminator sequence (1249 bp, SEQ ID NO: 121) was amplified with PrimeSTAR HS DNA Polymerase (available from Takara Bio Inc.). The PCR primers used were as shown below. TMO36 includes the artificially synthesized neomycin resistance gene sequence and the EF1α terminator sequence. TMO37 was set within the EF1α terminator. [TMO36: 46mer: 5′-TCC TGG ACG AGT TCT TTT GAG ATC CGC GCC GGC TAT GCG CCC GTG C-3′ (SEQ ID NO: 122), TMO37: 30mer: 5′-CAC TGC AGC GAA AGA CGG GCC GTA AGG ACG-3′ (SEQ ID NO: 123)]. [PCR cycles: 98° C. 10 sec/98° C. 10 sec, 58° C. 30 sec, 72° C. 2 min, 30 cycles/72° C. 2 min].

Using SEQ ID NOS: 112, 115, 118, and 121 as templates, fusion PCR was performed according to the method described in Non-patent Document 9. LA Taq Hot Start Version (available from Takara Bio Inc.) was used for the enzymes. In the first amplification, the set of TMO30 (SEQ ID NO: 113) and TMO33 (SEQ ID NO: 117) and the set of TMO34 (SEQ ID NO: 119) and TMO37 (SEQ ID NO: 123) were used. In the second amplification, the set of TMO30 (SEQ ID NO: 113) and TMO37 (SEQ ID NO: 123) was used. As conditions of the PCR reaction, denaturation was performed at 98° C. for 10 sec, and annealing and the elongation reaction were performed while adjusting as appropriate according to Tm of the primers and the lengths of the amplification fragments (FIG. 42).

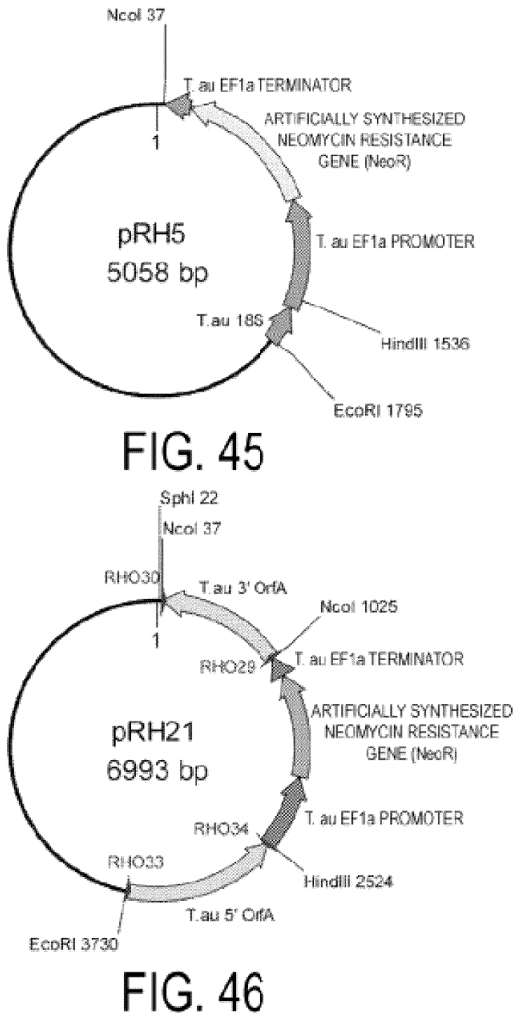

The DNA fragment ligated in this manner (FIG. 44, SEQ ID NO: 124, 4453 bp) was cut at the EcoRI site in T. aureum 18S rDNA and at the NcoI site in the T. aureum EF1α terminator, and was bound to the vector derived from pGEM-T Easy Vector. This was named pRH5 (FIG. 45).

Using Thraustochytrium aureum ATCC 34304 genome DNA as a template, the PCR primers were set in the upstream sequence clarified in Comparative Example 2-1 (SEQ ID NO: 104) and the PUFA-PKS pathway associated gene: OrfA described in Patent Document 4, and DNA was amplified with PrimeSTAR HS DNA Polymerase with GC Buffer (available from Takara Bio Inc.). A 1218 bp DNA fragment (SEQ ID NO: 125) was obtained by this amplification. This was used as the 5′ homologous region of the targeting vector. The PCR primers used are as shown below. As a linker sequence, an EcoRI site or a HindIII site was appended to each. [RH033: 32mer: 5′-CCC GAA TTC GGA CGA TGA CTG ACT GAC TGA TT-3′ (SEQ ID NO: 126), RH034: 28mer: 5′-CCC AAG CTT GTC TGC CTC GGC TCT TGG T-3′ (SEQ ID NO: 127)]. [PCR cycles: 98° C. 2 min/98° C. 30 sec, 57° C. 30 sec, 72° C. 1 min, 30 cycles/72° C. 3 min].

Using Thraustochytrium aureum ATCC 34304 genome DNA as a template, the PCR primers were set in the downstream sequence clarified in Comparative Example 2-2 (SEQ ID NO: 111), and DNA was amplified with PrimeSTAR HS DNA Polymerase with GC Buffer (available from Takara Bio Inc.). A 1000 bp DNA fragment (SEQ ID NO: 128) was obtained by this amplification. This was used as the 3′ homologous region of the targeting vector. The PCR primers used were as shown below. An NcoI site as a linker sequence was appended to each. [RHO29: 28mer: 5′-CCC CCA TGG TGT TGC TGT GGG ATT GGT C-3′ (SEQ ID NO: 129), RHO30: 30mer: 5′-CCC CCA TGG CTC GGT TAC ATC TCT GAG GAA-3′ (SEQ ID NO: 130)]. [PCR cycles: 98° C. 2 min/98° C. 30 sec, 57° C. 30 sec, 72° C. 1 min, 30 cycles/72° C. 3 min].

The amplified upstream sequence was ligated to the EcoRI site and the HindIII site in pRH5 illustrated in FIG. 43. The amplified downstream sequence was ligated to the NcoI site. This vector was named pRH21.

The targeting vector (pRH21) obtained using the produced artificially synthesized neomycin resistance gene is illustrated in FIG. 44.

### [Comparative Example 2-4]: Production of PUFA-PKS Pathway Associated Gene: OrfA Targeting Vector (Hygromycin Resistance Gene)

Using pRH32 (FIG. 6) described in Example 2-3 as a template, a ubiquitin promoter—hygromycin resistance gene fragment (1632 bp, SEQ ID NO: 131) was amplified with PrimeSTAR HS DNA Polymerase with GC Buffer (available from Takara Bio Inc.). The PCR primers used were as shown below. RH059 was set on the ubiquitin promoter, and a HindIII site was appended as a linker sequence. RHO60 contains a stop codon of the hygromycin resistance gene sequence, and has the linker sequences SphI and SalI. [RH059: 36mer: 5′-CCC AAG CTT GCC GCA GCG CCT GGT GCA CCC GCC GGG-3′ (SEQ ID NO: 132), RHO60: 43mer: 5′-CCC GCA TGC GTC GAC TAT TCC TTT GCC CTC GGA CGA GTG CTG G-3′ (SEQ ID NO: 133)]. [PCR cycles: 98° C. 2 min/98° C. 30 sec, 68° C. 2 min, 30 cycles/68° C. 2 min].

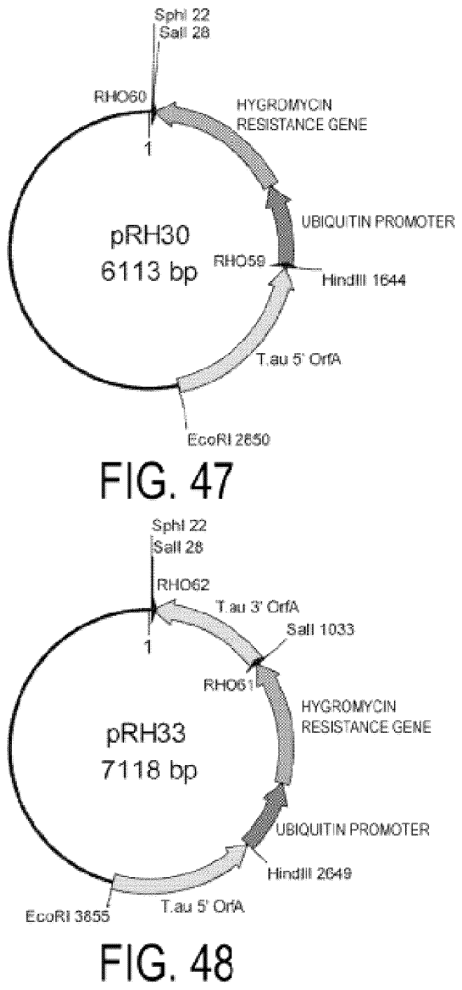

The amplified fragment was ligated to the HindIII and SphI sites of pRH21 (FIG. 46) described in Comparative Example 2-3 (FIG. 47, pRH30).

Using Thraustochytrium aureum ATCC 34304 genome DNA as a template, the DNA was amplified with PrimeSTAR HS DNA Polymerase with GC Buffer (available from Takara Bio Inc.) using the produced PCR primers in the downstream sequence (SEQ ID NO: 111) clarified in Comparative Example 2-2. A 1000 bp DNA fragment (SEQ ID NO: 134) was obtained by this amplification. This was used as the 3′ homologous region of the targeting vector. The PCR primers used were as shown below. A SalI site as a linker sequence was also appended. [RHO61: 29mer: 5′-CCC GTC GAC GTG TTG CTG TGG GAT TGG TC-3′ (SEQ ID NO: 135), RH062: 29mer: 5′-CCC GTC GAC TCG GTT ACA TCT CTG AGG AA-3′ (SEQ ID NO: 136)]. [PCR cycles: 98° C. 2 min/98° C. 30 sec, 57° C. 30 sec, 72° C. 1 min, 30 cycles/72° C. 3 min].

The amplified downstream sequence was ligated to the SalI site of pRH30 (FIG. 45). This was named pRH33. The targeting vector (pRH33) obtained using the produced hygromycin resistance gene is illustrated in FIG. 48.

### [Comparative Example 2-5]: PUFA-PKS Pathway Associated Gene: OrfA Targeting Vector Transfer

Using the targeting vectors produced in Comparative Examples 2-3 and 2-4 as templates, the genes were amplified with PrimeSTAR Max DNA Polymerase (available from Takara Bio Inc.) using RHO30 (described in Comparative Example 2-3, SEQ ID NO: 130) and RHO33 (described in Comparative Example 2-3, SEQ ID NO: 126) as primers. [PCR cycles: 98° C. 2 min/98° C. 30 sec, 60° C. 30 sec, 72° C. 1 min, 30 cycles/72° C. 3 min]. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH21 (FIG. 46) described in Comparative Example 2-3 was used as a template was 3705 bp, and resulted in a sequence including Thraustochytrium aureum OrfA gene upstream—EF1α promoter sequence artificially synthesized neomycin resistance gene sequence—Thraustochytrium aureum OrfA gene downstream (SEQ ID NO: 137). The transfer fragment obtained when pRH33 (FIG. 46) described in Comparative Example 2-4 was used as a template was 3826 bp, and resulted in a sequence including Thraustochytrium aureum OrfA gene upstream—ubiquitin promoter sequence—hygromycin resistance gene sequence—Thraustochytrium aureum OrfA gene downstream (SEQ ID NO: 138).

The Thraustochytrium aureum ATCC 34304 strain was cultured for 4 days in a GY culture medium, and cells in the logarithmic growth phase were used in gene transfer. To cells corresponding to OD600=1 to 1.5, 0.625 μg of DNA fragment was transformed by the gene gun method (microcarrier: 0.6 micron gold particles, target distance: 6 cm, chamber vacuum: 26 mmHg, rupture disk: 1100 psi). After a recovery time of 4 to 6 hr, the transgenic cells were spread on a PDA agar plate culture medium (containing 2 mg/mL of G418 or containing 2 mg/mL of hygromycin). As a result, from 100 to 200 cells of drug resistant strain per shot were obtained.

### [Comparative Example 2-6]: Identification of PUFA-PKS Pathway Associated Gene: OrfA Gene Targeting Homologous Recombinant

After genome DNA was extracted from Thraustochytrium aureum ATCC 34304 and a hetero homologous recombinant and homo homologous recombinant (PKS pathway associated gene disruption strain) by the method described in Example 2-2, A260/280 was measured and the DNA concentration was calculated.

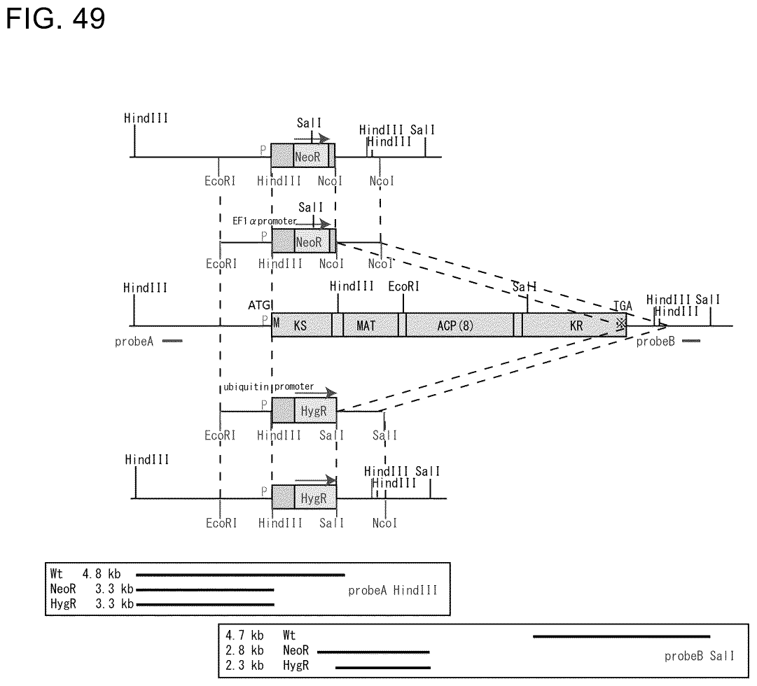

After the genome DNA was cut with a restriction enzyme, the obtained genome DNA underwent electrophoresis in approximately 2 to 3 μg per well of 0.7% SeaKem GTG agarose gel (available from Takara Bio Inc.). This was transformed to a nylon membrane, and hybridized at 54° C. for 16 hr with a probe produced using DIG System (available from Roche Applied Science, Inc.). The primers used in probe production were as follows. 5′ side [RHO37: 22mer: 5′-GAA GCG TCC CGT AGA TGT GGT C-3′ (SEQ ID NO: 139), RHO38: 21mer: 5′-GCC CGA GAG GTC AAA GTA CGC-3′ (SEQ ID NO: 140)]; 3′ side [RHO39: 20mer: 5′-GCG AGC CCA GGT CCA CTT GC-3′ (SEQ ID NO: 141), RHO40: 22mer: 5′-CAG CCC GAT GAA AAA CTT GGT C-3′ (SEQ ID NO: 142)]. [PCR cycles: 98° C. 2 min/98° C. 30 sec, 60° C. 30 sec, 72° C. 2 min, 30 cycles/72° C. 3 min]. The positions of the restriction enzymes and the probes used are illustrated in FIG. 49. The hybridized probes were detected using the color development method (NBT/BCIP solution).

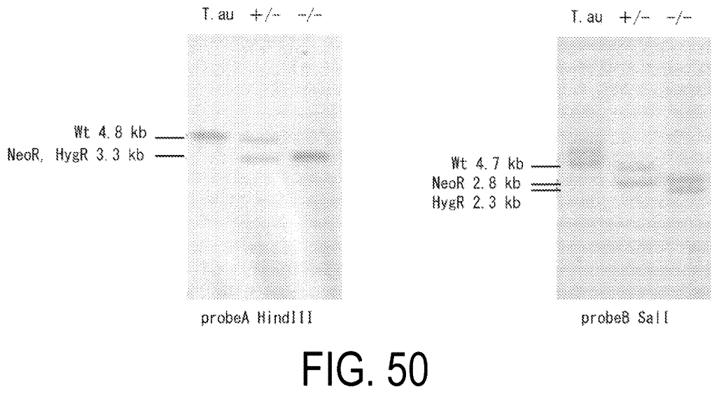

In analysis of both the 5′ side and the 3′ side, bands were observed at the expected sizes when the drug resistance genes caused homologous recombination (FIG. 50).

### Comparative Example 2-7

Thraustochytrium aureum ATCC 34304 and the gene disruption strain were cultured according to the method described in Example 2-9, and after freeze drying, the fatty acids were methyl-esterified and analyzed using GC.

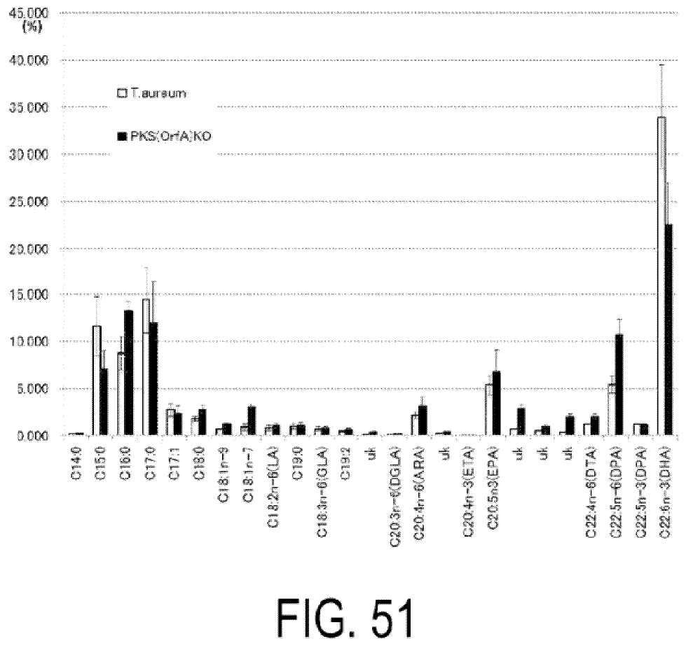

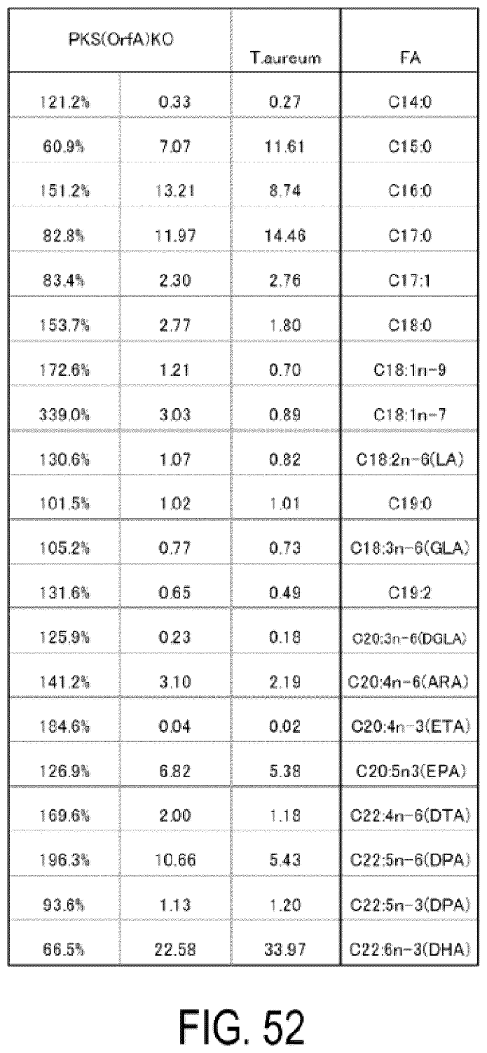

The changes in the fatty acid composition are shown in FIG. 51. FIG. 52 shows the proportion when the wild-type strain is taken as 100%. FIG. 52 shows that, of the total fatty acid composition, ARA is 3.10%, DGLA is 0.23%, ETA is 0.04%, EPA is 6.82%, n-6 DPA is 10.66%, and DHA is 22.58%. FIG. 52 shows that, by GC area, LA/DHA is 0.05, GLA/DHA is 0.03, DGLA/DHA is 0.01, ARA/DHA is 0.1, EPA/DHA is 0.3, LA/EPA is 0.16, GLA/EPA is 0.11, DTA/EPA is 0.29, DTA/ARA is 0.65, DTA/DGLA is 8.7, LA/n-6 DPA is 0.1, GLA/n-6 DPA is 0.07, DGLA/n-6 DPA is 0.02, ARA/n-6 DPA is 0.3, EPA/n-6 DPA is 0.6, DGLA/LA is 0.2, ARA/LA is 2.9, EPA/LA is 6.4, DTA/LA is 1.9, DGLA/GLA is 0.3, ARA/GLA is 4.0, n-6 DPA/DTA is 5.3, DHA/n-3 DPA is 20.0, C20 PUFA/C22 PUFA is 0.3, and n-6 PUFA/n-3 PUFA is 0.52.

As a result, when the PUFA-PKS pathway associated gene: OrfA was disrupted in Thraustochytrium aureum, DPA (C22:5n-6) exhibited an increasing trend and DHA (C22:6n-3) exhibited a decreasing trend.

It is known that in the genus Schizochytrium and genus Aurantiochytrium, exogenous PUFAs become necessary when a PUFA-PKS pathway gene is disrupted, and breeding is not possible unless exogenous PUFAs are supplied (Non-patent Document 4). Unlike these organisms, however, Thraustochytrium aureum ATCC 34304 can be cultured without supplementing the culture medium with exogenous PUFAs when a PUFA-PKS pathway associated gene is disrupted. Furthermore, disruption of a PUFA-PKS pathway associated gene, decreased DHA only to approximately ⅔ and slightly increased DPA (C22:5n-6) compared to the wild-type strain.

The above results suggest the possibility that DHA and n-6 DPA are produced via another biosynthesis pathway in addition to the PUFA-PKS pathway in Thraustochytrium aureum ATCC 34304. The reason that Thraustochytrium aureum ATCC 34304 can be cultured without supplementing the culture medium with exogenous PUFAs is surmised to be that endogenous PUFAs are supplied via a biosynthesis pathway other than the PUFA-PKS pathway.

### Comparative Example 3

[Measurement of Fatty Acid Composition of Lipids Produced by PUFA-PKS Gene and C20 Elongase Gene Disruption and Transformation Strain of Thraustochytrium aureum ATCC 34304]

### [Comparative Example 3-1]: Cloning of Upstream Sequence of Thraustochytrium aureum C20 Elongase Gene

The genome cassette library produced in Comparative Example 2-1 was used as a template. A PCR lower primer [RHO71: 22mer: 5′-GGG AGC GCA GGG AAA ACG GTC T-3′ (SEQ ID NO: 143)] was produced on the C20 elongase gene upstream sequence (SEQ ID NO: 76) described in Comparative Example 1-4, and the gene was amplified by combining with the cassette primers contained in the kit described in Comparative Example 2-1. [1st PCR cycles: 98° C. 2 min/98° C. 30 sec, 56° C. 30 sec, 72° C. 4 min, 30 cycles/72° C. 5 min]. Then, the 1st PCR amplification product was diluted 100-fold, and the gene was amplified by combining a PCR lower primer [RHO72: 20mer: 5′-CCA GCC CAC GTC GTC GGA GC-3′ (SEQ ID NO: 144)] and the nested primers contained in the kit described in Comparative Example 2-1. [2nd PCR cycles: 98° C. 2 min/98° C. 30 sec, 56° C. 30 sec, 72° C. 4 min, 30 cycles/72° C. 5 min]. The obtained DNA fragment was cloned in pGEM-T Easy Vector, and after amplification with E. coli, the sequence was confirmed using a Dye Terminator Cycle Sequencing Kit (available from Beckman Coulter Inc.).

A 2297 bp DNA fragment (SEQ ID NO: 145) containing the 3277 bp to 981 bp region upstream of the C20 elongase gene was cloned.

### [Comparative Example 3-2]: Cloning of Downstream Sequence of C20 Elongase Gene

The genome cassette library produced in Comparative Example 2-1 was used as a template. A PCR upper primer [RHO87: 23mer: 5′-GCC GCT CAT GCC CAC GCT CAA AC-3′ (SEQ ID NO: 146)] was produced on the C20 elongase gene downstream sequence (SEQ ID NO: 77) described in Comparative Example 1-4, and the gene was amplified by combining with the cassette primers contained in the kit described in Comparative Example 2-1. [1st PCR cycles: 98° C. 2 min/98° C. 30 sec, 56° C. 30 sec, 72° C. 4 min, 30 cycles/72° C. 5 min]. Then, the 1st PCR amplification product was diluted 100-fold, and the gene was amplified by combining a PCR lower primer [RH073: 23mer: 5′-CTT TCG GCT GCC AGG AAT CTA CG-3′ (SEQ ID NO: 147)] and the nested primers contained in the kit described in Comparative Example 2-1. [2nd PCR cycles: 98° C. 2 min/98° C. 30 sec, 56° C. 30 sec, 72° C. 4 min, 30 cycles/72° C. 5 min]. The obtained DNA fragment was cloned in pGEM-T Easy Vector, and after amplification with E. coli, the sequence was confirmed using a Dye Terminator Cycle Sequencing Kit (available from Beckman Coulter Inc.).

A 2189 bp DNA fragment (SEQ ID NO: 148) containing the 1106 bp to 3294 bp region downstream of the C20 elongase gene was cloned.

### [Comparative Example 3-3]: Production of Blasticidin Resistance Gene Cassette

Using genome DNA from Thraustochytrium aureum ATCC 34304 as a template, an ubiquitin promoter sequence (618 bp, SEQ ID NO: 149) was amplified with PrimeSTAR HS DNA Polymerase with GC Buffer (available from Takara Bio Inc.). The PCR primers used were as shown below. RH053 was set on the ubiquitin promoter sequence, and includes a BglII linker sequence (Example 2-2, SEQ ID NO: 5). RH048 includes the ubiquitin promoter sequence and a blasticidin resistance gene sequence. [RH048: 58mer: 5′-CTT CTT GAG ACA AAG GCT TGG CCA TGT TGG CTA GTG TTG CTT AGG TCG CTT GCT GCT G-3′) (SEQ ID NO: 150)]. [PCR cycles: 98° C. 2 min/98° C. 10 sec, 68° C. 1 min, 30 cycles/68° C. 1 min].

Using pTracer-CMV/Bsd/lacZ (available from Invitrogen Corp.) as a template, a blasticidin resistance gene (432 bp, SEQ ID NO: 151) was amplified with PrimeSTAR HS DNA Polymerase with GC Buffer. The PCR primers used were as shown below. RHO47 includes the ubiquitin promoter sequence and the blasticidin resistance gene sequence. RHO49 includes the blasticidin resistance gene sequence and has a BglII linker sequence. [RHO47: 54mer: 5′-AGC GAC CTAAGC AAC ACT AGC CAA CAT GGC CAA GCC TTT GTC TCA AGA AGA ATC-3′ (SEQ ID NO: 152), RHO49: 38mer: 5′-CCC AGA TCT TAG CCC TCC CAC ACA TAA CCA GAG GGC AG-3′ (SEQ ID NO: 153)]. [PCR cycles: 98° C. 2 min/98° C. 10 sec, 68° C. 1 min, 30 cycles/68° C. 1 min].

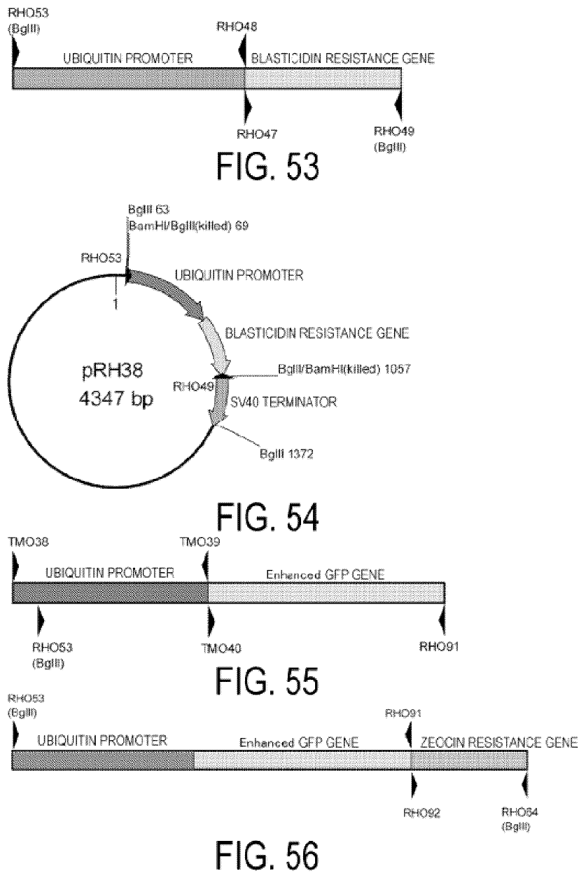

Using SEQ ID NOS: 149 and 151 as templates, fusion PCR was performed using RH053 (described in Example 2-2, SEQ ID NO: 5) and RH049 (SEQ ID NO: 153) according to the method described in Non-patent Document 9. Amplification was performed using LA Taq Hot Start Version (available from Takara Bio Inc.) as the enzyme under the following conditions, and then the amplified product was digested with BglII. [PCR cycles: 94° C. 2 min/94° C. 20 sec, 55° C. 30 sec, 68° C. 1 min, 30 cycles/68° C. 1 min (1° C./10 sec from 55° C. to 68° C.)]. (FIG. 53).

The Thraustochytrium aureum ATCC 34304-derived ubiquitin promoter—pTracer-CMV/Bsd/lacZ-derived blasticidin resistance gene sequence (1000 bp, SEQ ID NO: 154) fused as described above was digested with BglII, and the resultant was bound to the BamHI site of pRH27 (FIG. 2) described in Example 2-1. After amplification of the produced plasmid with E. coli, the sequence was confirmed using a Dye Terminator Cycle Sequencing Kit (available from Beckman Coulter Inc.). This was named pRH38.

The produced blasticidin resistance gene cassette (pRH38) is illustrated in FIG. 54.

### [Comparative Example 3-4]: Production of GFP Fusion Zeocin Resistance Gene Cassette

Using genome DNA from Thraustochytrium aureum ATCC 34304 as a template, a ubiquitin promoter sequence (812 bp, SEQ ID NO: 155) was amplified with PrimeSTAR HS DNA Polymerase with GC Buffer (available from Takara Bio Inc.). The PCR primers used were as shown below. TMO38 was set on the ubiquitin promoter sequence. TMO39 includes the ubiquitin promoter sequence and an enhanced GFP gene sequence. [TMO38: 29mer: 5′-TCG GTA CCC GTT AGA ACG CGT AAT ACG AC-3′ (SEQ ID NO: 156), TMO39: 41mer: 5′-TCC TCG CCC TTG CTC ACC ATG TTG GCT AGT GTT GCT TAG GT-3′ (SEQ ID NO: 157)]. [PCR cycles: 98° C. 10 sec/98° C. 10 sec, 58° C. 30 sec, 72° C. 1 min, 30 cycles/72° C. 1 min].

Using an enhanced GFP gene sequence (available from Clontech Laboratories, Inc.) as a template, the enhanced GFP gene sequence (748 bp, SEQ ID NO: 158) was amplified with PrimeSTAR HS DNA Polymerase (available from Takara Bio Inc.). The PCR primers used were as shown below. TMO40 includes the ubiquitin promoter sequence and the enhanced GFP gene sequence. RHO91 includes the enhanced GFP sequence and a zeocin resistance gene sequence. [TMO40: 41mer: 5′-ACC TAA GCA ACA CTA GCC AAC ATG GTG AGC AAG GGC GAG GA-3′ (SEQ ID NO: 159), RHO91: 58mer: 5′-GAA CGG CAC TGG TCA ACT TGG CGT CCA TGC CGA GAG TGA TCC CGG CGG CGG TCA CGA A-3′ (SEQ ID NO: 160)]. [PCR cycles: 98° C. 10 sec/98° C. 10 sec, 58° C. 30 sec, 72° C. 2 min, 30 cycles/72° C. 2 min].

Using SEQ ID NOS: 156 and 158 as templates, fusion PCR was performed by LA Taq Hot Start Version (available from Takara Bio Inc.) according to the method described in Non-patent Document 9. TMO38 (SEQ ID NO: 156) and RHO91 (SEQ ID NO: 160) were used as primers, and the conditions were as follows: PCR cycles: 94° C. 2 min/94° C. 20 sec, 55° C. 30 sec, 68° C. 2 min, 30 cycles/68° C. 2 min (1° C./10 sec from 55° C. to 68° C.) (FIG. 55, 1519 bp, SEQ ID NO: 161).

Using SEQ ID NO: 161 as a template, the ubiquitin promoter sequence—enhanced GFP gene sequence (1319 bp, SEQ ID NO: 162) was amplified with PrimeSTAR HS DNA Polymerase (available from Takara Bio Inc.). The primers used were as follows. RHO53 (Example 2-2, SEQ ID NO: 5) contains the ubiquitin promoter sequence and has a BglII site. RHO91 (SEQ ID NO: 160) includes the enhanced GFP sequence and a zeocin resistance gene sequence. [PCR cycles: 98° C. 2 min/98° C. 10 sec, 68° C. 2 min, 30 cycles/68° C. 2 min].

Using pcDNA 3.1 Zeo(+) as a template, a zeocin resistance gene sequence (408 bp, SEQ ID NO: 163) was amplified with PrimeSTAR HS DNA Polymerase (available from Takara Bio Inc.). RHO92 includes the enhanced GFP sequence and a zeocin resistance gene sequence. RH064 contains the zeocin resistance gene sequence and has a BglII site. [RHO92: 54mer: 5′-CGC CGC CGG GAT CAC TCT CGG CAT GGA CGC CAA GTT GAC CAG TGC CGT TCC GGT-3′ (SEQ ID NO: 164), RHO64: 38mer: 5′-CCC AGA TCT CAG TCC TGC TCC TCG GCC ACG AAG TGC AC-3′ (SEQ ID NO: 165)]. [PCR cycles: 98° C. 2 min/98° C. 10 sec, 68° C. 1 min, 30 cycles/68° C. 1 min].

Using SEQ ID NOS: 162 and 163 as templates, fusion PCR was performed by LA Taq Hot Start Version (available from Takara Bio Inc.) according to the method described in Non-patent Document 9. RHO53 (Example 2-2, SEQ ID NO: 5) and RH064 (SEQ ID NO: 165) were used as primers, and the conditions were as follows: PCR cycles: 94° C. 2 min/94° C. 20 sec, 68° C. 2 min, 30 cycles/68° C. 2 min (1° C./10 sec from 55° C. to 68° C.) (FIG. 56).

The Thraustochytrium aureum ATCC 34304-derived ubiquitin promoter—enhanced GFP gene—pcDNA 3.1 Zeo(+)-derived zeocin resistance gene (FIG. 56, 1677 bp, SEQ ID NO: 166) fused as described above was digested with BglII, and the resultant was bound to the BamHI site of pRH27 (FIG. 2) described in Example 2-1. After amplification of the produced plasmid with E. coli, the sequence was confirmed using a Dye Terminator Cycle Sequencing Kit (available from Beckman Coulter Inc.). This was named pRH51.

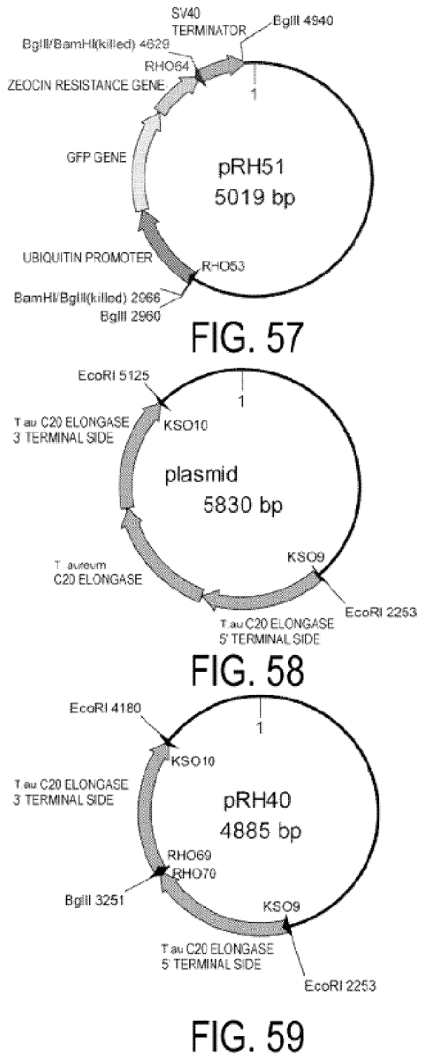

The produced GFP fusion zeocin resistance gene cassette (pRH51) is illustrated in FIG. 57.

### [Comparative Example 3-5]: Production of Plasmid Serving as Base for Production of C20 Elongase Gene Targeting Vector

Using Thraustochytrium aureum ATCC 34304 genome DNA as a template, a C20 elongase gene and its surrounding sequence were amplified with PCR by PrimeSTAR HS DNA Polymerase (available from Takara Bio Inc.) (2884 bp, SEQ ID NO: 167). The PCR primers used were as follows. Both primers contained an EcoI linker sequence. KSO9 was set in the C20 elongase gene upstream (SEQ ID NO: 76), and KSO10 was set in the C20 elongase gene downstream (SEQ ID NO: 77). [KSO9: 50mer: 5′-CCC GAA TTC ACT AGT GAT TCT CCC GGG TGG ACC TAG CGC GTG TGT CAC CT-3′ (SEQ ID NO: 168), KSO10: 40mer: 5′-CCC GAA TTC GAT TAT CCC GGG GCC GAG AAC GGG GTC GCC C-3′ (SEQ ID NO: 169)]. [PCR cycles: 98° C. 2 min/98° C. 10 sec, 68° C. 3.5 min, 30 cycles/68° C. 10 min]. PrimeSTAR HS DNA Polymerase (available from Takara Bio Inc.) was used for the enzymes, and after amplification, the amplified products were digested with EcoRI, and then cloned at the vector EcoRI site of pBlueScript (SK) (available from Stratagene Corp.). After amplification with E. coli, the sequence was confirmed using a Dye Terminator Cycle Sequencing Kit (available from Beckman Coulter Inc.) (FIG. 58).

A primer set that was set up in the reverse direction with the objective of deleting a portion of the C20 elongase gene sequence and inserting a BglII site (1939 bp, SEQ ID NO: 170) was prepared using the plasmid illustrated in FIG. 58 as a template, and the set was amplified with PrimeSTAR Max DNA Polymerase (available from Takara Bio Inc.). The PCR primers used were as shown below, both of which have a BglII linker sequence. [RHO69: 38mer: 5′-CCC AGA TCT ACC TGT TTC CGG CTG GCT CCC GAG CCA TG-3′ (SEQ ID NO: 171), RHO70: 38mer: 5′-CCC AGA TCT GGT CGC GTT TAC AAA GCA GCG CAG CAA CA-3′ (SEQ ID NO: 172)]. [PCR cycles: 98° C. 2 min/98° C. 10 sec, 68° C. 1.5 min, 30 cycles/68° C. 1.5 min]. After amplification under the above conditions, the amplified product was digested with BglII and then self-ligated. After the ligated sample was amplified with E. coli, the sequence was confirmed using a Dye Terminator Cycle Sequencing Kit (available from Beckman Coulter Inc.). This was named pRH40.

The produced plasmid (pRH40) serving as a base for production of a C20 elongase gene targeting vector is illustrated in FIG. 59.

### [Comparative Example 3-6]: Production of Targeting Vectors (Blasticidin Resistance Gene and GFP Fusion Zeocin Resistance Gene)

pRH38 (FIG. 52) described in Comparative Example 3-3 was digested with BglII, and a DNA fragment containing a blasticidin resistance gene cassette was bound to the BglII site of pRH40 (FIG. 59) described in Comparative Example 3-5. This was named pRH43.

pRH51 (FIG. 55) described in Comparative Example 3-4 was digested with BglII, and a DNA fragment containing a GFP fusion zeocin resistance gene cassette was bound to the BglII site of pRH40 (FIG. 57) described in Comparative Example 3-5. This was named pRH54.

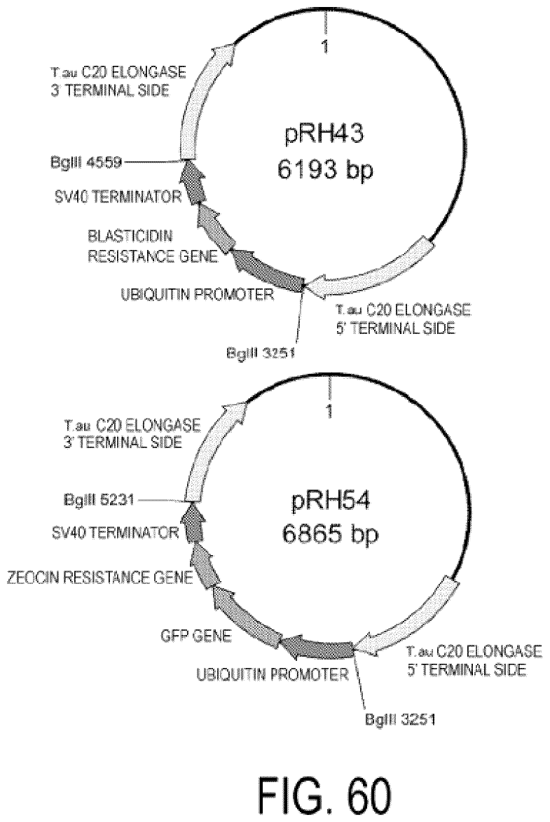

The two produced targeting vectors (pRH43 and 54) are illustrated in FIG. 60.

### [Comparative Example 3-7]: Transfer of C20 Elongase Gene Targeting Vectors into Thraustochytrium aureum OrfA Disruption Strain

Using the two targeting vectors produced in Comparative Example 3-6 as templates, the genes were amplified with PrimeSTAR Max DNA Polymerase (available from Takara Bio Inc.) using KSO11 and KSO12 as primers. KSO11 was set upstream of the Thraustochytrium aureum C20 elongase gene, and KSO12 was set downstream of the Thraustochytrium aureum C20 elongase gene. [KSO11: 31mer: 5′-CTC CCG GGT GGA CCT AGC GCG TGT GTC ACC T-3′ (SEQ ID NO: 173), KSO12: 27mer: 5′-ATC CCG GGG CCG AGA ACG CCC TCG CCC-3′ (SEQ ID NO: 174)]. [PCR cycles: 98° C. 2 min/98° C. 30 sec, 68° C. 2 min, 30 cycles/68° C. 2 min]. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pRH43 (FIG. 60) described in Comparative Example 3-6 was used as a template was 3215 bp, and resulted in a sequence including Thraustochytrium aureum C20 elongase gene upstream—ubiquitin promoter—blasticidin resistance gene sequence—SV40 terminator sequence—Thraustochytrium aureum C20 elongase gene downstream (SEQ ID NO: 175). The transfer fragment obtained when pRH54 (FIG. 60) described in Comparative Example 3-6 was used as a template was 3887 bp, and resulted in a sequence including Thraustochytrium aureum C20 elongase gene upstream—ubiquitin promoter—enhanced GFP gene sequence—zeocin resistance gene sequence—SV40 terminator sequence—Thraustochytrium aureum C20 elongase gene downstream (SEQ ID NO: 176).

The PUFA-PKS pathway associated gene: OrfA gene disruption strain described in Comparative Example 2 was cultured for 4 days in a GY culture medium, and cells in the logarithmic growth phase were used in gene transfer. To cells corresponding to OD600=1 to 1.5, 0.625 μg of DNA fragment was transformed by the gene gun method (microcarrier: 0.6 micron gold particles, target distance: 6 cm, chamber vacuum: 26 mmHg, rupture disk: 1100 psi). After a recovery time of 4 to 6 hr, the transgenic cells were spread on a PDA agar plate culture medium (containing 2 mg/mL of G418 or containing 2 mg/mL of hygromycin). As a result, from 100 to 200 cells of drug resistant strain per shot were obtained.

### [Comparative Example 3-8]: Identification of C20 Elongase Gene Targeting Homologous Recombinant

After genome DNA was extracted from the C20 elongase gene disruption strain in the Thraustochytrium aureum OrfA disruption strain and Thraustochytrium aureum by the method described in Example 2-2, A260/A280 was measured and the DNA concentration was calculated.

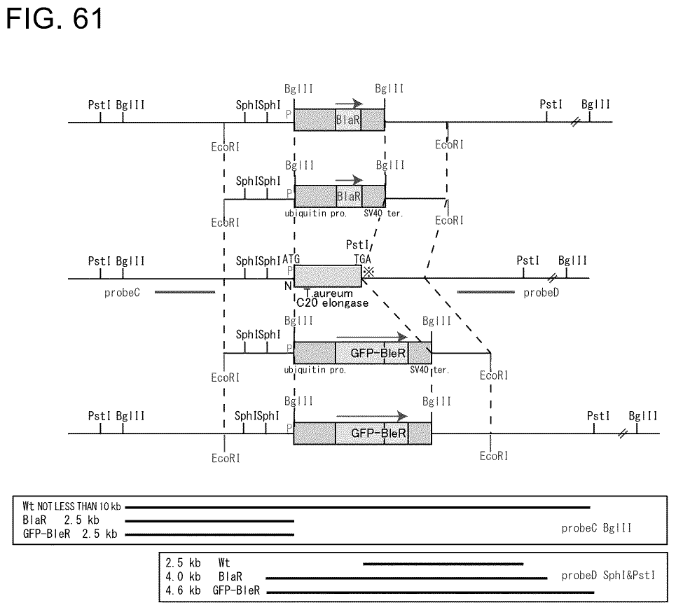

After the genome DNA was cut with a restriction enzyme, and underwent electrophoresis in approximately 2 to 3 μg per well of 0.7% SeaKem GTG agarose gel (available from Takara Bio Inc.). This was transformed to a nylon membrane, and hybridized for at 51° C. 16 hr with a probe produced using DIG System (available from Roche Applied Science, Inc.). The primers used in probe production were as follows. 5′ side [RHO94: 21mer: 5′-ACG TCC GCT TCA AAC ACC TCG-3′ (SEQ ID NO: 177), RHO95: 24mer: 5′-TCG GAA CAA CTG GAA CAA CTA AAG-3′ (SEQ ID NO: 178)]; 3′ side [RHO96: 22mer: 5′-ATG TCG CTC TCC TTC TTC TCA G-3′ (SEQ ID NO: 179), RHO97: 21mer: 5′-TCG GCT CCT GGA AAG TGC TCT-3′ (SEQ ID NO: 180)]. [PCR cycles: 98° C. 2 min/98° C. 30 sec, 58° C. 30 sec, 72° C. 1 min, 30 cycles/72° C. 3 min]. The positions of the restriction enzymes and the probes used are illustrated in FIG. 61. The hybridized probes were detected using the color development method (NBT/BCIP solution).

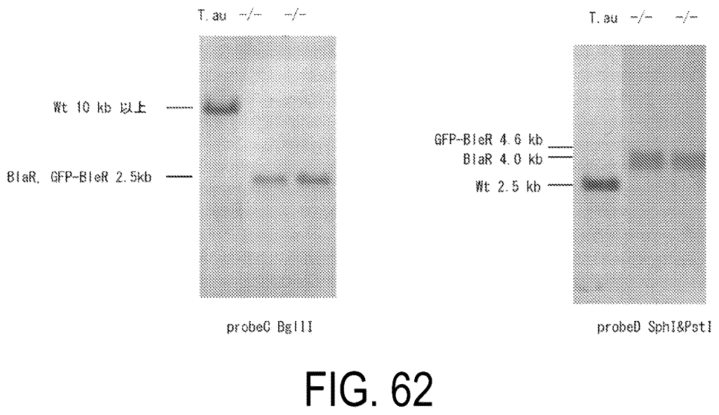

In analysis of both the 5′ side and the 3′ side, bands were observed at the expected sizes when the drug resistance genes caused homologous recombination (FIG. 62). The experiment reveals that the Thraustochytrium aureum ATCC 34304 strain does not require nutrients even when the PKS pathway associated gene: OrfA and the C20 elongase gene are deleted.

### [Comparative Example 3-9]: Change in Fatty Acid Composition by C20 Elongase Gene Disruption in Thraustochytrium aureum OrfA Disruption Strain

Thraustochytrium aureum ATCC 34304 and the gene disruption strain were cultured according to the method described in Example 2-9, and after freeze drying, the fatty acids were methyl-esterified and analyzed using GC. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min).

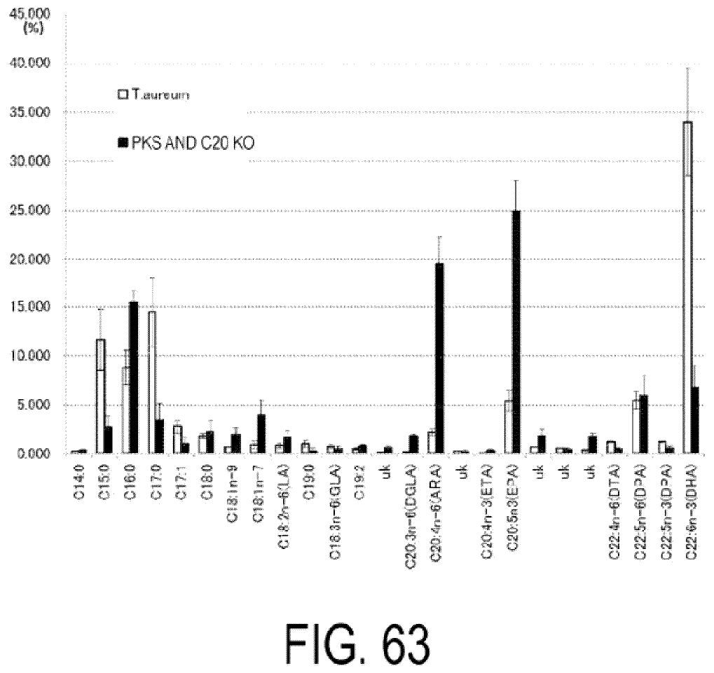

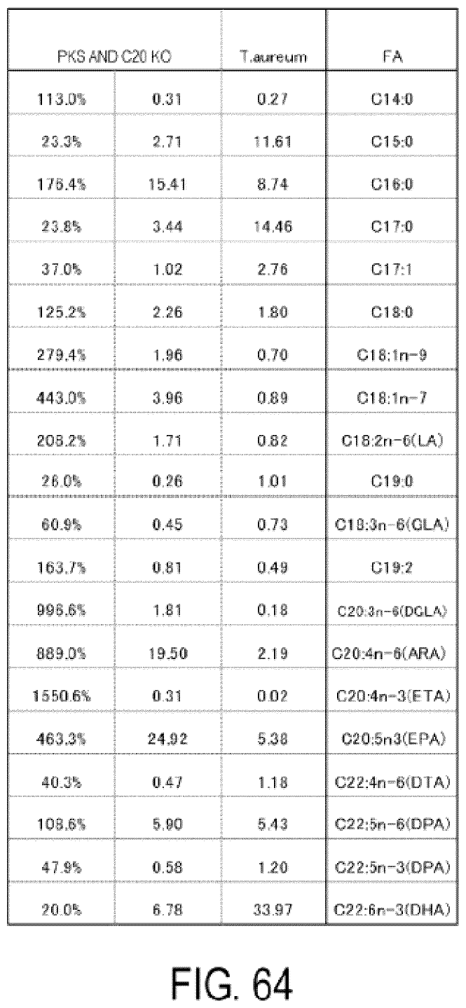

The changes in the fatty acid composition are shown in FIG. 63. Furthermore, FIG. 64 shows the proportion when the wild-type strain is taken as 100%. FIG. 64 shows that, of the total fatty acid composition, ARA is 19.50%, DGLA is 1.81%, ETA is 0.31%, EPA is 24.92%, n-6 DPA is 5.90%, and DHA is 6.78%. FIG. 64 shows that, by GC area, LA/DHA is 0.25, GLA/DHA is 0.07, DGLA/DHA is 0.27, ARA/DHA is 2.88, EPA/DHA is 3.68, LA/EPA is 0.07, GLA/EPA is 0.02, DTA/EPA is 0.02, DTA/ARA is 0.02, DTA/DGLA is 0.26, LA/n-6 DPA is 0.29, GLA/n-6 DPA is 0.08, DGLA/n-6 DPA is 0.31, ARA/n-6 DPA is 3.31, EPA/n-6 DPA is 4.22, DGLA/LA is 1.06, ARA/LA is 11.40, EPA/LA is 14.57, DTA/LA is 0.27, DGLA/GLA is 4.02, ARA/GLA is 43.33, n-6 DPA/DTA is 12.55, DHA/n-3 DPA is 11.69, C20 PUFA/C22 PUFA is 3.39, and n-6 PUFA/n-3 PUFA is 0.85.

As a result, when the C20 elongase gene was disrupted in the Thraustochytrium aureum OrfA disruption strain, C20:4n-6 (ARA) increased approximately 8-fold, C20:5n-3 (EPA) increased approximately 4-fold, and C22:6n-3 (DHA) decreased to approximately ⅕.

Thus, it was demonstrated that in order to create a strain in which the produced quantity of DHA and DPA n-6 are markedly reduced from Thraustochytrium aureum ATCC 34304, which has both an endogenous elongase-desaturase pathway and an endogenous PUFA-PKS pathway, both a gene of an enzyme constituting the elongase-desaturase pathway (for example, the C20 elongase gene) and a PUFA-PKS pathway associated gene need to be disrupted.

### Comparative Example 4

[Measurement of Fatty Acid Composition of Lipids Produced by PUFA-PKS Gene and Δ4 Desaturase Gene Disruption and Transformation Strain of Thraustochytrium aureum ATCC 34304]

### [Comparative Example 4-1]: Cloning Sequence of 1071 bp Upstream of Δ4 Desaturase Gene to 1500 bp within Δ4 Desaturase Gene of Thraustochytrium aureum ATCC 34304 Strain

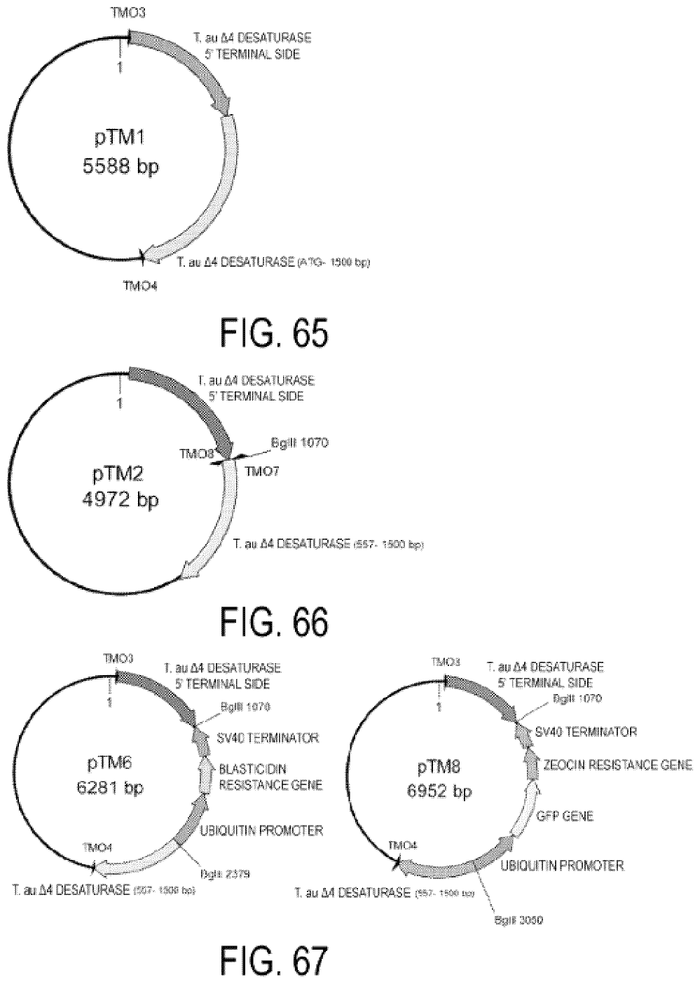

Genome DNA of the Thraustochytrium aureum ATCC 34304 strain extracted by the method described in Example 2-2 was read, and a gene sequence having high homology to known Δ4 desaturase was searched for. Two PCR primers were designed based on the search results. TMO3 is a sequence located at 1071 to 1049 bp upstream of the Δ4 desaturase gene of the Thraustochytrium aureum ATCC 34304 strain, and TMO4 is a sequence within the protein coding region located at 1477 to 1500 bp counting from the start codon. [TMO3: 23mer: 5′-GGC GGA GCG AAG TGT GAA AGT TA-3′ (SEQ ID NO: 181), TMO4: 24mer: 5′-GCG ACA GCA TCT TGA AAT AGG CAG-3′ (SEQ ID NO: 182)]. Using genome DNA of the Thraustochytrium aureum ATCC 34304 strain as a primer, the sequence of 1071 bp upstream of the Δ4 desaturase gene to 1500 bp within the Δ4 desaturase gene (2571 bp, SEQ ID NO: 183) of Thraustochytrium aureum ATCC 34304 strain was amplified using these two primers. The amplification conditions were as follows. [PCR cycles: 98° C. 2 min/98° C. 20 sec, 60° C. 30 sec, 72° C. 3 min, 30 cycles/72° C. 8 min]. The obtained DNA fragment was cloned in pGEM-T Easy Vector, and after amplification with E. coli, the sequence was confirmed using a Dye Terminator Cycle Sequencing Kit (available from Beckman Coulter Inc.). This was named pTM1 (FIG. 65).

### [Comparative Example 4-2]: Production of Plasmid Serving as Base for Production of Δ4 Desaturase Gene Targeting Vector

Using pTM1 (FIG. 65) produced in Comparative Example 4-1 as a template, a primer set set in the reverse direction so as to delete a 556 bp sequence (616 bp, SEQ ID NO: 184) containing 60 bp upstream of the Δ4 desaturase gene and the start codon within the Δ4 desaturase gene and to produce a BglII site in the deleted portion was prepared. TMO7 and TMO8 both contain a BglII sequence. PrimeSTAR Max DNA Polymerase (available from Takara Bio Inc.) was used in amplification. [TMO7: 25mer: 5′-CAG GAG ATC TCC AAG TCG CGA TTC A-3′ (SEQ ID NO: 185), TMO8: 26mer: 5′-CTT GGA GAT CTC CTG CCC GTC CCG AA-3′ (SEQ ID NO: 186)]. [PCR cycles: 98° C. 3 min/98° C. 10 sec, 55° C. 15 sec, 72° C. 30 sec, 30 cycles/72° C. 30 sec]. After amplification under the above conditions, the amplified product was purified by electrophoresis using agarose gel. After transforming the obtained DNA fragment into E. coli and amplifying, the sequence was confirmed using a Dye Terminator Cycle Sequencing Kit (available from Beckman Coulter Inc.). This was named pTM2.

The produced plasmid (pTM2) serving as a base for production of a Δ4 desaturase gene targeting vector is illustrated in FIG. 66.

### [Comparative Example 4-3]: Production of Targeting Vectors (Blasticidin Resistance Gene and GFP Fusion Zeocin Resistance Gene)

pRH38 (FIG. 54) described in Comparative Example 3-3 was digested with BglII, and a DNA fragment containing a blasticidin resistance gene cassette was bound to the BglII site of pTM2 (FIG. 66) described in Comparative Example 4-2. This was named pTM6.

pRH51 (FIG. 57) described in Comparative Example 3-4 was digested with BglII, and a DNA fragment containing a GFP fusion zeocin resistance gene cassette was bound to the BglII site of pTM2 (FIG. 66) described in Comparative Example 4-2. This was named pTM8.

The two produced targeting vectors (pTM6 and 8) are illustrated in FIG. 67.

### [Comparative Example 4-4]: Transfer of Δ4 Desaturase Gene Targeting Vectors into Thraustochytrium aureum OrfA Disruption Strain

Using the two targeting vectors produced in Comparative Example 4-3 as templates, the genes were amplified with PrimeSTAR Max DNA Polymerase (available from Takara Bio Inc.) using TMO3 (described in Comparative Example 4-1, SEQ ID NO: 181) and TMO4 (described in Comparative Example 4-1, SEQ ID NO: 182) as primers. [PCR cycles: 98° C. 3 min/98° C. 10 sec, 55° C. 5 sec, 72° C. 4 min, 30 cycles/72° C. 3 min]. After phenol chloroform extraction and chloroform extraction, the DNA underwent ethanol precipitation, and the precipitate was dissolved in 0.1×TE. A260/280 was measured and the DNA concentration was calculated. The transfer fragment obtained when pTM6 (FIG. 67) described in Comparative Example 4-3 was used as a template was 3264 bp, and resulted in a sequence within Thraustochytrium aureum Δ4 desaturase gene upstream—SV40 terminator sequence—blasticidin resistance gene sequence—ubiquitin promoter—Thraustochytrium aureum Δ4 desaturase gene (SEQ ID NO: 187). The transfer fragment obtained when pTM8 (FIG. 67) described in Comparative Example 4-3 was used as a template was 3935 bp, and resulted in a sequence within Thraustochytrium aureum Δ4 desaturase gene upstream—SV40 terminator sequence—zeocin resistance gene sequence—enhanced GFP gene sequence—ubiquitin promoter—Thraustochytrium aureum Δ4 desaturase gene (SEQ ID NO: 188).

The PUFA-PKS pathway associated gene: OrfA gene disruption strain described in Comparative Example 2 was cultured for 4 days in a GY culture medium, and cells in the logarithmic growth phase were used in gene transfer. To cells corresponding to OD600=1 to 1.5, 0.625 μg of DNA fragment was transformed by the gene gun method (microcarrier: 0.6 micron gold particles, target distance: 6 cm, chamber vacuum: 26 mmHg, rupture disk: 1100 psi). After a recovery time of 4 to 6 hr, the transgenic cells were spread on a PDA agar plate culture medium (containing 20 mg/mL of zeocin or containing 0.2 mg/mL of blasticidin). As a result, from 100 to 200 cells of drug resistant strain per shot were obtained.

### [Comparative Example 4-5]: Identification of Δ4 Desaturase Gene Targeting Homologous Recombinant

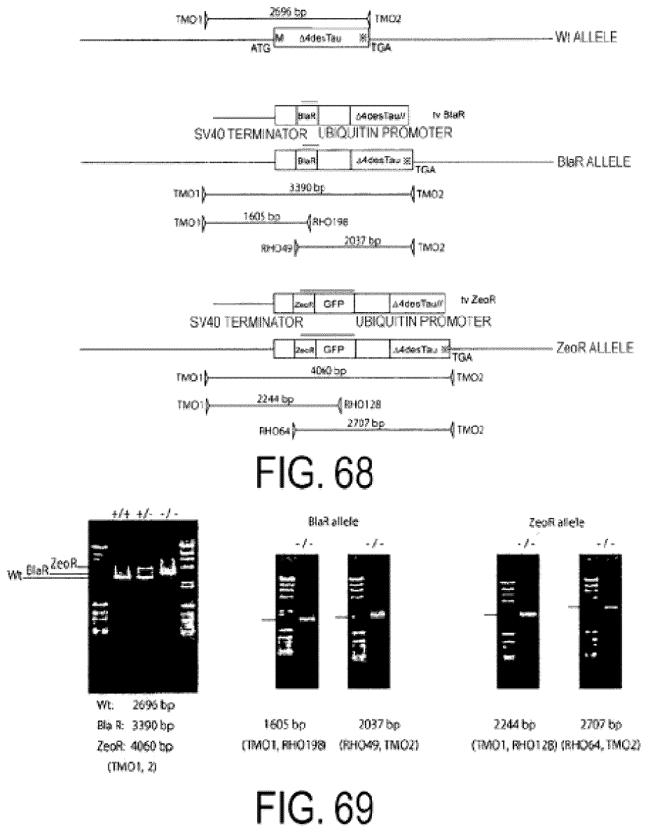

After genome DNA was extracted from the Δ4 desaturase gene disruption strain in the Thraustochytrium aureum OrfA disruption strain and Thraustochytrium aureum by the method described in Example 2-2, A260/A280 was measured and the DNA concentration was calculated. Using the genome DNA as templates, PCR for genome structure confirmation was performed using Mighty Amp DNA Polymerase (available from Takara Bio Inc.). The positions of the primers used, the combinations used in amplification, and the expected sizes of the amplification products are illustrated in FIG. 68. TMO1 was set upstream of the Δ4 desaturase gene, TMO2 was set downstream of the Δ4 desaturase gene, RHO198 (SEQ ID NO: 191) and RH049 (described in Comparative Example 3-3, SEQ ID NO: 153) were set on the blasticidin resistance gene, RHO128 was set on the enhanced GFP gene, and RH064 (described in Comparative Example 3-4, SEQ ID NO: 165) was set on the zeocin resistance gene. [TMO1: 23mer: 5′-AAA AGA ACA AGC CCT CTC CTG GA-3′ (SEQ ID NO: 189), TMO2: 23mer: 5′-GAG GTT TGT ATG TTC GGC GGT TT-3′ (SEQ ID NO: 190), RHO198: 26mer: 5′-TGG GGG ACC TTG TGC AGA ACT CGT GG-3′ (SEQ ID NO: 191), RHO128: 22mer: 5′-GAC CTA CGG CGT GCA GTG CTT C-3′ (SEQ ID NO: 192)]. [PCR cycles: 98° C. 2 min/98° C. 10 sec, 68° C. 4 min 30 sec, 30 cycles/68° C. 4 min]. A Δ4 desaturase gene disruption strain was obtained, wherein there was no amplification in the wild-type allele (Wt allele), and there was amplification in the blasticidin resistance gene allele (BlaR allele) and the zeocin resistance gene allele (ZeoR allele) (FIG. 69). The experiment reveals that the Thraustochytrium aureum ATCC 34304 strain does not require nutrients even when the PKS pathway associated gene: OrfA and the Δ4 desaturase gene are deleted.

### [Comparative Example 4-6]: Change in Fatty Acid Composition by Δ4 Desaturase Gene Disruption in Thraustochytrium aureum OrfA Disruption Strain

Thraustochytrium aureum ATCC 34304 and the gene disruption strain were cultured according to the method described in Example 2-9, and after freeze drying, the fatty acids were methyl-esterified and analyzed using GC. In GC analysis, measurement was performed using a gas chromatograph GC-2014 (available from Shimadzu Corporation) under the following conditions. Column: HR-SS-10 (30 m×0.25 mm; available from Shinwa Chemical Industries Ltd.); column temperature: 150° C.→(5° C./min)→220° C. (10 min); carrier gas: He (1.3 mL/min).

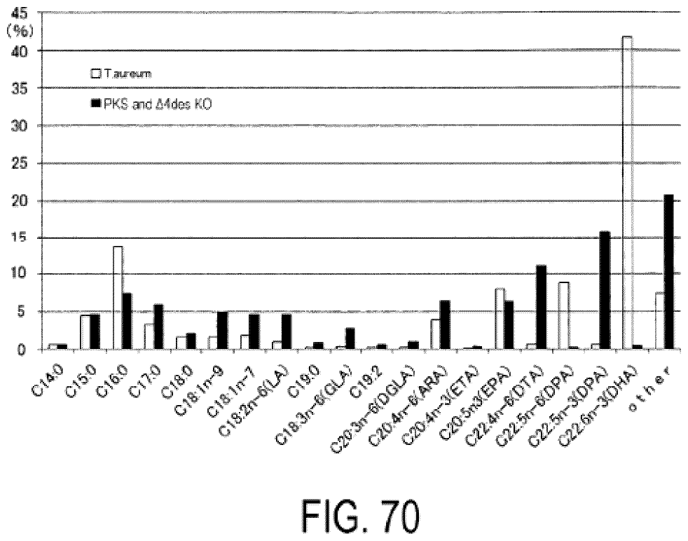

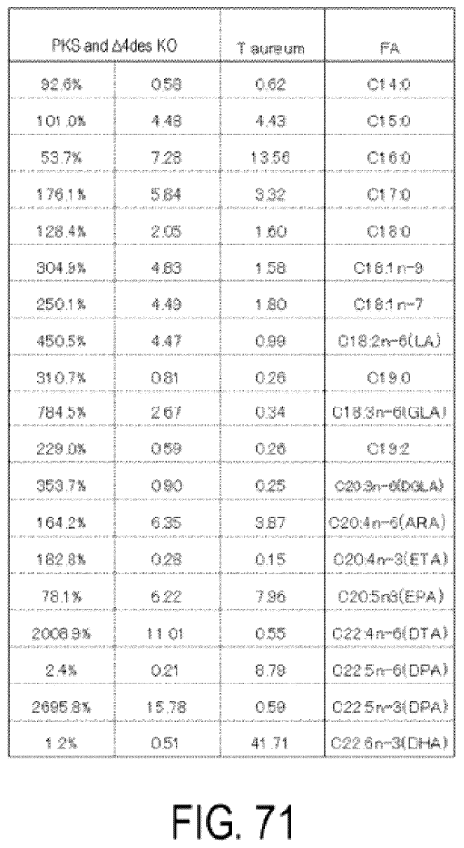

The changes in the fatty acid composition are shown in FIG. 70. Furthermore, FIG. 71 shows the proportion when the wild-type strain is taken as 100%. FIG. 71 shows that, of the total fatty acid composition, ARA is 6.35%, DGLA is 0.90%, ETA is 0.28%, EPA is 6.22%, n-6 DPA is 0.21%, and DHA is 0.51%. FIG. 71 shows that, by GC area, LA/DHA is 8.76, GLA/DHA is 1.59, DGLA/DHA is 1.76, ARA/DHA is 12.45, EPA/DHA is 12.20, LA/EPA is 0.72, GLA/EPA is 0.13, DTA/EPA is 1.77, DTA/ARA is 1.73, DTA/DGLA is 12.23, LA/n-6 DPA is 21.29, GLA/n-6 DPA is 3.86, DGLA/n-6 DPA is 4.29, ARA/n-6 DPA is 30.24, EPA/n-6 DPA is 29.62, DGLA/LA is 0.20, ARA/LA is 1.42, EPA/LA is 1.39, DTA/LA is 2.46, DGLA/GLA is 1.11, ARA/GLA is 7.84, n-6 DPA/DTA is 0.02, DHA/n-3 DPA is 0.03, C20 PUFA/C22 PUFA is 0.50, and n-6 PUFA/n-3 PUFA is 0.81.

As a result, when the Δ4 desaturase gene is disrupted in the Thraustochytrium aureum OrfA disruption strain, C22:5n-6 (DPA) and C22:6n-3 (DHA) are not substantially biosynthesized, and the Δ4 desaturase substrates C22:4n-6 (DTA) and C22:5n-3 (DPA) are accumulated.

Thus, it was demonstrated that in order to create a strain that cannot substantially biosynthesize DHA and n-6 DPA from Thraustochytrium aureum ATCC 34304, which has both an endogenous elongase-desaturase pathway and an endogenous PUFA-PKS pathway, both a gene of an enzyme constituting the elongase-desaturase pathway (for example, the Δ4 desaturase gene) and a PUFA-PKS pathway associated gene needs to be disrupted.

By using the microbial oil obtained in this manner, it is possible to obtain microbial oil having a fatty acid composition in which the composition ratios of PUFAs other than DHA and n-6 DPA are increased. It is possible to produce any PUFAs by modifying the genes of a microorganism that produces a large amount of DHA. Furthermore, by producing microbial oil that contains particularly little DHA and n-6 DPA, it is possible to produce microbial oil that requires little refinement. Additionally, by transforming elongase and desaturase into a microorganism in this manner, it is possible to obtain a microorganism that produces microbial oil.

### INDUSTRIAL APPLICABILITY

A new “pattern” of biosynthesis pathway of polyunsaturated fatty acids (PUFAs) was discovered in microorganisms called labyrinthulids. Because it is possible to provide labyrinthulea that produce PUFAs via only the elongase-desaturase pathway, it is anticipated that PUFAs will be mass produced using only the elongase-desaturase pathway.

1. A method for producing a microbial oil, comprising:
genetically modifying a labyrinthulid which belongs to the genus Parietichytrium or the genus Schizochytrium by disrupting and/or silencing a Δ4 desaturase gene, a C20 elongase gene, and/or a C18 elongase gene, or by being transformed with an ω3 desaturase gene;
culturing the labyrinthulid; and
collecting the microbial oil from the labyrinthulid,
wherein the labyrinthulid has an activity of synthesizing DHA via a PUFA-PKS pathway in an amount of not more than 1/100 of a total amount of DHA synthesized in the labyrinthulid or no activity of producing PUFAs via the PUFA-PKS pathway, and the labyrinthulid has an activity of producing PUFAs via an endogenous elongase-desaturase pathway.

2. The method according to claim 1, wherein the microbial oil comprises an increased amount of DTA as compared with the microbial oil collected from the labyrinthulid whose Δ4 desaturase gene is not disrupted or silenced.

3. The method according to claim 1, wherein the microbial oil comprises an increased amount of n-3 DPA as compared with the microbial oil collected from the labyrinthulid whose Δ4 desaturase gene is not disrupted or silenced.

4. The method according to claim 1, wherein the microbial oil comprises an increased amount of EPA as compared with the microbial oil collected from the labyrinthulid whose C20 elongase gene is not disrupted or silenced.

5. The method according to claim 1, wherein the microbial oil comprises an increased amount of ARA as compared with the microbial oil collected from the labyrinthulid whose C20 elongase gene is not disrupted or silenced.

6. The method according to claim 1, wherein the microbial oil comprises an increased amount of STA as compared with the microbial oil collected from the labyrinthulid whose C18 elongase gene is not disrupted or silenced.

7. The method according to claim 1, wherein the microbial oil comprises an increased amount of GLA as compared with the microbial oil collected from the labyrinthulid whose C18 elongase gene is not disrupted or silenced.

8. The method according to claim 1, wherein the microbial oil comprises an increased amount of EPA as compared with the microbial oil collected from the labyrinthulid which is not transformed with the ω3 desaturase gene.

9. The method according to claim 1, wherein the microbial oil comprises an increased amount of ETA as compared with the microbial oil collected from the labyrinthulid which is not transformed with the ω3 desaturase gene.

10. The method according to claim 1, wherein the labyrinthulid belonging to the genus Parietichytrium is Parietichytrium sp. SEK358 (FERM BP-11405), Parietichytrium sarkarianum SEK364 (FERM BP-11298), or Parietichytrium sp. SEK571 (FERM BP11406), and/or wherein the labyrinthulid belonging to the genus Schizochytrium is Schizochytrium aggregatum ATCC 28209.

11. A method of obtaining a genetically modified labyrinthulid, comprising the steps of:
isolating a labyrinthulid which can lose a DHA and/or n-6 DPA production activity when a Δ4 desaturase gene or a C20 elongase gene of the labyrinthulid is disrupted or silenced;
genetically modifying the labyrinthulid by disrupting or silencing the Δ4 desaturase gene or the C20 elongase gene such that the labyrinthulid produces a decreased amount of DHA and/or n-6 DPA as compared with the labyrinthulid whose Δ4 desaturase and C20 elongase gene are not disrupted or silenced.

12. The method according to claim 11, wherein the labyrinthulid belonging to the genus Parietichytrium is Parietichytrium sp. SEK358 (FERM BP-11405), Parietichytrium sarkarianum SEK364 (FERM BP-11298), or Parietichytrium sp. SEK571 (FERM BP11406), and/or wherein the labyrinthulid belonging to the genus Schizochytrium is Schizochytrium aggregatum ATCC 28209.


SEQUENCE LISTING (192 sequences)

SEQ ID NO: 1 - DNA, 34 bp, synthetic construct
  misc_feature 1..34: note = primer
  source 1..34: mol_type = other DNA
cagatctggatccgcgaaatgaccgaccaagcga

SEQ ID NO: 2 - DNA, 24 bp, synthetic construct
  misc_feature 1..24: note = primer
  source 1..24: mol_type = other DNA
acgcaattaatgtgagatctagct

SEQ ID NO: 3 - DNA, 342 bp, synthetic construct
  misc_feature 1..342: note = SV40 terminator
  source 1..342: mol_type = other DNA
cagatctggatccgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagatctcacattaattgcgt

SEQ ID NO: 4 - DNA, 619 bp, synthetic construct
  misc_feature 1..619: note = ubiquitin promoter
  source 1..619: mol_type = other DNA
cccagatctgccgcagcgcctggtgcacccgccgggcgttgttggtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcgggagcgagctggttgctggaaaagtcgcgaacgctgggctgtgtcacgcgtccacttcgggcagaccccaaacgacaagcagaacaagcaacaccagcagcagcaagcgacctaagcaacactagccaacatgattgaacaggacggccttcacg

SEQ ID NO: 5 - DNA, 36 bp, synthetic construct
  misc_feature 1..36: note = primer
  source 1..36: mol_type = other DNA
cccagatctgccgcagcgcctggtgcacccgccggg

SEQ ID NO: 6 - DNA, 58 bp, synthetic construct
  misc_feature 1..58: note = primer
  source 1..58: mol_type = other DNA
cgtgaaggccgtcctgttcaatcatgttggctagtgttgcttaggtcgcttgctgctg

SEQ ID NO: 7 - DNA, 826 bp, synthetic construct
  misc_feature 1..826: note = Neomycin resistance gene (Neor)
  source 1..826: mol_type = other DNA
agcgacctaagcaacactagccaacatgattgaacaggacggccttcacgctggctcgcccgctgcttgggtggaacggctgttcggctacgactgggctcagcagacgatcggctgctcggacgcggccgtgttccgccttagcgcgcagggccggccggtcctgtttgtcaagaccgaccttagcggcgccctcaacgagctccaggacgaagctgcccgcctcagctggcttgccacgacgggggttccgtgcgccgctgtgctcgacgtcgtcaccgaagccggccgcgactggctgctcctcggggaagtgcccggccaggacctcctcagcagccacctcgcgcccgctgagaaggtgtccatcatggccgacgccatgcgccgcctgcacaccctcgaccccgccacctgccccttcgaccaccaggcgaagcacaggatcgaacgcgcccgcacgcggatggaggctggcctcgtcgaccaagacgacctcgacgaggagcaccagggcctcgcgccggcggaactgttcgccaggcttaaggctaggatgccggacggcgaggacctcgtggtcacgcacggcgacgcctgcctccccaacatcatggtcgagaacggccgcttctcgggctttatcgactgcgggcgcctgggcgtggcggaccgctaccaagacatcgcgctcgccacgcgggacatcgccgaggagcttggcggcgagtgggccgaccgctttctcgtgctctacggcatcgccgccccggacagccagaggattgcgttctaccgcctcctggacgagttcttttgagatctg

SEQ ID NO: 8 - DNA, 54 bp, synthetic construct
  misc_feature 1..54: note = primer
  source 1..54: mol_type = other DNA
agcgacctaagcaacactagccaacatgattgaacaggacggccttcacgctgg

SEQ ID NO: 9 - DNA, 26 bp, synthetic construct
  misc_feature 1..26: note = primer
  source 1..26: mol_type = other DNA
cagatctcaaaagaactcgtccagga

SEQ ID NO: 10 - DNA, 1395 bp, synthetic construct
  misc_feature 1..1395: note = fusion DNA (T. aureum ATCC 34304 ubiquitin promoter/Neor)
  source 1..1395: mol_type = other DNA
cccagatctgccgcagcgcctggtgcacccgccgggcgttgttggtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcgggagcgagctggttgctggaaaagtcgcgaacgctgggctgtgtcacgcgtccacttcgggcagaccccaaacgacaagcagaacaagcaacaccagcagcagcaagcgacctaagcaacactagccaacatgattgaacaggacggccttcacgctggctcgcccgctgcttgggtggaacggctgttcggctacgactgggctcagcagacgatcggctgctcggacgcggccgtgttccgccttagcgcgcagggccggccggtcctgtttgtcaagaccgaccttagcggcgccctcaacgagctccaggacgaagctgcccgcctcagctggcttgccacgacgggggttccgtgcgccgctgtgctcgacgtcgtcaccgaagccggccgcgactggctgctcctcggggaagtgcccggccaggacctcctcagcagccacctcgcgcccgctgagaaggtgtccatcatggccgacgccatgcgccgcctgcacaccctcgaccccgccacctgccccttcgaccaccaggcgaagcacaggatcgaacgcgcccgcacgcggatggaggctggcctcgtcgaccaagacgacctcgacgaggagcaccagggcctcgcgccggcggaactgttcgccaggcttaaggctaggatgccggacggcgaggacctcgtggtcacgcacggcgacgcctgcctccccaacatcatggtcgagaacggccgcttctcgggctttatcgactgcgggcgcctgggcgtggcggaccgctaccaagacatcgcgctcgccacgcgggacatcgccgaggagcttggcggcgagtgggccgaccgctttctcgtgctctacggcatcgccgccccggacagccagaggattgcgttctaccgcctcctggacgagttcttttgagatctg

SEQ ID NO: 11 - DNA, 617 bp, synthetic construct
  misc_feature 1..617: note = ubiquitin promoter
  source 1..617: mol_type = other DNA
cccagatctgccgcagcgcctggtgcacccgccgggcgttgttgtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgacctaagcaacactagccaacatgaaaaagcctgaactcaccgcga

SEQ ID NO: 12 - DNA, 58 bp, synthetic construct
  misc_feature 1..58: note = primer
  source 1..58: mol_type = other DNA
tcgcggtgagttcaggctttttcatgttggctagtgttgcttaggtcgcttgctgctg

SEQ ID NO: 13 - DNA, 1058 bp, synthetic construct
  misc_feature 1..1058: note = Hygromycin resistance gene (Hygr)
  source 1..1058: mol_type = other DNA
agcgacctaagcaacactagccaacatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagatctg

SEQ ID NO: 14 - DNA, 56 bp, synthetic construct
  misc_feature 1..56: note = primer
  source 1..56: mol_type = other DNA
agcgacctaagcaacactagccaacatgaaaaagcctgaactcaccgcgacgtctg

SEQ ID NO: 15 - DNA, 36 bp, synthetic construct
  misc_feature 1..36: note = primer
  source 1..36: mol_type = other DNA
cagatctctattcctttgccctcggacgagtgctgg

SEQ ID NO: 16 - DNA, 1625 bp, synthetic construct
  misc_feature 1..1625: note = fusion DNA (Thraustochytrium aureum ATCC 34304 ubiquitin promoter|pcDNA 3.1/ Hygr)
  source 1..1625: mol_type = other DNA
cccagatctgccgcagcgcctggtgcacccgccgggcgttgttgtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgacctaagcaacactagccaacatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagatctg

SEQ ID NO: 17 - DNA, 25 bp, synthetic construct
  misc_feature 1..25: note = primer
  source 1..25: mol_type = other DNA
ccttcggcgctcctcttatgtatgt

SEQ ID NO: 18 - DNA, 25 bp, synthetic construct
  misc_feature 1..25: note = primer
  source 1..25: mol_type = other DNA
caatgcaagaggcgaactgggagag

SEQ ID NO: 19 - DNA, 25 bp, synthetic construct
  misc_feature 1..25: note = primer
  source 1..25: mol_type = other DNA
tggggctctggaaccgctgcttacg

SEQ ID NO: 20 - DNA, 25 bp, synthetic construct
  misc_feature 1..25: note = primer
  source 1..25: mol_type = other DNA
cttccagctctcccagttcgcctct

SEQ ID NO: 21 - DNA, 25 bp, synthetic construct
  misc_feature 1..25: note = primer
  source 1..25: mol_type = other DNA
cgggttgttgatgttgagcgaggtg

SEQ ID NO: 22 - DNA, 25 bp, synthetic construct
  misc_feature 1..25: note = primer
  source 1..25: mol_type = other DNA
cccacgccatccacgagcacaccac

SEQ ID NO: 23 - DNA, 957 bp, synthetic construct
  misc_feature 1..957: note = cDNA (Parietichytrium genomic DNA contains C20 elongase coding region)
  source 1..957: mol_type = other DNA
cccggatccatggcagctcgcgtggagaaacagcaggcacctgcgaaggccgccaagaaggtggggtcgcgtgtggaccgcagtgatgggttctttcgcactttcaacctctgtgcgctgtacggaagcgcgttcgcgtacgcttacaacaatgggccagtggacaacgacggcaagggcttgtacttttcaaagtctccattctacgcattcctcgtctcggacgccatgaccttcggcgctcctcttatgtatgtaattgctgtcatggcactcagccgatacatggcagacaagcagcccctcactggcttcattaaaagctacattcagccagtttacaacattgtgcaaatcgtggtgtgctcgtggatggcgtggggccttttgccacaggtggacatcttcaacctcaacccattcggtctcaacaagcagcgtgatgccaacatcgagttctttgtcatggtccacctcctgacaaagttcctcgactggaccgacaccttcatcatgattttcaagaagaactatgcacaggtctcttttctccaggtgttccaccatgccaccatcggaatggtgtggtccttcctcctccagcgcggctggggctctggaaccgctgcttacggagcgttcatcaactcggtcacccatgtcatcatgtacactcattactttgtcacctcgctcaacatcaacaacccgttcaagaggtacatcaccggcttccagctctcccagttcgcctcttgcattgtacatgctctcctcgtccttgccttcgaggaggtgtaccccctcgagtacgcttaccttcagatcagctaccacatcatcatgctctacctcttcggcaggagaatgaactggagccctctctggtgcactggcgaggtcgacgggcttgacgtcaacgtcgagacctccaagaaggctcagtaaggatccggg

SEQ ID NO: 24 - DNA, 32 bp, synthetic construct
  misc_feature 1..32: note = primer
  source 1..32: mol_type = other DNA
cccggatccatggcagctcgcgtggagaaaca

SEQ ID NO: 25 - DNA, 33 bp, synthetic construct
  misc_feature 1..33: note = primer
  source 1..33: mol_type = other DNA
cccggatccttactgagccttcttggaggtctc

SEQ ID NO: 26 - DNA, 936 bp, synthetic construct
  misc_feature 1..936: note = genomic DNA (Parietichytrium C20 elongase gene)
  source 1..936: mol_type = other DNA
atggcagctcgcgtggagaaacagcaggcacctgcgaaggccgccaagaaggtggggtcgcgtgtggaccgcagtgatgggttctttcgcactttcaacctctgtgcgctgtacggaagcgcgttcgcgtacgcttacaacaatgggccagtggacaacgacggcaagggcttgtacttttcaaagtctccattctacgcattcctcgtctcggacgccatgaccttcggcgctcctcttatgtatgtaattgctgtcatggcactcagccgatacatggcagacaagcagcccctcactggcttcattaaaagctacattcagccagtttacaacattgtgcaaatcgtggtgtgctcgtggatggcgtggggccttttgccacaggtggacatcttcaacctcaacccattcggtctcaacaagcagcgtgatgccaacatcgagttctttgtcatggtccacctcctgacaaagttcctcgactggaccgacaccttcatcatgattttcaagaagaactatgcacaggtctcttttctccaggtgttccaccatgccaccatcggaatggtgtggtccttcctcctccagcgcggctggggctctggaaccgctgcttacggagcgttcatcaactcggtcacccatgtcatcatgtacactcattactttgtcacctcgctcaacatcaacaacccgttcaagaggtacatcaccggcttccagctctcccagttcgcctcttgcattgtacatgctctcctcgtccttgccttcgaggaggtgtaccccctcgagtacgcttaccttcagatcagctaccacatcatcatgctctacctcttcggcaggagaatgaactggagccctctctggtgcactggcgaggtcgacgggcttgacgtcaacgtcgagacctccaagaaggctcag

SEQ ID NO: 27 - protein, 312 aa, synthetic construct
MAARVEKQQAPAKAAKKVGSRVDRSDGFFRTFNLCALYGSAFAYAYNNGPVDNDGKGLYFSKSPFYAFLVSDAMTFGAPLMYVIAVMALSRYMADKQPLTGFIKSYIQPVYNIVQIVVCSWMAWGLLPQVDIFNLNPFGLNKQRDANIEFFVMVHLLTKFLDWTDTFIMIFKKNYAQVSFLQVFHHATIGMVWSFLLQRGWGSGTAAYGAFINSVTHVIMYTHYFVTSLNINNPFKRYITGFQLSQFASCIVHALLVLAFEEVYPLEYAYLQISYHIIMLYLFGRRMNWSPLWCTGEVDGLDVNVETSKKAQ

SEQ ID NO: 28 - DNA, 26 bp, synthetic construct
  misc_feature 1..26: note = primer
  source 1..26: mol_type = other DNA
acaaagatctcgactggaccgacacc

SEQ ID NO: 29 - DNA, 27 bp, synthetic construct
  misc_feature 1..27: note = primer
  source 1..27: mol_type = other DNA
agtcgagatctttgtcaggaggtggac

SEQ ID NO: 30 - DNA, 935 bp, synthetic construct
  misc_feature 1..935: note = BglII@inserted C20 elongase
  source 1..935: mol_type = other DNA
atggcagctcgcgtggagaaacagcaggcacctgcgaaggccgccaagaaggtggggtcgcgtgtggaccgcagtgatgggttctttcgcactttcaacctctgtgcgctgtacggaagcgcgttcgcgtacgcttacaacaatgggccagtggacaacgacggcaagggcttgtacttttcaaagtctccattctacgcattcctcgtctcggacgccatgaccttcggcgctcctcttatgtatgtaattgctgtcatggcactcagccgatacatggcagacaagcagcccctcactggcttcattaaaagctacattcagccagtttacaacattgtgcaaatcgtggtgtgctcgtggatggcgtggggccttttgccacaggtggacatcttcaacctcaacccattcggtctcaacaagcagcgtgatgccaacatcgagttctttgtcatggtccacctcctgacaaagatctcgactggaccgacaccttcatcatgattttcaagaagaactatgcacaggtctcttttctccaggtgttccaccatgccaccatcggaatggtgtggtccttcctcctccagcgcggctggggctctggaaccgctgcttacggagcgttcatcaactcggtcacccatgtcatcatgtacactcattactttgtcacctcgctcaacatcaacaacccgttcaagaggtacatcaccggcttccagctctcccagttcgcctcttgcattgtacatgctctcctcgtccttgccttcgaggaggtgtaccccctcgagtacgcttaccttcagatcagctaccacatcatcatgctctacctcttcggcaggagaatgaactggagccctctctggtgcactggcgaggtcgacgggcttgacgtcaacgtcgagacctccaagaaggctcag

SEQ ID NO: 31 - DNA, 2661 bp, synthetic construct
  misc_feature 1..2661: note = fusion DNA (Parietichytrium C20 elongase 5' region/SV40 terminator/Neor/ubiquitin promoter/Parietichytrium C20 elongase 3' region)
  source 1..2661: mol_type = other DNA
cccggatccatggcagctcgcgtggagaaacagcaggcacctgcgaaggccgccaagaaggtggggtcgcgtgtggaccgcagtgatgggttctttcgcactttcaacctctgtgcgctgtacggaagcgcgttcgcgtacgcttacaacaatgggccagtggacaacgacggcaagggcttgtacttttcaaagtctccattctacgcattcctcgtctcggacgccatgaccttcggcgctcctcttatgtatgtaattgctgtcatggcactcagccgatacatggcagacaagcagcccctcactggcttcattaaaagctacattcagccagtttacaacattgtgcaaatcgtggtgtgctcgtggatggcgtggggccttttgccacaggtggacatcttcaacctcaacccattcggtctcaacaagcagcgtgatgccaacatcgagttctttgtcatggtccacctcctgacaaagatctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgcggatctcaaaagaactcgtccaggaggcggtagaacgcaatcctctggctgtccggggcggcgatgccgtagagcacgagaaagcggtcggcccactcgccgccaagctcctcggcgatgtcccgcgtggcgagcgcgatgtcttggtagcggtccgccacgcccaggcgcccgcagtcgataaagcccgagaagcggccgttctcgaccatgatgttggggaggcaggcgtcgccgtgcgtgaccacgaggtcctcgccgtccggcatcctagccttaagcctggcgaacagttccgccggcgcgaggccctggtgctcctcgtcgaggtcgtcttggtcgacgaggccagcctccatccgcgtgcgggcgcgttcgatcctgtgcttcgcctggtggtcgaaggggcaggtggcggggtcgagggtgtgcaggcggcgcatggcgtcggccatgatggacaccttctcagcgggcgcgaggtggctgctgaggaggtcctggccgggcacttccccgaggagcagccagtcgcggccggcttcggtgacgacgtcgagcacagcggcgcacggaacccccgtcgtggcaagccagctgaggcgggcagcttcgtcctggagctcgttgagggcgccgctaaggtcggtcttgacaaacaggaccggccggccctgcgcgctaaggcggaacacggccgcgtccgagcagccgatcgtctgctgagcccagtcgtagccgaacagccgttccacccaagcagcgggcgagccagcgtgaaggccgtcctgttcaatcatgttggctagtgttgcttaggtcgcttgctgctgctggtgttgcttgttctgcttgtcgtttggggtctgcccgaagtggacgcgtgacacagcccagcgttcgcgacttttccagcaaccagctcgctccgcgtcagggtcactctctgctcactcccctgtctagttcggcagaactggagaggcaacccgcgcttccagagggttctcctccggaatcagttcagaattcagaattcagaagagggaatcgcagaaccggccgcttcgggagttggttcgctgcaccatagcaggtgggcgatgaggccaaccgccctgctggctggattctctcgggttggcacatcgaacgaccgggacccgcgcgcgagttgtccctgccagttgctaccagcctgccagcgtcggagagttatgcgctgcctgccggccggagagagagcgggcgtggaaagtggcgtgtggggcgaacgcatggccctcccgcgtggcccgatttcctatgcatccgctccgctctctctctcggaggcaagaagagcacaccaacaacgcccggcgggtgcaccaggcgctgcggcagatccagatctcgactggaccgacaccttcatcatgattttcaagaagaactatgcacaggtctcttttctccaggtgttccaccatgccaccatcggaatggtgtggtccttcctcctccagcgcggctggggctctggaaccgctgcttacggagcgttcatcaactcggtcacccatgtcatcatgtacactcattactttgtcacctcgctcaacatcaacaacccgttcaagaggtacatcaccggcttccagctctcccagttcgcctcttgcattgtacatgctctcctcgtccttgccttcgaggaggtgtaccccctcgagtacgcttaccttcagatcagctaccacatcatcatgctctacctcttcggcaggagaatgaactggagccctctctggtgcactggcgaggtcgacgggcttgacgtcaacgtcgagacctccaagaaggctcagtaaggatccggg

SEQ ID NO: 32 - DNA, 2892 bp, synthetic construct
  misc_feature 1..2892: note = fusion DNA (Parietichytrium C20 elongase 5' region/SV40 terminator/Hygr/ubiquitin promoter/Parietichytrium C20 elongase 3' region)
  source 1..2892: mol_type = other DNA
cccggatccatggcagctcgcgtggagaaacagcaggcacctgcgaaggccgccaagaaggtggggtcgcgtgtggaccgcagtgatgggttctttcgcactttcaacctctgtgcgctgtacggaagcgcgttcgcgtacgcttacaacaatgggccagtggacaacgacggcaagggcttgtacttttcaaagtctccattctacgcattcctcgtctcggacgccatgaccttcggcgctcctcttatgtatgtaattgctgtcatggcactcagccgatacatggcagacaagcagcccctcactggcttcattaaaagctacattcagccagtttacaacattgtgcaaatcgtggtgtgctcgtggatggcgtggggccttttgccacaggtggacatcttcaacctcaacccattcggtctcaacaagcagcgtgatgccaacatcgagttctttgtcatggtccacctcctgacaaagatctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgcggatctctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatgttggctagtgttgcttaggtcgcttgctgctgctggtgttgcttgttctgcttgtcgtttggggtctgcccgaagtggacgcgtgacacagcccagcgttcgcgacttttccagcaaccagctcgctccgcgtcagggtcactctctgctcactcccctgtctagttcggcagaactggagaggcaacccgcgcttccagagggttctcctccggaatcagttcagaattcagaattcagaagagggaatcgcagaaccggccgcttcgggagttggttcgctgcaccatagcaggtgggcgatgaggccaaccgccctgctggctggattctctcgggttggcacatcgaacgaccgggacccgcgcgcgagttgtccctgccagttgctaccagcctgccagcgtcggagagttatgcgctgcctgccggccggagagagagcgggcgtggaaagtggcgtgtggggcgaacgcatggccctcccgcgtggcccgatttcctatgcatccgctccgctctctctctcggaggcaagaagagcacacaacaacgcccggcgggtgcaccaggcgctgcggcagatccagatctcgactggaccgacaccttcatcatgattttcaagaagaactatgcacaggtctcttttctccaggtgttccaccatgccaccatcggaatggtgtggtccttcctcctccagcgcggctggggctctggaaccgctgcttacggagcgttcatcaactcggtcacccatgtcatcatgtacactcattactttgtcacctcgctcaacatcaacaacccgttcaagaggtacatcaccggcttccagctctcccagttcgcctcttgcattgtacatgctctcctcgtccttgccttcgaggaggtgtaccccctcgagtacgcttaccttcagatcagctaccacatcatcatgctctacctcttcggcaggagaatgaactggagccctctctggtgcactggcgaggtcgacgggcttgacgtcaacgtcgagacctccaagaaggctcagtaaggatccggg

SEQ ID NO: 33 - DNA, 20 bp, synthetic construct
  misc_feature 1..20: note = primer
  source 1..20: mol_type = other DNA
ggttgacggcaatttcgatg

SEQ ID NO: 34 - DNA, 22 bp, synthetic construct
  misc_feature 1..22: note = primer
  source 1..22: mol_type = other DNA
cctcctacatcgaagctgaaag

SEQ ID NO: 35 - DNA, 21 bp, synthetic construct
  misc_feature 1..21: note = primer
  source 1..21: mol_type = other DNA
cttctcgggctttatcgactg

SEQ ID NO: 36 - DNA, 22 bp, synthetic construct
  misc_feature 1..22: note = primer
  source 1..22: mol_type = other DNA
taaggtcggtcttgacaaacag

SEQ ID NO: 37 - DNA, 24 bp, synthetic construct
  misc_feature 1..24: note = primer
  source 1..24: mol_type = other DNA
agtagtccccgatttggtagttga

SEQ ID NO: 38 - DNA, 22 bp, synthetic construct
  misc_feature 1..22: note = primer
  source 1..22: mol_type = other DNA
ggcagagagcaaaaacacgagc

SEQ ID NO: 39 - DNA, 5003 bp, synthetic construct
  misc_feature 1..5003: note = cDNA contains delta4-desaturase
  source 1..5003: mol_type = other DNA
gtttgaggagcgaggcatttcttcatttcttcgtggagaagaaagggcacacctgcagactaaacagccaagtaagaaaagttatttatcatcatcacaatatcctttctggcctgtctccaagagaaagaaacttttaaactttttaaattttaacttttgcttgtgctcgtatgcttacaaaagcttgcaacgaaacacagcggaagcaagttcagaacacgccaatgacatctttgctctccaaaattgaaaattgttttgtcaaattttaaaaactgattctgtaagaaaccttaatttttgcaaacaatcaaaaaatcgccgttgactgagtgtgattgaactcactccaacgaaagcgttagtccctcttcctatactttaccataataatatccctcttagattccatcccgacctgttcctactacgagcttataaacccctacgccccaagccaggtaagagagactagtaaattcttgacaatctgactgactatcctaaaccttctcgactactatgcaaaggactagtcgctgtagtcccagttgtgtgtactatagcagtaaagcgttggtgtacttaacagtaaaacagtaaaaccaaagcccccctaaattccccaaacagaatgagttgtctcagtccgaaactttctttgcaatgaaatacgattggtttggaaagttgacagtgagtgaaaagcctttcgattgattgatttgcttcggtcattcattcactcattcattcactcattcaaattgaaatgagcatgaaagtggtcgaagtggagtatctaggcgatctacatgaacgacttcaaggagcaagagtgcgagaggtaaaaagtatgttaaagcgttaaaagctgcaaaagctgtaaaagtgaaagcccatctggaaaccatagaaacgttctcaattcctcgattcctcgattcctcgtgcagactgcagaagaagactgacaaagcatcggcactggcactgacatgcgggtacgagtaggtacggacagcgagtgcccacacagccccaagcccacacattcattacattcattcattgaacacattgaacgcattgaacaatcaatcactccacattgtcactgtcaactgatgtcaactggtaaacttgtaacttgtattgtattgacctcgcttggtgcatagagtgcgtagagtgcgtagagtgcgtagccgtcgccccggagagacgctgttgactgacaactgtgttaaggtgtcgattacaacaaacaaacaaataaacaaacaaccaaacaaacacctcccaacacacgagctttgacgtcctcaccacccggtttgccctcgcagtcgcggtcagttcagtgccacgagtgccacggggcgtaaggaagtttaggaaggcgtgtttcgaatccctttacagcgcggcgctcgcgaactgaagacgcggagatacgcagtagtgagtgggtggagcaagaaaagtagagggcccccgtacgtcccggaggagagaccgattgtggattggactgctggatcgtggaataggcaagatgacggccggatttgaagaagtgatcaccatgaagcaggtgaaggaccggaatacgccggacgatgcgtggtgcgtggtgcatggcaaggtgtacgacatcaccaagttcaagaacgctcaccccggtggagatataatcatgttggcggctggcaaggacgccaccatcctgttcgagacttaccacatccgcggtgtgcccgatgccgtgttgcgcaagtatcagatcggcaaacttccggacggaaagaacaaagagggcggcaacggcctcgacagcgcctcgtactactcctgggacagcgagttttaccgcgtccttcgcgagcgcgtcttgaagcgcctgaacgagctcaagctgtccagacgcggaggcttcgagatttgggccaaggctatctttctcttgaccggcttctggtcttgcctctacctcatgtgcacactcaacccaaatgggcttgcgattcctgccgccatgatgttgggaatctttgctgccttcgtaggaacctgcattcagcacgacgggaatcacggtgcgttcgcccaatcttcgtggcttaacaaggccgctggttggactttggacatgattggttccagcgccatgacctgggagatgcagcacgtgcttggacatcatccgtacaccaacttgattgaaatggagaatggcaatcaaaaggtctccggcaagcctgttgacaccaagactgtcgaccaggagagcgaccctgatgtctttagcacctaccctatgcttcgccttcacccttggcacagcaaaaagtggtaccacaaataccagcacatctatgcaccattcatctttgggttcatgaccatcaacaaggtcattgcacaggacgtcggcgttatcacacgcaagcgtctcttccagattgacgccaactgccgctacgcttctccgacttacgtcgctcgcttctggatcatgaaggttcttaccgttctctacatggttggcctgccaatgtacatgcaaggtccatgggagggtctcaagttgttctttattgcgcactttacttgcggcgagctgctggccacaatgttcatcgtaaaccacatcatcgagggtgtcagctacgcaagcaaagatgccatcaagggcgagatggctccaccgaaaacggtccgcggtgtcaccccaatgcacgagacgcaaaaggttctcgaccagcgcgagaaagacatggacgaaacttctaagaagagccgcatccctctcaacgactgggccgctgtacagtgccagacctccgtgaactgggctatcggttcttggttctggaaccacttttccgggggcctcaatcatcagattgagcatcatctgttccccggcttgactcacaccacctatgttcactttcacgatgtggtcaaagatacttgcgctgagtacggggttccataccagcacgaggagagtctatacactgcctactttaagatgttgaatcatctcaagaccctaggcaacgagccaatgcctgcctgggacaagaactaacagtaaacaaacatcacgcgtcctcaaagcctgactatcgcgggctgcgctttcactttgcctgccgttttggaggatatctgcttttgcgatcaaaactaggaatggagtgtacagccagccactcgttcttcagtacgaatcaacgaagactttgtctcctttcctaaaaaagaggatgtttttcttacctagctaacaaggcggtgtctggccattgtacctttggattcgctacatacacacatacatacacgcctttcgttgcgctgctcgaaatttgtagtgacgtgagatgaaagataaaaaaaatagaattcaggtctcgttcaaaagttcgtaattctcttctccaattccattcgtctccggagaaagtctaggtcggcattctgtattgaagcaatgtgcagcagcgaaacagaaaaaacgagcagtcaaaagactagctactagtcttcgagtggatcgcccccagccaatctcaaatacatctttgcgaaaacagacacttttgtttcctgagagcttttgatgaaggtttccgtcaaagtatctgacgaggcgttgcacgggttctcgaaacccggcaaagcaggtagatctaactttctgaggctctccataaaaccctgcacacgatctttggcttttggctcattcgtcaaacgaaacaaaatgccaggcttgtcgttggttccaactacggttgtaaagttgtatggcgagtgcactcgtttttcgtcgaaccagtctggatgcttctgcatcatggctttcttgaaggcaacagtttccgtgaaagtagactccagggctgtataagaacctggagcaagggtggtcttcatgccttgctccatcatgtggatgacactgtgactcacaaaattgcatccaatctcagacatgtacatattgaggttgtagttttccgggtccgtcacgttggagatggagattccgctcgccgaagcatcgcagcagtaccaatgacttgagcgatccgccacccagtgtaggtatagtccaaatcgtacaatctcttcggggacaggatccccgttgataagtgaataaggtgcgctcgagttgagatattccccaagctggcttgccagtactacatccgtcatcttgtagttttcgttatggtctgggtcctcgcaggtcccgttgcaagcgcaagccggatcacagttgtagttaacgtattgctcacctaacatcatatcgcctccggcgagagggattggaccgctcaccaatttggagatgtgtttagaagtgacggttccgcttttagggcaggtctcgccagtgaatggagatccatcagggccaatgctagtatagcctccaacacaaagcagatccgtctctccaaaagcccatcgtcgagcagaagcgagcgcaccctcgtaaaagttgtcgtccatatttggggccaggcccgggcaatgcgcacggtaaaactcatacgactttttgaagcgatacctctgacagtcaccctccttgccacccgtgtgctccttggcgctctcggttaaaatggagccattgaagtgtccgatgtacgggattcctaagtgccgctgtgttcctcccagcttggtgtttgtacggagaaaacctctcataggtggagtccaccactccaatcccatgtccgcgccacagctgtcaattgcgcggtactgtacgaaatcaatcgcctgagaatacgcggccatgaagtaagcgtactctgctttgaggccctgtgccacagcaatataatagatcgcgtaaaagtgaatgaagcctctgcaggcggaggtatcgttgaacccggttgtcatctgtgggagcagacttgccaggatgcactttggacgccagtcattgtacgagcact

SEQ ID NO: 40 - DNA, 23 bp, synthetic construct
  misc_feature 1..23: note = primer
  source 1..23: mol_type = other DNA
gtttgaggagcgaggcatttctt

SEQ ID NO: 41 - DNA, 23 bp, synthetic construct
  misc_feature 1..23: note = primer
  source 1..23: mol_type = other DNA
agtgctcgtacaatgactggcgt

SEQ ID NO: 42 - DNA, 8020 bp, synthetic construct
  misc_feature 1..8020: note = plasmid
  source 1..8020: mol_type = other DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcggccgcgaattcactagtgattgtttgaggagcgaggcatttcttcatttcttcgtggagaagaaagggcacacctgcagactaaacagccaagtaagaaaagttatttatcatcatcacaatatcctttctggcctgtctccaagagaaagaaacttttaaactttttaaattttaacttttgcttgtgctcgtatgcttacaaaagcttgcaacgaaacacagcggaagcaagttcagaacacgccaatgacatctttgctctccaaaattgaaaattgttttgtcaaattttaaaaactgattctgtaagaaaccttaatttttgcaaacaatcaaaaaatcgccgttgactgagtgtgattgaactcactccaacgaaagcgttagtccctcttcctatactttaccataataatatccctcttagattccatcccgacctgttcctactacgagcttataaacccctacgccccaagccaggtaagagagactagtaaattcttgacaatctgactgactatcctaaaccttctcgactactatgcaaaggactagtcgctgtagtcccagttgtgtgtactatagcagtaaagcgttggtgtacttaacagtaaaacagtaaaaccaaagcccccctaaattccccaaacagaatgagttgtctcagtccgaaactttctttgcaatgaaatacgattggtttggaaagttgacagtgagtgaaaagcctttcgattgattgatttgcttcggtcattcattcactcattcattcactcattcaaattgaaatgagcatgaaagtggtcgaagtggagtatctaggcgatctacatgaacgacttcaaggagcaagagtgcgagaggtaaaaagtatgttaaagcgttaaaagctgcaaaagctgtaaaagtgaaagcccatctggaaaccatagaaacgttctcaattcctcgattcctcgattcctcgtgcagactgcagaagaagactgacaaagcatcggcactggcactgacatgcgggtacgagtaggtacggacagcgagtgcccacacagccccaagcccacacattcattacattcattcattgaacacattgaacgcattgaacaatcaatcactccacattgtcactgtcaactgatgtcaactggtaaacttgtaacttgtattgtattgacctcgcttggtgcatagagtgcgtagagtgcgtagagtgcgtagccgtcgccccggagagacgctgttgactgacaactgtgttaaggtgtcgattacaacaaacaaacaaataaacaaacaaccaaacaaacacctcccaacacacgagctttgacgtcctcaccacccggtttgccctcgcagtcgcggtcagttcagtgccacgagtgccacggggcgtaaggaagtttaggaaggcgtgtttcgaatccctttacagcgcggcgctcgcgaactgaagacgcggagatacgcagtagtgagtgggtggagcaagaaaagtagagggcccccgtacgtcccggaggagagaccgattgtggattggactgctggatcgtggaataggcaagatgacggccggatttgaagaagtgatcaccatgaagcaggtgaaggaccggaatacgccggacgatgcgtggtgcgtggtgcatggcaaggtgtacgacatcaccaagttcaagaacgctcaccccggtggagatataatcatgttggcggctggcaaggacgccaccatcctgttcgagacttaccacatccgcggtgtgcccgatgccgtgttgcgcaagtatcagatcggcaaacttccggacggaaagaacaaagagggcggcaacggcctcgacagcgcctcgtactactcctgggacagcgagttttaccgcgtccttcgcgagcgcgtcttgaagcgcctgaacgagctcaagctgtccagacgcggaggcttcgagatttgggccaaggctatctttctcttgaccggcttctggtcttgcctctacctcatgtgcacactcaacccaaatgggcttgcgattcctgccgccatgatgttgggaatctttgctgccttcgtaggaacctgcattcagcacgacgggaatcacggtgcgttcgcccaatcttcgtggcttaacaaggccgctggttggactttggacatgattggttccagcgccatgacctgggagatgcagcacgtgcttggacatcatccgtacaccaacttgattgaaatggagaatggcaatcaaaaggtctccggcaagcctgttgacaccaagactgtcgaccaggagagcgaccctgatgtctttagcacctaccctatgcttcgccttcacccttggcacagcaaaaagtggtaccacaaataccagcacatctatgcaccattcatctttgggttcatgaccatcaacaaggtcattgcacaggacgtcggcgttatcacacgcaagcgtctcttccagattgacgccaactgccgctacgcttctccgacttacgtcgctcgcttctggatcatgaaggttcttaccgttctctacatggttggcctgccaatgtacatgcaaggtccatgggagggtctcaagttgttctttattgcgcactttacttgcggcgagctgctggccacaatgttcatcgtaaaccacatcatcgagggtgtcagctacgcaagcaaagatgccatcaagggcgagatggctccaccgaaaacggtccgcggtgtcaccccaatgcacgagacgcaaaaggttctcgaccagcgcgagaaagacatggacgaaacttctaagaagagccgcatccctctcaacgactgggccgctgtacagtgccagacctccgtgaactgggctatcggttcttggttctggaaccacttttccgggggcctcaatcatcagattgagcatcatctgttccccggcttgactcacaccacctatgttcactttcacgatgtggtcaaagatacttgcgctgagtacggggttccataccagcacgaggagagtctatacactgcctactttaagatgttgaatcatctcaagaccctaggcaacgagccaatgcctgcctgggacaagaactaacagtaaacaaacatcacgcgtcctcaaagcctgactatcgcgggctgcgctttcactttgcctgccgttttggaggatatctgcttttgcgatcaaaactaggaatggagtgtacagccagccactcgttcttcagtacgaatcaacgaagactttgtctcctttcctaaaaaagaggatgtttttcttacctagctaacaaggcggtgtctggccattgtacctttggattcgctacatacacacatacatacacgcctttcgttgcgctgctcgaaatttgtagtgacgtgagatgaaagataaaaaaaatagaattcaggtctcgttcaaaagttcgtaattctcttctccaattccattcgtctccggagaaagtctaggtcggcattctgtattgaagcaatgtgcagcagcgaaacagaaaaaacgagcagtcaaaagactagctactagtcttcgagtggatcgcccccagccaatctcaaatacatctttgcgaaaacagacacttttgtttcctgagagcttttgatgaaggtttccgtcaaagtatctgacgaggcgttgcacgggttctcgaaacccggcaaagcaggtagatctaactttctgaggctctccataaaaccctgcacacgatctttggcttttggctcattcgtcaaacgaaacaaaatgccaggcttgtcgttggttccaactacggttgtaaagttgtatggcgagtgcactcgtttttcgtcgaaccagtctggatgcttctgcatcatggctttcttgaaggcaacagtttccgtgaaagtagactccagggctgtataagaacctggagcaagggtggtcttcatgccttgctccatcatgtggatgacactgtgactcacaaaattgcatccaatctcagacatgtacatattgaggttgtagttttccgggtccgtcacgttggagatggagattccgctcgccgaagcatcgcagcagtaccaatgacttgagcgatccgccacccagtgtaggtatagtccaaatcgtacaatctcttcggggacaggatccccgttgataagtgaataaggtgcgctcgagttgagatattccccaagctggcttgccagtactacatccgtcatcttgtagttttcgttatggtctgggtcctcgcaggtcccgttgcaagcgcaagccggatcacagttgtagttaacgtattgctcacctaacatcatatcgcctccggcgagagggattggaccgctcaccaatttggagatgtgtttagaagtgacggttccgcttttagggcaggtctcgccagtgaatggagatccatcagggccaatgctagtatagcctccaacacaaagcagatccgtctctccaaaagcccatcgtcgagcagaagcgagcgcaccctcgtaaaagttgtcgtccatatttggggccaggcccgggcaatgcgcacggtaaaactcatacgactttttgaagcgatacctctgacagtcaccctccttgccacccgtgtgctccttggcgctctcggttaaaatggagccattgaagtgtccgatgtacgggattcctaagtgccgctgtgttcctcccagcttggtgtttgtacggagaaaacctctcataggtggagtccaccactccaatcccatgtccgcgccacagctgtcaattgcgcggtactgtacgaaatcaatcgcctgagaatacgcggccatgaagtaagcgtactctgctttgaggccctgtgccacagcaatataatagatcgcgtaaaagtgaatgaagcctctgcaggcggaggtatcgttgaacccggttgtcatctgtgggagcagacttgccaggatgcactttggacgccagtcattgtacgagcactaatcgaattcccgcggccgccatggcggccgggagcatgcgacgtcgggcccaattcgccc

SEQ ID NO: 43 - DNA, 1542 bp, synthetic construct
  source 1..1542: mol_type = other DNA
atgacggccggatttgaagaagtgatcaccatgaagcaggtgaaggaccggaatacgccggacgatgcgtggtgcgtggtgcatggcaaggtgtacgacatcaccaagttcaagaacgctcaccccggtggagatataatcatgttggcggctggcaaggacgccaccatcctgttcgagacttaccacatccgcggtgtgcccgatgccgtgttgcgcaagtatcagatcggcaaacttccggacggaaagaacaaagagggcggcaacggcctcgacagcgcctcgtactactcctgggacagcgagttttaccgcgtccttcgcgagcgcgtcttgaagcgcctgaacgagctcaagctgtccagacgcggaggcttcgagatttgggccaaggctatctttctcttgaccggcttctggtcttgcctctacctcatgtgcacactcaacccaaatgggcttgcgattcctgccgccatgatgttgggaatctttgctgccttcgtaggaacctgcattcagcacgacgggaatcacggtgcgttcgcccaatcttcgtggcttaacaaggccgctggttggactttggacatgattggttccagcgccatgacctgggagatgcagcacgtgcttggacatcatccgtacaccaacttgattgaaatggagaatggcaatcaaaaggtctccggcaagcctgttgacaccaagactgtcgaccaggagagcgaccctgatgtctttagcacctaccctatgcttcgccttcacccttggcacagcaaaaagtggtaccacaaataccagcacatctatgcaccattcatctttgggttcatgaccatcaacaaggtcattgcacaggacgtcggcgttatcacacgcaagcgtctcttccagattgacgccaactgccgctacgcttctccgacttacgtcgctcgcttctggatcatgaaggttcttaccgttctctacatggttggcctgccaatgtacatgcaaggtccatgggagggtctcaagttgttctttattgcgcactttacttgcggcgagctgctggccacaatgttcatcgtaaaccacatcatcgagggtgtcagctacgcaagcaaagatgccatcaagggcgagatggctccaccgaaaacggtccgcggtgtcaccccaatgcacgagacgcaaaaggttctcgaccagcgcgagaaagacatggacgaaacttctaagaagagccgcatccctctcaacgactgggccgctgtacagtgccagacctccgtgaactgggctatcggttcttggttctggaaccacttttccgggggcctcaatcatcagattgagcatcatctgttccccggcttgactcacaccacctatgttcactttcacgatgtggtcaaagatacttgcgctgagtacggggttccataccagcacgaggagagtctatacactgcctactttaagatgttgaatcatctcaagaccctaggcaacgagccaatgcctgcctgggacaagaactaa

SEQ ID NO: 44 - DNA, 26 bp, synthetic construct
  misc_feature 1..26: note = primer
  source 1..26: mol_type = other DNA
ggcaagatctaactttctgaggctct

SEQ ID NO: 45 - DNA, 26 bp, synthetic construct
  misc_feature 1..26: note = primer
  source 1..26: mol_type = other DNA
aagttagatcttgcctattccacgat

SEQ ID NO: 46 - DNA, 26 bp, synthetic construct
  misc_feature 1..26: note = primer
  source 1..26: mol_type = other DNA
gtgcagacgcagaagaagactgacaa

SEQ ID NO: 47 - DNA, 25 bp, synthetic construct
  misc_feature 1..25: note = primer
  source 1..25: mol_type = other DNA
cttctgcgtctgcacgaggaatcga

SEQ ID NO: 48 - DNA, 7579 bp, synthetic construct
  misc_feature 1..7579: note = plasmid
  source 1..7579: mol_type = other DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcggccgcgaattcactagtgattgtttgaggagcgaggcatttcttcatttcttcgtggagaagaaagggcacacctgcagactaaacagccaagtaagaaaagttatttatcatcatcacaatatcctttctggcctgtctccaagagaaagaaacttttaaactttttaaattttaacttttgcttgtgctcgtatgcttacaaaagcttgcaacgaaacacagcggaagcaagttcagaacacgccaatgacatctttgctctccaaaattgaaaattgttttgtcaaattttaaaaactgattctgtaagaaaccttaatttttgcaaacaatcaaaaaatcgccgttgactgagtgtgattgaactcactccaacgaaagcgttagtccctcttcctatactttaccataataatatccctcttagattccatcccgacctgttcctactacgagcttataaacccctacgccccaagccaggtaagagagactagtaaattcttgacaatctgactgactatcctaaaccttctcgactactatgcaaaggactagtcgctgtagtcccagttgtgtgtactatagcagtaaagcgttggtgtacttaacagtaaaacagtaaaaccaaagcccccctaaattccccaaacagaatgagttgtctcagtccgaaactttctttgcaatgaaatacgattggtttggaaagttgacagtgagtgaaaagcctttcgattgattgatttgcttcggtcattcattcactcattcattcactcattcaaattgaaatgagcatgaaagtggtcgaagtggagtatctaggcgatctacatgaacgacttcaaggagcaagagtgcgagaggtaaaaagtatgttaaagcgttaaaagctgcaaaagctgtaaaagtgaaagcccatctggaaaccatagaaacgttctcaattcctcgattcctcgattcctcgtgcagacgcagaagaagactgacaaagcatcggcactggcactgacatgcgggtacgagtaggtacggacagcgagtgcccacacagccccaagcccacacattcattacattcattcattgaacacattgaacgcattgaacaatcaatcactccacattgtcactgtcaactgatgtcaactggtaaacttgtaacttgtattgtattgacctcgcttggtgcatagagtgcgtagagtgcgtagagtgcgtagccgtcgccccggagagacgctgttgactgacaactgtgttaaggtgtcgattacaacaaacaaacaaataaacaaacaaccaaacaaacacctcccaacacacgagctttgacgtcctcaccacccggtttgccctcgcagtcgcggtcagttcagtgccacgagtgccacggggcgtaaggaagtttaggaaggcgtgtttcgaatccctttacagcgcggcgctcgcgaactgaagacgcggagatacgcagtagtgagtgggtggagcaagaaaagtagagggcccccgtacgtcccggaggagagaccgattgtggattggactgctggatcgtggaataggcaagatctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgcggatctcaaaagaactcgtccaggaggcggtagaacgcaatcctctggctgtccggggcggcgatgccgtagagcacgagaaagcggtcggcccactcgccgccaagctcctcggcgatgtcccgcgtggcgagcgcgatgtcttggtagcggtccgccacgcccaggcgcccgcagtcgataaagcccgagaagcggccgttctcgaccatgatgttggggaggcaggcgtcgccgtgcgtgaccacgaggtcctcgccgtccggcatcctagccttaagcctggcgaacagttccgccggcgcgaggccctggtgctcctcgtcgaggtcgtcttggtcgacgaggccagcctccatccgcgtgcgggcgcgttcgatcctgtgcttcgcctggtggtcgaaggggcaggtggcggggtcgagggtgtgcaggcggcgcatggcgtcggccatgatggacaccttctcagcgggcgcgaggtggctgctgaggaggtcctggccgggcacttccccgaggagcagccagtcgcggccggcttcggtgacgacgtcgagcacagcggcgcacggaacccccgtcgtggcaagccagctgaggcgggcagcttcgtcctggagctcgttgagggcgccgctaaggtcggtcttgacaaacaggaccggccggccctgcgcgctaaggcggaacacggccgcgtccgagcagccgatcgtctgctgagcccagtcgtagccgaacagccgttccacccaagcagcgggcgagccagcgtgaaggccgtcctgttcaatcatgttggctagtgttgcttaggtcgcttgctgctgctggtgttgcttgttctgcttgtcgtttggggtctgcccgaagtggacgcgtgacacagcccagcgttcgcgacttttccagcaaccagctcgctccgcgtcagggtcactctctgctcactcccctgtctagttcggcagaactggagaggcaacccgcgcttccagagggttctcctccggaatcagttcagaattcagaattcagaagagggaatcgcagaaccggccgcttcgggagttggttcgctgcaccatagcaggtgggcgatgaggccaaccgccctgctggctggattctctcgggttggcacatcgaacgaccgggacccgcgcgcgagttgtccctgccagttgctaccagcctgccagcgtcggagagttatgcgctgcctgccggccggagagagagcgggcgtggaaagtggcgtgtggggcgaacgcatggccctcccgcgtggcccgatttcctatgcatccgctccgctctctctctcggaggcaagaagagcacacaacaacgcccggcgggtgcaccaggcgctgcggcagatccagatctaactttctgaggctctccataaaaccctgcacacgatctttggcttttggctcattcgtcaaacgaaacaaaatgccaggcttgtcgttggttccaactacggttgtaaagttgtatggcgagtgcactcgtttttcgtcgaaccagtctggatgcttctgcatcatggctttcttgaaggcaacagtttccgtgaaagtagactccagggctgtataagaacctggagcaagggtggtcttcatgccttgctccatcatgtggatgacactgtgactcacaaaattgcatccaatctcagacatgtacatattgaggttgtagttttccgggtccgtcacgttggagatggagattccgctcgccgaagcatcgcagcagtaccaatgacttgagcgatccgccacccagtgtaggtatagtccaaatcgtacaatctcttcggggacaggatccccgttgataagtgaataaggtgcgctcgagttgagatattccccaagctggcttgccagtactacatccgtcatcttgtagttttcgttatggtctgggtcctcgcaggtcccgttgcaagcgcaagccggatcacagttgtagttaacgtattgctcacctaacatcatatcgcctccggcgagagggattggaccgctcaccaatttggagatgtgtttagaagtgacggttccgcttttagggcaggtctcgccagtgaatggagatccatcagggccaatgctagtatagcctccaacacaaagcagatccgtctctccaaaagcccatcgtcgagcagaagcgagcgcaccctcgtaaaagttgtcgtccatatttggggccaggcccgggcaatgcgcacggtaaaactcatacgactttttgaagcgatacctctgacagtcaccctccttgccacccgtgtgctccttggcgctctcggttaaaatggagccattgaagtgtccgatgtacgggattcctaagtgccgctgtgttcctcccagcttggtgtttgtacggagaaaacctctcataggtggagtccaccactccaatcccatgtccgcgccacagctgtcaattgcgcggtactgtacgaaatcaatcgcctgagaatacgcggccatgaagtaagcgtactctgctttgaggccctgtgccacagcaatataatagatcgcgtaaaagtgaatgaagcctctgcaggcggaggtatcgttgaacccggttgtcatctgtgggagcagacttgccaggatgcactttggacgccagtcattgtacgagcactaatcgaattcccgcggccgccatggcggccgggagcatgcgacgtcgggcccaattcgccc

SEQ ID NO: 49 - DNA, 20 bp, synthetic construct
  misc_feature 1..20: note = primer
  source 1..20: mol_type = other DNA
gtggtcgaagtggagtatct

SEQ ID NO: 50 - DNA, 20 bp, synthetic construct
  misc_feature 1..20: note = primer
  source 1..20: mol_type = other DNA
actcgccatacaactttaca

SEQ ID NO: 51 - DNA, 25 bp, synthetic construct
  misc_feature 1..25: note = primer
  source 1..25: mol_type = other DNA
cggagctcggagaacaacatagaag

SEQ ID NO: 52 - DNA, 23 bp, synthetic construct
  misc_feature 1..23: note = primer
  source 1..23: mol_type = other DNA
gtgcaaccaggtggcaagattgt

SEQ ID NO: 53 - DNA, 21 bp, synthetic construct
  misc_feature 1..21: note = primer
  misc_feature 6: note = n is a, c, g, or t
  misc_feature 12: note = n is a, c, g, or t
  source 1..21: mol_type = other DNA
ttyytncaygtntaycaycay

SEQ ID NO: 54 - DNA, 23 bp, synthetic construct
  misc_feature 1..23: note = primer
  misc_feature 12: note = n is a, c, g, or t
  misc_feature 18: note = n is a, c, g, or t
  source 1..23: mol_type = other DNA
gcrtgrtgrtanacrtgnarraa

SEQ ID NO: 55 - DNA, 190 bp, synthetic construct
  misc_feature 1..190: note = cDNA (DNA fragment contains elo1)
  source 1..190: mol_type = other DNA
cgccaccatctttgctatctggtttatgatcgccaagtacgccccgggcggcgacgcatactttagcgtcatcctgaactcgttcgtgcacaccgtcatgtacgcgtactacttcttctcgtcgcagggcttcgggttcgtcaagccgatcaagccgtacatcacctcgctgcagatgacgcagttcatg

SEQ ID NO: 56 - DNA, 210 bp, synthetic construct
  misc_feature 1..210: note = cDNA (DNA fragment contains elo2)
  source 1..210: mol_type = other DNA
ccgacgaccagcacaccgagtgggtctcgtgcgtgcgcttctcgccctcgaccaccaacccgctgatcgtgtcgtgcggctgggacaagctcgtcaaggtctggaacctctcgaactgcaagcttcgggccaacctcatcggccacgacggctacctcaactcggtcaccgtcagcccggacggctccctgtgcgcttcgggcggcaagg

SEQ ID NO: 57 - DNA, 200 bp, synthetic construct
  misc_feature 1..200: note = cDNA (DNA fragment contains elo3)
  source 1..200: mol_type = other DNA
aagctaacctgggcgtagtttttcttgaggatcatcatgaacgtgtcgctccagtcgagaaactttgtcaggaggtgcacgaacacgaaaaactcgatgttcgagtcgcgcgacttgttgaggccgaaagggttgccgttggccaggtcgacctgcggccagaggccccacaccatccagccgcacaccgcgatttggac

SEQ ID NO: 58 - DNA, 21 bp, synthetic construct
  misc_feature 1..21: note = primer
  source 1..21: mol_type = other DNA
tatgatcgccaagtacgcccc

SEQ ID NO: 59 - DNA, 21 bp, synthetic construct
  misc_feature 1..21: note = primer
  source 1..21: mol_type = other DNA
gaactgcgtcatctgcagcga

SEQ ID NO: 60 - DNA, 20 bp, synthetic construct
  misc_feature 1..20: note = primer
  source 1..20: mol_type = other DNA
tctcgccctcgaccaccaac

SEQ ID NO: 61 - DNA, 22 bp, synthetic construct
  misc_feature 1..22: note = primer
  source 1..22: mol_type = other DNA
cggtgaccgagttgaggtagcc

SEQ ID NO: 62 - DNA, 22 bp, synthetic construct
  misc_feature 1..22: note = primer
  source 1..22: mol_type = other DNA
caaccctttcggcctcaacaag

SEQ ID NO: 63 - DNA, 26 bp, synthetic construct
  misc_feature 1..26: note = primer
  source 1..26: mol_type = other DNA
ttcttgaggatcatcatgaacgtgtc

SEQ ID NO: 64 - DNA, 1139 bp, synthetic construct
  misc_feature 1..1139: note = cDNA (elo1)
  source 1..1139: mol_type = other DNA
ctaatacgactcactatagggcaagcagtggtaacaacgcagagtacgcggggaccccaaacgcccgacgacaaccaagaagacagccagccgaacaatcggacgaagatgacgagcaacatgagcgcgtggggcgtcgccgtcgaccagacgcagcaggtcgtcgaccagatcatgggcggcgccgagccgtacaagctgacagaagggcgcatgacgaacgtcgagacgatgctggcgatcgagtgcggctacgccgccatgctgctgttcctgaccccgatcatgaagcaggccgagaagcccttcgagctcaagtccttcaagctcgcccacaacctgttcctgttcgtcctgtccgcctacatgtgcctcgagaccgtccgccaggcctaccttgcgggctactcggtgttcggcaacgacatggagaagggcagcgagccgcacgcgcacggcatggcccaaatcgtgtggatcttttacgtgtccaaggcgtacgagttcgtggacacgctgatcatgatcctgtgcaaaaagttcaaccaggtctccgtcctgcacgtgtaccaccacgccaccatctttgctatctggtttatgatcgccaagtacgccccgggcggcgacgcatactttagcgtcatcctgaactcgttcgtgcacaccgtcatgtacgcgtactacttcttctcgtcgcagggcttcgggttcgtcaagccgatcaagccgtacatcacctcgctgcagatgacgcagttcatggcgatgctcgtgcagtcgctgtacgactacctttacccgtgcgactacccgcaggggctcgtcaagctcctcggcgtgtacatgctcaccctgcttgcgctcttcggcaactttttcgtgcagagctacctcaagaagtcgaacaagcccaaggccaagtcggcctaagccgacccgctcgccggcaaccgagcagcacctaggcgcatctcggcccggaaccttttcgacctgctgtggagcgcgcgacgcgtttcgcgaccgtccgcgcgttcttgacactctttgctctgtgtgtttcgcacttgacaacctggaacagacacatacacgatacaaatcatcagaacagacaaaaaacaacctcaaattat

SEQ ID NO: 65 - DNA, 1261 bp, synthetic construct
  misc_feature 1..1261: note = cDNA (elo3)
  source 1..1261: mol_type = other DNA
ctaatacgactcactatagggcaagcagtggtatcaacgcagagtacgcggggaccccgaacgtgtttctcccaggacgtgccgctgtcgctcgctgatccacccgaagcgcggtcggctggcacggtcgctcggctggaagttgagtagtttgctttctgttactgcgctgctttgtaaacgcgaccatggcgacgcgcacctcgaagagcgctccggcggtttccaagtcggccaaggttgccgcgccggcgaagaagcggtcggtcgacaggagcgacggtttcttccgcacgttcaacctgtgcgccctgtacgggtctgccctcgcctatgcgtacaagcacggcccggtggacaatgacggccaggggctgtactttcacaagtcgcccatgtacgcgttcgccgtgtcggacgtcatgaccttcggcgcgccgctgatgtacgtgctcggtgtgatgctgctcagcaggtacatggcggacaaaaagcccctgactggcttcatcaagacctacatccagcccgtctacaacgtggtccaaatcgcggtgtgcggctggatggtgtggggcctctggccgcaggtcgacctggccaacggcaaccctttcggcctcaacaagtcgcgcgactcgaacatcgagtttttcgtgttcgtgcacctcctgacaaagtttctcgactggagcgacacgttcatgatgatcctcaagaaaaactacgcccaggttagctttctgcaggtgttccaccacgcaacgatcggcatggtgtggtcgttccttcttcagcgtggctggggctcgggcaccgccgcgtacggtgctttcatcaactcggtcacgcacgtgatcatgtactcgcactactttgccacctcgctcaacatcaacaacccgttcaagcggtacatcacgagcttccagctcgcccagtttgcaagctgcatcgtgcatgccctactggtgcttgccttcgaggaggtgtacccgctcgagtacgcttacctgcagatcagctaccacatcatcatgctctacctgttcggacgccgcatgaactggagccccgagtggtgcaccggtgagatcgacggccttgacgccccaagcgcccccaccaagtccgagtaaacctgtttccggctggctcccgagccatgcttaccatgaatgaacctgcaaacagtctgaggtccttgtgcaaaccgctcagtgggacgtcgacgaagaaagaaacaatgtgtactcgtccc

SEQ ID NO: 66 - protein, 275 aa, synthetic construct
  SITE 275: note = misc_feature - Xaa can be any naturally occurring amino acid
MTSNMSAWGVAVDQTQQVVDQIMGGAEPYKLTEGRMTNVETMLAIECGYAAMLLFLTPIMKQAEKPFELKSFKLAHNLFLFVLSAYMCLETVRQAYLAGYSVFGNDMEKGSEPHAHGMAQIVWIFYVSKAYEFVDTLIMILCKKFNQVSVLHVYHHATIFAIWFMIAKYAPGGDAYFSVILNSFVHTVMYAYYFFSSQGFGFVKPIKPYITSLQMTQFMAMLVQSLYDYLYPCDYPQGLVKLLGVYMLTLLALFGNFFVQSYLKKSNKPKAKSAX

SEQ ID NO: 67 - DNA, 825 bp, synthetic construct
  misc_feature 1..825: note = cDNA (T. aureum ATCC 34304 elo1)
  source 1..825: mol_type = other DNA
atgacgagcaacatgagcgcgtggggcgtcgccgtcgaccagacgcagcaggtcgtcgaccagatcatgggcggcgccgagccgtacaagctgacagaagggcgcatgacgaacgtcgagacgatgctggcgatcgagtgcggctacgccgccatgctgctgttcctgaccccgatcatgaagcaggccgagaagcccttcgagctcaagtccttcaagctcgcccacaacctgttcctgttcgtcctgtccgcctacatgtgcctcgagaccgtccgccaggcctaccttgcgggctactcggtgttcggcaacgacatggagaagggcagcgagccgcacgcgcacggcatggcccaaatcgtgtggatcttttacgtgtccaaggcgtacgagttcgtggacacgctgatcatgatcctgtgcaaaaagttcaaccaggtctccgtcctgcacgtgtaccaccacgccaccatctttgctatctggtttatgatcgccaagtacgccccgggcggcgacgcatactttagcgtcatcctgaactcgttcgtgcacaccgtcatgtacgcgtactacttcttctcgtcgcagggcttcgggttcgtcaagccgatcaagccgtacatcacctcgctgcagatgacgcagttcatggcgatgctcgtgcagtcgctgtacgactacctttacccgtgcgactacccgcaggggctcgtcaagctcctcggcgtgtacatgctcaccctgcttgcgctcttcggcaactttttcgtgcagagctacctcaagaagtcgaacaagcccaaggccaagtcggcctaa

SEQ ID NO: 68 - protein, 317 aa, synthetic construct
  SITE 317: note = misc_feature - Xaa can be any naturally occurring amino acid
MATRTSKSAPAVSKSAKVAAPAKKRSVDRSDGFFRTFNLCALYGSALAYAYKHGPVDNDGQGLYFHKSPMYAFAVSDVMTFGAPLMYVLGVMLLSRYMADKKPLTGFIKTYIQPVYNVVQIAVCGWMVWGLWPQVDLANGNPFGLNKSRDSNIEFFVFVHLLTKFLDWSDTFMMILKKNYAQVSFLQVFHHATIGMVWSFLLQRGWGSGTAAYGAFINSVTHVIMYSHYFATSLNINNPFKRYITSFQLAQFASCIVHALLVLAFEEVYPLEYAYLQISYHIIMLYLFGRRMNWSPEWCTGEIDGLDAPSAPTKSEX

SEQ ID NO: 69 - DNA, 951 bp, synthetic construct
  misc_feature 1..951: note = cDNA (T. aureum ATCC 34304 elo3)
  source 1..951: mol_type = other DNA
atggcgacgcgcacctcgaagagcgctccggcggtttccaagtcggccaaggttgccgcgccggcgaagaagcggtcggtcgacaggagcgacggtttcttccgcacgttcaacctgtgcgccctgtacgggtctgccctcgcctatgcgtacaagcacggcccggtggacaatgacggccaggggctgtactttcacaagtcgcccatgtacgcgttcgccgtgtcggacgtcatgaccttcggcgcgccgctgatgtacgtgctcggtgtgatgctgctcagcaggtacatggcggacaaaaagcccctgactggcttcatcaagacctacatccagcccgtctacaacgtggtccaaatcgcggtgtgcggctggatggtgtggggcctctggccgcaggtcgacctggccaacggcaaccctttcggcctcaacaagtcgcgcgactcgaacatcgagtttttcgtgttcgtgcacctcctgacaaagtttctcgactggagcgacacgttcatgatgatcctcaagaaaaactacgcccaggttagctttctgcaggtgttccaccacgcaacgatcggcatggtgtggtcgttccttcttcagcgtggctggggctcgggcaccgccgcgtacggtgctttcatcaactcggtcacgcacgtgatcatgtactcgcactactttgccacctcgctcaacatcaacaacccgttcaagcggtacatcacgagcttccagctcgcccagtttgcaagctgcatcgtgcatgccctactggtgcttgccttcgaggaggtgtacccgctcgagtacgcttacctgcagatcagctaccacatcatcatgctctacctgttcggacgccgcatgaactggagccccgagtggtgcaccggtgagatcgacggccttgacgccccaagcgcccccaccaagtccgagtaa

SEQ ID NO: 70 - DNA, 38 bp, synthetic construct
  misc_feature 1..38: note = primer
  source 1..38: mol_type = other DNA
ataagcttaaaatgtctagcaacatgagcgcgtggggc

SEQ ID NO: 71 - DNA, 28 bp, synthetic construct
  misc_feature 1..28: note = primer
  source 1..28: mol_type = other DNA
tgtctagaacgcgcggacggtcgcgaaa

SEQ ID NO: 72 - DNA, 40 bp, synthetic construct
  misc_feature 1..40: note = primer
  source 1..40: mol_type = other DNA
taaagcttaaaatgtctacgcgcacctcgaagagcgctcc

SEQ ID NO: 73 - DNA, 31 bp, synthetic construct
  misc_feature 1..31: note = primer
  source 1..31: mol_type = other DNA
catctagactcggacttggtgggggcgcttg

SEQ ID NO: 74 - DNA, 949 bp, synthetic construct
  misc_feature 1..949: note = cDNA (TaELO1 coding region)
  source 1..949: mol_type = other DNA
ataagcttaaaatgacgagcaacatgagcgcgtggggcgtcgccgtcgaccagacgcagcaggtcgtcgaccagatcatgggcggcgccgagccgtacaagctgacagaagggcgcatgacgaacgtcgagacgatgctggcgatcgagtgcggctacgccgccatgctgctgttcctgaccccgatcatgaagcaggccgagaagcccttcgagctcaagtccttcaagctcgcccacaacctgttcctgttcgtcctgtccgcctacatgtgcctcgagaccgtccgccaggcctaccttgcgggctactcggtgttcggcaacgacatggagaagggcagcgagccgcacgcgcacggcatggcccaaatcgtgtggatcttttacgtgtccaaggcgtacgagttcgtggacacgctgatcatgatcctgtgcaaaaagttcaaccaggtctccgtcctgcacgtgtaccaccacgccaccatctttgctatctggtttatgatcgccaagtacgccccgggcggcgacgcatactttagcgtcatcctgaactcgttcgtgcacaccgtcatgtacgcgtactacttcttctcgtcgcagggcttcgggttcgtcaagccgatcaagccgtacatcacctcgctgcagatgacgcagttcatggcgatgctcgtgcagtcgctgtacgactacctttacccgtgcgactacccgcaggggctcgtcaagctcctcggcgtgtacatgctcaccctgcttgcgctcttcggcaactttttcgtgcagagctacctcaagaagtcgaacaagcccaaggccaagtcggcctaagccgacccgctcgccggcaaccgagcagcacctaggcgcatctcggcccggaaccttttcgacctgctgtggagcgcgcgacgcgtttcgcgaccgtccgcgcgttctagaca

SEQ ID NO: 75 - DNA, 967 bp, synthetic construct
  misc_feature 1..967: note = cDNA (TaELO2 coding region)
  source 1..967: mol_type = other DNA
taaagcttaaaatggcgacgcgcacctcgaagagcgctccggcggtttccaagtcggccaaggttgccgcgccggcgaagaagcggtcggtcgacaggagcgacggtttcttccgcacgttcaacctgtgcgccctgtacgggtctgccctcgcctatgcgtacaagcacggcccggtggacaatgacggccaggggctgtactttcacaagtcgcccatgtacgcgttcgccgtgtcggacgtcatgaccttcggcgcgccgctgatgtacgtgctcggtgtgatgctgctcagcaggtacatggcggacaaaaagcccctgactggcttcatcaagacctacatccagcccgtctacaacgtggtccaaatcgcggtgtgcggctggatggtgtggggcctctggccgcaggtcgacctggccaacggcaaccctttcggcctcaacaagtcgcgcgactcgaacatcgagtttttcgtgttcgtgcacctcctgacaaagtttctcgactggagcgacacgttcatgatgatcctcaagaaaaactacgcccaggttagctttctgcaggtgttccaccacgcaacgatcggcatggtgtggtcgttccttcttcagcgtggctggggctcgggcaccgccgcgtacggtgctttcatcaactcggtcacgcacgtgatcatgtactcgcactactttgccacctcgctcaacatcaacaacccgttcaagcggtacatcacgagcttccagctcgcccagtttgcaagctgcatcgtgcatgccctactggtgcttgccttcgaggaggtgtacccgctcgagtacgcttacctgcagatcagctaccacatcatcatgctctacctgttcggacgccgcatgaactggagccccgagtggtgcaccggtgagatcgacggccttgacgccccaagcgcccccaccaagtccgagtctagatg

SEQ ID NO: 76 - DNA, 1122 bp, synthetic construct
  misc_feature 1..1122: note = cDNA (TaELO2 ORF upstream region)
  source 1..1122: mol_type = other DNA
cgttagaacgcgtaatacgactcactatagggatatcccccgcgaggcgatggctgctccgacgacgtgggctggcgacgtcgctcgcaaaggcgttccgcaaccgcgcgttccgctgtaacgagaccgttttccctgcgctgctgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgacggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatacggctgaccactacgcagcgcgatatcctccatccgcgtatatatccgtaaacaacggaacattctccctctcaacgaggcgtggttttcgaagccatgcctttcttccttcctacttgccttccttctttctttctttctttccttcttttgcaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgctcgctcgcggcgactcatgatgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgtgccgctgtcgctcgctgatccacccgaagcgcggtcggctggcacggtcgctcggctggaagttgagtagtttgctttctgttgctgcgctgctttgtaaacgcgacc

SEQ ID NO: 77 - DNA, 1204 bp, synthetic construct
  misc_feature 1..1204: note = cDNA (TaELO2 downstream region)
  source 1..1204: mol_type = other DNA
acctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccaggacgcgcacgacgagcagcgctgctttgatcgaccttgcctgcgtcaccgcctcggctgctttgatcgatcgttgtcaccggccgagtgaccgcgaacgcattgcccgcacggctcggctcggctcggaccggaccggctcgccttggcggcgcggcgcgatggcgacccagacgcgaccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccgactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgtgatcgtcgagctagcagaggagggccgcatcgactgggccggctatctctcaaccacttcggtctacggcaaccacgacggcgagtgggtggacgagaccacgccgctcatgcccacgctcaaacgcggcgagcagcgcgtcatggtggagcgcgccttcctgtacgagtcgggcctcccggcccatatctttcggctgccaggaatctacggcccagggcgcggcccgatatcacgaattctctccctatagtgagtcgtattacgcgttctaacgacaatatgtac

SEQ ID NO: 78 - DNA, 31 bp, synthetic construct
  misc_feature 1..31: note = primer
  source 1..31: mol_type = other DNA
ctcccgggtggacctagcgcgtgtgtcacct

SEQ ID NO: 79 - DNA, 25 bp, synthetic construct
  misc_feature 1..25: note = primer
  source 1..25: mol_type = other DNA
ggtcgcgtttacaaagcagcgcagc

SEQ ID NO: 80 - DNA, 52 bp, synthetic construct
  misc_feature 1..52: note = primer
  source 1..52: mol_type = other DNA
gctgcgctgctttgtaaacgcgaccatgattgaacaggacggccttcacgct

SEQ ID NO: 81 - DNA, 52 bp, synthetic construct
  misc_feature 1..52: note = primer
  source 1..52: mol_type = other DNA
tcgggagccagccggaaacaggttcaaaagaactcgtccaggaggcggtaga

SEQ ID NO: 82 - DNA, 23 bp, synthetic construct
  misc_feature 1..23: note = primer
  source 1..23: mol_type = other DNA
acctgtttccggctggctcccga

SEQ ID NO: 83 - DNA, 27 bp, synthetic construct
  misc_feature 1..27: note = primer
  source 1..27: mol_type = other DNA
atcccggggccgagaacggggtcgccc

SEQ ID NO: 84 - DNA, 2696 bp, synthetic construct
  misc_feature 1..2696: note = cDNA (TaELO2 ORF upstream/Neor/ TaELO2 ORF downstream)
  source 1..2696: mol_type = other DNA
ctcccgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgatggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatacggctgaccactacgcagcgcgatatcctccatccgcgtatatatccgtaaacaacggaacattctccctctcaacgaggcgtggttttcgaagccatgcctttcttccttcctacttgccttccttctttctttctttctttccttcttttgcaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgcttgctcgcggcgactcatgatgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgcgccgctgtcgctcgctgatccacccgaagcgcggtcggctggcacggtcgctcggctggaagttgagtagtttgctttctgttgctgcgctgctttgtaaacgcgaccatgattgaacaggacggccttcacgctggctcgcccgctgcttgggtggaacggctgttcggctacgactgggctcagcagacgatcggctgctcggacgcggccgtgttccgccttagcgcgcagggccggccggtcctgtttgtcaagaccgaccttagcggcgccctcaacgagctccaggacgaagctgcccgcctcagctggcttgccacgacgggggttccgtgcgccgctgtgctcgacgtcgtcaccgaagccggccgcgactggctgctcctcggggaagtgcccggccaggacctcctcagcagccacctcgcgcccgctgagaaggtgtccatcatggccgacgccatgcgccgcctgcacaccctcgaccccgccacctgccccttcgaccaccaggcgaagcacaggatcgaacgcgcccgcacgcggatggaggctggcctcgtcgaccaagacgacctcgacgaggagcaccagggcctcgcgccggcggaactgttcgccaggcttaaggctaggatgccggacggcgaggacctcgtggtcacgcacggcgacgcctgcctccccaacatcatggtcgagaacggccgcttctcgggctttatcgactgcgggcgcctgggcgtggcggaccgctaccaagacatcgcgctcgccacgcgggacatcgccgaggagcttggcggcgagtgggccgaccgctttctcgtgctctacggcatcgccgccccggacagccagaggattgcgttctaccgcctcctggacgagttcttttgaacctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccgggacgcgcacgacgagcagcgctgctttgatcgaccttgcctgcgtcaccgcctcggctgctttgatcgatcgttgtcaccggccgagtgaccgcgaacgcattgcccgcacggctcggctcggcccggaccggaccggctcgccttggcggcgcggcgcgatggcgacccagacgcggccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccgactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgggat

SEQ ID NO: 85 - DNA, 30 bp, synthetic construct
  misc_feature 1..30: note = primer
  source 1..30: mol_type = other DNA
ggatatcccccgcgaggcgatggctgctcc

SEQ ID NO: 86 - DNA, 30 bp, synthetic construct
  misc_feature 1..30: note = primer
  source 1..30: mol_type = other DNA
tgatatcgggccgcgccctgggccgtagat

SEQ ID NO: 87 - DNA, 25 bp, synthetic construct
  misc_feature 1..25: note = primer
  source 1..25: mol_type = other DNA
gtacgtgctcggtgtgatgctgctc

SEQ ID NO: 88 - DNA, 24 bp, synthetic construct
  misc_feature 1..24: note = primer
  source 1..24: mol_type = other DNA
gcggcgtccgaacaggtagagcat

SEQ ID NO: 89 - DNA, 35 bp, synthetic construct
  misc_feature 1..35: note = primer
  source 1..35: mol_type = other DNA
atccgcgtatatatccgtaaacaacggaacattct

SEQ ID NO: 90 - DNA, 26 bp, synthetic construct
  misc_feature 1..26: note = primer
  source 1..26: mol_type = other DNA
cttcgggtggatcagcgagcgacagc

SEQ ID NO: 91 - DNA, 27 bp, synthetic construct
  misc_feature 1..27: note = primer
  source 1..27: mol_type = other DNA
gccgcagcgcctggtgcacccgccggg

SEQ ID NO: 92 - DNA, 59 bp, synthetic construct
  misc_feature 1..59: note = primer
  source 1..59: mol_type = other DNA
tcgcgggtgagttcaggctttttcatgttggctagtgttgcttaggtcgcttgctgctg

SEQ ID NO: 93 - DNA, 57 bp, synthetic construct
  misc_feature 1..57: note = primer
  source 1..57: mol_type = other DNA
agcgacctaagcaacactaggccaacatgaaaaagcctgaactcaccgcgacgtctg

SEQ ID NO: 94 - DNA, 29 bp, synthetic construct
  misc_feature 1..29: note = primer
  source 1..29: mol_type = other DNA
ctattcctttgccctcggacgagtgctgg

SEQ ID NO: 95 - DNA, 1636 bp, synthetic construct
  misc_feature 1..1636: note = cDNA (T. aureum ATCC 34304 ubiruitin promoter/ Hygr)
  source 1..1636: mol_type = other DNA
gctagccgcagcgcctggtgcacccgccgggcgttggttgtgtgtgctatttactatgcctaccgagagagagagcggagcggatgcataggaaatcgggccacgcgggagggccatgcgttcgccccacacgccacttataccacgcccgctctctctccggccggcaggcagcgcataactataccgacgctggcaggcttggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttccgcgattccctcttctgaattctgaattctgaactgattccggaggagaaccctctggaagcgcgggttgcctctccagttctgccgaactagacaggggagtgagcatgatgagtgaccctgacgcgtgagctgagctggttgctggaatatagtcgctgaacgctgggctgtgtcacgcgtccacttcgggcagaccccaaacgacaagcagaacaagcaacaccagcagcagcaagcgacctaagcaacactagccaacatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtctag

SEQ ID NO: 96 - DNA, 33 bp, synthetic construct
  misc_feature 1..33: note = primer
  source 1..33: mol_type = other DNA
gtgctagccgcagcgcctggtgcacccgccggg

SEQ ID NO: 97 - DNA, 37 bp, synthetic construct
  misc_feature 1..37: note = primer
  source 1..37: mol_type = other DNA
gttctagactattcctttgccctcggacgagtgctgg

SEQ ID NO: 98 - DNA, 37 bp, synthetic construct
  misc_feature 1..37: note = primer
  source 1..37: mol_type = other DNA
gttctagacctgtttccggctggctcccgagccatgc

SEQ ID NO: 99 - DNA, 40 bp, synthetic construct
  misc_feature 1..40: note = primer
  source 1..40: mol_type = other DNA
gtgctagcggtcgcgtttacaaagcagcgcagcaacagaa

SEQ ID NO: 100 - DNA, 3537 bp, synthetic construct
  misc_feature 1..3537: note = cDNA (TaELO2 ORF upstream region/T. aureum ATCC 34304 ubiquitin promotor/ Hygr /TaELO2 ORF downstream region)
  source 1..3537: mol_type = other DNA
ctcccgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgacggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatacggctgaccactacgcagcgcgatatcctccatccgcgtatatatccgtaaacaacggaacattctccctctcaacgaggcgtggttttcgaagccatgcctttcttccttcctacttgccttccttctttctttctttctttctttcttttgtaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgctcgctcgcggcgactcatggtgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgtgccgctgtcgctcgctgatccacccgaagcgcggtcggctggcacggtcgctcggctggaagttgagtagtttgctttctgttgctgcgctgctttgtaaacgcgaccgctagccgcagcgcctggtgcacccgccgggcgttggttgtgtgtgctatttactatgcctaccgagagagagagcggagcggatgcataggaaatcgggccacgcgggagggccatgcgttcgccccacacgccacttataccacgcccgctctctctccggccggcaggcagcgcataactataccgacgctggcaggcttggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttccgcgattccctcttctgaattctgaattctgaactgattccggaggagaaccctctggaagcgcgggttgcctctccagttctgccgaactagacaggggagtgagcatgatgagtgaccctgacgcgtgagctgagctggttgctggaatatagtcgctgaacgctgggctgtgtcacgcgtccacttcgggcagaccccaaacgacaagcagaacaagcaacaccagcagcagcaagcgacctaagcaacactagccaacatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtctagacctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccgggacgcgcacgacgagcagcgctgctttgatcgaccttgcctgcgtcaccgcctcggctgctttgatcgatcgttgtcaccggccgagtgaccgcgaacgcattgcccgcacggctcggctcggcccggaccggaccggctcgccttggcggcgcggcgcgatggcgacccagacgcggccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccgactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgggat

SEQ ID NO: 101 - DNA, 30 bp, synthetic construct
  misc_feature 1..30: note = primer
  source 1..30: mol_type = other DNA
atggcgacgcgcacctcgaagagcgctccg

SEQ ID NO: 102 - DNA, 30 bp, synthetic construct
  misc_feature 1..30: note = primer
  source 1..30: mol_type = other DNA
aggatcatcatgaacgtgtcgctccagtcg

SEQ ID NO: 103 - DNA, 23 bp, synthetic construct
  misc_feature 1..23: note = primer
  source 1..23: mol_type = other DNA
cgatgaaaggtcacagaagagtc

SEQ ID NO: 104 - DNA, 3181 bp, synthetic construct
  misc_feature 1..3181: note = genomic DNA (T. aureum ATCC 34304 OrfA upstream)
  source 1..3181: mol_type = other DNA
aagcttttgctctgcggctctgcttgttcgaagccaacgcgcctcgcgaagtatctgcaatctgcactcctccggagagtaagtacgtaagtacgtgcgtggtgcgcgcggattgcggtgacgaaagagagggttgggttggagatgctgcggcatgccgggcgactcgagcagcatgtcgccgcgagaggacctggaaagctttcggtttggtccgctgccgaggcgaggctggcagagtactgcgggcggagctctcgagggaatatgctcctcaaagacggcgtgcgcgtttgtgcccccgaatccgaatgcggagagtcctgcgcgtttcggcccgccgcgcgtatccggccacgggagcgccgctgtgacgacgaggggatcaatctgggtgccagagtcaacgcccagggtcggggggatcacgccgcgctccattgcaaggagaaccttgcacattcccgcaaagccggccgcgacgagggtgtgtccgaagttgcctttcgtggacccgatccgggggcttgcgccctcaaagcaggctttgacggcttggagctcgacggtgtcgccctgcggcgtgccggtggcgtggcactcgacgtactggacgtctcggggcggcacgccgacgagctcgtaggtggctttcaagcaggcctcctcgcttggctggtgcggcttgagaggaagcccgcagcctgcgttgctcaagctggccccaagaagcgtcccgtagatgtggtctccgtcgcgctcggcgtccgcgaggcgcttgagcaccatcaccgagccgccctcgccgggcgtcagcccttgcgtgtcccgatgaaacggcatcgagacaccgttctcaccgactggcatcgcgtggaacgtgctaaacccagtcaggatgaagaagggctctgggaagcacgtcgctccgcacagcatcaagtcagcctcgcccgagaggaggtggtcctgagcgagtcgcagaacgtaaagggccgaggcgcaggcggcgtcgagcgagtagtgcagcgggccgaggccgagctgtccggcgacgaaggaggctgggtcgcggtgggtcctcgggtccccgggcagcgggtgaagcgctctggttcgcgtcgaccagggcgtttggtccgcgaagcaatgcttgccaatccgcctctcagcatgggcttggtaaaggttgagcagctcgccttgcaggttgtccatcgggaaggacaggcagccgctgacaatgccgcagcgcttgagctgcgctgggtcgaacttgccgccgtcgctgcgcctgtcctgcgcgtcttgaagcgcagccgcggcgaggccgaggagcaggtcgtgctcgttgtcgactttgggatcgatgcatccgtacctctcgttgcagaacgtatcggcgtactttgacctctcgggcgcatagtgctcttctcgtcgtgctgacccgaggcgatcgtctgagatacaggcagagttgattttgccgttcatgagcgtgtcccagaacgcttccttgccgcggcaccctgcatactcgaccgccatgcccacgacagcgatccgcgtgtcagggcatgggtccgcgcgcgccaccgagacgccctcgtcatttctcccgccctggttcatctctttctgagccttgtggcctcttgctttcgatttcgatcggcagctccaacgcgcagctcgatggccgattgcttgctaaggcggcgtgcagacaaccgctgctcgaggttctgggcaagccgaggttcgccgcagggttcgtggaggcactgggatgttgttttgcggcagctgcagcgcttgcggagcgagcggcgcaggacgacgttttccgcgttcgcgcgaagctgcctctcggctattgtgacccgccgccgacgctggcagagacgtcgccgtcggccaccgcacctcgagatcaattcgcagaggctggcagagccggtagcattgcggcgtggcattgcgtgtgccatgtgcatgtgtggcaaacaaatccagccaacctccgagtcgggcagaccgaccgtgtgagttctcgctgttgactgatctcttgattgagcccaataatgatcacggcctgagatccttcgcgctgagagatgcatgcgggcgctcgttcctgggttggcgacccaacggcgagtcacgtcgcccactccgccacgccccacacatggccgccgatccctcccgccacacgaacggcgggccaagatcgcacgcctccgtcggacgatgactgactgactgattggctgacgacggccgccctcgtgcgcggcgtcgggcgtcgtcgcaaaccaggcaggcaggcaggaaggaaggaaggaagggccaggccctggtgcgaaacgctggcctgctccgctgcaagccaagccgcgctcgcaggtgtacttccgagtcctcgcgatgattaggcaagcctgagcgagcacgtaagctgcactgcggctgttcaaccagagagagagttggctctcttgcgtcaaggcggcgcgcagcccacttgcgtcgcggctgagggcccctggaggggaggaaggaggccggcgagcggcgagtggcggccctcactggcaccaggtcgcaggaggccaggcagcccgccacggacaggaatcctcagggcgcagcagcgcactacgtagtgcagagacgcagagcgggccggatccgcagtgcggtcgcgccaccccgccgcgcagctcgctcgcggacggggtccgtggccgcgcgaaaacggacacggtgtgggagcggacatgggatcgagaacgccgttcgccctgctcgcgctgccagcagcaggagccgtccgaaggacgagcggccggccgcctgtcccccctccgcgcactcgaagcgcgcccggcagcgccccattgcgtgcgcggatggcgtcttggctggtccctctcgaggcgcttgctcgtgctcgccacgccttgtccgcctcctcgctgagcaagcgatgagctgagcacggaccgcctgcaagtgcaagtgttcttgtgctgcagggcgccgaagaattggattctggcccatgatcagtttgattgggccgagggagggagggaggctgggcgagtgggcgacaccagcaagccggactgcgagaggggcggggcaggatgtgagcgcaggaaagtga

SEQ ID NO: 105 - DNA, 3377 bp, synthetic construct
  misc_feature 1..3377: note = genomic DNA (T. aureum ATCC 34304 OrfA upstream genomic DNA fragment)
  source 1..3377: mol_type = other DNA
gaattcgattaatacgactcactatagggagaagcttttgctctgcggctctgcttgttcgaagccaacgcgcctcgcgaagtatctgcaatctgcactcctccggagagtaagtacgtaagtacgtgcgtggtgcgcgcggattgcggtgacgaaagagagggttgggttggagatgctgcggcatgccgggcgactcgagcagcatgtcgccgcgagaggacctggaaagctttcggtttggtccgctgccgaggcgaggctggcagagtactgcgggcggagctctcgagggaatatgctcctcaaagacggcgtgcgcgtttgtgcccccgaatccgaatgcggagagtcctgcgcgtttcggcccgccgcgcgtatccggccacgggagcgccgctgtgacgacgaggggatcaatctgggtgccagagtcaacgcccagggtcggggggatcacgccgcgctccattgcaaggagaaccttgcacattcccgcaaagccggccgcgacgagggtgtgtccgaagttgcctttcgtggacccgatccgggggcttgcgccctcaaagcaggctttgacggcttggagctcgacggtgtcgccctgcggcgtgccggtggcgtggcactcgacgtactggacgtctcggggcggcacgccgacgagctcgtaggtggctttcaagcaggcctcctcgcttggctggtgcggcttgagaggaagcccgcagcctgcgttgctcaagctggccccaagaagcgtcccgtagatgtggtctccgtcgcgctcggcgtccgcgaggcgcttgagcaccatcaccgagccgccctcgccgggcgtcagcccttgcgtgtcccgatgaaacggcatcgagacaccgttctcaccgactggcatcgcgtggaacgtgctaaacccagtcaggatgaagaagggctctgggaagcacgtcgctccgcacagcatcaagtcagcctcgcccgagaggaggtggtcctgagcgagtcgcagaacgtaaagggccgaggcgcaggcggcgtcgagcgagtagtgcagcgggccgaggccgagctgtccggcgacgaaggaggctgggtcgcggtgggtcctcgggtccccgggcagcgggtgaagcgctctggttcgcgtcgaccagggcgtttggtccgcgaagcaatgcttgccaatccgcctctcagcatgggcttggtaaaggttgagcagctcgccttgcaggttgtccatcgggaaggacaggcagccgctgacaatgccgcagcgcttgagctgcgctgggtcgaacttgccgccgtcgctgcgcctgtcctgcgcgtcttgaagcgcagccgcggcgaggccgaggagcaggtcgtgctcgttgtcgactttgggatcgatgcatccgtacctctcgttgcagaacgtatcggcgtactttgacctctcgggcgcatagtgctcttctcgtcgtgctgacccgaggcgatcgtctgagatacaggcagagttgattttgccgttcatgagcgtgtcccagaacgcttccttgccgcggcaccctgcatactcgaccgccatgcccacgacagcgatccgcgtgtcagggcatgggtccgcgcgcgccaccgagacgccctcgtcatttctcccgccctggttcatctctttctgagccttgtggcctcttgctttcgatttcgatcggcagctccaacgcgcagctcgatggccgattgcttgctaaggcggcgtgcagacaaccgctgctcgaggttctgggcaagccgaggttcgccgcagggttcgtggaggcactgggatgttgttttgcggcagctgcagcgcttgcggagcgagcggcgcaggacgacgttttccgcgttcgcgcgaagctgcctctcggctattgtgacccgccgccgacgctggcagagacgtcgccgtcggccaccgcacctcgagatcaattcgcagaggctggcagagccggtagcattgcggcgtggcattgcgtgtgccatgtgcatgtgtggcaaacaaatccagccaacctccgagtcgggcagaccgaccgtgtgagttctcgctgttgactgatctcttgattgagcccaataatgatcacggcctgagatccttcgcgctgagagatgcatgcgggcgctcgttcctgggttggcgacccaacggcgagtcacgtcgcccactccgccacgccccacacatggccgccgatccctcccgccacacgaacggcgggccaagatcgcacgcctccgtcggacgatgactgactgactgattggctgacgacggccgccctcgtgcgcggcgtcgggcgtcgtcgcaaaccaggcaggcaggcaggaaggaaggaaggaagggccaggccctggtgcgaaacgctggcctgctccgctgcaagccaagccgcgctcgcaggtgtacttccgagtcctcgcgatgattaggcaagcctgagcgagcacgtaagctgcactgcggctgttcaaccagagagagagttggctctcttgcgtcaaggcggcgcgcagcccacttgcgtcgcggctgagggcccctggaggggaggaaggaggccggcgagcggcgagtggcggccctcactggcaccaggtcgcaggaggccaggcagcccgccacggacaggaatcctcagggcgcagcagcgcactacgtagtgcagagacgcagagcgggccggatccgcagtgcggtcgcgccaccccgccgcgcagctcgctcgcggacggggtccgtggccgcgcgaaaacggacacggtgtgggagcggacatgggatcgagaacgccgttcgccctgctcgcgctgccagcagcaggagccgtccgaaggacgagcggccggccgcctgtcccccctccgcgcactcgaagcgcgcccggcagcgccccattgcgtgcgcggatggcgtcttggctggtccctctcgaggcgcttgctcgtgctcgccacgccttgtccgcctcctcgctgagcaagcgatgagctgagcacggaccgcctgcaagtgcaagtgttcttgtgctgcagggcgccgaagaattggattctggcccatgatcagtttgattgggccgagggagggagggaggctgggcgagtgggcgacaccagcaagccggactgcgagaggggcggggcaggatgtgagcgcaggaaagtgacgcaagtgcatccggccatcattgggccatcattgggccatcattggtgttttgggccgcgctttgcggatcgtccggccgatcaggtacgaggccacgaacctacgtcgtttgccgcgctcaggctggttggttgcacttggactcttctgtgacctttcatcg

SEQ ID NO: 106 - DNA, 21 bp, synthetic construct
  misc_feature 1..21: note = primer
  source 1..21: mol_type = other DNA
cagggcgagcgagtgtggttc

SEQ ID NO: 107 - DNA, 1160 bp, synthetic construct
  misc_feature 1..1160: note = genomic DNA (T. aureum ATCC 34304 OrfA downstream)
  source 1..1160: mol_type = other DNA
tggctctcgaccaaagccgagtagagtactctactcagtactcttttcacataccggcaggcagtgttgctgtgggattggtccgggggctcttctgcacgcggcctccgtcgcgcgcagaaatgccccgtcactggctgcccaggaggcagccgaatccctctagctagctagctaggctagagcgtcttttccgtagtttttcacaaagccagtatcacatggataacgaacgaaggtttcgggctcgcgctcgcaggcgttaggacgaagttgatcgccccacgtcacttcaaacgagtgaaccaagatcacgttgcatctgctcgcaagatcttcttcttccacgccgcatcgatgcgatggatttcaaactcttttcagggcttttaggtgagtatggcagcgctgtttgcgtggcagcgctgtttgcgtggttgtactctctaaaggtgcttccacgcatgcgcgcacaaaggggcatggcatggttggcggcgcactctggccctcatttgaagcagactatcgaagggtccagttggtactgcggcaggtccggcgagagcaagcgcggcggtcgctcccactcgtccctgcacagttgctggactggcgacggctggcgcacctgactacgagaagactcgagacgcacagaggtagtcagggacgaccgaccgcaaagcacaaaccgctccaaaacggccgcaccaggcagggcagtaaactaaaaacgaatgtacctccatcgcgcgtatctgccgagcctcctcccacgcttcggctgggcttgattcaccagtgtccgcaagctgaaccgaccgtcttcgatgtcatgaagcttggcgcggcattagtcagacgacgcggcacgccaggattctgtcggtttctgggaaatgggcatctatatagctgattccctctgtcatgaggcggccttgttctggccctgggccgccgttcggatgatctatgatgtcgttgtacgcataaagcttgtcgaaaacgtcggccatgtcttcctcagagatgtaaccgagcggcgcgtcgtggcgattgatgccgatgctacaaaagccgccgagttagctcgaatgtcagatgcattgcgggctggcccgcatggcgcgggcgcagcagcgagaggttctaga

SEQ ID NO: 108 - DNA, 1204 bp, synthetic construct
  misc_feature 1..1204: note = genomic DNA (T. aureum ATCC 34304 OrfA downstream genomic DNA fragment)
  source 1..1204: mol_type = other DNA
cagggcgagcgagtgtggttctgaacaaggctctttcgttttgatggctctcgaccaaagccgagtagagtactctactcagtactcttttcacataccggcaggcagtgttgctgtgggattggtccgggggctcttctgcacgcggcctccgtcgcgcgcagaaatgccccgtcactggctgcccaggaggcagccgaatccctctagctagctagctaggctagagcgtcttttccgtagtttttcacaaagccagtatcacatggataacgaacgaaggtttcgggctcgcgctcgcaggcgttaggacgaagttgatcgccccacgtcacttcaaacgagtgaaccaagatcacgttgcatctgctcgcaagatcttcttcttccacgccgcatcgatgcgatggatttcaaactcttttcagggcttttaggtgagtatggcagcgctgtttgcgtggcagcgctgtttgcgtggttgtactctctaaaggtgcttccacgcatgcgcgcacaaaggggcatggcatggttggcggcgcactctggccctcatttgaagcagactatcgaagggtccagttggtactgcggcaggtccggcgagagcaagcgcggcggtcgctcccactcgtccctgcacagttgctggactggcgacggctggcgcacctgactacgagaagactcgagacgcacagaggtagtcagggacgaccgaccgcaaagcacaaaccgctccaaaacggccgcaccaggcagggcagtaaactaaaaacgaatgtacctccatcgcgcgtatctgccgagcctcctcccacgcttcggctgggcttgattcaccagtgtccgcaagctgaaccgaccgtcttcgatgtcatgaagcttggcgcggcattagtcagacgacgcggcacgccaggattctgtcggtttctgggaaatgggcatctatatagctgattccctctgtcatgaggcggccttgttctggccctgggccgccgttcggatgatctatgatgtcgttgtacgcataaagcttgtcgaaaacgtcggccatgtcttcctcagagatgtaaccgagcggcgcgtcgtggcgattgatgccgatgctacaaaagccgccgagttagctcgaatgtcagatgcattgcgggctggcccgcatggcgcgggcgcagcagcgagaggttctaga

SEQ ID NO: 109 - DNA, 20 bp, synthetic construct
  misc_feature 1..20: note = primer
  source 1..20: mol_type = other DNA
tgatgccgatgctacaaaag

SEQ ID NO: 110 - DNA, 1488 bp, synthetic construct
  misc_feature 1..1488: note = genomic DNA (T. aureum ATCC 34304 OrfA downstream genomic DNA fragment)
  source 1..1488: mol_type = other DNA
aagcttgtacggtgaaaagccctttggcgcagcccgaaacaagtcttgcttctcctgccccgtcaaactcgcaaactctggcagcaactcccgcacgctctgtaccacggcgaacccaagggcaggcacgcggtgaaacgacttgcatgcttgcacaacaacccccttgccgacgtcgacgcggtcgccttcggagagcccaaacacagcgaacgccggatccgcctgcgcctctgcatgcgcctctgcatgcgcctcgacatgcgcctcggcctccgtgcctgcttgccgggccggcggggcagcaggaagtgcgtggccgaggtccatcgcatcaaaggctcgcttcgcggcgtgaaaggcctcgagcgcctccgccggcaagtacaccttggtcttgcacttgagcatgctcctgatccgcgcgtagaggaagacggccgcgcagtggtccaggtgcccgtgcgacaagaacacgtgctccgccctcgccgcggccttgtccggctcgtccccgagcgacccgcagtcgaactgcaagcagacccgcgagcccaggtccacttgcagcgccgtgccgcagccggccctcgactgccccgtcacgcggacgtgcgaggccatctcccgccgcgagcctggagcgccagagcctcctgctgctgccgtgccgcctcggggggcgcgaggagggtctcgcctgatgcagcgcgcggggccgacgcagcagcgcgggtggaggaagactgcgctgtgggcggcggccctcgggctgctgctcttgtggctcctgtccgtgcgctcgttcgtgcacggcgtggcggacagggaggcggacgccgtcgccccgcgcgagggccccagggcgccggcgccaaagaggactggcgggaggaatgatatgcccgctgagcctgccgctggtaggcccgcgcacagctcgcctcgagggacgcccgacggcaacgcggtcgagtgctccacgaccaagggcccgttccgcgtggtcctcacgcctagcctagcgccgaacgggaccaagtttttcatcgggctggtggaagcaggctatttcgaccaaggcatcgccttctttcgcgtcaacaaggccatcacgcagttcgggatcaccaagcgaaggccacgcgatgaggatccgttcgtgcagttcagaggcggggcccagcgcgacgagaaccctttcggtggcgtggaggatgacgaggagagtgtccatcgcaggcacatgcacccgtggcggcgcggcacgattgcctcgataggcggcttccactttgttgtcacgatccgcggggacaaaaagtaagttcttgaatgttgtgaagtgcgccaactcgcgttcggagcggacctggaccgatattcagcaatctagaacctctcgctgctgcgcccgcgccatgcgggccagcccgcaatgcatctgacattcgagctaactcggcggcttttgtagcatcggcatca

SEQ ID NO: 111 - DNA, 2551 bp, synthetic construct
  misc_feature 1..2551: note = genomic DNA (T. aureum ATCC 34304 OrfA downstream genomic DNA fragment)
  source 1..2551: mol_type = other DNA
tggctctcgaccaaagccgagtagagtactctactcagtactcttttcacataccggcaggcagtgttgctgtgggattggtccgggggctcttctgcacgcggcctccgtcgcgcgcagaaatgccccgtcactggctgcccaggaggcagccgaatccctctagctagctagctaggctagagcgtcttttccgtagtttttcacaaagccagtatcacatggataacgaacgaaggtttcgggctcgcgctcgcaggcgttaggacgaagttgatcgccccacgtcacttcaaacgagtgaaccaagatcacgttgcatctgctcgcaagatcttcttcttccacgccgcatcgatgcgatggatttcaaactcttttcagggcttttaggtgagtatggcagcgctgtttgcgtggcagcgctgtttgcgtggttgtactctctaaaggtgcttccacgcatgcgcgcacaaaggggcatggcatggttggcggcgcactctggccctcatttgaagcagactatcgaagggtccagttggtactgcggcaggtccggcgagagcaagcgcggcggtcgctcccactcgtccctgcacagttgctggactggcgacggctggcgcacctgactacgagaagactcgagacgcacagaggtagtcagggacgaccgaccgcaaagcacaaaccgctccaaaacggccgcaccaggcagggcagtaaactaaaaacgaatgtacctccatcgcgcgtatctgccgagcctcctcccacgcttcggctgggcttgattcaccagtgtccgcaagctgaaccgaccgtcttcgatgtcatgaagcttggcgcggcattagtcagacgacgcggcacgccaggattctgtcggtttctgggaaatgggcatctatatagctgattccctctgtcatgaggcggccttgttctggccctgggccgccgttcggatgatctatgatgtcgttgtacgcataaagcttgtcgaaaacgtcggccatgtcttcctcagagatgtaaccgagcggcgcgtcgtggcgattgatgccgatgctacaaaagccgccgagttagctcgaatgtcagatgcattgcgggctggcccgcatggcgcgggcgcagcagcgagaggttctagattgctgaatatcggtccaggtccgctccgaacgcgagttggcgcacttcacaacattcaagaacttactttttgtccccgcggatcgtgacaacaaagtggaagccgcctatcgaggcaatcgtgccgcgccgccacgggtgcatgtgcctgcgatggacactctcctcgtcatcctccacgccaccgaaagggttctcgtcgcgctgggccccgcctctgaactgcacgaacggatcctcatcgcgtggccttcgcttggtgatcccgaactgcgtgatggccttgttgacgcgaaagaaggcgatgccttggtcgaaatagcctgcttccaccagcccgatgaaaaacttggtcccgttcggcgctaggctaggcgtgaggaccacgcggaacgggcccttggtcgtggagcactcgaccgcgttgccgtcgggcgtccctcgaggcgagctgtgcgcgggcctaccagcggcaggctcagcgggcatatcattcctcccgccagtcctctttggcgccggcgccctggggccctcgcgcggggcgacggcgtccgcctccctgtccgccacgccgtgcacgaacgagcgcacggacaggagccacaagagcagcagcccgagggccgccgcccacagcgcagtcttcctccacccgcgctgctgcgtcggccccgcgcgctgcatcaggcgagaccctcctcgcgccccccgaggcggcacggcagcagcaggaggctctggcgctccaggctcgcggcgggagatggcctcgcacgtccgcgtgacggggcagtcgagggccggctgcggcacggcgctgcaagtggacctgggctcgcgggtctgcttgcagttcgactgcgggtcgctcggggacgagccggacaaggccgcggcgagggcggagcacgtgttcttgtcgcacgggcacctggaccactgcgcggccgtcttcctctacgcgcggatcaggagcatgctcaagtgcaagaccaaggtgtacttgccggcggaggcgctcgaggcctttcacgccgcgaagcgagcctttgatgcgatggacctcggccacgcacttcctgctgccccgccggcccggcaagcaggcacggaggccgaggcgcatgtcgaggcgcatgcagaggcgcatgcagaggcgcaggcggatccggcgttcgctgtgtttgggctctccgaaggcgaccgcgtcgacgtcggcaagggggttgttgtgcaagcatgcaagtcgtttcaccgcgtgcctgcccttgggttcgccgtggtacagagcgtgcgggagttgctgccagagtttgcgagtttgacggggcaggagaagcaagacttgtttcgggctgcgccaaagggcttttcaccgtacaagctt

SEQ ID NO: 112 - DNA, 1835 bp, synthetic construct
  misc_feature 1..1835: note = 18S rDNA (T. aureum ATCC 34304)
  source 1..1835: mol_type = other DNA
cgaatattcctggttgatcctgccagtagtcatacgcttatctcaaagattaagccatgcatgtctaagtataaaggcttatactctgaaactgcgaacggctcattatatcagttatagtttctttgatagtgttttttctacatggatacttgtggcaaatctagaaacaatacatgcgtacaggcctgactttgggggagggctgcatttatttgacttaagccaatacccctcggggttgttttggtgattcagaataactgagcgaatcgcatagctttcgggcggcgatgaatcatttcaagtttctgccccatcagctgtcgatggtagggtataggcctaccatggctgtcacgggtgacggagaattagggttcgattccggagagggagcctgagagacggctaccacatccaaggaaggcagcaggcgcgtaaattactcaatgttgactcgacgaagtagtgacgagaattaacaatgcggagcgctcagcgttttgcaattggaatgagagcaatgtaaaagcctcatcgaggatccattggagggcaagtctggtgccagcagccgcggtaattccagctccaatagcgtatactaaagttgttgcagttaaaaagctcgtagttgaacctctggtagggccgaccttggcgcgcggtgaatgccgcgtcgtttagaagcgtcgtgcccggccatcctcccccggtcttttgggctgggggtcgtttactgtaaaaaaaatagagtgttccaagcagggggtaatatcccggtatatagtagtatggaataatgagataggactttggtactattttgttggtttgcatgccaaggtaatgattaagagggacagttgggggtattcgtatttagatgtcagaggtgaaattcttggattttcgaaagacgaactactgcgaaagcatttaccaaggatgttttcattaatcaagaacgaaagttaggggatcgaagatgattagataccatcgtagtcttaaccgtaaactatgccgacttgcgattgtccggcgtcgcttttagatgacctgggcagcagcacatgagaaatcaaagtctttgggttccggggggagtatggtcgcaaggctgaaacttaaaggaattgacggaagggcaccaccaggagtggagcctgcggcttaatttgactcaacacgggaaaacttaccaggtccggacataggaaggattgacagattgagagctctttcttgattctatgggtggtggtgcatggccgttcttagttggtggagtgatttgtctggttaattccgttaacgaacgagaccacagcctactaaatagtggccgttatggcgacatagcggtgaacttcttagagggacatttcgggtataccggaaggaagtttgtggcaataacaggtctgtgatgcccttagatgttctgggccgcacgcgcgctacactgatcggttcaacgagtatttgtttttttctcattttgggagggggcagagtccttggccggaaggtctgggtaatcttttgaatgccgatcgtgatggggctagatttttgcaattattaatctccaacgaggaattcctagtagacgcaagtcatcagcttgcatcgattacgtccctgccctttgtacacaccgcccgtcgcacctaccgattgaacgatccggtgagaccttgggattctgttgtggctgattcattttggctgcgatgggagaacttgagcaaaccttatcgtttagaggaaggtgaagtcgtaacaaggtttccgtagtgaacctgcaattcaaaaaaagccgttac

SEQ ID NO: 113 - DNA, 30 bp, synthetic construct
  misc_feature 1..30: note = primer
  source 1..30: mol_type = other DNA
cgaatattcctggttgatcctgccagtagt

SEQ ID NO: 114 - DNA, 46 bp, synthetic construct
  misc_feature 1..46: note = primer
  source 1..46: mol_type = other DNA
gtaacggctttttttgaattgcaggttcactacgcttgttagaaac

SEQ ID NO: 115 - DNA, 661 bp, synthetic construct
  misc_feature 1..661: note = EF1 alpha promoter (T. aureum ATCC 34304)
  source 1..661: mol_type = other DNA
ggtttccgtagtgaacctgcaattcaaaaaaagccgttactcacatcaggccgccactcatccgggcgaaagcttcgcgcattcgtcctcgtcacctcgggtcccctgtgtcgtgacggaaagcgcgacgagacgcggccgcagcagagagccccgggggcccgcgtcacggggggcctggcggcggtcctccttaagccaaaccgagggttagggctccaggctgttcggcggggtcgcgggcgcggtggacgcgcggggccgcctagcacctcctagcgcgcgactaccaggatagcccccgcgagtgcgcagggcggtccgcggggcggagggcggcccagcagcgcggcgcggcgggcgggtgcggctgcgtaaggtggcggcgggcgcgggcggttagtgttggtgttaggtcgcggcggggctgtgttccgggcatccgccttacggcggtgcatactggttggctgggaggcggtttgcggggttagataggcggccaaggtgagctgcgttgggcggataaatccgtggaggcgctcgttgacggcgcggcagagacggaacgcggagcagcacggagtagcaagcaggagtagcaggagtagcaagcagcggcaaaggaaggctagatgattgaacaggacggccttcacgc

SEQ ID NO: 116 - DNA, 46 bp, synthetic construct
  misc_feature 1..46: note = primer
  source 1..46: mol_type = other DNA
ggtttccgtagtgaacctgcaattcaaaaaaagccgttactcacat

SEQ ID NO: 117 - DNA, 46 bp, synthetic construct
  misc_feature 1..46: note = primer
  source 1..46: mol_type = other DNA
gcgtgaaggccgtcctgttcaatcatctagccttcctttgccgctg

SEQ ID NO: 118 - DNA, 835 bp, synthetic construct
  misc_feature 1..835: note = Neomycin resistance gene (Neor)
  source 1..835: mol_type = other DNA
catcggcaaaggaaggctagatgattgaacaggacggccttcacgctggctcgcccgctgcttgggtggaacggctgttcggctacgactgggctcagcagacgatcggctgctcggacgcggccgtgttccgccttagcgcgcagggccggccggtcctgtttgtcaagaccgaccttagcggcgccctcaacgagctccaggacgaagctgcccgcctcagctggcttgccacgacgggggttccgtgcgccgctgtgctcgacgtcgtcaccgaagccggccgcgactggctgctcctcggggaagtgcccggccaggacctcctcagcagccacctcgcgcccgctgagaaggtgtccatcatggccgacgccatgcgccgcctgcacaccctcgaccccgccacctgccccttcgaccaccaggcgaagcacaggatcgaacgcgcccgcacgcggatggaggctggcctcgtcgaccaagacgacctcgacgaggagcaccagggcctcgcgccggcggaactgttcgccaggcttaaggctaggatgccggacggcgaggacctcgtggtcacgcacggcgacgcctgcctccccaacatcatggtcgagaacggccgcttctcgggctttatcgactgcgggcgcctgggcgtggcggaccgctaccaagacatcgcgctcgccacgcgggacatcgccgaggagcttggcggcgagtgggccgaccgctttctcgtgctctacggcatcgccgccccggacagccagaggattgcgttctaccgcctcctggacgagttcttttgagatccgcgccggctatgcgc

SEQ ID NO: 119 - DNA, 45 bp, synthetic construct
  misc_feature 1..45: note = primer
  source 1..45: mol_type = other DNA
catcggcaaaggaaggctagatgattgaacaggacggccttcacg

SEQ ID NO: 120 - DNA, 46 bp, synthetic construct
  misc_feature 1..46: note = primer
  source 1..46: mol_type = other DNA
gcgcatagccggcgcggatctcaaaagaactcgtccaggaggcggt

SEQ ID NO: 121 - DNA, 1249 bp, synthetic construct
  misc_feature 1..1249: note = EF1 alpha terminator (T. aureum ATCC 34304)
  source 1..1249: mol_type = other DNA
tcctggacgagttcttttgagatccgcgccggctatgcgcccgtgctcgactgccacactgcccacattgcctgcaagttcgctgagctccagaacaagatggaccgccgctcgggcaagattctcgaggagacccccaagttcatcaagtcgggtggactctgccatggtcaagatgtatcccctccaagcgcatgtgcgtcgagtccttcaccgagtacccgccgctcggccgctttgccgtgcgcgacatgcgcgtcaccgtcgctgtcggcgtcatcaagtccgtcaccaagggcgacaaataaattctacgaaagatttttttcctcaagaagcgccctaaagttgacccctagcagcgacgactgtgtgtgccgttgtgagtcgagttgcgatgtcgtgcagcgcccgtcgcgtcccatgctcgcgcgcgactccgtctctgcttttcatctcaagtcaagagtgggaagttcccttgctttatctcactatttagaggtcgctcacggctgctggttcctcgtcgcatgtagcacagcctcgtccaatcgcagcctgcaccaccccgctcgcctgggaaaatgcgctcagcggattcgcactggcactcctctcctcggacaggtgcgatgtggaagcggtcacatcctcggcgccctcggccacgccagcatctgcgcaatcgctctcctcgttctcagccgcaaccgcaggcaggccgacgtcgtttacctcggaatccaccgagcatttcgagcccatcgcgctggcgtccacctcgatcataccttctccatcgccgtccgctgcggcttccgattcttctgctgccgcaaccgcgacgtcggcccccgtctcctccgttctttccgatgccggcgcagtggccgcgccctctgctcgaaccggctcgtgttcagcgtcagggcctgcgcttgagctcgggcggctcttccgagtgatccggccccgcgaggcaaggaatcggcggctctggagtgtcggggcagccgctctcactgccggtctttggctggctgcctgtcctgcctcgcgttggcctttgcttttgcctaggctttcgccttggtgacggcgtttgcctgctgcggcgacttggcgcggccgcggaatagcgcctcaaagtcctgctcgaggcgccccagctctgacttgatttgcgaggtcccggtggcatgagctccgctgccctcgtccttacggcccgtctttcgctgcagtg

SEQ ID NO: 122 - DNA, 46 bp, synthetic construct
  misc_feature 1..46: note = primer
  source 1..46: mol_type = other DNA
tcctggacgagttcttttgagatccgcgccggctatgcgcccgtgc

SEQ ID NO: 123 - DNA, 30 bp, synthetic construct
  misc_feature 1..30: note = primer
  source 1..30: mol_type = other DNA
cactgcagcgaaagacgggccgtaaggacg

SEQ ID NO: 124 - DNA, 4453 bp, synthetic construct
  misc_feature 1..4453: note = fusion DNA (T. aureum ATCC 34304 18S rDNA/T. aureum ATCC 34304 EF1 alpha promoter/Neor/T. aureum ATCC 34304 EF1 alpha terminator)
  source 1..4453: mol_type = other DNA
cgaatattcctggttgatcctgccagtagtcatacgcttatctcaaagattaagccatgcatgtctaagtataaaggcttatactctgaaactgcgaacggctcattatatcagttatagtttctttgatagtgttttttctacatggatacttgtggcaaatctagaaacaatacatgcgtacaggcctgactttgggggagggctgcatttatttgacttaagccaatacccctcggggttgttttggtgattcagaataactgagcgaatcgcatagctttcgggcggcgatgaatcatttcaagtttctgccccatcagctgtcgatggtagggtataggcctaccatggctgtcacgggtgacggagaattagggttcgattccggagagggagcctgagagacggctaccacatccaaggaaggcagcaggcgcgtaaattactcaatgttgactcgacgaagtagtgacgagaattaacaatgcggagcgctcagcgttttgcaattggaatgagagcaatgtaaaagcctcatcgaggatccattggagggcaagtctggtgccagcagccgcggtaattccagctccaatagcgtatactaaagttgttgcagttaaaaagctcgtagttgaacctctggtagggccgaccttggcgcgcggtgaatgccgcgtcgtttagaagcgtcgtgcccggccatcctcccccggtcttttgggctgggggtcgtttactgtaaaaaaaatagagtgttccaagcagggggtaatatcccggtatatagtagtatggaataatgagataggactttggtactattttgttggtttgcatgccaaggtaatgattaagagggacagttgggggtattcgtatttagatgtcagaggtgaaattcttggattttcgaaagacgaactactgcgaaagcatttaccaaggatgttttcattaatcaagaacgaaagttaggggatcgaagatgattagataccatcgtagtcttaaccgtaaactatgccgacttgcgattgtccggcgtcgcttttagatgacctgggcagcagcacatgagaaatcaaagtctttgggttccggggggagtatggtcgcaaggctgaaacttaaaggaattgacggaagggcaccaccaggagtggagcctgcggcttaatttgactcaacacgggaaaacttaccaggtccggacataggaaggattgacagattgagagctctttcttgattctatgggtggtggtgcatggccgttcttagttggtggagtgatttgtctggttaattccgttaacgaacgagaccacagcctactaaatagtggccgttatggcgacatagcggtgaacttcttagagggacatttcgggtataccggaaggaagtttgtggcaataacaggtctgtgatgcccttagatgttctgggccgcacgcgcgctacactgatcggttcaacgagtatttgtttttttctcattttgggagggggcagagtccttggccggaaggtctgggtaatcttttgaatgccgatcgtgatggggctagatttttgcaattattaatctccaacgaggaattcctagtagacgcaagtcatcagcttgcatcgattacgtccctgccctttgtacacaccgcccgtcgcacctaccgattgaacgatccggtgagaccttgggattctgttgtggctgattcattttggctgcgatgggagaacttgagcaaaccttatcgtttagaggaaggtgaagtcgtaacaaggtttccgtagtgaacctgcaattcaaaaaaagccgttactcacatcaggccgccactcatccgggcgaaagcttcgcgcattcgtcctcgtcacctcgggtcccctgtgtcgtgacggaaagcgcgacgagacgcggccgcagcagagagccccgggggcccgcgtcacggggggcctggcggcggtcctccttaagccaaaccgagggttagggctccaggctgttcggcggggtcgcgggcgcggtggacgcgcggggccgcctagcacctcctagcgcgcgactaccaggatagcccccgcgagtgcgcagggcggtccgcggggcggagggcggcccagcagcgcggcgcggcgggcgggtgcggctgcgtaaggtggcggcgggcgcgggcggttagtgttggtgttaggtcgcggcggggctgtgttccgggcatccgccttacggcggtgcatactggttggctgggaggcggtttgcggggttagataggcggccaaggtgagctgcgttgggcggataaatccgtggaggcgctcgttgacggcgcggcagagacggaacgcggagcagcacggagtagcaagcaggagtagcaggagtagcaagcatggcaaaggaaggctagatgattgaacaggacggccttcacgctggctcgcccgctgcttgggtggaacggctgttcggctacgactgggctcagcagacgatcggctgctcggacgcggccgtgttccgccttagcgcgcagggccggccggtcctgtttgtcaagaccgaccttagcggcgccctcaacgagctccaggacgaagctgcccgcctcagctggcttgccacgacgggggttccgtgcgccgctgtgctcgacgtcgtcaccgaagccggccgcgactggctgctcctcggggaagtgcccggccaggacctcctcagcagccacctcgcgcccgctgagaaggtgtccatcatggccgacgccatgcgccgcctgcacaccctcgaccccgccacctgccccttcgaccaccaggcgaagcacaggatcgaacgcgcccgcacgcggatggaggctggcctcgtcgaccaagacgacctcgacgaggagcaccagggcctcgcgccggcggaactgttcgccaggcttaaggctaggatgccggacggcgaggacctcgtggtcacgcacggcgacgcctgcctccccaacatcatggtcgagaacggccgcttctcgggctttatcgactgcgggcgcctgggcgtggcggaccgctaccaagacatcgcgctcgccacgcgggacatcgccgaggagcttggcggcgagtgggccgaccgctttctcgtgctctacggcatcgccgccccggacagccagaggattgcgttctaccgcctcctggacgagttcttttgagatccgcgccggctatgcgcccgtgctcgactgccacactgcccacattgcctgcaagttcgctgagctccagaacaagatggaccgccgctcgggcaagattctcgaggagacccccaagttcatcaagtcgggtggactctgccatggtcaagatgtatcccctccaagcgcatgtgcgtcgagtccttcaccgagtacccgccgctcggccgctttgccgtgcgcgacatgcgcgtcaccgtcgctgtcggcgtcatcaagtccgtcaccaagggcgacaaataaattctacgaaagatttttttcctcaagaagcgccctaaagttgacccctagcagcgacgactgtgtgtgccgttgtgagtcgagttgcgatgtcgtgcagcgcccgtcgcgtcccatgctcgcgcgcgactccgtctctgcttttcatctcaagtcaagagtgggaagttcccttgctttatctcactatttagaggtcgctcacggctgctggttcctcgtcgcatgtagcacagcctcgtccaatcgcagcctgcaccaccccgctcgcctgggaaaatgcgctcagcggattcgcactggcactcctctcctcggacaggtgcgatgtggaagcggtcacatcctcggcgccctcggccacgccagcatctgcgcaatcgctctcctcgttctcagccgcaaccgcaggcaggccgacgtcgtttacctcggaatccaccgagcatttcgagcccatcgcgctggcgtccacctcgatcataccttctccatcgccgtccgctgcggcttccgattcttctgctgccgcaaccgcgacgtcggcccccgtctcctccgttctttccgatgccggcgcagtggccgcgccctctgctcgaaccggctcgtgttcagcgtcagggcctgcgcttgagctcgggcggctcttccgagtgatccggccccgcgaggcaaggaatcggcggctctggagtgtcggggcagccgctctcactgccggtctttggctggctgcctgtcctgcctcgcgttggcctttgcttttgcctaggctttcgccttggtgacggcgtttgcctgctgcggcgacttggcgcggccgcggaatagcgcctcaaagtcctgctcgaggcgccccagctctgacttgatttgcgaggtcccggtggcatgagctccgctgccctcgtccttacggcccgtctttcgctgcagtg

SEQ ID NO: 125 - DNA, 1218 bp, synthetic construct
  misc_feature 1..1218: note = genomic DNA (T. aureum ATCC 34304 OrfA upstream genomic DNA fragment)
  source 1..1218: mol_type = other DNA
cccgaattcggacgatgactgactgactgattggctgacgacggccgccctcgtgcgcggcgtcgggcgtcgtcgcaaaccaggcaggcaggcaggaaggaaggaaggaagggccaggccctggtgcgaaacgctggcctgctccgctgcaagccaagccgcgctcgcaggtgtacttccgagtcctcgcgatgattaggcaagcctgagcgagcacgtaagctgcactgcggctgttcaaccagagagagagttggctctcttgcgtcaaggcggcgcgcagcccacttgcgtcgcggctgagggcccctggaggggaggaaggaggccggcgagcggcgagtggcggccctcactggcaccaggtcgcaggaggccaggcagcccgccacggacaggaatcctcagggcgcagcagcgcactacgtagtgcagagacgcagagcgggccggatccgcagtgcggtcgcgccaccccgccgcgcagctcgctcgcggacggggtccgtggccgcgcgaaaacggacacggtgtgggagcggacatgggatcgagaacgccgttcgccctgctcgcgctgccagcagcaggagccgtccgaaggacgagcggccggccgcctgtcccccctccgcgcactcgaagcgcgcccggcagcgccccattgcgtgcgcggatggcgtcttggctggtccctctcgaggcgcttgctcgtgctcgccacgccttgtccgcctcctcgctgagcaagcgatgagctgagcacggaccgcctgcaagtgcaagtgttcttgtgctgcagggcgccgaagaattggattctggcccatgatcagtttgattgggccgagggagggagggaggctgggcgagtgggcgacaccagcaagccggactgcgagaggggcggggcaggatgtgagcgcaggaaagtgacgcaagtgcatccggccatcattgggccatcattgggccatcattggtgttttgggccgcgctttgcggatcgtccggccgatcaggtacgaggccacgaacctacgtcgtttgccgcgctcaggctggttggttgcacttggactcttctgtgacctttcatcgtgtgcaggcaaactcgatttgcagacccgagacacggcgaaggatccgtgctgcaaacgcaagtggagtgcgtcgagagcaccgccgagaccaagagccgaggcagacaagcttggg

SEQ ID NO: 126 - DNA, 32 bp, synthetic construct
  misc_feature 1..32: note = primer
  source 1..32: mol_type = other DNA
cccgaattcggacgatgactgactgactgatt

SEQ ID NO: 127 - DNA, 28 bp, synthetic construct
  misc_feature 1..28: note = primer
  source 1..28: mol_type = other DNA
cccaagcttgtctgcctcggctcttggt

SEQ ID NO: 128 - DNA, 1000 bp, synthetic construct
  misc_feature 1..1000: note = genomic DNA (T. aureum ATCC 34304 OrfA downstream genomic DNA fragment)
  source 1..1000: mol_type = other DNA
cccccatggtgttgctgtgggattggtccgggggctcttctgcacgcggcctccgtcgcgcgcagaaatgccccgtcactggctgcccaggaggcagccgaatccctctagctagctagctaggctagagcgtcttttccgtagtttttcacaaagccagtatcacatggataacgaacgaaggtttcgggctcgcgctcgcaggcgttaggacgaagttgatcgccccacgtcacttcaaacgagtgaaccaagatcacgttgcatctgctcgcaagatcttcttcttccacgccgcatcgatgcgatggatttcaaactcttttcagggcttttaggtgagtatggcagcgctgtttgcgtggcagcgctgtttgcgtggttgtactctctaaaggtgcttccacgcatgcgcgcacaaaggggcatggcatggttggcggcgcactctggccctcatttgaagcagactatcgaagggtccagttggtactgcggcaggtccggcgagagcaagcgcggcggtcgctcccactcgtccctgcacagttgctggactggcgacggctggcgcacctgactacgagaagactcgagacgcacagaggtagtcagggacgaccgaccgcaaagcacaaaccgctccaaaacggccgcaccaggcagggcagtaaactaaaaacgaatgtacctccatcgcgcgtatctgccgagcctcctcccacgcttcggctgggcttgattcaccagtgtccgcaagctgaaccgaccgtcttcgatgtcatgaagcttggcgcggcattagtcagacgacgcggcacgccaggattctgtcggtttctgggaaatgggcatctatatagctgattccctctgtcatgaggcggccttgttctggccctgggccgccgttcggatgatctatgatgtcgttgtacgcataaagcttgtcgaaaacgtcggccatgtcttcctcagagatgtaaccgagccatggggg

SEQ ID NO: 129 - DNA, 28 bp, synthetic construct
  misc_feature 1..28: note = primer
  source 1..28: mol_type = other DNA
cccccatggtgttgctgtgggattggtc

SEQ ID NO: 130 - DNA, 30 bp, synthetic construct
  misc_feature 1..30: note = primer
  source 1..30: mol_type = other DNA
cccccatggctcggttacatctctgaggaa

SEQ ID NO: 131 - DNA, 1632 bp, synthetic construct
  misc_feature 1..1632: note = fusion DNA (T. aureum ATCC 34304 ubiqitin promoter/Hygr)
  source 1..1632: mol_type = other DNA
cccaagcttgccgcagcgcctggtgcacccgccgggcgttgttgtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgacctaagcaacactagccaacatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgacgcatgcggg

SEQ ID NO: 132 - DNA, 36 bp, synthetic construct
  misc_feature 1..36: note = primer
  source 1..36: mol_type = other DNA
cccaagcttgccgcagcgcctggtgcacccgccggg

SEQ ID NO: 133 - DNA, 43 bp, synthetic construct
  misc_feature 1..43: note = primer
  source 1..43: mol_type = other DNA
cccgcatgcgtcgactattcctttgccctcggacgagtgctgg

SEQ ID NO: 134 - DNA, 1000 bp, synthetic construct
  misc_feature 1..1000: note = genomic DNA (T. aureum ATCC 34304 OrfA downstream genomic DNA fragment)
  source 1..1000: mol_type = other DNA
cccgtcgacgtgttgctgtgggattggtccgggggctcttctgcacgcggcctccgtcgcgcgcagaaatgccccgtcactggctgcccaggaggcagccgaatccctctagctagctagctaggctagagcgtcttttccgtagtttttcacaaagccagtatcacatggataacgaacgaaggtttcgggctcgcgctcgcaggcgttaggacgaagttgatcgccccacgtcacttcaaacgagtgaaccaagatcacgttgcatctgctcgcaagatcttcttcttccacgccgcatcgatgcgatggatttcaaactcttttcagggcttttaggtgagtatggcagcgctgtttgcgtggcagcgctgtttgcgtggttgtactctctaaaggtgcttccacgcatgcgcgcacaaaggggcatggcatggttggcggcgcactctggccctcatttgaagcagactatcgaagggtccagttggtactgcggcaggtccggcgagagcaagcgcggcggtcgctcccactcgtccctgcacagttgctggactggcgacggctggcgcacctgactacgagaagactcgagacgcacagaggtagtcagggacgaccgaccgcaaagcacaaaccgctccaaaacggccgcaccaggcagggcagtaaactaaaaacgaatgtacctccatcgcgcgtatctgccgagcctcctcccacgcttcggctgggcttgattcaccagtgtccgcaagctgaaccgaccgtcttcgatgtcatgaagcttggcgcggcattagtcagacgacgcggcacgccaggattctgtcggtttctgggaaatgggcatctatatagctgattccctctgtcatgaggcggccttgttctggccctgggccgccgttcggatgatctatgatgtcgttgtacgcataaagcttgtcgaaaacgtcggccatgtcttcctcagagatgtaaccgagtcgacggg

SEQ ID NO: 135 - DNA, 29 bp, synthetic construct
  misc_feature 1..29: note = primer
  source 1..29: mol_type = other DNA
cccgtcgacgtgttgctgtgggattggtc

SEQ ID NO: 136 - DNA, 29 bp, synthetic construct
  misc_feature 1..29: note = primer
  source 1..29: mol_type = other DNA
cccgtcgactcggttacatctctgaggaa

SEQ ID NO: 137 - DNA, 3705 bp, synthetic construct
  misc_feature 1..3705: note = fusion DNA (T. aureum OrfA upstream/EF1 alpha promoter/Neor/T. aureum OrfA downstream)
  source 1..3705: mol_type = other DNA
cccccatggctcggttacatctctgaggaagacatggccgacgttttcgacaagctttatgcgtacaacgacatcatagatcatccgaacggcggcccagggccagaacaaggccgcctcatgacagagggaatcagctatatagatgcccatttcccagaaaccgacagaatcctggcgtgccgcgtcgtctgactaatgccgcgccaagcttcatgacatcgaagacggtcggttcagcttgcggacactggtgaatcaagcccagccgaagcgtgggaggaggctcggcagatacgcgcgatggaggtacattcgtttttagtttactgccctgcctggtgcggccgttttggagcggtttgtgctttgcggtcggtcgtccctgactacctctgtgcgtctcgagtcttctcgtagtcaggtgcgccagccgtcgccagtccagcaactgtgcagggacgagtgggagcgaccgccgcgcttgctctcgccggacctgccgcagtaccaactggacccttcgatagtctgcttcaaatgagggccagagtgcgccgccaaccatgccatgcccctttgtgcgcgcatgcgtggaagcacctttagagagtacaaccacgcaaacagcgctgccacgcaaacagcgctgccatactcacctaaaagccctgaaaagagtttgaaatccatcgcatcgatgcggcgtggaagaagaagatcttgcgagcagatgcaacgtgatcttggttcactcgtttgaagtgacgtggggcgatcaacttcgtcctaacgcctgcgagcgcgagcccgaaaccttcgttcgttatccatgtgatactggctttgtgaaaaactacggaaaagacgctctagcctagctagctagctagagggattcggctgcctcctgggcagccagtgacggggcatttctgcgcgcgacggaggccgcgtgcagaagagcccccggaccaatcccacagcaacaccatggcagagtcgcccgacttgatgaacttgggggtctcctcgagaatcttgcccgagcggcggtccatcttgttctggagctcagcgaacttgcaggcaatgtgggcagtgtggcggtcgagcacgggcgcatagccggcgcggatctcaaaagaactcgtccaggaggcggtagaacgcaatcctctggctgtccggggcggcgatgccgtagagcacgagaaagcggtcggcccactcgccgccaagctcctcggcgatgtcccgcgtggcgagcgcgatgtcttggtagcggtccgccacgcccaggcgcccgcagtcgataaagcccgagaagcggccgttctcgaccatgatgttggggaggcaggcgtcgccgtgcgtgaccacgaggtcctcgccgtccggcatcctagccttaagcctggcgaacagttccgccggcgcgaggccctggtgctcctcgtcgaggtcgtcttggtcgacgaggccagcctccatccgcgtgcgggcgcgttcgatcctgtgcttcgcctggtggtcgaaggggcaggtggcggggtcgagggtgtgcaggcggcgcatggcgtcggccatgatggacaccttctcagcgggcgcgaggtggctgctgaggaggtcctggccgggcacttccccgaggagcagccagtcgcggccggcttcggtgacgacgtcgagcacagcggcgcacggaacccccgtcgtggcaagccagctgaggcgggcagcttcgtcctggagctcgttgagggcgccgctaaggtcggtcttgacaaacaggaccggccggccctgcgcgctaaggcggaacacggccgcgtccgagcagccgatcgtctgctgagcccagtcgtagccgaacagccgttccacccaagcagcgggcgagccagcgtgaaggccgtcctgttcaatcatctagccttcctttgccgctgcttgctactcctgctactcctgcttgttacttcgtgttgctccgcgttccgtctctgccgcgccgtccacgagcgcctccacggatttatccgcccaacgcggctcaccttggccgcctatctaaccccgcaaaccgcctcccagccaaccattgcgccgccgtaaggcggattcccagaacacagccccgccgcgacctaacccaacctaaccgcccgcgcccgccgccaccttacgcagccgcacccgcccgccgcgccgcgctgctgggccgccctcgccccgcagaccgccctgcgcgctcgcgggggctatcctggtagtcgcgcgctaggaggtgctaggcggccccgtgcttccacctcgcccgcgaccccgccgaacagcctggagccctaaccctcggtttggcttaaggaggactgccgccaggccccccgtgacgcgggcccccggggctctctgctgcggccgcgtctcgtcgcactttccgtcccgacacaggggacccgaggtgacgaggacgaatgcgcgaagcttgtctgcctcggctcttggtctcggcggtgctctcgacgcactccacttgcgtttgcagcacggatccttcgccgtgtctcgggtctgcaaatcgagtttgcctgcacacgatgaaaggtcacagaagagtccaagtgcaaccaaccagcctgagcgcggcaaacgacgtaggttcgtggcctcgtacctgatcggccggacgatccgcaaagcgcggcccaaaacaccaatgatggcccaatgatggcccaatgatggccggatgcacttgcgtcactttcctgcgctcacatcctgccccgcccctctcgcagtccggcttgctggtgtcgcccactcgcccagcctccctccctccctcggcccaatcaaactgatcatgggccagaatccaattcttcggcgccctgcagcacaagaacacttgcacttgcaggcggtccgtgctcagctcatcgcttgctcagcgaggaggcggacaaggcgtggcgagcacgagcaagcgcctcgagagggaccagccaagacgccatccgcgcacgcaatggggcgctgccgggcgcgcttcgagtgcgcggaggggggacaggcggccggccgctcgtccttcggacggctcctgctgctggcagcgcgagcagggcgaacggcgttctcgatcccatgtccgctcccacaccgtgtccgttttcgcgcggccacggaccccgtccgcgagcgagctgcgcggcggggtggcgcgaccgcactgcggatccggcccgctctgcgtctctgcactacgtagtgcgctgctgcgccctgaggattcctgtccgtggcgggctgcctggcctcctgcgacctggtgccagtgagggccgccactcgccgctcgccggcctccttcctcccctccaggggccctcagccgcgacgcaagtgggctgcgcgccgccttgacgcaagagagccaactctctctctggttgaacagccgcagtgcagcttacgtgctcgctcaggcttgcctaatcatcgcgaggactcggaagtacacctgcgagcgcggcttggcttgcagcggagcaggccagcgtttcgcaccagggcctggcccttccttccttccttcctgcctgcctgcctggtttgcgacgacgcccgacgccgcgcacgagggcggccgtcgtcagccaatcagtcagtcagtcatcgtccgaattcggg

SEQ ID NO: 138 - DNA, 3826 bp, synthetic construct
  misc_feature 1..3826: note = fusion DNA (T. aureum OrfA upstream/ubiquitin promoter/Hygr/T. aureum OrfA downstream)
  source 1..3826: mol_type = other DNA
cccccatggctcggttacatctctgaggaagacatggccgacgttttcgacaagctttatgcgtacaacgacatcatagatcatccgaacggcggcccagggccagaacaaggccgcctcatgacagagggaatcagctatatagatgcccatttcccagaaaccgacagaatcctggcgtgccgcgtcgtctgactaatgccgcgccaagcttcatgacatcgaagacggtcggttcagcttgcggacactggtgaatcaagcccagccgaagcgtgggaggaggctcggcagatacgcgcgatggaggtacattcgtttttagtttactgccctgcctggtgcggccgttttggagtggtttgtgctttgcggtcggtcgtccctgactacctctgtgcgtctcgagtcttctcgtagtcaggtgcgccagccgtcgccagtccagcaactgtgcagggacgagtgggagcgaccgccgcgcttgctctcgccggacctgccgcagtaccaactggacccttcgatagtctgcttcaaatgagggccagagtgcgccgccaaccatgccatgcccctttgtgcgcgcatgcgtggaagcacctttagagagtacaaccacgcaaacagcgctgccacgcaaacagcgctgccacgcaaacagcgctgccatactcacctaaaagccctgaaaagagtttgaaatccatcgcgtcgatgcggcgtggaagaagaagatcttgcgagcagacgcaacgtgatcttggttcactcgtttgaagtgacgcgggacgatcaacttcgtcctaacgcctgcgagcgcgagcccgaaaccttcgttcgttatccatgtgatactggctttgtgaaaaactacggaaaagacgctagctagagggattcggctgcctccttgggcagccagtgacggggcatttctgcgcgcgacggaggccgcgtgcaaaagagcccccggaccaatcccacagcaacacgtcgactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatgttggctagtgttgcttaggtcgcttgctgctgctggtgttgcttgttctgcttgtcgtttggggtctgcccgaagtggacgcgtgacacagcccagcgttcgcgacttttccagcaaccagctcgctcccgcgtcagggtcactctctgctcactcccctgtctagttcggcagaactggagaggcaacccgcgcttccagagggttctcctccggaatcagttcagaattcagaattcagaagagggaatcgcagaaccggccgcttcgggagttggttcgctgcaccatagcaggtgggcgatgaggccaaccgccctgctggctggattctctcgggttggcacatcgaacgaccgggacccgcgcgcgagttgtccctgccagttgctaccagcctgccagcgtcggagagttatgcgctgcctgccggccggagagagagcgggcgtggaaagtggcgtgtggggcgaacgcatggccctcccgcgtggcccgatttcctatgcatccgctccgctctctctctcggaggcaagaagagcacaccaacaacgcccggcgggtgcaccaggcgctgcggcaagcttgtctgcctcggctcttggtctcggcggtgctctcgacgcactccacttgcgtttgcagcacggatccttcgccgtgtctcgggtctgcaaatcgagtttgcctgcacacgatgaaaggtcacagaagagtccaagtgcaaccaaccagcctgagcgcggcaaacgacgtaggttcgtggcctcgtacctgatcggccggacgatccgcaaagcgcggcccaaaacaccaatgatggcccaatgatggcccaatgatggccggatgcacttgcgtcactttcctgcgctcacatcctgccccgcccctctcgcagtccggcttgctggtgtcgcccactcgcccagcctccctccctccctcggcccaatcaaactgatcatgggccagaatccaattcttcggcgccctgcagcacaagaacacttgcacttgcaggcggtccgtgctcagctcatcgcttgctcagcgaggaggcggacaaggcgtggcgagcacgagcaagcgcctcgagagggaccagccaagacgccatccgcgcacgcaatggggcgctgccgggcgcgcttcgagtgcgcggaggggggacaggcggccggccgctcgtccttcggacggctcctgctgctggcagcgcgagcagggcgaacggcgttctcgatcccatgtccgctcccacaccgtgtccgttttcgcgcggccacggaccccgtccgcgagcgagctgcgcggcggggtggcgcgaccgcactgcggatccggcccgctctgcgtctctgcactacgtagtgcgctgctgcgccctgaggattcctgtccgtggcgggctgcctggcctcctgcgacctggtgccagtgagggccgccactcgccgctcgccggcctccttcctcccctccaggggccctcagccgcgacgcaagtgggctgcgcgccgccttgacgcaagagagccaactctctctctggttgaacagccgcagtgcagcttacgtgctcgctcaggcttgcctaatcatcgcgaggactcggaagtacacctgcgagcgcggcttggcttgcagcggagcaggccagcgtttcgcaccagggcctggcccttccttccttccttcctgcctgcctgcctggtttgcgacgacgcccgacgccgcgcacgagggcggccgtcgtcagccaatcagtcagtcagtcatcgtccgaattcggg

SEQ ID NO: 139 - DNA, 22 bp, synthetic construct
  misc_feature 1..22: note = primer
  source 1..22: mol_type = other DNA
gaagcgtcccgtagatgtggtc

SEQ ID NO: 140 - DNA, 21 bp, synthetic construct
  misc_feature 1..21: note = primer
  source 1..21: mol_type = other DNA
gcccgagaggtcaaagtacgc

SEQ ID NO: 141 - DNA, 20 bp, synthetic construct
  misc_feature 1..20: note = primer
  source 1..20: mol_type = other DNA
gcgagcccaggtccacttgc

SEQ ID NO: 142 - DNA, 22 bp, synthetic construct
  misc_feature 1..22: note = primer
  source 1..22: mol_type = other DNA
cagcccgatgaaaaacttggtc

SEQ ID NO: 143 - DNA, 22 bp, synthetic construct
  misc_feature 1..22: note = primer
  source 1..22: mol_type = other DNA
gggagcgcagggaaaacggtct

SEQ ID NO: 144 - DNA, 20 bp, synthetic construct
  misc_feature 1..20: note = primer
  source 1..20: mol_type = other DNA
ccagcccacgtcgtcggagc

SEQ ID NO: 145 - DNA, 2297 bp, synthetic construct
  misc_feature 1..2297: note = genomic DNA (T. aureum ATCC 34304 C20 elongase upstream genomic DNA fragment)
  source 1..2297: mol_type = other DNA
ggccggggcagcccgcccagcacgccgctgcgctgctttcggtcatgcgaacctggctccccacagcaatgctgcgcggtcgctgcgcctcttgaggctcggcgacgttggcccggtttggggcaccctgacgttgcacgaacgtccgctgcatctcaggcgcactcggatcgacaactgtgcaaccggtcagcctttcgcggcagattgggcacttgccgcgctcgcgtatccgcgtggcgcattcttcgcacacgcaggcgtgccggcagggaagcaggagggtgtttatcgtggcgtccatgtagaccttgcacagccgcggctcactttccctcggcgcagtcccgtgcccaacgtcggggccgggcgccggcgccggcgagggcgtcggttctgggatgggatcaggatccgccgaggctgcagattgctgtgcgggggtgccggggcgcggcccattagcaccgtcctgcggaatatccaggagggtgctcatcacggaagccatgtccgggcgctggctgccgtcttcgtgcgtgcaacgatccaccaggtcaaggagcacccgagccacgttttggcgggtgcgaaaagcggcttggtcaacgcagcgctccgggtcgaagccgttgtcgcgcatccagtacggaagcaggtcgaggccgcggcggcagggaaagcaaggcggcttgccggacacaagctcgccgagcacgacgccaaaggcgtaaacgtccacggggcgattgtactcgacatgggtggcgccctccaggtcggagatctcgggcgccatgtagccaagcgtgcccacttgtgtcatcgtttgcatggtgtggagcgtggtactggcggccaccttggacacgccaaagtccgtccagcacagccttaggcccgcgcccttgttcaggttgaccagagcgttgtcgctcttggatgtctctgtgcagcacgccagcagcgtgcagcgccctgaggccgtgcgccgcctggtatgccagcgcctcgcgagcgctgccgtccaaaagcggcgcgctccccgaatcatcgcggagctggatggccttcttgagcgacatttccatgcggggcatgatgatcgcaaacctgccgccgctctgtggctcgagcgcggtcgccaggaccgtgagcacgttctcgtgcgttgcgctcgccatccgcgaggcctcagcgcgaaagtcatcgaggacgccgagcgtctgcccgggccgcggtaccttgacagcgcagcgcccgaacggcgccacgtccgcttcaaacacctcgccaaaggcgccttgtccgagcagctcgccccagccgcagccgcgaccactcgatctcgggcacgcgtgccatcgacccttgcagcgtggccttgccaagtcacagtccagcgcgcagttcagtgtctgccgcgccaggtccaccacgatcaattcatccgagtcggctgcgaactggacaagtgccatttgggtgcgggcaacgcgcaagatcacccagcactggcatgaagaccatgaatgaatgaatgaccgtgcgcgagtgaccgaccaacacgagtccagccgactccttcttcttctccttcttcttcttcttcttcttctcgtagcgggcgtcaacagcatcaatcaggcatggcggcattcactctgcgcgatggatggcacgagcgctggaggtgatgaacgcactgcccggattggctctcggtcactgtcagcacatgatgcctgtgcttgcgcggagcgcgctatgtctcgttctgtgtcaagacacaggcgcaactcttgatggattcttgaagcgcatgtaactgaagtctgacagactcggaagtccattgtgaacaatgttgttccacaattgctccaattgttccgattattccacaattgttgttccaattgttccaattgttccgattattccgattattccactttagttgttccagttgttccgattgttccacaattgttgttccgattattccagttgttccagttgttccaattattccaattgttccagttccttactcttgacatcgggggaataacgggtgtgtatttaggggttcggcgaaagcagaatggccgaacgtaacagcggagaggaacctctttagcggggtttgcgtatcggggaaaccaggtgttgtgctggcgaggaggatcccccgcgaggcgatggctgctccgacgacgtgggctggcgacgtcgctcgcaaaggcgttccgcaaccgcgcgttccgctgtaacgagaccgttttccctgcgct

SEQ ID NO: 146 - DNA, 23 bp, synthetic construct
  misc_feature 1..23: note = primer
  source 1..23: mol_type = other DNA
gccgctcatgcccacgctcaaac

SEQ ID NO: 147 - DNA, 23 bp, synthetic construct
  misc_feature 1..23: note = primer
  source 1..23: mol_type = other DNA
ctttcggctgccaggaatctacg

SEQ ID NO: 148 - DNA, 2189 bp, synthetic construct
  misc_feature 1..2189: note = genomic DNA (T. aureum ATCC 34304 OrfA downstream genomic DNA fragment)
  source 1..2189: mol_type = other DNA
ctttcggctgccaggaatctacggcccagggcgcggcccgatctcacgaattcgcaagggccaggcccgcagtatcgtcaaggagggccaggtcttctcgcgggcgcacgtcgacgatatcaccggtgcgatccgcgcttcgctggccaacccaaacccgggccgcgcctacaacgtttgcgacgacgagcctgcaatgaaccatgtcgtgacagagtttgcctgcgaactcatggacgtcccgcccccgaagcgcgaagactttgacaaggtgcgcgagaccatgtcaagcatgtcgctctccttcttctcagagagcaagcgggtcttcaacaagcggctcaaggaagagctgcggtacgcgctattgtacccgacctaccgcgaagggatcaaagcccaactggaggaggagcttgccaacggctggacgctcatcgacgcctcgggtgcttctgctggaaccgactcccctgcctcgcccaaagcgcccgcccccatcgccgcctcaagtgacgagtcgagcgggcagagcgcgacagcggccgagccggtgcgccggcgcaggcgccccgagcgcaaggcgctcccgcctgctgggccgagtgggccgtcggtcttgcagagggtttctcgggcaatttatgggccgttcagttggctcctcggtcgcctgtttgggccactttcgagccgcgctgtcggcttgtttcgcggctgggcgcactggctgttgcgtctcgtggggctgcgcgcatccgcgccgggcggcggccgtacaacctgcctccttgttgacaacggctcgctcaaaccagagccttttcgccagctgcgcgtgcacgcggcgaacctcgaagagtctcttaggagcgacgcgcgtgccccacatcccgtgcaggtggtggccgtcagcgcgaggtacagcgaccgcatcgacgcctcccttctggacggcaagcccggcgtcgccctcgccgggttcctgagttccttcaaggccgacgccgagtcgcagccagcaaccagcgaggttggccgcatcatcgcgctcccctactttctgggcccaagcaagacggccacgtcgtatgttgcttcccagctcgcagagcactttccaggagccgagcgcaccattgccgctccgctcgtgtcgcgggacggcgccattgcgcagctcctcgctgacatggtccatgacgtcgctcgggcgcgcgcgctgcaggccccgtacgcggtagttctcgtcgaccacgggtccccgagccgagcggtcaaccgcgttcggcgggccatcgctgcgcggatgcgccgccgccttggcccgaacgcgcgctgcgttgtcgactgctccatggagcgccgcgagggcgacgctttcgccttcaacgagcctctgctagagtcggttttcaccaagggtggtctcgactctggcgacgtcattctcgcgatggcatttttggcgcctggtcgccacgctggcgagggcggcgatatcgcggagatccttgacgaggctatcgcaaagtcggctggcaagctgcgcgttcaccaaacgcggttgattggtgacgtggacaggaacggtacgcagatttgcgccctcctcaagaacaggccgcttgccgcgctgtaacggcaagagcatccacaattcctgacctgagcaaaccagcccacgcgagagaccgaacacgtcaagccgatgaggcgcagaaaacaaagaaaaaaagcaaaaagaacaaaaacccaaggcaaaatgatggcaattttcttggtatggaaagccgatgatcgccgagtgtcgctggctatttgctctggtggggcatcgagctcgatgaccgaaatccaccaattatctgcgtgtcaatcatttggagcataagacccgggaaggccttgagcaagcgaagaaaccggcgcgtgttcacacgatagtacgagacgtcgctctctgcgcggatctcaatctgagccttcttgtctccgcggatgaaagtgttcatgtccccgacaagggcgccgcgcccaacccctcgtttgggctgcgccgcgctactggaaatggtgattccgcgaaacgtgcccgattcgcctttctcaacagggctcaccgtgacagaaccctcagcgacaagaacgatgccgtcaatcttttcgccgggcgaggctttctgcag

SEQ ID NO: 149 - DNA, 618 bp, synthetic construct
  misc_feature 1..618: note = genomic DNA (T. aureum ATCC 34304 ubiquitin promoter)
  source 1..618: mol_type = other DNA
cccagatctgccgcagcgcctggtgcacccgccgggcgttgttggtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagtccccaaacgacaagcagaacaagcaacaccagcagcagcaagcgacctaagcaacactagccaacatggccaagcctttgtctcaagaag

SEQ ID NO: 150 - DNA, 58 bp, synthetic construct
  misc_feature 1..58: note = primer
  source 1..58: mol_type = other DNA
cttcttgagacaaaggcttggccatgttggctagtgttgcttaggtcgcttgctgctg

SEQ ID NO: 151 - DNA, 432 bp, synthetic construct
  misc_feature 1..432: note = Blasticidin resistance gene (Blar)
  source 1..432: mol_type = other DNA
agcgacctaagcaacactagccaacatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagatctggg

SEQ ID NO: 152 - DNA, 54 bp, synthetic construct
  misc_feature 1..54: note = primer
  source 1..54: mol_type = other DNA
agcgacctaagcaacactagccaacatggccaagcctttgtctcaagaagaatc

SEQ ID NO: 153 - DNA, 38 bp, synthetic construct
  misc_feature 1..38: note = primer
  source 1..38: mol_type = other DNA
cccagatcttagccctcccacacataaccagagggcag

SEQ ID NO: 154 - DNA, 1000 bp, synthetic construct
  misc_feature 1..1000: note = fusion DNA (T. aureum ATCC 34304 ubiquitin promoter/pTracer-CMV/Bsd/lacZ Blar)
  source 1..1000: mol_type = other DNA
cccagatctgccgcagcgcctggtgcacccgccgggcgttgttggtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagtccccaaacgacaagcagaacaagcaacaccagcagcagcaagcgacctaagcaacactagccaacatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagatctggg

SEQ ID NO: 155 - DNA, 812 bp, synthetic construct
  misc_feature 1..812: note = cDNA (T.aureum ATCC 34304 ubiquitin promoter)
  source 1..812: mol_type = other DNA
tcggtacccgttagaacgcgtaatacgactcactatagggagagtcgactgagcacaactctgctgcgagcgggcctcgagagcgtttgcttcgagccgcggagcaagggggatggatcgctcatgcggtcgtgcggccctcggtcacccggtgggtcctgcactgacgcatctgttctgatcagacacacgaacgaacaaaccgaggagccgcagcgcctggtgcacccgccgggcgttgttgtgtgctcttcttgcctccgagagagagagcggagcggatgcataggaaatcgggccacgcgggagggccatgcgtttgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagctgccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgatctaagcaacactagccaacatggtgagcaagggcgagga

SEQ ID NO: 156 - DNA, 29 bp, synthetic construct
  misc_feature 1..29: note = primer
  source 1..29: mol_type = other DNA
tcggtacccgttagaacgcgtaatacgac

SEQ ID NO: 157 - DNA, 41 bp, synthetic construct
  misc_feature 1..41: note = primer
  source 1..41: mol_type = other DNA
tcctcgcccttgctcaccatgttggctagtgttgcttaggt

SEQ ID NO: 158 - DNA, 748 bp, synthetic construct
  misc_feature 1..748: note = Enhanced GFP gene (Enhanced GFP DNA fragment)
  source 1..748: mol_type = other DNA
acctaagcaacactagccaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgccaagttgaccagtgccgttc

SEQ ID NO: 159 - DNA, 41 bp, synthetic construct
  misc_feature 1..41: note = primer
  source 1..41: mol_type = other DNA
acctaagcaacactagccaacatggtgagcaagggcgagga

SEQ ID NO: 160 - DNA, 58 bp, synthetic construct
  misc_feature 1..58: note = primer
  source 1..58: mol_type = other DNA
gaacggcactggtcaacttggcgtccatgccgagagtgatcccggcggcggtcacgaa

SEQ ID NO: 161 - DNA, 1519 bp, synthetic construct
  misc_feature 1..1519: note = fusion DNA (T. aureum ATCC 34304 ubiquitin promoter/ Enhanced GFP)
  source 1..1519: mol_type = other DNA
tcggtacccgttagaacgcgtaatacgactcactatagggagagtcgactgagcacaactctgctgcgagcgggcctcgagagcgtttgcttcgagccgcggagcaagggggatggatcgctcatgcggtcgtgcggccctcggtcacccggtgggtcctgcactgacgcatctgttctgatcagacacacgaacgaacaaaccgaggagccgcagcgcctggtgcacccgccgggcgttgttgtgtgctcttcttgcctccgagagagagagcggagcggatgcataggaaatcgggccacgcgggagggccatgcgtttgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagctgccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgacctaagcaacactagccaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgccaagttgaccagtgccgttc

SEQ ID NO: 162 - DNA, 1319 bp, synthetic construct
  misc_feature 1..1319: note = fusion DNA (T. aureum ATCC 34304 ubiquitin promoter/ Enhanced GFP)
  source 1..1319: mol_type = other DNA
cccagatctgccgcagcgcctggtgcacccgccgggcgttgttgtgtgctcttcttgcctccgagagagagagcggagcggatgcataggaaatcgggccacgcgggagggccatgcgtttgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagctgccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgacctaagcaacactagccaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgccaagttgaccagtgccgttc

SEQ ID NO: 163 - DNA, 408 bp, synthetic construct
  misc_feature 1..408: note = cDNA (Zeor)
  source 1..408: mol_type = other DNA
cgccgccgggatcactctcggcatggacgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagatctggg

SEQ ID NO: 164 - DNA, 54 bp, synthetic construct
  misc_feature 1..54: note = primer
  source 1..54: mol_type = other DNA
cgccgccgggatcactctcggcatggacgccaagttgaccagtgccgttccggt

SEQ ID NO: 165 - DNA, 38 bp, synthetic construct
  misc_feature 1..38: note = primer
  source 1..38: mol_type = other DNA
cccagatctcagtcctgctcctcggccacgaagtgcac

SEQ ID NO: 166 - DNA, 1677 bp, synthetic construct
  misc_feature 1..1677: note = fusion DNA (T. aureum ATCC 34304 ubiquitin promoter/Enhanced GFP/pcDNA3.1 Zeo(+) Zeor)
  source 1..1677: mol_type = other DNA
cccagatctgccgcagcgcctggtgcacccgccgggcgttgttgtgtgctcttcttgcctccgagagagagagcggagcggatgcataggaaatcgggccacgcgggagggccatgcgtttgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagctgccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgacctaagcaacactagccaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagatctggg

SEQ ID NO: 167 - DNA, 2884 bp, synthetic construct
  misc_feature 1..2884: note = genomic DNA (T. aureum ATCC 34304 C20 elongase upstream/C20 elongase/C20 elongase downstream)
  source 1..2884: mol_type = other DNA
cccgaattcactagtgattctcccgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgatggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatacggctgaccactacgcagcgcgatatcctccatccgcgtatatatccgtaaacaacggaacattctccctctcaacgaggcgtggttttcgaagtcatgcctttcttccttcctactttccttccttctttctttctttctttccttcttttgcaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgcttgctcgcggcgactcatgatgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgcgccgctgtcgctcgctaatccacccgaagcgcggtcggctggcacggtcgctcggctggaagttgagtagtttgctttctgttgctgcgctgctttgtaaacgcgaccatggcgacgcgcacctcgaagagcgctccggcggtttccaagtcggccaaggttgccgcgccggcgaagaagcggtcggtcgacaggagcgacggtttcttccgcacgttcaacctgtgcgccctgtacgggtctgccctcgcctatgcgtacaagcacggcccggtggacaatgacggccaggggctgtactttcacaagtcgcccatgtacgcgttcgccgtgtcggacgtcatgaccttcggcgcgccgctgatgtacgtgctcggtgtgatgctgctcagcaggtacatggcggacaaaaagcccctgactggcttcatcaagacctacatccagcccgtctacaacgtggtccaaatcgcggtgtgcggctggatggtgtggggcctctggccgcaggtcgacctggccaacggcaaccctttcggcctcaacaagtcgcgcgactcgaacatcgagtttttcgtgttcgtgcacctcctgacaaagtttctcgactggagcgacacgttcatgatgatcctcaagaaaaactacgcccaggttagctttctgcaggtgttccaccacgcaacgatcggcatggtgtggtcgttccttcttcagcgtggctggggctcgggcaccgccgcgtacggtgctttcatcaactcggtcacgcacgtgatcatgtactcgcactactttgccacctcgctcaacatcaacaacccgttcaagcggtacatcacgagcttccagctcgcccagtttgcaagctgcatcgtgcatgccctactggtgcttgccttcgaggaggtgtacccgctcgagtacgcttacctgcagatcagctaccacatcatcatgctctacctgttcggacgccgcatgaactggagccccgagtggtgcaccggtgagatcgacggccttgacgccccaagcgcccccaccaagtccgagtaaacctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccgggacgcgcacgacgagcagcgctgctttgatcgaccttgcctgcgtcaccgcctcggctgctttgatcgatcgttgtcaccggccgagtgaccgcgaacgcattgcccgcacggctcggctcggcccggaccggaccggctcgccttggcggcgcggcgcgatggcgacccagacgcggccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccgactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgggataatcgaattcggg

SEQ ID NO: 168 - DNA, 50 bp, synthetic construct
  misc_feature 1..50: note = primer
  source 1..50: mol_type = other DNA
cccgaattcactagtgattctcccgggtggacctagcgcgtgtgtcacct

SEQ ID NO: 169 - DNA, 40 bp, synthetic construct
  misc_feature 1..40: note = primer
  source 1..40: mol_type = other DNA
cccgaattcgattatcccggggccgagaacggggtcgccc

SEQ ID NO: 170 - DNA, 1939 bp, synthetic construct
  misc_feature 1..1939: note = fusion DNA (T. aureum ATCC 34304 C20 elongase upstream/C20 elongase downstream)
  source 1..1939: mol_type = other DNA
cccgaattcactagtgattctcccgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgatggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatacggctgaccactacgcagcgcgatatcctccatccgcgtatatatccgtaaacaacggaacattctccctctcaacgaggcgtggttttcgaagtcatgcctttcttccttcctactttccttccttctttctttctttctttccttcttttgcaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgcttgctcgcggcgactcatgatgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgcgccgctgtcgctcgctaatccacccgaagcgcggtcggctggcacggtcgctcggctggaagttgagtagtttgctttctgttgctgcgctgctttgtaaacgcgaccagatctacctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccgggacgcgcacgacgagcagcgctgctttgatcgaccttgcctgcgtcaccgcctcggctgctttgatcgatcgttgtcaccggccgagtgaccgcgaacgcattgcccgcacggctcggctcggcccggaccggaccggctcgccttggcggcgcggcgcgatggcgacccagacgcggccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccgactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgggataatcgaattcggg

SEQ ID NO: 171 - DNA, 38 bp, synthetic construct
  misc_feature 1..38: note = primer
  source 1..38: mol_type = other DNA
cccagatctacctgtttccggctggctcccgagccatg

SEQ ID NO: 172 - DNA, 38 bp, synthetic construct
  misc_feature 1..38: note = primer
  source 1..38: mol_type = other DNA
cccagatctggtcgcgtttacaaagcagcgcagcaaca

SEQ ID NO: 173 - DNA, 31 bp, synthetic construct; identical to SEQ ID NO: 78
  misc_feature 1..31: note = primer
  source 1..31: mol_type = other DNA

SEQ ID NO: 174 - DNA, 27 bp, synthetic construct
  misc_feature 1..27: note = primer
  source 1..27: mol_type = other DNA
atcccggggccgagaacgccctcgccc

SEQ ID NO: 175 - DNA, 3215 bp, synthetic construct
  misc_feature 1..3215: note = fusion DNA (T. aureum C20 elongase upstream/ubiquitin promoter/Blar/SV40 terminator/T. aureum C20 elongase downstream)
  source 1..3215: mol_type = other DNA
ctcccgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgatggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatgcggctgaccactacgcagcgcgatatcctccatccgcgtatatatccgtaaacaacggaacattctccctctcaacgaggcgtggttttcgaagtcatgcctttcttccttcctactttccttccttctttctttctttctttccttcttttgcaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgcttgctcgcggcgactcatgatgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgcgccgctgtcgctcgctaatccacccgaagcgcggtcggctggcacggtcgctcggctggaagttgagtagtttgctttctgttgctgcgctgctttgtaaacgcgaccagatctggatctgccgcagcgcctggtgcacccgccgggcgttgttgtgtgctcttcttgcctccgagagagagagcggagcggatgcataggaaatcgggccacgcgggagggccatgcgttcgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagcggccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagtccccaaacgacaagcagaacaagcaacaccagcagcagcaagcgacctaagcaacactagccaacatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagatccgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagatctacctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccgggacgcgcacgacgagcagcgctgctttgatcgaccttgcctgcgtcaccgcctcggctgctttgatcgatcgttgtcaccggccgagtgaccgcgaacgcattgcccgcacggctcggctcggcccggaccggaccggctcgccttggcggcgcggcgcgatggcgacccagacgcggccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccaactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgggat

SEQ ID NO: 176 - DNA, 3887 bp, synthetic construct
  misc_feature 1..3887: note = fusion DNA (T. aureum C20 elongase upstream/ubiquitin promoter/Enhanced GFP/Zeor/SV40 terminator/T. aureum C20 elogase downstream)
  source 1..3887: mol_type = other DNA
ctcccgggtggacctagcgcgtgtgtcacctgccggcccccgttgcgtgcaaccgaattgatcgataatagaattacataacaaacaacttgctggatgagtacaagaccagcgtagtgtggctgtgggacgttgaacggagcgggtcctgtgatggcgcagaaaggaactccgcccgaggtgaaaccccgatgcgcaggactctgcggccacagcccctccgccagtattccactaaaaatccgccccctttgacaaagatcgcaaccccgtcccatcaactcctcacaataggctttccactggcggaaacgtccccggcacaggagtgcctcccgcggttctgcgcatacggctgaccactacgcagcgcgatatcctccatccgcgtatatatccgtaaacaacggaacattctccctctcaacgaggcgtggttttcgaagtcatgcctttcttccttcctactttccttccttctttctttctttctttccttcttttgcaagcgtgcgcgaacttgaaggtactacttacacttgacagagagagatagagacggcaattcgaccaagtactttccacgattttttttttttttgttttggtcgctttcgttggtcgtgcatgatggatggccgggatttttacaattggatgcgccaggctgccacgcatgccgtgacgcttgctcgcggcgactcatgatgcttgccagtggcagtgcatccagctcttccctctgctcgtcgtgtactcactggcgatgctctcggcgctcgttcaagggccatcgatcgatcgatcgatcgatcgatcgatcaatcacgtttggtggactcggcagaccccgaacgtgtttctcccaggacgcgccgctgtcgctcgctaatccacccgaagcgcggtcggctggcacggtcgctcggctggaagttgagtagtttgctttctgttgctgcgctgctttgtaaacgcgaccagatctgccgcagcgcctggtgcacccgccgggcgttgttgtgtgctcttcttgcctccgagagagagagcggagcggatgcataggaaatcgggccacgcgggagggccatgcgtttgccccacacgccactttccacgcccgctctctctccggccggcaggcagcgcataactctccgacgctggcaggctggtagcaactggcagggacaactcgcgcgcgggtcccggtcgttcgatgtgccaacccgagagaatccagccagcagggcggttggcctcatcgcccacctgctatggtgcagcgaaccaactcccgaagctgccggttctgcgattccctcttctgaattctgaattctgaactgattccggaggagaaccctctggaagcgcgggttgcctctccagttctgccgaactagacaggggagtgagcagagagtgaccctgacgcggagcgagctggttgctggaaaagtcgcgaacgctgggctgtgtcacgcgtccacttcgggcagaccccaaacgacaagcagaacaagcaacaccagcagcagcaagcgatctaagcaacactagccaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagatccgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagatctacctgtttccggctggctcccgagccatgcttaccatgaatgaacctgcaaacagtctgaggtccttgtgcaaaccgctcagtgggacgtcgacgaagaaagaaacaatgtgtactcgtcttgctctgctcccgcgccgttttttatcgttgttgagacctctcgcgcagttttgggaatcaaccaaaacaagagcccggcgtcagcgtttgcttcgccctcggctgcactcgctcggcacgcaggtataactgggtgagtaccaagccccgcatttgtctgtccgcgatccgcgcacgctgcgggtcaggacgacatcgcgctgcacgtcacagtgggtcccttttgacgtggctgcggcgatgaggaggcttggctcggcttcatggcaaggcaacagactcgcttccgggacgcgcacgacgagcagcgctgctttgatcgaccttgcctgcgtcaccgcctcggctgctttgatcgatcgttgtcaccggccgagtgaccgcgaacgcattgcccgcacggctcggctcggcccggaccggaccggctcgccttggcggcgcggcgcgatggcgacccagacgcggccggagccgcgcgcggaggacaaggccatgttcatcttcgggctcgggtacgttgggagcaggctcgccaaccagctggcggaacaggggtggcgcgtcgcggggtcggtgagggagctcgggcgcgaggacgactttgccgagttcgaaaagtccaagctgagcggcaaggtgcaggtgttccgactcccgcttgagggcgaggacaacacgcccgctcgcgcgcgggagatacttagcgggtaccagcacctgctgttcacggcgccagtggaccgcgcccggaactgtgaccccttcttgggcgaccccgttctcggccccgggat

SEQ ID NO: 177 - DNA, 21 bp, synthetic construct
  misc_feature 1..21: note = primer
  source 1..21: mol_type = other DNA
acgtccgcttcaaacacctcg

SEQ ID NO: 178 - DNA, 24 bp, synthetic construct
  misc_feature 1..24: note = primer
  source 1..24: mol_type = other DNA
tcggaacaactggaacaactaaag

SEQ ID NO: 179 - DNA, 22 bp, synthetic construct
  misc_feature 1..22: note = primer
  source 1..22: mol_type = other DNA
atgtcgctctccttcttctcag

SEQ ID NO: 180 - DNA, 21 bp, synthetic construct
  misc_feature 1..21: note = primer
  source 1..21: mol_type = other DNA
tcggctcctggaaagtgctct

SEQ ID NO: 181 - DNA, 23 bp, synthetic construct
  misc_feature 1..23: note = primer
  source 1..23: mol_type = other DNA
ggcggagcgaagtgtgaaagtta

SEQ ID NO: 182 - DNA, 24 bp, synthetic construct
  misc_feature 1..24: note = primer
  source 1..24: mol_type = other DNA
gcgacagcatcttgaaataggcag

SEQ ID NO: 183 - DNA, 2571 bp, Thraustochytrium aureum
  source 1..2571: mol_type = genomic DNA
ggcggagcgaagtgtgaaagttacaacccagttactgcccattcccgggaaaagttgcgcagctcacgcggttcgcttttctggtggcctggcgacgttcgccgcttgccggatactccctcgtgcccccgcgccaggtttgcccgctgtcgctcgaggagtggactcgcgagtcgcgacagcagcagcaccaagggggatggatcctcgttgacagcaccaagatgctctctgcctttcaggtgaaatcgatcgatcaattgatcaatcaagatcattggaagcaaatgggaagcaaatgcgaagggggaagaccctcggtctctgctcgggaacccgacacgaggctgagggcgcgcttctacaggttgtgcagcggccgcactgcgagcttgcgccgggccaaggcgctcgccagaattgctgcgtctgccgcctcgggatcagccactcggtttttcgtcatcagggtccaccttcaacctggaagtggactcggcaagtcggcagatccactccggaattccaagatccccggtcgatcggtgctggtgcgaattaggatggacccaggctatgtgagagtcggagggtggcggttgtctccaccgtgacagcgcgcgtgtggtgagtaacgcgaagcgcgtggtggagaaatggggggagattcgtaggacgcgatgcgctcgtcactgagggtgcgccggtgacgaagcttcggacccagattccgtcggtatggctcgtgttcgcacaccttcaggaacccgcatgacgagaccactggagttttcaacgtcacgaagccgctctgtgtgacgagaattggcttgcgagtgacgtgaggcgccgagcatgtcgttggtttgcctcttcacaacagaatcagacgactgggaggctgcacgaggctaaggccaagggcactcactgactcggacgtgaagcagaagcagaagcagagcgctcgacggcacgtggcggcagaccggcttcgggacgggcaggagacgcaaggcgcgcaacactagggggctggacgtggaccactggctaaggagcgctggaaagatgacggtcgggtttgacgaaacggtgactatggacacggtccgcaaccacaacatgccggacgacgcctggtgcgcgatccacggcaccgtgtacgacatcaccaagttcagcaaggtgcaccccggcggggacatcatcatgctggccgctggcaaggaggccaccatcctgttcgagacctaccacatcaagggcgtcccggacgcggtgctgcgcaagtacaaggtcggcaagctcccccagggcaagaagggcgaaacgagccacgtgcccaccgggctcgactcggcctcctactactcgtgggacagcgagttttacagggtgctccgcgagcgcgtcgccaagaagctggccgagcccggcctcatgcagcgcgcgcgcatggagctctgggccaaggcgatcttcctcctggcaggtttctggggctccctttacgccatgtgcgtgctagacccgcacggcggtgccatggtagccgccgttacgctcggcgtgttcgctgcctttgtcggaacttgcatccagcacgacggcagccacggcgccttctccaagtcgcgattcatgaacaaggcggcgggctggaccctcgacatgatcggcgcgagcgcgatgacctgggagatgcagcacgttcttggccaccacccgtacaccaacctcatcgagatggagaacggtttggccaaggtcaagggcgccgacgtcgacccgaagaaggtcgaccaggagagcgacccggacgtcttcagtacgtacccgatgcttcgcctgcacccgtggcaccgccagcggttttaccacaagttccagcacctgtacgccccgtttatctttgggtttatgacgattaacaaggtgatttcccaggatgtcggggttgtgctgcgcaagcgcctgttccagatcgacgccaactgccggtatggcagcccctggtacgtggcccgcttctggatcatgaagctcctcaccacgctctacatggtggcgcttcccatgtacatgcaggggcctgctcagggcttgaagcttttcttcatggcccacttcacctgcggagaggtcctcgccaccatgtttattgtcaaccacatcatcgagggcgtcagctacgcttccaaggacgcggtcaagggcgtcatggctccgccgcgcactgtgcacggtgtcaccccgatgcaggtgacgcaaaaggcgctcagtgcggccgagtcgaccaagtcggacgccgacaagacgaccatgatccccctcaacgactgggccgctgtgcagtgccagacctctgtgaactgggctgtcgggtcgtggttttggaaccacttttcgggcggcctcaaccaccagattgagcaccactgcttccccaaaacccccacacggtcaacgtctacatctcgggcatcgtcaaggagacctgcgaagaatacggcgtgccgtaccaggctgagatcagcctcttctctgcctatttcaagatgctgtcgc

SEQ ID NO: 184 - DNA, 616 bp, Thraustochytrium aureum
  source 1..616: mol_type = genomic DNA
cgcaaggcgcgcaacactagggggctggacgtggaccactggctaaggagcgctggaaagatgacggtcgggtttgacgaaacggtgactatggacacggtccgcaaccacaacatgccggacgacgcctggtgcgcgatccacggcaccgtgtacgacatcaccaagttcagcaaggtgcaccccggcggggacatcatcatgctggccgctggcaaggaggccaccatcctgttcgagacctaccacatcaagggcgtcccggacgcggtgctgcgcaagtacaaggtcggcaagctcccccagggcaagaagggcgaaacgagccacgtgcccaccgggctcgactcggcctcctactactcgtgggacagcgagttttacagggtgctccgcgagcgcgtcgccaagaagctggccgagcccggcctcatgcagcgcgcgcgcatggagctctgggccaaggcgatcttcctcctggcaggtttctggggctccctttacgccatgtgcgtgctagacccgcacggcggtgccatggtagccgccgttacgctcggcgtgttcgctgcctttgtcggaacttgcatccagcacgacggcagccacggcgcct

SEQ ID NO: 185 - DNA, 25 bp, synthetic construct
  misc_feature 1..25: note = primer
  source 1..25: mol_type = other DNA
caggagatctccaagtcgcgattca

SEQ ID NO: 186 - DNA, 26 bp, synthetic construct
  misc_feature 1..26: note = primer
  source 1..26: mol_type = other DNA
cttggagatctcctgcccgtcccgaa

SEQ ID NO: 187 - DNA, 3264 bp, synthetic construct
  misc_feature 1..3264: note = fusion DNA (T. aureum delta 4 desaturase upstream/SV40 terminator/BlaR/ubiquitin promoter/T. aureum delta 4 desaturase)
  source 1..3264: mol_type = other DNA
ggcggagcgaagtgtgaaagttacaacccagttactgcccattcccgggaaaagttgcgcagctcacgcggttcgcttttctggtggcctggcgacgttcgccgcttgccggatactccctcgtgcccccgcgccaggtttgcccgctgtcgctcgaggagtggactcgcgagtcgcgacagcagcagcaccaagggggatggatcctcgttgacagcaccaagatgctctctgcctttcaggtgaaatcgatcgatcaattgatcaatcaagatcattggaagcaaatgggaagcaaatgcgaagggggaagaccctcggtctctgctcgggaacccgacacgaggctgagggcgcgcttctacaggttgtgcagcggccgcactgcgagcttgcgccgggccaaggcgctcgccagaattgctgcgtctgccgcctcgggatcagccactcggtttttcgtcatcagggtccaccttcaacctggaagtggactcggcaagtcggcagatccactccggaattccaagatccccggtcgatcggtgctggtgcgaattaggatggacccaggctatgtgagagtcggagggtggcggttgtctccaccgtgacagcgcgcgtgtggtgagtaacgcgaagcgcgtggtggagaaatggggggagattcgtaggacgcgatgcgctcgtcactgagggtgcgccggtgacgaagcttcggacccagattccgtcggtatggctcgtgttcgcacaccttcaggaacccgcatgacgagaccactggagttttcaacgtcacgaagccgctctgtgtgacgagaattggcttgcgagtgacgtgaggcgccgagcatgtcgttggtttgcctcttcacaacagaatcagacgactgggaggctgcacgaggctaaggccaagggcactcactgactcggacgtgaagcagaagcagaagcagagcgctcgacggcacgtggcggcagaccggcttcgggacgggcaggagatctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgcggatcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgttggctagtgttgcttaggtcgcttgctgctgctggtgttgcttgttctgcttgtcgtttggggactgcccgaagtggacgcgtgacacagcccagcgttcgcgacttttccagcaaccagctcgctccgcgtcagggtcactctctgctcactcccctgtctagttcggcagaactggagaggcaacccgcgcttccagagggttctcctccggaatcagttcagaattcagaattcagaagagggaatcgcagaaccggccgcttcgggagttggttcgctgcaccatagcaggtgggcgatgaggccaaccgccctgctggctggattctctcgggttggcacatcgaacgaccgggacccgcgcgcgagttgtccctgccagttgctaccagcctgccagcgtcggagagttatgcgctgcctgccggccggagagagagcgggcgtggaaagtggcgtgtggggcgaacgcatggccctcccgcgtggcccgatttcctatgcatccgctccgctctctctctcggaggcaagaagagcacaccaacaacgcccggcgggtgcaccaggcgctgcggcagatccagatctccaagtcgcgattcatgaacaaggcggcgggctggaccctcgacatgatcggcgcgagcgcgatgacctgggagatgcagcacgttcttggccaccacccgtacaccaacctcatcgagatggagaacggtttggccaaggtcaagggcgccgacgtcgacccgaagaaggtcgaccaggagagcgacccggacgtcttcagtacgtacccgatgcttcgcctgcacccgtggcaccgccagcggttttaccacaagttccagcacctgtacgccccgtttatctttgggtttatgacgattaacaaggtgatttcccaggatgtcggggttgtgctgcgcaagcgcctgttccagatcgacgccaactgccggtatggcagcccctggtacgtggcccgcttctggatcatgaagctcctcaccacgctctacatggtggcgcttcccatgtacatgcaggggcctgctcagggcttgaagcttttcttcatggcccacttcacctgcggagaggtcctcgccaccatgtttattgtcaaccacatcatcgagggcgtcagctacgcttccaaggacgcggtcaagggcgtcatggctccgccgcgcactgtgcacggtgtcaccccgatgcaggtgacgcaaaaggcgctcagtgcggccgagtcgaccaagtcggacgccgacaagacgaccatgatccccctcaacgactgggccgctgtgcagtgccagacctctgtgaactgggctgtcgggtcgtggttttggaaccacttttcgggcggcctcaaccaccagattgagcaccactgcttccccaaaacccccacacggtcaacgtctacatctcgggcatcgtcaaggagacctgcgaagaatacggcgtgccgtaccaggctgagatcagcctcttctctgcctatttcaagatgctgtcgc

SEQ ID NO: 188 - DNA, 3935 bp, synthetic construct
  misc_feature 1..3935: note = fusion DNA (T. aureum delta 4 desaturase upstream/SV40 terminator /ZeoR/Enhanced GFP/ubiquitin promoter/T. aureum delta 4 desaturase)
  source 1..3935: mol_type = other DNA
ggcggagcgaagtgtgaaagttacaacccagttactgcccattcccgggaaaagttgcgcagctcacgcggttcgcttttctggtggcctggcgacgttcgccgcttgccggatactccctcgtgcccccgcgccaggtttgcccgctgtcgctcgaggagtggactcgcgagtcgcgacagcagcagcaccaagggggatggatcctcgttgacagcaccaagatgctctctgcctttcaggtgaaatcgatcgatcaattgatcaatcaagatcattggaagcaaatgggaagcaaatgcgaagggggaagaccctcggtctctgctcgggaacccgacacgaggctgagggcgcgcttctacaggttgtgcagcggccgcactgcgagcttgcgccgggccaaggcgctcgccagaattgctgcgtctgccgcctcgggatcagccactcggtttttcgtcatcagggtccaccttcaacctggaagtggactcggcaagtcggcagatccactccggaattccaagatccccggtcgatcggtgctggtgcgaattaggatggacccaggctatgtgagagtcggagggtggcggttgtctccaccgtgacagcgcgcgtgtggtgagtaacgcgaagcgcgtggtggagaaatggggggagattcgtaggacgcgatgcgctcgtcactgagggtgcgccggtgacgaagcttcggacccagattccgtcggtatggctcgtgttcgcacaccttcaggaacccgcatgacgagaccactggagttttcaacgtcacgaagccgctctgtgtgacgagaattggcttgcgagtgacgtgaggcgccgagcatgtcgttggtttgcctcttcacaacagaatcagacgactgggaggctgcacgaggctaaggccaagggcactcactgactcggacgtgaagcagaagcagaagcagagcgctcgacggcacgtggcggcagaccggcttcgggacgggcaggagatctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgcggatctcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgttggctagtgttgcttagatcgcttgctgctgctggtgttgcttgttctgcttgtcgtttggggtctgcccgaagtggacgcgtgacacagcccagcgttcgcgacttttccagcaaccagctcgctccgcgtcagggtcactctctgctcactcccctgtctagttcggcagaactggagaggcaacccgcgcttccagagggttctcctccggaatcagttcagaattcagaattcagaagagggaatcgcagaaccggcagcttcgggagttggttcgctgcaccatagcaggtgggcgatgaggccaaccgccctgctggctggattctctcgggttggcacatcgaacgaccgggacccgcgcgcgagttgtccctgccagttgctaccagcctgccagcgtcggagagttatgcgctgcctgccggccggagagagagcgggcgtggaaagtggcgtgtggggcaaacgcatggccctcccgcgtggcccgatttcctatgcatccgctccgctctctctctcggaggcaagaagagcacacaacaacgcccggcgggtgcaccaggcgctgcggcagatctccaagtcgcgattcatgaacaaggcggcgggctggaccctcgacatgatcggcgcgagcgcgatgacctgggagatgcagcacgttcttggccaccacccgtacaccaacctcatcgagatggagaacggtttggccaaggtcaagggcgccgacgtcgacccgaagaaggtcgaccaggagagcgacccggacgtcttcagtacgtacccgatgcttcgcctgcacccgtggcaccgccagcggttttaccacaagttccagcacctgtacgccccgtttatctttgggtttatgacgattaacaaggtgatttcccaggatgtcggggttgtgctgcgcaagcgcctgttccagatcgacgccaactgccggtatggcagcccctggtacgtggcccgcttctggatcatgaagctcctcaccacgctctacatggtggcgcttcccatgtacatgcaggggcctgctcagggcttgaagcttttcttcatggcccacttcacctgcggagaggtcctcgccaccatgtttattgtcaaccacatcatcgagggcgtcagctacgcttccaaggacgcggtcaagggcgtcatggctccgccgcgcactgtgcacggtgtcaccccgatgcaggtgacgcaaaaggcgctcagtgcggccgagtcgaccaagtcggacgccgacaagacgaccatgatccccctcaacgactgggccgctgtgcagtgccagacctctgtgaactgggctgtcgggtcgtggttttggaaccacttttcgggcggcctcaaccaccagattgagcaccactgcttccccaaaacccccacacggtcaacgtctacatctcgggcatcgtcaaggagacctgcgaagaatacggcgtgccgtaccaggctgagatcagcctcttctctgcctatttcaagatgctgtcgc

SEQ ID NO: 189 - DNA, 23 bp, synthetic construct
  misc_feature 1..23: note = primer
  source 1..23: mol_type = other DNA
aaaagaacaagccctctcctgga

SEQ ID NO: 190 - DNA, 23 bp, synthetic construct
  misc_feature 1..23: note = primer
  source 1..23: mol_type = other DNA
gaggtttgtatgttcggcggttt

SEQ ID NO: 191 - DNA, 26 bp, synthetic construct
  misc_feature 1..26: note = primer
  source 1..26: mol_type = other DNA
tgggggaccttgtgcagaactcgtgg

SEQ ID NO: 192 - DNA, 22 bp, synthetic construct
  misc_feature 1..22: note = primer
  source 1..22: mol_type = other DNA
gacctacggcgtgcagtgcttc